(19) United States
(12) Patent Application Publication
(10) Pub. No.: US 2024/0360229 A1
(43) Pub. Date: Oct. 31, 2024

(54) MULTISPECIFIC ANTIBODIES AND USES THEREOF

(71) Applicant: Genentech, Inc., South San Francisco, CA (US)

(72) Inventors: Christoph SPIESS, Mountain View, CA (US); Baomei WANG, Millbrae, CA (US); Ambrose Jon WILLIAMS, Pleasant Hill, CA (US); Cecilia Pui Chi CHIU, Redwood City, CA (US); Michael Andrew DILLON, San Francisco, CA (US); Teemu Tapani JUNTTILA, San Mateo, CA (US); James Thomas KOERBER, San Mateo, CA (US)

(21) Appl. No.: 18/416,525

(22) Filed: Jan. 18, 2024

Related U.S. Application Data
(60) Provisional application No. 63/480,469, filed on Jan. 18, 2023.

(51) Int. Cl. C07K 16/28; A61K 39/395; A61K 45/06

(52) U.S. Cl. CPC C07K 16/2866; A61K 39/3955; A61K 45/06; C07K 16/2809; C07K 2317/31; C07K 2317/35; C07K 2317/565; C07K 2317/567; C07K 2317/73

(57) ABSTRACT

The present invention provides anti-C-C motif chemokine receptor 8 (CCR8) antigen-binding molecules (e.g., bispecific antigen-binding molecules) and compositions thereof. The invention also features polynucleotides, vectors, host cells, methods of production, pharmaceutical compositions, methods of treating a disease or disorder, such as cancer, methods of depleting regulatory T cells, related uses and compositions for use, and kits for use with the one or more methods.

### SEQUENCE LISTING

The instant application contains a Sequence Listing which has been submitted electronically in XML format and is hereby incorporated by reference in its entirety. Said XML copy, created on Jan. 17, 2024, is named 50474-313002_Sequence_Listing_01_17_24 and is 124,506 bytes in size.

### TECHNICAL FIELD

The present invention relates to antigen-binding molecules that bind to C-C motif chemokine receptor 8 (CCR8), including multispecific antibodies, compositions thereof, and methods for treating diseases such as cancer.

### BACKGROUND

Cell proliferative disorders, such as cancer, are characterized by the uncontrolled growth of cell subpopulations. They are the leading cause of death in the developed world and the second leading cause of death in developing countries, with over 17 million new cancer cases diagnosed and 9.5 million cancer deaths estimated as occurring each year as of 2018. As the elderly population has grown, the incidence of cancer has concurrently risen, as the probability of developing cancer is more than two-fold higher after the age of seventy. Cancer care thus represents a significant and ever-increasing societal burden.

Regulatory T (Treg) cells expressing the transcription factor Foxp3 are important for maintaining peripheral immune tolerance and preventing autoimmunity. Treg cells also constitute a major component of the immune infiltrate of solid cancers, promoting tumor development and progression by establishing an immunosuppressive tumor microenvironment and dampening anti-tumor immune responses. Treg cells also hamper the efficacy of immunotherapies. An increased proportion of Treg cells among tumor-infiltrating lymphocytes is associated with poorer outcomes in several cancer indications.

Several strategies directed to Treg cell depletion or inhibition have been shown to enhance anti-tumor immunity and result in tumor growth inhibition in pre-clinical breast, melanoma, and colon cancer models. However, strategies targeting surface receptors expressed on both Treg cells and effector T cells, have shown limited efficacy in established tumors likely due to the concomitant depletion of effector T cells critical for anti-tumor immunity.

Thus, there is an unmet need in the field for the development of effective therapeutic agents for depletion of Treg cells for use in cancer treatment.

### SUMMARY

The present invention provides, inter alia, antigen-binding molecules, including multispecific antigen-binding molecules, e.g., bispecific antigen-binding molecules (e.g., bispecific antibodies; including 2+1 T cell-dependent bispecific antibodies (TDBs); e.g., anti-CCR8/anti-CD3 bispecific antigen-binding molecules; e.g., anti-CCR8/anti-CD3 bispecific antibodies) that bind C-C motif chemokine receptor 8 (CCR8) and an activating T cell antigen (e.g., cluster of differentiation 3 (CD3)), compositions comprising the bispecific antigen-binding molecules (e.g., pharmaceutical compositions), polynucleotides encoding the antigen-binding molecules, vectors, host cells, methods of production, and methods and uses thereof.

In one aspect, the invention provides a bispecific antigen-binding molecule comprising: (a) a first antigen-binding domain that binds C-C motif chemokine receptor 8 (CCR8), wherein the first antigen-binding domain comprises the following six complementarity-determining regions (CDRs): (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds cluster of differentiation 3 (CD3), wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22).

In some embodiments, the first antigen-binding domain comprises a light chain variable region (VL) domain and a heavy chain variable region (VH) domain, and wherein: (a) the VL domain comprises a proline residue at position 12 (numbering according to Kabat); and/or (b) the VL domain comprises a lysine residue at position 38 and the VH domain comprises a glutamic acid residue at position 39 (numbering according to Kabat). In some embodiments, the second antigen-binding domain comprises a VL domain and a VH domain, and wherein the VL domain comprises a glutamic acid residue at position 38 and the VH domain comprises a lysine residue at position 39 (numbering according to Kabat). In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 10; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14; (vii) an FR-L3 comprising the amino acid sequence of SEQ ID NO: 15; and/or (viii) an FR-L4 comprising the amino acid sequence of SEQ ID NO: 16; and/or (b) the second antigen-binding domain comprises one or more of the following eight FRs: (i) an FR-H1 comprising the amino acid sequence of SEQ ID NO: 25; (ii) an FR-H2 comprising the amino acid sequence of SEQ ID NO: 26; (iii) an FR-H3 comprising the amino acid sequence of SEQ ID NO: 27; (iv) an FR-H4 comprising the amino acid sequence of SEQ ID NO: 28; (v) an FR-L1 comprising the amino acid sequence of SEQ ID NO: 29; (vi) an FR-L2 comprising the amino acid sequence of SEQ ID NO: 30; (vii) an FR-L3 comprising the amino acid sequence of SEQ ID NO: 31; and/or (viii) an FR-L4 comprising the amino acid sequence of SEQ ID NO: 32.

In some embodiments, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 7; (b) a VL domain comprising the amino acid sequence of SEQ ID NO: 8; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 7 and a VL domain comprising the amino acid sequence of SEQ ID NO: 8; and the second antigen-binding domain comprises: (b) a VH domain comprising the amino acid sequence of SEQ ID NO: 23 and a VL domain comprising the amino acid sequence of SEQ ID NO: 24.

In some embodiments, the first antigen-binding domain is a Fab molecule comprising a Fab light chain and a Fab heavy chain and/or the second antigen-binding domain is a Fab molecule comprising a Fab light chain and a Fab heavy chain. In some embodiments, the first antigen-binding domain is a Fab molecule comprising a Fab light chain and a Fab heavy chain and the second antigen-binding domain is a Fab molecule comprising a Fab light chain and a Fab heavy chain, and wherein: (a) the Fab light chain of the first antigen-binding domain comprises a glutamic acid residue at position 133, and the Fab heavy chain of the first antigen-binding domain comprises a lysine residue at position 183 (numbering according to Kabat); and/or (b) the Fab light chain of the second antigen-binding domain comprises a lysine residue at position 133, and the Fab heavy chain of the second antigen-binding domain comprises a glutamic acid residue at position 183 (numbering according to Kabat).

In some embodiments, the bispecific antigen-binding molecule further comprises an Fc domain comprising a first subunit and a second subunit. In some embodiments, the Fc domain is an IgG Fc domain. In some embodiments, the Fc domain is an IgG₁ Fc domain. In some embodiments, the Fc domain is a human IgG Fc domain. In some embodiments, the Fc domain comprises a modification promoting the association of the first subunit and the second subunit of the Fc domain.

In some embodiments, the bispecific antigen-binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain. In some embodiments, the first subunit comprises one or more heavy chain constant domains selected from a first CH2 (CH2₁) domain and/or a first CH3 (CH3₁) domain; and the second subunit comprises one or more heavy chain constant domains selected from a second CH2 (CH2₂) domain and/or a second CH3 (CH3₂) domain. In some embodiments, at least one of the one or more heavy chain constant domains is paired with another heavy chain constant domain. In some embodiments, the CH3₁ and CH3₂ domains each comprise a protuberance or cavity, and wherein the protuberance or cavity in the CH3₁ domain is positionable in the cavity or protuberance, respectively, in the CH3₂ domain. In some embodiments, the CH3₁ and CH3₂ domains meet at an interface between said protuberance and cavity. In some embodiments, the CH2₁ and CH2₂ domains each comprise a protuberance or cavity, and wherein the protuberance or cavity in the CH2₁ domain is positionable in the cavity or protuberance, respectively, in the CH2₂ domain. In some embodiments, the CH2₁ and CH2₂ domains meet at an interface between said protuberance and cavity. In some embodiments, the first antigen-binding domain and the second antigen-binding domain are each a Fab molecule and the bispecific antigen-binding molecule comprises an Fc domain comprising a first subunit and a second subunit; and wherein the first antigen-binding domain is fused at the C-terminus of the Fab heavy chain to the N-terminus of the first subunit and the second antigen-binding domain is fused at the C-terminus of the Fab heavy chain to the N-terminus of the second subunit.

In some embodiments, the first subunit comprises a tryptophan residue at position 366; and the second subunit comprises a serine residue at position 366, an alanine residue at position 368, and a valine residue at position 407 (numbered according to Kabat EU index).

In some embodiments, each of the first subunit and the second subunit comprises an alanine residue at position 234, an alanine residue at position 235, and a glycine residue at position 329 (numbering according to Kabat EU index).

In some embodiments, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the sequence GASNLAS (SEQ ID NO: 5); and (vi) a CDR-L3 comprising the amino acid sequence QQAYYGNSFVEGT (SEQ ID NO: 6).

In some embodiments, the third antigen-binding domain comprises a VL domain and a VH domain, and wherein: (a) the VL domain comprises a proline residue at position 12 (numbering according to Kabat); and/or (b) the VL domain comprises a lysine residue at position 38 and the VH domain comprises a glutamic acid residue at position 39 (numbering according to Kabat). In some embodiments, the third antigen-binding domain comprises one or more of the following eight FRs: (i) an FR-H1 comprising the amino acid sequence of SEQ ID NO: 9; (ii) an FR-H2 comprising the amino acid sequence of SEQ ID NO: 10; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14; (vii) an FR-L3 comprising the amino acid sequence of SEQ ID NO: 15; and/or (viii) an FR-L4 comprising the amino acid sequence of SEQ ID NO: 16.

In some embodiments, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b). In some embodiments, the third antigen-binding domain comprises (a) a VH domain comprising the amino acid sequence of SEQ ID NO: 7; (b) a VL domain comprising the amino acid sequence of SEQ ID NO: 8; or (c) a VH domain as in (a) and a VL domain as in (b). In some embodiments, the third antigen-binding domain comprises a VH domain comprising the amino acid sequence of SEQ ID NO: 7 and a VL domain comprising the amino acid sequence of SEQ ID NO: 8.

In some embodiments, the third antigen-binding domain is a Fab molecule. In some embodiments, the third antigen-binding domain is a Fab molecule comprising a Fab light chain and a Fab heavy chain, and wherein the Fab light chain of the third antigen-binding domain comprises a glutamic acid residue at position 133, and the Fab heavy chain of the third antigen-binding domain comprises a lysine residue at position 183 (numbering according to Kabat).

In some embodiments, the second antigen-binding domain and the third antigen-binding domain are fused to each other. In some embodiments, the second antigen-binding domain and the third antigen-binding domain are fused to each other via a peptide linker. In some embodiments, the peptide linker comprises the amino acid sequence of SEQ ID NO: 37. In some embodiments, the second antigen-binding domain and the third antigen-binding domain are each a Fab molecule, and wherein the third antigen-binding domain is fused at the C-terminus of the Fab heavy chain to the N-terminus of the Fab heavy chain of the second antigen-binding domain.

In some embodiments, the bispecific antigen-binding molecule comprises an Fc domain comprising of a first subunit and a second subunit; wherein the first antigen-binding domain, the second antigen-binding domain, and the third antigen-binding domain are each a Fab molecule; wherein the first antigen-binding domain is fused at the C-terminus of the Fab heavy chain to the N-terminus of the first subunit; wherein the second antigen-binding domain is fused at the C-terminus of the Fab heavy chain to the N-terminus of the second subunit; and wherein the third antigen-binding domain is fused at the C-terminus of the Fab heavy chain to the N-terminus of the Fab heavy chain of the second antigen-binding domain.

In some embodiments, the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 33, a polypeptide comprising an amino acid sequence having at least 80%, 85%, 90%, 95%, 96%, 97%, 98%, 99%, or 100% sequence identity to the amino acid sequence of SEQ ID NO: 34, a polypeptide comprising an amino acid sequence having at least 80%, 85%, 90%, 95%, 96%, 97%, 98%, 99%, or 100% sequence identity to the amino acid sequence of SEQ ID NO: 35, and a polypeptide comprising an amino acid sequence having at least 80%, 85%, 90%, 95%, 96%, 97%, 98%, 99%, or 100% sequence identity to the amino acid sequence of SEQ ID NO: 36. In some embodiments, the bispecific antigen-binding molecule comprises a polypeptide comprising the amino acid sequence of SEQ ID NO: 33, a first polypeptide and a second polypeptide each comprising the amino acid sequence of SEQ ID NO: 34, a polypeptide comprising the amino acid sequence of SEQ ID NO: 35, and a polypeptide comprising the amino acid sequence of SEQ ID NO: 36. In some embodiments, (i) the polypeptide comprising the amino acid sequence of SEQ ID NO: 33 is connected to the first polypeptide comprising the amino acid sequence of SEQ ID NO: 34 via a Fab heavy chain and Fab light chain interaction; (ii) the polypeptide comprising the amino acid sequence of SEQ ID NO: 35 is connected to the second polypeptide comprising the amino acid sequence of SEQ ID NO: 34 via a Fab heavy chain and Fab light chain interaction; (iii) the polypeptide comprising the amino acid sequence of SEQ ID NO: 35 is connected to the polypeptide comprising the amino acid sequence of SEQ ID NO: 36 via a Fab heavy chain and Fab light chain interaction; and (iv) the polypeptide comprising the amino acid sequence of SEQ ID NO: 33 is connected to the polypeptide comprising the amino acid sequence of SEQ ID NO: 35 via a first subunit and a second subunit of an Fc domain.

In one aspect, the invention provides an isolated polynucleotide or a set of isolated polynucleotides encoding any one of the bispecific antigen-binding molecules described herein.

In one aspect, the invention provides an isolated polynucleotide or a set of isolated polynucleotides comprising a nucleic acid sequence that is at least 85%, at least 90%, at least 95%, or at least 99% identical to the nucleic acid sequence of any one of SEQ ID NOs: 85-89.

In one aspect, the invention provides isolated polynucleotide or a set of isolated polynucleotides comprising the nucleic acid sequence of any one of SEQ ID NOs: 85-89.

In one aspect, the invention provides a set of isolated polynucleotides comprising an isolated polynucleotide comprising the nucleic acid sequence of SEQ ID NO 85, an isolated polynucleotide comprising the nucleic acid sequence of SEQ ID NO 86, an isolated polynucleotide comprising the nucleic acid sequence of SEQ ID NO 87, and an isolated polynucleotide comprising the nucleic acid sequence of SEQ ID NO 88.

In one aspect, the invention provides a vector or a set of vectors comprising any one of the isolated polynucleotides or any one of the sets of isolated polynucleotides described herein.

In one aspect, the invention provides a host cell or a set of host cells comprising (i) any one of the isolated polynucleotides or any one of the sets of isolated polynucleotides described herein or (ii) any one of the vectors or any one of the sets of vectors described herein.

In one aspect, the invention provides a method of producing a bispecific antigen-binding molecule that binds to CCR8 and CD3, comprising the steps of (a) culturing any one of the host cells or any one of the sets of host cells described herein under conditions suitable for the expression of the bispecific antigen-binding molecule. In some embodiments, the method further comprises recovering the bispecific antigen-binding molecule.

In one aspect, the invention provides a bispecific antigen-binding molecule that binds to CCR8 and CD3 produced by any one of the methods described herein.

In one aspect, the invention provides pharmaceutical composition comprising any one of the bispecific antigen-binding molecules described herein and a pharmaceutically acceptable carrier.

In one aspect, the invention provides any one of the bispecific antigen-binding molecules described herein or any one of the pharmaceutical compositions described herein for use as a medicament.

In one aspect, the invention provides use of any one of the bispecific antigen-binding molecules described herein or any one of the pharmaceutical compositions described herein in the manufacture of a medicament.

In one aspect, the invention provides any one of the bispecific antigen-binding molecules described herein or any one of the pharmaceutical compositions described herein for use in the treatment of a cancer.

In one aspect, the invention provides use of any one of the bispecific antigen-binding molecules described herein or any one of the pharmaceutical compositions described herein for the treatment of a cancer in a subject in need thereof.

In one aspect, the invention provides use of any one of the bispecific antigen-binding molecules described herein or any one of the pharmaceutical compositions described herein for treating a cancer in a subject in need thereof.

In one aspect, the invention provides a method of treating a cancer in a subject, comprising administering to the subject an effective amount of any one of the bispecific antigen-binding molecules described herein or any one of the pharmaceutical compositions described herein.

In some embodiments, the cancer is selected from the group consisting of bladder cancer, blastoma, blood cancer, bone cancer, brain cancer, breast cancer, cervical cancer, colorectal cancer, endometrial cancer, esophageal cancer, gastric cancer, head and neck cancer, kidney cancer, liver cancer, lung cancer, ovarian cancer, pancreatic cancer, prostate cancer, sarcoma, skin cancer, testicular cancer, and uterine cancer.

In one aspect, the invention provides use of any one of the bispecific antigen-binding molecules described herein or any one of the pharmaceutical compositions described herein for depleting regulatory T cells.

In one aspect, the invention provides a method of depleting regulatory T cells in a tumor microenvironment in a subject having cancer comprising administering to the subject an effective amount of any one of the bispecific antigen-binding molecules described herein or any one of the pharmaceutical compositions described herein sufficient to deplete the regulatory T cells in the tumor microenvironment.

In one aspect, the invention provides a method of depleting regulatory T cells outside of a tumor microenvironment in a subject having cancer comprising administering to the subject an effective amount of any one of the bispecific antigen-binding molecules described herein or any one of the pharmaceutical compositions described herein sufficient to deplete the regulatory T cells outside of the tumor microenvironment.

In some embodiments, the regulatory T cells present in the tumor microenvironment of the cancer are depleted. In some embodiments, the regulatory T cells outside of the tumor microenvironment of the cancer are depleted.

In one aspect, the invention provides an in vitro method of depleting regulatory T cells from a cancer cell population, comprising contacting the cell population with any one of the bispecific antigen-binding molecules described herein or any one of the pharmaceutical compositions described herein in an amount sufficient to deplete the regulatory T cells from the cell population.

In some embodiments, the subject has reduced CCR8 mRNA expression.

In one aspect, the invention provides a method of reducing CCR8 mRNA expression, comprising contacting the cell population with any one of the bispecific antigen-binding molecules described herein or any one of the pharmaceutical compositions described herein in an amount sufficient to reduce CCR8 mRNA expression. In some embodiments, the method reduces CCR8 mRNA expression in a subject having a cancer.

In some embodiments, any one of the bispecific antigen-binding molecules for use, pharmaceutical compositions for use, uses, or methods described herein, further comprises administering an additional therapeutic agent to the subject.

In some embodiments, the additional therapeutic agent is an anti-cancer agent. In some embodiments, the anti-cancer agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a toll-like receptor (TLR) agent, a bispecific antibody, a cellular therapy, and an immune cell engager. In some embodiments, the anti-cancer agent is a PD-L1 binding antagonist. In some embodiments, the PD-L1 binding antagonist is atezolizumab.

In some embodiments, the additional therapeutic agent is tocilizumab or a corticosteroid.

In one aspect, the invention provides of a bispecific antigen-binding molecule or a pharmaceutical composition comprising the bispecific antigen-binding molecule thereof for depleting regulatory T cells, wherein the bispecific antigen-binding molecule comprises: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds an activating T cell antigen.

In one aspect, the invention provides a method of depleting regulatory T cells in a tumor microenvironment in a subject having cancer comprising administering to the subject an effective amount of a bispecific antigen-binding molecule or a pharmaceutical composition comprising the bispecific antigen-binding molecule thereof sufficient to deplete the regulatory T cells in the tumor microenvironment, wherein the bispecific antigen-binding molecule comprises: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds an activating T cell antigen.

In one aspect, the invention provides a method of depleting regulatory T cells outside of a tumor microenvironment in a subject having cancer comprising administering to the subject an effective amount of a bispecific antigen-binding molecule or a pharmaceutical composition comprising the bispecific antigen-binding molecule thereof sufficient to deplete the regulatory T cells outside of the tumor microenvironment, wherein the bispecific antigen-binding molecule comprises: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds an activating T cell antigen.

In some embodiments, regulatory T cells present in the tumor microenvironment of the cancer are depleted. In some embodiments, the regulatory T cells outside of the tumor microenvironment of the cancer are depleted.

In one aspect, the invention provides an in vitro method of depleting regulatory T cells from a cancer cell population, comprising contacting the cell population with a bispecific antigen-binding molecule or a pharmaceutical composition comprising the bispecific antigen-binding molecule thereof in an amount sufficient to deplete the regulatory T cells from the cell population, wherein the bispecific antigen-binding molecule comprises: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds an activating T cell antigen.

In some embodiments, the activating T cell antigen is CD3.

In one aspect, the invention provides a method of reducing CCR8 mRNA expression in the blood of a subject comprising administering to the subject an effective amount of a bispecific antigen-binding molecule or a pharmaceutical composition comprising the bispecific antigen-binding molecule sufficient to reduce CCR8 mRNA expression, wherein the bispecific antigen-binding molecule comprises: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds an activating T cell antigen. In some embodiments, the method reduces CCR8 mRNA expression in a subject having a cancer.

### BRIEF DESCRIPTION OF THE DRAWINGS

FIG. 1A-FIG. 1E depict different structural formats of bispecific antigen-binding molecules that bind to CCR8 and CD3. FIG. 1A depicts a 1+1 A (CCR8)/B (CD3) bispecific antigen-binding molecule (e.g., TDB). FIG. 1B depicts a 2+0 AB bispecific antigen-binding molecule (e.g., TDB). FIG. 1C depicts a 2+1 A/AB bispecific antigen-binding molecule (e.g., TDB). FIG. 1D depicts a 2+1 A/BA bispecific antigen-binding molecule (e.g., TDB). FIG. 1E depicts a 2+1 B/AA bispecific antigen-binding molecule (e.g., TDB).

FIG. 2 is a graph comparing the hCCR8+ CHO cell killing potency of 1+1, 2+1, and 2+0 bispecific antigen-binding molecule (e.g., TDB) formats. All TDB formats compared used the 1889 CCR8 antigen-binding domain and the 38E4v1.MD1 (i.e., MD1) CD3 antigen-binding domain.

FIG. 3A-FIG. 3E are a series of graphs comparing the ability of various 1+1 and 2+1 bispecific antigen-binding molecule (e.g., TDB) formats to deplete CCR8+ Treg cells. All TDB formats compared used the 1889 CCR8 antigen-binding domain and the 38E4v1.MD1 (i.e., MD1) or the 40G5c CD3 antigen-binding domain. FIG. 3A-FIG. 3C depict absolute number of Treg cell depletion, while FIG. 3D and FIG. 3E depict the % Treg cell depletion of FIG. 3B and FIG. 3C, respectively.

FIG. 4A-FIG. 4E compare the two different orientations for 2+1 bispecific antigen-binding molecule (e.g., TDB) formats, A/AB and A/BA. FIG. 4A-FIG. 4D are a series of graphs comparing the ability of A/AB versus A/BA formats to deplete Treg cells in Donor 1 (FIG. 4A and FIG. 4C) and Donor 2 (FIG. 4B and FIG. 4D). FIG. 4A and FIG. 4B depict Treg cell depletion for CCR8+ Treg cells. FIG. 4C and FIG. 4D depict Treg cell depletion for non-CCR8+ Treg cells. FIG. 4E depicts the orientation of A/AB and A/BA format TDBs.

FIG. 5A-FIG. 5F are a series of graphs comparing the ability of A/AB 2+1 bispecific antigen-binding molecule (e.g., TDB) formats, either having the high affinity CD3 binder MD1 or the low affinity CD3 binder 40G5c, to deplete CCR8+ Treg cells. All TDB formats compared used the 1889 CCR8 antigen-binding domain and the 38E4v1.MD1 (i.e., MD1) or the 40G5c CD3 antigen-binding domain. FIG. 5A, FIG. 5C, and FIG. 5E depict the absolute number of Treg cell depletion, while FIG. 5B, FIG. 5D, and FIG. 5F depict the % Treg cell depletion of FIG. 5A, FIG. 5C, and FIG. 5E, respectively.

FIG. 6A-FIG. 6C are a series of graphs comparing the ability of A/AB 2+1 bispecific antigen-binding molecule (e.g., TDB) formats, either having the high affinity CD3 binder MD1 or the low affinity CD3 binder 40G5c, to reduce tumor size in mouse models of either breast cancer (E0771 breast model) or colorectal cancer (MD-38 colorectal model). Experiments were performed in hu.CD3.tg.B6N mice. Parenthetical values report dose level in mg/kg. All TDB formats compared used the 1356 mouse CCR8 antigen-binding domain and the 38E4v1.MD1 (i.e., MD1) or the 40G5c CD3 antigen-binding domain.

FIG. 7 contains a series of graphs depicting the in vivo cytokine release triggered by different CCR8 antigen-binding molecules. Experiments were performed in MD-38 colorectal model mice. 1+1 format TDBs were administered at a dose of 0.5 mg/kg, while 2+1 format TDBs were administered at 0.67 mg/kg. Within each panel, bars from left to right represent vehicle, anti-CCR8 (1294), anti-CCR8/CD3 (38E4), anti-CCR8/CD3 (40G5c), anti-CCR8-CD3 (38E4)/CCR8, anti-CCR8-CD3 (40G5c)/CCR8, and anti-CCR8-CD3 (2C11)/CCR8 treatment groups, respectively.

FIG. 8 is a graph comparing the off-rates of monospecific anti-CCR8 antibodies on human CCR8+ CHO cells, as determined by flow cytometry, of affinity-maturation variants of the anti-CCR8.1889 antibody. In particular, five variants showed improved off-rates as compared to WT 1889.

FIG. 9A-FIG. 9E are a series of graphs comparing the ability of various CCRB TDB formats having different CCR8 and CD3 binders to deplete CCR8+ Treg cells. Both 1+1 and 2+1 formats are tested. TDB formats compared used the 1889 WT or 1889 PVS variant CCR8 antigen-binding domain and the 38E4v1.MD1 (i.e., MD1) or the 40G5c CD3 antigen-binding domain.

FIG. 10 is a graph comparing the pharmacokinetics (PK) of various CCR8 TDB formats having either the 1889 WT or 1889 PVS variant CCR8 antigen-binding domain. Severe compromised immunodeficiency (SCID) mice were dosed with a single intravenous (IV) dose of 5 mg/kg of the tested CCR8 TDB. The serum concentration time profile is depicted.

FIG. 11A-FIG. 11C are a series of graphs showing the results of three experiments (Runs 1 to 3) evaluating the use of Protein L affinity chromatography to purify the generated S12P variant 2+1 1889WT/1889WT.40G5c 2+1 CCR8 TDB. For each experiment (Runs 1-3, corresponding to FIG. 11A, FIG. 11B, and FIG. 11C, respectively), the initial protein fraction (i.e., prior to Protein L affinity chromatography; “Protein L Load”), the flowthrough fraction (“Flowthrough”, and the bound pool fraction (“Pool”) were then evaluated using size-exclusion ultra-high performance liquid chromatography (SE-UPLC) elution chromatograms to determine the effect of Protein L purification. The two peaks evaluated via SE-UPLC were the main peak and the low molecular weight species fraction (LMWS).

FIG. 12A and FIG. 12B are diagrams depicting the structural components of the 2+1 1889WT/1889WT.40G5c 2+1 CCR8 TDB. FIG. 12A depicts the charge mutations on each domain of the monovalent and bivalent arms, as well as which arm comprises the knob or hole mutation. FIG. 12B further provides the molecular weights of each chain, as well as the amino acid mutations that comprise the charge mutations depicted in FIG. 12A, as well as the domains comprising the LALA-PG mutations and the knob (“K”) and hole (“H”) mutations.

FIG. 13A-FIG. 13C are a series of graphs showing the depletion of CCR8+ Treg cells incubated with human dissociated tumor cells in the presence of 2+1 1889WT/1889WT.40G5c 2+1 CCR8 TDB for 72 hours.

FIG. 14A and FIG. 14B depict the amino acid sequence alignment of the anti-CCR8 1889 antibody variants relative to the 1889 wild type (WT) antibody. FIG. 14A depicts the amino acid sequence alignment of the light chain variable region (VL domain) of the 1889 P, PV, PI, PS, PVI, PVS, PIS, and PVIS variants relative to the VL domain of the 1889 WT antibody. FIG. 14B depicts the amino acid sequence alignment of the heavy chain variable region (VH domain) of the 1889 A and DA variants against the 1889 WT antibody. Amino acid substitutions are highlighted. Underlining shows corresponding CDR regions as defined by Kabat.

FIG. 15A and FIG. 15B depict the amino acid sequence alignment of the anti-CD3 40G5c antibody and the 38E4v1.MD1 (MD1) antibody. FIG. 15A depicts the amino acid sequence alignment of the light chain variable region (VL domain) of the 40G5c antibody and the MD1 antibody. FIG. 15B depicts the amino acid sequence alignment of the heavy chain variable region (VH domain) of the 40G5c antibody and the MD1 antibody.

FIG. 16A and FIG. 16B are a series of graphs showing binding of CCR8 TDB to sulfated G protein-coupled receptors (GPCRs) CCR2, CCR3, CCR4, CCR5, CXCR4, ACKR2, ACKR4, and CCR8 assessed by flow cytometry. Light grey curves indicate binding assessed using Myc-negative (Myc-) cells, and dark grey curves indicate binding assessed using Myc-positive (Myc+) cells.

FIG. 17A is a diagram depicting the monitoring of CCR8 activation by Ca²⁺ influx using Fluorescent Imaging Plate Reader (FLIPR™). CCR8 is activated by treating a cell with CCL1, and the Ca²⁺ release triggered by CCR8 activation is visualized by a fluorescent Ca²⁺-chelating dye.

FIG. 17B is a line graph showing percentage blocking of CCL1-mediated activation of CCR8 by CCR8 TDB (1889/1889.40G5c) or a control antibody (BD 433H). 433H inhibited CCL1-mediated CCR8 activation with an IC₅₀ of 8.4 nM, whereas the CCR8 TDB had no effect on the activation of CCR8.

FIG. 18 is a line graph showing the percentage of CCR8+ Treg cells in the peripheral blood of cynomolgus monkeys treated with control or CCR8 TDB over time. A7, A8, and A9 received vehicle control, while A10, A11, and A12 received CCR8 TDB. Vehicle control or CCR8 TDB was administered intravenously on Day 1 and Day 11.

FIG. 19 is a dot plot depicting the average mRNA expression level of CCR8 in the blood of cynomolgus monkeys treated with CCR8 TDB or vehicle control at various time points. Horizontal lines indicate the mean of the three animals. * Denotes p<0.05 as determined by one-way ANOVA, followed by Sidak's multiple comparison test.

FIG. 20A-FIG. 20D are a series of line graphs showing the level of cytokines IL-1 RA (FIG. 20A), IL-8 (FIG. 20B), IL-6 (FIG. 20C), and MCP-1 (FIG. 20D) in cynomolgus monkeys treated with CCR8 TDB or vehicle control at various time points. The symbols represent individual animals (circles: control; squares: 10 mg/kg of CCR8 TDB), and the lines are on the mean values.

### DETAILED DESCRIPTION

The present invention is based, at least in part, on Applicant's discovery that anti-CCR8/anti-CD3 multispecific (e.g., bispecific) antigen-binding molecules (e.g., T cell-dependent bispecific antibodies (TDBs)) as described herein result in unexpectedly efficient Treg depletion and tumor cell killing with an acceptable safety profile. See, e.g., Examples 1-6 disclosed herein.

Without wishing to be bound by any particular theory, it is expected that the multispecific antigen-binding molecules disclosed herein deplete Treg cells via formation of immunological synapses between Tregs that express CCR8 and T effector (Teff) cells. Prior to Applicant's discovery, it was unexpected whether the bispecific antigen binding formats disclosed herein would deplete Treg cells, because Treg cells suppress Teff cells, and both CCR8 and CD3 are expressed on Treg cells. In particular, without wishing to be bound by any particular theory, the expression of both CCR8 and CD3 on Treg cells gave rise to potential challenges for the bispecific antigen binding molecules to avoid binding only to Tregs in cis via binding to both CCR8 and CD3 present on the Tregs, which would prevent the CD3 arm from binding and activating CD3+ Teff cells. Thus, the present invention provides for Treg depletion using multispecific antigen-binding molecules that bind to Treg cells (e.g., via binding to CCR8 or other Treg markers) and Teff cells (e.g., via binding to activating T cell antigens (e.g., CD3)).

The invention is also based, at least in part, on Applicant's discovery that bispecific 2+1 TDB molecules having the “A/AB” orientation disclosed herein were unexpectedly superior compared to other tested orientations of TDB cells, e.g., in terms of Treg depletion, and allow for using relatively lower affinity anti-CD3 arms (e.g., 40G5c) having acceptable levels of cytokine release, e.g., are more likely to possess an acceptable safety profile and more beneficial risk-benefit profile.

### I. DEFINITIONS

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3, particularly the epsilon subunit of CD3 (see UniProt no. P07766 (version 130), NCBI RefSeq no. NP_000724.1 for the human sequence; or UniProt no. Q95LI5 (version 49), NCBI GENBANK® no. BAB71849.1 for the cynomolgus (Macaca fascicularis) sequence).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating multispecific (e.g., bispecific) antigen-binding molecules disclosed herein are capable of inducing T cell activation. Suitable assays to measure T cell activation are known in the art and described herein.

By “administering” is meant a method of giving a dosage of a compound (e.g., an anti-CCR8/anti-CD3 multispecific antigen-binding molecule disclosed herein or a nucleic acid encoding an anti-CCR8/anti-CD3 multispecific antigen-binding molecule disclosed herein) or a composition (e.g., a pharmaceutical composition, e.g., a pharmaceutical composition including an anti-CCR8/anti-CD3 multispecific antigen-binding molecule disclosed herein) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are also described herein.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

For the purposes herein, “atezolizumab” is an Fc-engineered, humanized, non-glycosylated IgG₁ kappa immunoglobulin that binds PD-L1. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances (proposed INN)) List 112, Vol. 28, No. 4, 2014, p. 488.

The terms “anti-CCR8 antibody” and “an antibody that binds to CCR8” refer to an antibody that is capable of binding CCR8 with sufficient affinity such that the antibody is useful as a diagnostic and/or therapeutic agent in targeting CCR8. In one aspect, the extent of binding of an anti-CCR8 antibody to an unrelated, non-CCR8 protein is less than about 10% of the binding of the antibody to CCR8 as measured, e.g., by surface plasmon resonance (SPR). In certain aspects, an antibody that binds to CCR8 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻¹³ M to 10⁻⁸ M, e.g., from 10⁻¹³ M to 10⁻⁹ M). In certain aspects, an antibody that binds to CCR8 has a K_{D} of from about 1×10⁻¹² M to about 1×10⁻¹⁰ M, from about 1×10⁻¹² M to about 1×10⁻¹¹ M, or from about 1×10⁻¹¹ M to about 5×10⁻¹¹ M. In certain aspects, an antibody that binds to CCR8 has a K_{D} of about 2×10⁻¹¹ M. In certain aspects, an antibody that binds to CCR8 has a K_{D} of about 5×10⁻¹² M. An antibody is said to “specifically bind” to CCR8 when the antibody has a K_{D} of 1 μM or less. In certain embodiments, an anti-CCR8 antibody binds to an epitope of CCR8 in at least two different species (e.g., human and cynomolgus (cyno)).

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M). An antibody is said to “specifically bind” to CD3 when the antibody has a K_{D} of 1 μM or less. In certain embodiments, an anti-CD3 antibody binds to an epitope of CD3 that is conserved among CD3 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology (2005) 23:1126-1136.

By “antigen-binding domain” is meant a part of a compound or a molecule that specifically binds to a target epitope, antigen, ligand, or receptor. Molecules featuring antigen-binding moieties include, but are not limited to, antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

As used herein, the term “monovalent,” for example, in the context of a monovalent arm of a bispecific antigen-binding molecule, refers to a molecule or a portion thereof (e.g., a portion of an antigen-binding molecule, e.g., one of two arms of a bispecific antigen-binding molecule) that has a single antigen-binding domain. Thus, a monovalent molecule or portion thereof is capable of specific binding to exactly one antigen. The “monovalent binding affinity” or “monovalent K_{D}” of one of the two antigen-binding moieties of a bivalent arm of a bispecific antibody (e.g., one of the CCR8 or CD3 antigen-binding moieties of a 2+1 TDB) refers to the binding affinity of the antigen-binding domain in monovalent form, i.e., as a monovalent arm of a bispecific antibody capable of specific binding to two different antigens or as a Fab molecule.

As used herein, the term “bivalent,” for example, in the context of a bivalent arm of a bispecific antigen-binding molecule, refers to a molecule or a portion thereof (e.g., a portion of an antigen-binding molecule, e.g., one of two arms of a bispecific antigen-binding molecule) that has exactly two antigen-binding moieties, each of which is capable of specific binding to an antigen. Thus, a bivalent molecule or portion thereof is capable of specific binding to two antigens or two different epitopes on the same antigen.

The term “epitope” denotes the site on an antigen, either proteinaceous or non-proteinaceous, to which an anti-CCR8 antibody, an anti-CD3 antibody, or a binding domain of an anti-CCR8/anti-CD3 multispecific antigen-binding molecule (e.g., an anti-CCR8/anti-CD3 bispecific antigen-binding molecule) described herein binds. Epitopes can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an anti-CCR8 antibody, an anti-CD3 antibody, or a binding domain of an anti-CCR8/anti-CD3 multispecific antigen-binding molecule (e.g., a bispecific antigen-binding molecule) described herein after exposure of the proteinaceous antigen to denaturing agents, whereas conformational epitopes are typically destroyed upon treatment with denaturing agents. An epitope comprises at least 3, at least 4, at least 5, at least 6, at least 7, at least 8, at least 10, at least 15, at least 20, at least 30, or at least 35, or 3-25, 3-20, 3-15, 3-10, 3-5, 30-40, 35-40, or 5-10 amino acids in a unique spatial conformation.

Screening for antibodies binding to a particular epitope (i.e., those binding to the same epitope) can be done using methods routine in the art such as, e.g., without limitation, alanine scanning, peptide blots (see, e.g., Kobeissy et al., Meth. Mol. Biol. (2004) 248:443-463), peptide cleavage analysis, epitope excision, epitope extraction, chemical modification of antigens (see Hochleitner et al., Prot. Sci. 9 (2000) 487-496), and cross-blocking (see “Antibodies”, Harlow and Lane (Cold Spring Harbor Press, Cold Spring Harb., NY).

Competitive binding can be used to easily determine whether an antibody binds to the same epitope of CCR8 or CD3 as, or competes for binding with, a reference anti-CCR8 or anti-CD3 antibody. For example, an “antibody that binds to the same epitope” as a reference anti-CCR8 or anti-CD3 antibody refers to an antibody that blocks binding of the reference anti-CCR8 or anti-CD3 antibody to its antigen in a competition assay by 50% or more, and conversely, the reference antibody blocks binding of the antibody to its antigen in a competition assay by 50% or more. Also for example, to determine if an antibody binds to the same epitope as a reference anti-CCR8 or anti-CD3 antibody, the reference antibody is allowed to bind to CCR8 or CD3 under saturating conditions. After removal of the excess of the reference antibody, the ability of an anti-CCR8 or anti-CD3 antibody in question to bind to CCR8 or CD3, respectively is assessed. If the anti-CCR8 or anti-CD3 antibody is able to bind to CCR8 or CD3, respectively, after saturation binding of the reference anti-CCR8 or anti-CD3 antibody, it can be concluded that the antibody in question binds to a different epitope than the reference antibody. But, if the antibody in question is not able to bind to the target epitope after saturation binding of the reference antibody, then the anti-CCR8 or anti-CD3 antibody in question may bind to the same epitope as the epitope bound by the reference anti-CCR8 or anti-CD3 antibody. To confirm whether the antibody in question binds to the same epitope or is just hampered from binding by steric reasons routine experimentation can be used (e.g., peptide mutation and binding analyses using enzyme-linked immunosorbent assay (ELISA), radioimmunoassay (RIA), surface plasmon resonance, flow cytometry or any other quantitative or qualitative antibody-binding assay available in the art). This assay should be carried out in two set-ups, i.e., with both of the antibodies being the saturating antibody. If, in both set-ups, only the first (saturating) antibody is capable of binding to CCR8 (or CD3), then it can be concluded that the anti-CCR8 antibody (or anti-CD3 antibody) in question and the reference anti-CCR8 antibody (or reference anti-CD3 antibody) compete for binding to CCR8 (or CD3).

In some aspects, two antibodies are deemed to bind to the same or an overlapping epitope if a 1-, 5-, 10-, 20-, or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used herein denotes a constant heavy chain region of a human antibody of the subclass IgG₁, IgG₂, IgG₃, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody (e.g., a bispecific antigen-binding molecule, e.g., 2+1 TDB), which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of a compound, for example, a bispecific antigen-binding molecule disclosed herein or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein. It should be understood that the full-length antibody comprises a heavy chain variable domain and light chain variable domain, as defined herein, and an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

In certain aspects, antibodies comprise six CDRs: three in the VH (CDR-H1, CDR-H2, CDR-H3), and three in the VL (CDR-L1, CDR-L2, CDR-L3). In certain aspects, the antibodies comprising six CDRs are full-length antibodies. In certain aspects, the antibodies comprising six CDRs are antibody fragments. Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to McCallum, supra, Chothia, supra, or any other scientifically accepted nomenclature system.

In one exemplary aspect, CDR residues of bispecific antigen-binding molecules disclosed herein comprise those identified in Table 2.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody as described herein in vitro and/or in vivo, e.g., in a host or subject. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding a bispecific antigen-binding molecule,” e.g., “isolated nucleic acid encoding an anti-CCR8 antibody” or “isolated nucleic acid encoding an anti-CCR8 bispecific antigen-binding molecule” refers to one or more nucleic acid molecules encoding a bispecific antigen-binding molecule (e.g., an anti-CCR8 bispecific antigen-binding molecule, e.g., an anti-CCR8/anti-CD3 TDB″) heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, MEGALIGN™ (DNASTAR) software or the FASTA program package. Those skilled in the art can determine appropriate parameters for aligning sequences, including any algorithms needed to achieve maximal alignment over the full-length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from fasta.bioch.virginia.edu/fasta_www2/fasta_down.shtml or ebi.ac.uk/Tools/sss/fasta. Alternatively, a public server accessible at fasta.bioch.virginia.edu/fasta www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The terms “pharmaceutical composition” and “pharmaceutical formulation” are used interchangeably herein, and refer to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “CCR8,” as used herein, refers to any native CCR8 from any vertebrate source, including mammals such as primates (e.g., humans, monkeys (cyno)), and rodents (e.g., mice and rats), unless otherwise indicated. The term encompasses “full-length”, unprocessed CCR8 as well as any form of CCR8 that results from processing in the cell. The term also encompasses naturally occurring variants of CCR8, e.g., splice variants or allelic variants. In certain aspects, the CCR8 is a human CCR8 (“hCCR8” or “huCCR8”). The amino acid sequence of an exemplary human CCR8 is set forth in SEQ ID NO: 79, as shown in the below Table 1. In certain aspects, the CCR8 is a cynomolgus monkey (“cyno”) CCR8. The amino acid sequence of an exemplary cyno CCR8 is set forth in SEQ ID NO: 80, as shown in the below Table 1. In certain aspects, the CCR8 is a mouse CCR8 (“mCCR8”). The amino acid sequence of an exemplary mouse CCR8 is set forth in SEQ ID NO: 87, as shown in the below Table 1.

TABLE 1
Exemplary CCR8 sequences

| Description | Sequence |
|---|---|
| human CCR8 | MDYTLDLSVTTVTDYYYPDIFSSPCDAELIQTNGKLLLAVFYCLLFVFSLLGNS |
| SEQ ID NO: 79 | LVILVLVVCKKLRSITDVYLLNLALSDLLFVFSFPFQTYYLLDQWVFGTVMCKV |
| | VSGFYYIGFYSSMFFITLMSVDRYLAVVHAVYALKVRTIRMGTTLCLAVWLTAI |
| | MATIPLLVFYQVASEDGVLQCYSFYNQQTLKWKIFTNFKMNILGLLIPFTIFMF |
| | CYIKILHQLKRCQNHNKTKAIRLVLIVVIASLLFWVPFNVVLFLTSLHSMHILDG |
| | CSISQQLTYATHVTEIISFTHCCVNPVIYAFVGEKFKKHLSEIFQKSCSQIFNYL |
| | GRQMPRESCEKSSSCQQHSSRSSSVDYIL |
| cyno CCR8 | MDYTLDPSMTTMTDYYYPDSLSSPCDGELIQRNDKLLLAVFYCLLFVFSLLGN |
| SEQ ID NO: 80 | SLVILVLVVCKKLRNITDIYLLNLALSDLLFVFSFPFQTYYQLDQWVFGTVMCK |
| | VVSGFYYIGFYSSMFFITLMSVDRYLAVVHAVYAIKVRTIRMGTTLSLVVWLTA |
| | IMATIPLLVFYQVASEDGVLQCYSFYNQQTLKWKIFTNFEMNILGLLIPFTIFMF |
| | CYIKILHQLKRCQNHNKTKAIRLVLIVVIASLLFWVPFNVVLFLTSLHSMHILDG |
| | CSISQQLNYATHVTEIISFTHCCVNPVIYAFVGEKFKKHLSEIFQKSCSHIFIYL |
| | GRQMPRESCEKSSSCQQHSFRSSSIDYIL |
| mouse CCR8 | MDYTMEPNVTMTDYYPDFFTAPCDAEFLLRGSMLYLAILYCVLFVLGLLGNSL |
| SEQ ID NO: 81 | VILVLVGCKKLRSITDIYLLNLAASDLLFVLSIPFQTHNLLDQWVFGTAMCKVV |
| | SGLYYIGFFSSMFFITLMSVDRYLAIVHAVYAIKVRTASVGTALSLTVWLAAVT |
| | ATIPLMVFYQVASEDGMLQCFQFYEEQSLRWKLFTHFEINALGLLLPFAILLFC |
| | YVRILQQLRGCLNHNRTRAIKLVLTVVIVSLLFWVPFNVALFLTSLHDLHILDG |
| | CATRQRLALAIHVTEVISFTHCCVNPVIYAFIGEKFKKHLMDVFQKSCSHIFLYL |
| | GRQMPVGALERQLSSNQRSSHSSTLDDIL |

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term 5 encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3& protein (NCBI RefSeq No. NP_000724), which is 207 amino acids in length, and human CD3γ protein (NCBI RefSeq No. NP_000064), which is 182 amino acids in length.

As used herein, “treatment” (and grammatical variations thereof such as “treat” or “treating”) refers to clinical intervention in an attempt to alter the natural course of a disease (e.g., cancer) in the subject being treated, and can be performed either for prophylaxis (“preventative treatment” or “prophylactically treating”) or during the course of clinical pathology (“therapeutic treatment” or “therapeutically treating”). Desirable effects of therapeutic treatment include, but are not limited to, alleviation of symptoms, diminishment of any direct or indirect pathological consequences of the disease, preventing metastasis of the cancer, decreasing the rate of disease progression, amelioration or palliation of the disease state, and remission or improved prognosis. Desirable effects of preventative treatment include, but are not limited to, preventing occurrence or recurrence of disease. In some aspects, antibodies as described herein are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. COMPOSITIONS AND METHODS

In one aspect, the present disclosure is based, in part, on multispecific (e.g., bispecific) antigen-binding molecules (e.g., bispecific antibodies; e.g., bispecific antibodies that bind to CCR8 and CD3). In some embodiments, the bispecific antigen-binding molecules have a monovalent arm capable of specific binding to a first antigen (e.g., CCR8) and a bivalent arm capable of specific binding to two additional antigens (e.g., one antigen-binding domain that specifically binds CD3 and one antigen-binding domain that specifically binds CCR8). For example, the bivalent arm may comprise two antigen-binding moieties, each capable of specific binding to a target antigen (e.g., CCR8 or CD3). Also provided herein are anti-CCR8 antibodies. Multispecific (e.g., bispecific) antigen-binding molecules and antibodies as described herein are useful, e.g., for the treatment of cancer.

### A. Bispecific Antigen-Binding Molecules

In one aspect, the present disclosure provides bispecific antigen-binding molecules (e.g., bispecific antibodies; e.g., 2+1 TDBs; e.g., anti-CCR8/anti-CD3 bispecific antigen-binding molecules; e.g., anti-CCR8/anti-CD3 bispecific antibodies) bind to CCR8 and CD3. In one aspect, the bispecific antigen-binding molecules provided are isolated bispecific antigen-binding molecules that bind to CD3 and CCR8. In one aspect, the present disclosure provides bispecific antigen-binding molecules that include one or more antigen-binding moieties that specifically bind to a Treg cell antigen (e.g., CCR8) and one or more antigen-binding moieties that specifically bind to an activating T cell antigen (e.g., CD3). In one aspect, the present disclosure provides bispecific antigen-binding molecules that include one or more antigen-binding moieties that specifically bind to CCR8 and one or more antigen-binding moieties that specifically bind to CD3. In one aspect, the present disclosure provides bispecific antigen-binding molecules that include two antigen-binding moieties that specifically bind to CCR8 and one antigen-binding domain that specifically bind to CD3. In certain aspects, the CCR8 is a human CCR8. In certain aspects, the CD3 is a human CD3 or a cynomolgus monkey (cyno) CD3.

In one aspect, the invention provides isolated bispecific antigen-binding molecules that bind to CCR8 and CD3. In some embodiments, an antigen-binding domain of the bispecific antigen-binding molecule of the present invention comprises at least one, at least two, at least three, at least four, at least five, or all six CDRs (e.g., comprises one, two, three, four, five, or six CDRs) as illustrated in Table 2 (Kabat). In some instances, the antigen-binding molecule comprises a VH and/or a VL as illustrated in Table 2.

TABLE 2
Listing of SEQ ID NOs.

| | CDR (Kabat) | | | | | | V | |
|---|---|---|---|---|---|---|---|---|
| Antigen-binding domain: | H1 | H2 | H3 | L1 | L2 | L3 | VH | VL |
| CCR8 1889 WT | 1 | 2 | 3 | 38 | 39 | 40 | 68 | 69 |
| CCR8 1889 S12P (P) | 1 | 2 | 3 | 4 | 5 | 6 | 7 | 8 |
| CCR8 1889 S12P.I29V (PV) | 1 | 2 | 3 | 41 | 42 | 43 | 68 | 70 |
| CCR8 1889 S12P.A32I (PI) | 1 | 2 | 3 | 44 | 45 | 46 | 68 | 71 |
| CCR8 1889 S12P.E95dS (PS) | 1 | 2 | 3 | 47 | 48 | 49 | 68 | 72 |
| CCR8 1889 S12P.I29V.A32I (PVI) | 1 | 2 | 3 | 50 | 51 | 52 | 68 | 73 |
| CCR8 1889 S12P.I29V.E95dS (PVS) | 1 | 2 | 3 | 53 | 54 | 55 | 68 | 74 |
| CCR8 1889 S12P.A32I.E95dS (PIS) | 1 | 2 | 3 | 56 | 57 | 58 | 68 | 75 |
| CCR8 1889 S12P.I29V.A32I.E95dS (PVIS) | 1 | 2 | 3 | 59 | 60 | 61 | 68 | 76 |
| CCR8 1889 Y58A (A) | 62 | 63 | 64 | 38 | 39 | 40 | 77 | 69 |
| CCR8 1889 T57D.Y58A (DA) | 65 | 66 | 67 | 38 | 39 | 40 | 78 | 69 |
| CD3 40G5c | 17 | 18 | 19 | 20 | 21 | 22 | 23 | 24 |
| CD3 MD1 (38E4v1.MD1) | 91 | 92 | 93 | 94 | 95 | 96 | 97 | 98 |

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a light chain complementarity-determining region 1 (CDR-L1) comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds cluster of differentiation 3 (CD3), wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P (P) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 WT and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 41); (v) a CDR-L2 comprising the amino acid sequence GASNLAS (SEQ ID NO: 42); and (vi) a CDR-L3 comprising the amino acid sequence QQAYYGNSFVEGT (SEQ ID NO: 43); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P.I29V (PV) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 44); (v) a CDR-L2 comprising the amino acid sequence GASNLAS (SEQ ID NO: 45); and (vi) a CDR-L3 comprising the amino acid sequence QQAYYGNSFVEGT (SEQ ID NO: 46); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P.A32I (PI) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 47); (v) a CDR-L2 comprising the amino acid sequence GASNLAS (SEQ ID NO: 48); and (vi) a CDR-L3 comprising the amino acid sequence QQAYYGNSFVSGT (SEQ ID NO: 49); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P.E95dS (PS) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 50); (v) a CDR-L2 comprising the amino acid sequence GASNLAS (SEQ ID NO: 51); and (vi) a CDR-L3 comprising the amino acid sequence QQAYYGNSFVEGT (SEQ ID NO: 52); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P.I29V.A32I (PVI) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 53); (v) a CDR-L2 comprising the amino acid sequence GASNLAS (SEQ ID NO: 54); and (vi) a CDR-L3 comprising the amino acid sequence QQAYYGNSFVSGT (SEQ ID NO: 55); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P.I29V.E95dS (PVS) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 56); (v) a CDR-L2 comprising the amino acid sequence GASNLAS (SEQ ID NO: 57); and (vi) a CDR-L3 comprising the amino acid sequence QQAYYGNSFVSGT (SEQ ID NO: 58); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 CCR8 1889 S12P.A32I.E95dS (PIS) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 59); (v) a CDR-L2 comprising the amino acid sequence GASNLAS (SEQ ID NO: 60); and (vi) a CDR-L3 comprising the amino acid sequence QQAYYGNSFVSGT (SEQ ID NO: 61); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 S12P.I29V.A32I. E95dS (PVIS) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 62); (ii) a CDR-H2 comprising the amino acid sequence LIHRSGRTAYATWAKG (SEQ ID NO: 63); (iii) a CDR-H3 comprising the amino acid sequence SYPDYSATASI (SEQ ID NO: 64); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 Y58A (A) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 65); (ii) a CDR-H2 comprising the amino acid sequence LIHRSGRDAYATWAKG (SEQ ID NO: 66); (iii) a CDR-H3 comprising the amino acid sequence SYPDYSATASI (SEQ ID NO: 67); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40); and (b) a second antigen-binding domain that binds CD3, wherein the second antigen-binding domain comprises the following six CDRs: (i) a CDR-H1 comprising the amino acid sequence NYYIH (SEQ ID NO: 17); (ii) a CDR-H2 comprising the amino acid sequence WIYPGDGNTKYNEKFKG (SEQ ID NO: 18); (iii) a CDR-H3 comprising the amino acid sequence DSYSNYYFDY (SEQ ID NO: 19); (iv) a CDR-L1 comprising the amino acid sequence KSSQSLLNSRTRKNYLA (SEQ ID NO: 20); (v) a CDR-L2 comprising the amino acid sequence WASTRES (SEQ ID NO: 21); and (vi) a CDR-L3 comprising the amino acid sequence TQSFILRT (SEQ ID NO: 22). In some aspects, the first antigen-binding domain is CCR8 1889 T57D.Y58A (DA) and the second antigen-binding domain is CD3 40G5c.

In some aspects, the invention provides a bispecific antigen-binding molecule comprising: (a) a first antigen-binding domain that binds CCR8, wherein the first antigen-binding domain comprises the following six complementarity-determining regions (CDRs): (i) a heavy chain complementarity-determining region 1 (CDR-H1) comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a light chain complementarity-determining region 1 (CDR-L1) comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6); and (b) a second antigen-binding domain that binds cluster of differentiation 3 (CD3), wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P (P) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 WT and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 41); (v) a CDR-L2 comprising the amino acid sequence GASNLAS (SEQ ID NO: 42); and (vi) a CDR-L3 comprising the amino acid sequence QQAYYGNSFVEGT (SEQ ID NO: 43); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P.I29V (PV) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 44); (v) a CDR-L2 comprising the amino acid sequence GASNLAS (SEQ ID NO: 45); and (vi) a CDR-L3 comprising the amino acid sequence QQAYYGNSFVEGT (SEQ ID NO: 46); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P.A32I (PI) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 47); (v) a CDR-L2 comprising the amino acid sequence GASNLAS (SEQ ID NO: 48); and (vi) a CDR-L3 comprising the amino acid sequence QQAYYGNSFVSGT (SEQ ID NO: 49); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P.E95dS (PS) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 50); (v) a CDR-L2 comprising the amino acid sequence GASNLAS (SEQ ID NO: 51); and (vi) a CDR-L3 comprising the amino acid sequence QQAYYGNSFVEGT (SEQ ID NO: 52); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P.I29V.A32I (PVI) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 53); (v) a CDR-L2 comprising the amino acid sequence GASNLAS (SEQ ID NO: 54); and (vi) a CDR-L3 comprising the amino acid sequence QQAYYGNSFVSGT (SEQ ID NO: 55); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P.I29V.E95dS (PVS) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 56); (v) a CDR-L2 comprising the amino acid sequence GASNLAS (SEQ ID NO: 57); and (vi) a CDR-L3 comprising the amino acid sequence QQAYYGNSFVSGT (SEQ ID NO: 58); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 CCR8 1889 S12P.A32I.E95dS (PIS) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 59); (v) a CDR-L2 comprising the amino acid sequence GASNLAS (SEQ ID NO: 60); and (vi) a CDR-L3 comprising the amino acid sequence QQAYYGNSFVSGT (SEQ ID NO: 61); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 S12P.I29V.A32I.E95dS (PVIS) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 62); (ii) a CDR-H2 comprising the amino acid sequence LIHRSGRTAYATWAKG (SEQ ID NO: 63); (iii) a CDR-H3 comprising the amino acid sequence SYPDYSATASI (SEQ ID NO: 64); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 Y58A (A) and the second antigen-binding domain is CD3 38E4v1.MD1 (MD1).

In some aspects, the invention provides a bispecific antigen-binding molecule comprising: (a) a first antigen-binding domain that binds CCR8, wherein the first antigen-binding domain comprises the following six CDRs: (i) a CDR-H1 comprising the amino acid sequence TYAMG (SEQ ID NO: 65); (ii) a CDR-H2 comprising the amino acid sequence LIHRSGRDAYATWAKG (SEQ ID NO: 66); (iii) a CDR-H3 comprising the amino acid sequence SYPDYSATASI (SEQ ID NO: 67); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40); and (b) a second antigen-binding domain that binds CD3, wherein the second antigen-binding domain comprises the following six CDRs: (i) a CDR-H1 comprising the amino acid sequence SYYIH (SEQ ID NO: 91); (ii) a CDR-H2 comprising the amino acid sequence WIYPENDNTKYNEKFKD (SEQ ID NO: 92); (iii) a CDR-H3 comprising the amino acid sequence DGYSRYYFDY (SEQ ID NO: 93); (iv) a CDR-L1 comprising the amino acid sequence KSSQSLLNSRTRKNYLA (SEQ ID NO: 94); (v) a CDR-L2 comprising the amino acid sequence WASTRES (SEQ ID NO: 95); and (vi) a CDR-L3 comprising the amino acid sequence TQSFILRT (SEQ ID NO: 96). In some aspects, the first antigen-binding domain is CCR8 1889 T57D.Y58A (DA) and the second antigen-binding domain is CD3 38E4v1.MD1 (MD1).

In some aspects, the first antigen-binding domain comprises a light chain variable region (VL) domain and a heavy chain variable region (VH) domain, and wherein: (a) the VL domain comprises a proline residue at position 12 (numbering according to Kabat); and/or (b) the VL domain comprises a lysine residue at position 38 and the VH domain comprises a glutamic acid residue at position 39; or the VL domain comprises a glutamic acid residue at position 38 and the VH domain comprises a lysine residue at position 39 (numbering according to Kabat). In some embodiments, the second antigen-binding domain comprises a VL domain and a VH domain, and wherein the VL domain comprises a glutamic acid residue at position 38 and the VH domain comprises a lysine residue at position 39; or the VL domain comprises a lysine residue at position 38 and the VH domain comprises a glutamic acid residue at position 39 (numbering according to Kabat).

In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 10 or SEQ ID NO: 113; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14 or SEQ ID NO: 114; (vii) an FR-L3 comprising the amino acid sequence of SEQ ID NO: 15; and/or (viii) an FR-L4 comprising the amino acid sequence of SEQ ID NO: 16; and/or (b) the second antigen-binding domain comprises one or more of the following eight FRs: (i) an FR-H1 comprising the amino acid sequence of SEQ ID NO: 25; (ii) an FR-H2 comprising the amino acid sequence of SEQ ID NO: 26 or SEQ ID NO: 115; (iii) an FR-H3 comprising the amino acid sequence of SEQ ID NO: 27; (iv) an FR-H4 comprising the amino acid sequence of SEQ ID NO: 28; (v) an FR-L1 comprising the amino acid sequence of SEQ ID NO: 29; (vi) an FR-L2 comprising the amino acid sequence of SEQ ID NO: 30 or SEQ ID NO: 116; (vii) an FR-L3 comprising the amino acid sequence of SEQ ID NO: 31; and/or (viii) an FR-L4 comprising the amino acid sequence of SEQ ID NO: 32.

In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 10; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14; (vii) an FR-L3 comprising the amino acid sequence of SEQ ID NO: 15; and (viii) an FR-L4 comprising the amino acid sequence of SEQ ID NO: 16; and (b) the second antigen-binding domain comprises one or more of the following eight FRs: (i) an FR-H1 comprising the amino acid sequence of SEQ ID NO: 25; (ii) an FR-H2 comprising the amino acid sequence of SEQ ID NO: 26; (iii) an FR-H3 comprising the amino acid sequence of SEQ ID NO: 27; (iv) an FR-H4 comprising the amino acid sequence of SEQ ID NO: 28; (v) an FR-L1 comprising the amino acid sequence of SEQ ID NO: 29; (vi) an FR-L2 comprising the amino acid sequence of SEQ ID NO: 30; (vii) an FR-L3 comprising the amino acid sequence of SEQ ID NO: 31; and (viii) an FR-L4 comprising the amino acid sequence of SEQ ID NO: 32.

In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 113; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 114; (vii) an FR-L3 comprising the amino acid sequence of SEQ ID NO: 15; and (viii) an FR-L4 comprising the amino acid sequence of SEQ ID NO: 16; and (b) the second antigen-binding domain comprises one or more of the following eight FRs: (i) an FR-H1 comprising the amino acid sequence of SEQ ID NO: 25; (ii) an FR-H2 comprising the amino acid sequence of SEQ ID NO: 115; (iii) an FR-H3 comprising the amino acid sequence of SEQ ID NO: 27; (iv) an FR-H4 comprising the amino acid sequence of SEQ ID NO: 28; (v) an FR-L1 comprising the amino acid sequence of SEQ ID NO: 29; (vi) an FR-L2 comprising the amino acid sequence of SEQ ID NO: 116; (vii) an FR-L3 comprising the amino acid sequence of SEQ ID NO: 31; and (viii) an FR-L4 comprising the amino acid sequence of SEQ ID NO: 32.

In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 10 or SEQ ID NO: 113; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14 or SEQ ID NO: 114; (vii) an FR-L3 comprising the amino acid sequence of SEQ ID NO: 15; and/or (viii) an FR-L4 comprising the amino acid sequence of SEQ ID NO: 16; and/or (b) the second antigen-binding domain comprises one or more of the following eight FRs: (i) an FR-H1 comprising the amino acid sequence of SEQ ID NO: 99; (ii) an FR-H2 comprising the amino acid sequence of SEQ ID NO: 100 or SEQ ID NO: 115; (iii) an FR-H3 comprising the amino acid sequence of SEQ ID NO: 101; (iv) an FR-H4 comprising the amino acid sequence of SEQ ID NO: 102; (v) an FR-L1 comprising the amino acid sequence of SEQ ID NO: 103; (vi) an FR-L2 comprising the amino acid sequence of SEQ ID NO: 104 or SEQ ID NO: 116; (vii) an FR-L3 comprising the amino acid sequence of SEQ ID NO: 105; and/or (viii) an FR-L4 comprising the amino acid sequence of SEQ ID NO: 106.

In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 10; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14; (vii) an FR-L3 comprising the amino acid sequence of SEQ ID NO: 15; and (viii) an FR-L4 comprising the amino acid sequence of SEQ ID NO: 16; and (b) the second antigen-binding domain comprises one or more of the following eight FRs: (i) an FR-H1 comprising the amino acid sequence of SEQ ID NO: 99; (ii) an FR-H2 comprising the amino acid sequence of SEQ ID NO: 100; (iii) an FR-H3 comprising the amino acid sequence of SEQ ID NO: 101; (iv) an FR-H4 comprising the amino acid sequence of SEQ ID NO: 102; (v) an FR-L1 comprising the amino acid sequence of SEQ ID NO: 103; (vi) an FR-L2 comprising the amino acid sequence of SEQ ID NO: 104; (vii) an FR-L3 comprising the amino acid sequence of SEQ ID NO: 105; and (viii) an FR-L4 comprising the amino acid sequence of SEQ ID NO: 106.

In some embodiments, (a) the first antigen-binding domain comprises one or more of the following eight framework regions (FRs): (i) an FR-H1 comprising the amino acid sequence of SEQ ID NO: 9; (ii) an FR-H2 comprising the amino acid sequence of SEQ ID NO: 113; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 114; (vii) an FR-L3 comprising the amino acid sequence of SEQ ID NO: 15; and (viii) an FR-L4 comprising the amino acid sequence of SEQ ID NO: 16; and (b) the second antigen-binding domain comprises one or more of the following eight FRs: (i) an FR-H1 comprising the amino acid sequence of SEQ ID NO: 99; (ii) an FR-H2 comprising the amino acid sequence of SEQ ID NO: 115; (iii) an FR-H3 comprising the amino acid sequence of SEQ ID NO: 101; (iv) an FR-H4 comprising the amino acid sequence of SEQ ID NO: 102; (v) an FR-L1 comprising the amino acid sequence of SEQ ID NO: 103; (vi) an FR-L2 comprising the amino acid sequence of SEQ ID NO: 116; (vii) an FR-L3 comprising the amino acid sequence of SEQ ID NO: 105; and (viii) an FR-L4 comprising the amino acid sequence of SEQ ID NO: 106.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 7; (b) a VL domain comprising the amino acid sequence of SEQ ID NO: 8; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 7 and a VL domain comprising the amino acid sequence of SEQ ID NO: 8; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P (P) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 69; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 69; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 WT and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 70; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 70; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 70; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.I29V (PV) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 71; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 71; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 71; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.A32I (PI) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 72; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 72; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 72; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.E95dS (PS) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 73; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 73; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 73; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.I29V.A32I (PVI) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 74; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 74; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 74; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.I29V.E95dS (PVS) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 75; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 75; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 75; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.A32I.E95dS (PIS) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 76; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 76; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 76; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 S12P.I29V.A32I.E95dS (PVIS) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 77; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 77; (b) a VL domain comprising the amino acid sequence of SEQ ID NO: 69; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 77 and a VL domain comprising the amino acid sequence of SEQ ID NO: 69; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 Y58A (A) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 78; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 78; (b) a VL domain comprising the amino acid sequence of SEQ ID NO: 69; or (c) a VH domain as in (a) and a VL domain as in (b); and/or the second antigen-binding domain comprises: (a) a VH domain comprising the amino acid sequence of SEQ ID NO: 23; (b) a VL domain comprising the amino acid sequence of SEQ ID NO: 24; or (c) a VH domain as in (a) and a VL domain as in (b). In some embodiments, the first antigen-binding domain comprises: (a) a VH domain comprising the amino acid sequence of SEQ ID NO: 78 and a VL domain comprising the amino acid sequence of SEQ ID NO: 69; and the second antigen-binding domain comprises: (b) a VH domain comprising the amino acid sequence of SEQ ID NO: 23 and a VL domain comprising the amino acid sequence of SEQ ID NO: 24. In some aspects, the first antigen-binding domain is CCR8 1889 T57D.Y58A (DA) and the second antigen-binding domain is CD3 40G5c.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 7; (b) a VL domain comprising the amino acid sequence of SEQ ID NO: 8; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 7 and a VL domain comprising the amino acid sequence of SEQ ID NO: 8; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P (P) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 69; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 69; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 WT and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 70; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 70; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 70; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.I29V (PV) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 71; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 71; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 71; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.A32I (PI) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 72; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 72; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 72; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.E95dS (PS) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 73; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 73; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 73; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.I29V.A32I (PVI) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 74; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 74; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 74; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.I29V.E95dS (PVS) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 75; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 75; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 75; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.A32I.E95dS (PIS) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 76; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68; (b) a VL domain comprising the amino acid sequence of SEQ ID NO: 76; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 68 and a VL domain comprising the amino acid sequence of SEQ ID NO: 76; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 S12P.I29V.A32I.E95dS (PVIS) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 77; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 77; (b) a VL domain comprising the amino acid sequence of SEQ ID NO: 69; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 77 and a VL domain comprising the amino acid sequence of SEQ ID NO: 69; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 Y58A (A) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 78; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 97; (b) a VL domain comprising an amino acid sequence having at least 80%, 85%, 90%, 95%, 96%, 97%, 98%, 99%, or 100% sequence identity to the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 78; (b) a VL domain comprising the amino acid sequence of SEQ ID NO: 69; or (c) a VH domain as in (a) and a VL domain as in (b); and/or the second antigen-binding domain comprises: (a) a VH domain comprising the amino acid sequence of SEQ ID NO: 97; (b) a VL domain comprising the amino acid sequence of SEQ ID NO: 98; or (c) a VH domain as in (a) and a VL domain as in (b). In some embodiments, the first antigen-binding domain comprises: (a) a VH domain comprising the amino acid sequence of SEQ ID NO: 78 and a VL domain comprising the amino acid sequence of SEQ ID NO: 69; and the second antigen-binding domain comprises: (b) a VH domain comprising the amino acid sequence of SEQ ID NO: 97 and a VL domain comprising the amino acid sequence of SEQ ID NO: 98. In some aspects, the first antigen-binding domain is CCR8 1889 T57D.Y58A (DA) and the second antigen-binding domain is CD3 38E4v1.MD1 (MD1).

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 107; (b) a VL domain comprising an amino acid sequence having at least 80%, 85%, 90%, 95%, 96%, 97%, 98%, 99%, or 100% sequence identity to the amino acid sequence of SEQ ID NO: 108; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 109; (b) a VL domain comprising an amino acid sequence having at least 80%, 85%, 90%, 95%, 96%, 97%, 98%, 99%, or 100% sequence identity to the amino acid sequence of SEQ ID NO: 110; or (c) a VH domain as in (a) and a VL domain as in (b). In some embodiments, the first antigen-binding domain comprises: (a) a VH domain comprising the amino acid sequence of SEQ ID NO: 107; (b) a VL domain comprising the amino acid sequence of SEQ ID NO: 108; or (c) a VH domain as in (a) and a VL domain as in (b); and/or the second antigen-binding domain comprises: (a) a VH domain comprising the amino acid sequence of SEQ ID NO: 109; (b) a VL domain comprising the amino acid sequence of SEQ ID NO: 110; or (c) a VH domain as in (a) and a VL domain as in (b). In some embodiments, the first antigen-binding domain comprises: (a) a VH domain comprising the amino acid sequence of SEQ ID NO: 107 and a VL domain comprising the amino acid sequence of SEQ ID NO: 108; and the second antigen-binding domain comprises: (b) a VH domain comprising the amino acid sequence of SEQ ID NO: 109 and a VL domain comprising the amino acid sequence of SEQ ID NO: 110. In some aspects, the first antigen-binding domain is CCR8 1889 S12P (P) and the second antigen-binding domain is CD3 40G5c, wherein the bispecific antigen-binding molecule further comprises reversed charge modifications, i.e., wherein the charge modifications in the antibody are reversed in comparison to a bispecific antigen-binding molecule comprising the set of amino acid sequences of SEQ ID NOs: 7, 8, 23, and 24.

In some aspects, the first antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 107; (b) a VL domain comprising an amino acid sequence having at least 80%, 85%, 90%, 95%, 96%, 97%, 98%, 99%, or 100% sequence identity to the amino acid sequence of SEQ ID NO: 108; or (c) a VH domain as in (a) and a VL domain as in (b); and/or the second antigen-binding domain comprises: (a) a VH domain comprising an amino acid sequence having at least 80%, 85%, 90%, 95%, 96%, 97%, 98%, 99%, or 100% sequence identity to the amino acid sequence of SEQ ID NO: 111; (b) a VL domain comprising an amino acid sequence having at least 80%, 85%, 90%, 95%, 96%, 97%, 98%, 99%, or 100% sequence identity to the amino acid sequence of SEQ ID NO: 112; or (c) a VH domain as in (a) and a VL domain as in (b). In some embodiments, the first antigen-binding domain comprises: (a) a VH domain comprising the amino acid sequence of SEQ ID NO: 107; (b) a VL domain comprising the amino acid sequence of SEQ ID NO: 108; or (c) a VH domain as in (a) and a VL domain as in (b); and/or the second antigen-binding domain comprises: (a) a VH domain comprising the amino acid sequence of SEQ ID NO: 111; (b) a VL domain comprising the amino acid sequence of SEQ ID NO: 112; or (c) a VH domain as in (a) and a VL domain as in (b). In some embodiments, the first antigen-binding domain comprises: (a) a VH domain comprising the amino acid sequence of SEQ ID NO: 107 and a VL domain comprising the amino acid sequence of SEQ ID NO: 108; and the second antigen-binding domain comprises: (b) a VH domain comprising the amino acid sequence of SEQ ID NO: 111 and a VL domain comprising the amino acid sequence of SEQ ID NO: 112. In some aspects, the first antigen-binding domain is CCR8 1889 S12P (P) and the second antigen-binding domain is CD3 38E4v1.MD1 (MD1), wherein the bispecific antigen-binding molecule further comprises reversed charge modifications, i.e., wherein the charge modifications in the antibody are reversed in comparison to an anti-CCR8 TDB comprising the set of amino acid sequences of SEQ ID NOs: 7, 8, 97, and 98.

In some aspects, the first antigen-binding domain is a Fab molecule comprising a Fab light chain and a Fab heavy chain and/or the second antigen-binding domain is a Fab molecule comprising a Fab light chain and a Fab heavy chain. In some aspects, (a) the first antigen-binding domain is a Fab molecule comprising a Fab light chain and a Fab heavy chain; (b) the second antigen-binding domain is a Fab molecule comprising a Fab light chain and a Fab heavy chain; or (c) the first antigen-binding domain is a Fab molecule comprising a Fab light chain and a Fab heavy chain and the second antigen-binding domain is a Fab molecule comprising a Fab light chain and a Fab heavy chain. In some embodiments, the first antigen-binding domain is a Fab molecule comprising a Fab light chain and a Fab heavy chain and the second antigen-binding domain is a Fab molecule comprising a Fab light chain and a Fab heavy chain, wherein: (a) the Fab light chain of the first antigen-binding domain comprises a glutamic acid residue at position 133, and the Fab heavy chain of the first antigen-binding domain comprises a lysine residue at position 183; or the Fab light chain of the first antigen-binding domain comprises a lysine residue at position 133, and the Fab heavy chain of the first antigen-binding domain comprises a glutamic acid residue at position 183 (numbering according to Kabat) and/or (b) the Fab light chain of the second antigen-binding domain comprises a lysine residue at position 133, and the Fab heavy chain of the second antigen-binding domain comprises a glutamic acid residue at position 183; or the Fab light chain of the second antigen-binding domain comprises a glutamic acid residue at position 133, and the Fab heavy chain of the second antigen-binding domain comprises a lysine residue at position 183 (numbering according to Kabat). In some embodiments, the first antigen-binding domain is a Fab molecule comprising a Fab light chain and a Fab heavy chain and the second antigen-binding domain is a Fab molecule comprising a Fab light chain and a Fab heavy chain, wherein: (a) the Fab light chain of the first antigen-binding domain comprises a glutamic acid residue at position 133, and the Fab heavy chain of the first antigen-binding domain comprises a lysine residue at position 183; or the Fab light chain of the first antigen-binding domain comprises a lysine residue at position 133, and the Fab heavy chain of the first antigen-binding domain comprises a glutamic acid residue at position 183 (numbering according to Kabat); (b) the Fab light chain of the second antigen-binding domain comprises a lysine residue at position 133, and the Fab heavy chain of the second antigen-binding domain comprises a glutamic acid residue at position 183; or the Fab light chain of the second antigen-binding domain comprises a glutamic acid residue at position 133, and the Fab heavy chain of the second antigen-binding domain comprises a lysine residue at position 183 (numbering according to Kabat); or (c) the Fab light chain of the first antigen-binding domain comprises a glutamic acid residue at position 133, and the Fab heavy chain of the first antigen-binding domain comprises a lysine residue at position 183; or the Fab light chain of the first antigen-binding domain comprises a lysine residue at position 133, and the Fab heavy chain of the first antigen-binding domain comprises a glutamic acid residue at position 183 (numbering according to Kabat) and the Fab light chain of the second antigen-binding domain comprises a lysine residue at position 133, and the Fab heavy chain of the second antigen-binding domain comprises a glutamic acid residue at position 183; or the Fab light chain of the second antigen-binding domain comprises a glutamic acid residue at position 133, and the Fab heavy chain of the second antigen-binding domain comprises a lysine residue at position 183 (numbering according to Kabat).

In some aspects, the bispecific antigen-binding molecule further comprises an Fc domain comprising a first subunit and a second subunit. In some embodiments, the Fc domain is an IgG Fc domain. Any suitable IgG Fc domain may be used, e.g., an IgG₁ Fc domain, an IgG₂ Fc domain, an IgG₃ Fc domain, or an IgG₄ Fc domain. In some embodiments, the Fc domain is an IgG₁ Fc domain. In some embodiments, the Fc domain is a human IgG Fc domain. In some embodiments, the Fc domain comprises a modification promoting the association of the first subunit and the second subunit of the Fc domain.

In some aspects, the bispecific antigen-binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain. In some embodiments, the first subunit comprises one or more heavy chain constant domains selected from a first CH2 (CH2₁) domain and/or a first CH3 (CH3₁) domain; and the second subunit comprises one or more heavy chain constant domains selected from a second CH2 (CH2₂) domain and/or a second CH3 (CH3₂) domain. In some embodiments, at least one of the one or more heavy chain constant domains is paired with another heavy chain constant domain. In some embodiments, the CH3₁ and CH3₂ domains each comprise a protuberance or cavity, and wherein the protuberance or cavity in the CH3₁ domain is positionable in the cavity or protuberance, respectively, in the CH3₂ domain. In some embodiments, the CH3₁ and CH3₂ domains meet at an interface between said protuberance and cavity. In some embodiments, the CH2₁ and CH2₂ domains each comprise a protuberance or cavity, and wherein the protuberance or cavity in the CH2₁ domain is positionable in the cavity or protuberance, respectively, in the CH2₂ domain. In some embodiments, the CH2₁ and CH2₂ domains meet at an interface between said protuberance and cavity.

In some aspects, the first antigen-binding domain and the second antigen-binding domain are each a Fab molecule and the bispecific antigen-binding molecule comprises an Fc domain comprising a first subunit and a second subunit; and wherein the first antigen-binding domain is fused at the C-terminus of the Fab heavy chain to the N-terminus of the first subunit and the second antigen-binding domain is fused at the C-terminus of the Fab heavy chain to the N-terminus of the second subunit.

In some aspects, the first subunit comprises a tryptophan residue at position 366; and the second subunit comprises a serine residue at position 366, an alanine residue at position 368, and a valine residue at position 407 (numbered according to Kabat EU index).

In some aspects, each of the first subunit and the second subunit comprises an alanine residue at position 234, an alanine residue at position 235, and a glycine residue at position 329 (numbering according to Kabat EU index).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the sequence GASNLAS (SEQ ID NO: 5); and (vi) a CDR-L3 comprising the amino acid sequence QQAYYGNSFVEGT (SEQ ID NO: 6). In some aspects, the third antigen-binding domain is CCR8 1889 S12P (P).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40). In some aspects, the third antigen-binding domain is CCR8 1889 WT.

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 41); (v) a CDR-L2 comprising the amino acid sequence GASNLAS (SEQ ID NO: 42); and (vi) a CDR-L3 comprising the amino acid sequence QQAYYGNSFVEGT (SEQ ID NO: 43). In some aspects, the third antigen-binding domain is CCR8 1889 S12P.I29V (PV).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 44); (v) a CDR-L2 comprising the amino acid sequence GASNLAS (SEQ ID NO: 45); and (vi) a CDR-L3 comprising the amino acid sequence QQAYYGNSFVEGT (SEQ ID NO: 46). In some aspects, the third antigen-binding domain is CCR8 1889 S12P.A32I (PI).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 47); (v) a CDR-L2 comprising the amino acid sequence GASNLAS (SEQ ID NO: 48); and (vi) a CDR-L3 comprising the amino acid sequence QQAYYGNSFVSGT (SEQ ID NO: 49). In some aspects, the third antigen-binding domain is CCR8 1889 S12P.E95dS (PS).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 50); (v) a CDR-L2 comprising the amino acid sequence GASNLAS (SEQ ID NO: 51); and (vi) a CDR-L3 comprising the amino acid sequence QQAYYGNSFVEGT (SEQ ID NO: 52). In some aspects, the third antigen-binding domain is CCR8 1889 S12P.I29V.A32I (PVI).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 53); (v) a CDR-L2 comprising the amino acid sequence GASNLAS (SEQ ID NO: 54); and (vi) a CDR-L3 comprising the amino acid sequence QQAYYGNSFVSGT (SEQ ID NO: 55). In some aspects, the third antigen-binding domain is CCR8 1889 S12P.I29V.E95dS (PVS).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 56); (v) a CDR-L2 comprising the amino acid sequence GASNLAS (SEQ ID NO: 57); and (vi) a CDR-L3 comprising the amino acid sequence QQAYYGNSFVSGT (SEQ ID NO: 58). In some aspects, the third antigen-binding domain is CCR8 1889 S12P.A32I.E95dS (PIS).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 59); (v) a CDR-L2 comprising the amino acid sequence GASNLAS (SEQ ID NO: 60); and (vi) a CDR-L3 comprising the amino acid sequence QQAYYGNSFVSGT (SEQ ID NO: 61). In some aspects, the third antigen-binding domain is CCR8 1889 S12P.I29V.A32I.E95dS (PVIS).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 62); (ii) a CDR-H2 comprising the amino acid sequence LIHRSGRTAYATWAKG (SEQ ID NO: 63); (iii) a CDR-H3 comprising the amino acid sequence SYPDYSATASI (SEQ ID NO: 64); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40). In some aspects, the third antigen-binding domain is CCR8 1889 Y58A (A).

In some aspects, the bispecific antigen-binding molecule further comprises a third antigen-binding domain that binds to CCR8. In some embodiments, the third antigen-binding domain comprises the following six CDRs: (i) a CDR-H1 comprising the amino acid sequence TYAMG (SEQ ID NO: 65); (ii) a CDR-H2 comprising the amino acid sequence LIHRSGRDAYATWAKG (SEQ ID NO: 66); (iii) a CDR-H3 comprising the amino acid sequence SYPDYSATASI (SEQ ID NO: 67); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40). In some aspects, the third antigen-binding domain is CCR8 1889 T57D.Y58A (DA).

In some aspects, the third antigen-binding domain comprises a VL domain and a VH domain, and wherein: (a) the VL domain comprises a proline residue at position 12 (numbering according to Kabat); and/or (b) the VL domain comprises a lysine residue at position 38 and the VH domain comprises a glutamic acid residue at position 39; or the VL domain comprises a glutamic acid residue at position 38 and the VH domain comprises a lysine residue at position 39 (numbering according to Kabat). In some aspects, the third antigen-binding domain comprises a VL domain and a VH domain, and wherein: (a) the VL domain comprises a proline residue at position 12 (numbering according to Kabat); (b) the VL domain comprises a lysine residue at position 38 and the VH domain comprises a glutamic acid residue at position 39; or the VL domain comprises a glutamic acid residue at position 38 and the VH domain comprises a lysine residue at position 39 (numbering according to Kabat); or (c) the VL domain comprises a proline residue at position 12 (numbering according to Kabat) and the VL domain comprises a lysine residue at position 38 and the VH domain comprises a glutamic acid residue at position 39; or the VL domain comprises a proline residue at position 12 (numbering according to Kabat) and the VL domain comprises a glutamic acid residue at position 38 and the VH domain comprises a lysine residue at position 39 (numbering according to Kabat).

In some embodiments, the third antigen-binding domain comprises one or more of the following eight FRs: (i) an FR-H1 comprising the amino acid sequence of SEQ ID NO: 9; (ii) an FR-H2 comprising the amino acid sequence of SEQ ID NO: 10 or SEQ ID NO: 113; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14 or SEQ ID NO: 114; (vii) an FR-L3 comprising the amino acid sequence of SEQ ID NO: 15; and/or (viii) an FR-L4 comprising the amino acid sequence of SEQ ID NO: 16.

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b). In some embodiments, the third antigen-binding domain comprises (a) a VH domain comprising the amino acid sequence of SEQ ID NO: 7; (b) a VL domain comprising the amino acid sequence of SEQ ID NO: 8; or (c) a VH domain as in (a) and a VL domain as in (b). In some embodiments, the third antigen-binding domain comprises a VH domain comprising the amino acid sequence of SEQ ID NO: 7 and a VL domain comprising the amino acid sequence of SEQ ID NO: 8. In some aspects, the third antigen-binding domain is CCR8 1889 S12P (P).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 69; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 69. In some aspects, the third antigen-binding domain is CCR8 1889 WT.

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 70; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 70; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 70. In some aspects, the third antigen-binding domain is CCR8 1889 S12P.I29V (PV).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 71; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 71; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 71. In some aspects, the third antigen-binding domain is CCR8 1889 S12P.A32I (PI).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 72; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 72; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 72. In some aspects, the third antigen-binding domain is CCR8 1889 S12P.E95dS (PS).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 73; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 73; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 73. In some aspects, the third antigen-binding domain is CCR8 1889 S12P.I29V.A32I (PVI).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 74; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 74; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 74. In some aspects, the third antigen-binding domain is CCR8 1889 S12P.I29V.E95dS (PVS).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 75; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 75; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 75. In some aspects, the third antigen-binding domain is CCR8 1889 S12P.A32I. E95dS (PIS).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 76; or (c) a VH domain as in (a) and a VL domain as in (b). In some embodiments, the third antigen-binding domain comprises (a) a VH domain comprising the amino acid sequence of SEQ ID NO: 68; (b) a VL domain comprising the amino acid sequence of SEQ ID NO: 76; or (c) a VH domain as in (a) and a VL domain as in (b). In some embodiments, the third antigen-binding domain comprises a VH domain comprising the amino acid sequence of SEQ ID NO: 68 and a VL domain comprising the amino acid sequence of SEQ ID NO: 76. In some aspects, the third antigen-binding domain is CCR8 1889 S12P.I29V.A32I.E95dS (PVIS).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 77; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b). In some embodiments, the third antigen-binding domain comprises (a) a VH domain comprising the amino acid sequence of SEQ ID NO: 77; (b) a VL domain comprising the amino acid sequence of SEQ ID NO: 69; or (c) a VH domain as in (a) and a VL domain as in (b). In some embodiments, the third antigen-binding domain comprises a VH domain comprising the amino acid sequence of SEQ ID NO: 77 and a VL domain comprising the amino acid sequence of SEQ ID NO: 69. In some aspects, the third antigen-binding domain is CCR8 1889 Y58A (A).

In some aspects, the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 78; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b). In some embodiments, the third antigen-binding domain comprises (a) a VH domain comprising the amino acid sequence of SEQ ID NO: 78; (b) a VL domain comprising the amino acid sequence of SEQ ID NO: 69; or (c) a VH domain as in (a) and a VL domain as in (b). In some embodiments, the third antigen-binding domain comprises a VH domain comprising the amino acid sequence of SEQ ID NO: 78 and a VL domain comprising the amino acid sequence of SEQ ID NO: 69. In some aspects, the third antigen-binding domain is CCR8 1889 T57D.Y58A (DA).

In some aspects, the third antigen-binding domain is a Fab molecule. In some embodiments, the third antigen-binding domain is a Fab molecule comprising a Fab light chain and a Fab heavy chain, and wherein the Fab light chain of the third antigen-binding domain comprises a glutamic acid residue at position 133, and the Fab heavy chain of the third antigen-binding domain comprises a lysine residue at position 183; or the Fab light chain of the third antigen-binding domain comprises a lysine residue at position 133, and the Fab heavy chain of the third antigen-binding domain comprises a glutamic acid residue at position 183 (numbering according to Kabat).

In some aspects, the second antigen-binding domain and the third antigen-binding domain are fused to each other. In some embodiments, the second antigen-binding domain and the third antigen-binding domain are fused to each other via a peptide linker. In some embodiments, the peptide linker comprises the amino acid sequence of SEQ ID NO: 37. In some embodiments, the second antigen-binding domain and the third antigen-binding domain are each a Fab molecule, and wherein the third antigen-binding domain is fused at the C-terminus of the Fab heavy chain to the N-terminus of the Fab heavy chain of the second antigen-binding domain.

In some aspects, the bispecific antigen-binding molecule comprises an Fc domain comprising of a first subunit and a second subunit; wherein the first antigen-binding domain, the second antigen-binding domain, and the third antigen-binding domain are each a Fab molecule; wherein the first antigen-binding domain is fused at the C-terminus of the Fab heavy chain to the N-terminus of the first subunit; wherein the second antigen-binding domain is fused at the C-terminus of the Fab heavy chain to the N-terminus of the second subunit; and wherein the third antigen-binding domain is fused at the C-terminus of the Fab heavy chain to the N-terminus of the Fab heavy chain of the second antigen-binding domain. In some aspects, the bispecific antigen-binding molecule is a 2+1 A/AB format (A: CCR8-binding domain), B: CD3-binding domain) anti-CCR8 TDB.

In some embodiments, the multispecific antigen-binding molecule described herein comprises a first Fab molecule (Fab_{A}) and a third Fab molecule (Fab_{B2}) that each specifically binds to CCR8 comprising Q39E (Kabat numbering) and S183K (EU numbering) substitutions in the heavy chain and Q38K (Kabat numbering) and V133E (EU numbering) substitutions in the light chain; and a second Fab molecule (Fab_{B1}) that specifically binds to CD3 comprising Q39K (Kabat numbering) and S183E (EU numbering) substitution in the heavy chain and Q38E (Kabat numbering) and V133K (EU numbering) substitutions in the light chain.

In some embodiments, the multispecific antigen-binding molecule described herein comprises a first Fab molecule (Fab_{A}) and a third Fab molecule (Fab_{B2}) that each specifically binds to CCR8 comprising Q39K (Kabat numbering) and S183E (EU numbering) substitutions in the heavy chain and Q38E (Kabat numbering) and V133K (EU numbering) substitutions in the light chain; and a second Fab molecule (Fab_{B1}) that specifically binds to CD3 comprising Q39E (Kabat numbering) and S183K (EU numbering) substitution in the heavy chain and Q38K (Kabat numbering) and V133E (EU numbering) substitutions in the light chain.

In some aspects, the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 33, a polypeptide comprising an amino acid sequence having at least 80%, 85%, 90%, 95%, 96%, 97%, 98%, 99%, or 100% sequence identity to the amino acid sequence of SEQ ID NO: 34, a polypeptide comprising an amino acid sequence having at least 80%, 85%, 90%, 95%, 96%, 97%, 98%, 99%, or 100% sequence identity to the amino acid sequence of SEQ ID NO: 35, and a polypeptide comprising an amino acid sequence having at least 80%, 85%, 90%, 95%, 96%, 97%, 98%, 99%, or 100% sequence identity to the amino acid sequence of SEQ ID NO: 36. In some embodiments, the bispecific antigen-binding molecule comprises a polypeptide comprising the amino acid sequence of SEQ ID NO: 33, a first polypeptide and a second polypeptide each comprising the amino acid sequence of SEQ ID NO: 34, a polypeptide comprising the amino acid sequence of SEQ ID NO: 35, and a polypeptide comprising the amino acid sequence of SEQ ID NO: 36. In some embodiments, (i) the polypeptide comprising the amino acid sequence of SEQ ID NO: 33 is connected to the first polypeptide comprising the amino acid sequence of SEQ ID NO: 34 via a Fab heavy chain and Fab light chain interaction; (ii) the polypeptide comprising the amino acid sequence of SEQ ID NO: 35 is connected to the second polypeptide comprising the amino acid sequence of SEQ ID NO: 34 via a Fab heavy chain and Fab light chain interaction; (iii) the polypeptide comprising the amino acid sequence of SEQ ID NO: 35 is connected to the polypeptide comprising the amino acid sequence of SEQ ID NO: 36 via a Fab heavy chain and Fab light chain interaction; and (iv) the polypeptide comprising the amino acid sequence of SEQ ID NO: 33 is connected to the polypeptide comprising the amino acid sequence of SEQ ID NO: 35 via a first subunit and a second subunit of an Fc domain. In some aspects, the bispecific antigen-binding molecule is a 2+1 A/AB format (A: 1889 P, B: 40G5c) 1889/1889:40G5c anti-CCR8 TDB.

In some aspects, the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 117, a polypeptide comprising an amino acid sequence having at least 80%, 85%, 90%, 95%, 96%, 97%, 98%, 99%, or 100% sequence identity to the amino acid sequence of SEQ ID NO: 118, a polypeptide comprising an amino acid sequence having at least 80%, 85%, 90%, 95%, 96%, 97%, 98%, 99%, or 100% sequence identity to the amino acid sequence of SEQ ID NO: 119, and a polypeptide comprising an amino acid sequence having at least 80%, 85%, 90%, 95%, 96%, 97%, 98%, 99%, or 100% sequence identity to the amino acid sequence of SEQ ID NO: 120. In some embodiments, the bispecific antigen-binding molecule comprises a polypeptide comprising the amino acid sequence of SEQ ID NO: 117, a first polypeptide and a second polypeptide each comprising the amino acid sequence of SEQ ID NO: 118, a polypeptide comprising the amino acid sequence of SEQ ID NO: 119, and a polypeptide comprising the amino acid sequence of SEQ ID NO: 120. In some embodiments, (i) the polypeptide comprising the amino acid sequence of SEQ ID NO: 117 is connected to the first polypeptide comprising the amino acid sequence of SEQ ID NO: 118 via a Fab heavy chain and Fab light chain interaction; (ii) the polypeptide comprising the amino acid sequence of SEQ ID NO: 119 is connected to the second polypeptide comprising the amino acid sequence of SEQ ID NO: 118 via a Fab heavy chain and Fab light chain interaction; (iii) the polypeptide comprising the amino acid sequence of SEQ ID NO: 119 is connected to the polypeptide comprising the amino acid sequence of SEQ ID NO: 120 via a Fab heavy chain and Fab light chain interaction; and (iv) the polypeptide comprising the amino acid sequence of SEQ ID NO: 117 is connected to the polypeptide comprising the amino acid sequence of SEQ ID NO: 119 via a first subunit and a second subunit of an Fc domain. In some aspects, the bispecific antigen-binding molecule is a 2+1 A/AB format (A: 1889 P, B: 40G5c) 1889/1889:40G5c anti-CCR8 TDB, wherein the bispecific antigen-binding molecule further comprises reversed charge modifications, i.e., wherein the charge modifications in the antibody are reversed in comparison to an anti-CCR8 TDB comprising the set of amino acid sequences of SEQ ID NOs: 33-36.

In some aspects, the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 33, a polypeptide comprising an amino acid sequence having at least 80%, 85%, 90%, 95%, 96%, 97%, 98%, 99%, or 100% sequence identity to the amino acid sequence of SEQ ID NO: 34, a polypeptide comprising an amino acid sequence having at least 80%, 85%, 90%, 95%, 96%, 97%, 98%, 99%, or 100% sequence identity to the amino acid sequence of SEQ ID NO: 90, and a polypeptide comprising an amino acid sequence having at least 80%, 85%, 90%, 95%, 96%, 97%, 98%, 99%, or 100% sequence identity to the amino acid sequence of SEQ ID NO: 36. In some embodiments, the bispecific antigen-binding molecule comprises a polypeptide comprising the amino acid sequence of SEQ ID NO: 33, a first polypeptide and a second polypeptide each comprising the amino acid sequence of SEQ ID NO: 34, a polypeptide comprising the amino acid sequence of SEQ ID NO: 90, and a polypeptide comprising the amino acid sequence of SEQ ID NO: 36. In some embodiments, (i) the polypeptide comprising the amino acid sequence of SEQ ID NO: 33 is connected to the first polypeptide comprising the amino acid sequence of SEQ ID NO: 34 via a Fab heavy chain and Fab light chain interaction; (ii) the polypeptide comprising the amino acid sequence of SEQ ID NO: 90 is connected to the second polypeptide comprising the amino acid sequence of SEQ ID NO: 34 via a Fab heavy chain and Fab light chain interaction; (iii) the polypeptide comprising the amino acid sequence of SEQ ID NO: 90 is connected to the polypeptide comprising the amino acid sequence of SEQ ID NO: 36 via a Fab heavy chain and Fab light chain interaction; and (iv) the polypeptide comprising the amino acid sequence of SEQ ID NO: 33 is connected to the polypeptide comprising the amino acid sequence of SEQ ID NO: 90 via a first subunit and a second subunit of an Fc domain. In some aspects, the bispecific antigen-binding molecule is a 2+1 A/AB format (A: 1889 P, B: MD1) 1889/1889: MD1 anti-CCR8 TDB.

In some aspects, the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 117, a polypeptide comprising an amino acid sequence having at least 80%, 85%, 90%, 95%, 96%, 97%, 98%, 99%, or 100% sequence identity to the amino acid sequence of SEQ ID NO: 118, a polypeptide comprising an amino acid sequence having at least 80%, 85%, 90%, 95%, 96%, 97%, 98%, 99%, or 100% sequence identity to the amino acid sequence of SEQ ID NO: 121, and a polypeptide comprising an amino acid sequence having at least 80%, 85%, 90%, 95%, 96%, 97%, 98%, 99%, or 100% sequence identity to the amino acid sequence of SEQ ID NO: 120. In some embodiments, the bispecific antigen-binding molecule comprises a polypeptide comprising the amino acid sequence of SEQ ID NO: 117, a first polypeptide and a second polypeptide each comprising the amino acid sequence of SEQ ID NO: 118, a polypeptide comprising the amino acid sequence of SEQ ID NO: 121, and a polypeptide comprising the amino acid sequence of SEQ ID NO: 120. In some embodiments, (i) the polypeptide comprising the amino acid sequence of SEQ ID NO: 117 is connected to the first polypeptide comprising the amino acid sequence of SEQ ID NO: 118 via a Fab heavy chain and Fab light chain interaction; (ii) the polypeptide comprising the amino acid sequence of SEQ ID NO: 121 is connected to the second polypeptide comprising the amino acid sequence of SEQ ID NO: 118 via a Fab heavy chain and Fab light chain interaction; (iii) the polypeptide comprising the amino acid sequence of SEQ ID NO: 121 is connected to the polypeptide comprising the amino acid sequence of SEQ ID NO: 120 via a Fab heavy chain and Fab light chain interaction; and (iv) the polypeptide comprising the amino acid sequence of SEQ ID NO: 117 is connected to the polypeptide comprising the amino acid sequence of SEQ ID NO: 121 via a first subunit and a second subunit of an Fc domain. In some aspects, the bispecific antigen-binding molecule is a 2+1 A/AB format (A: 1889 P, B: MD1) 1889/1889: MD1 anti-CCR8 TDB, wherein the bispecific antigen-binding molecule further comprises reversed charge modifications, i.e., wherein the charge modifications in the antibody are reversed in comparison to an anti-CCR8 TDB comprising the set of amino acid sequences of SEQ ID NOs: 33, 34, 36, and 90.

The bispecific antigen-binding molecules described herein may include any one or combination of the properties described further in Section C below.

### B. Anti-CCR8 Antibodies

In other aspects, provided herein are anti-CCR8 antibodies. Any of the anti-CCR8 antibodies disclosed herein may be used in a multispecific (e.g., bispecific) antigen-binding molecule as described herein.

In some aspects, the invention provides isolated anti-CCR8 antibodies. In some embodiments, an anti-CCR8 antibody of the present invention comprises at least one, at least two, at least three, at least four, at least five, or all six CDRs (e.g., comprises one, two, three, four, five, or six CDRs) as illustrated in Table 2 (Kabat). In some instances, the anti-CCR8 antibody comprises a VH and/or a VL as illustrated in Table 2.

In some aspects, the invention provides an anti-CCR8 antibody comprising the following six CDRs: (i) a CDR-H1 comprising the amino acid sequence TYAMG (SEQ ID NO: 1); (ii) a CDR-H2 comprising the amino acid sequence LIHRSGRX₁X₂YATWAKG (SEQ ID NO: 82), wherein X₁ is T or D and X₂ is Y or A; (iii) a CDR-H3 comprising the amino acid sequence SYPDYSATASI (SEQ ID NO: 3); (iv) a CDR-L1 comprising the amino acid sequence QASENX₃ANX₄LA (SEQ ID NO: 83), wherein X₃ is I or V and X₄ is A or I; (v) a CDR-L2 comprising the amino acid sequence GASNLAS (SEQ ID NO: 5); and (vi) a CDR-L3 comprising the amino acid sequence QQAYYGNSFVX₅GT (SEQ ID NO: 84), wherein X₅ is E or S. In some examples, the anti-CCR8 antibody comprises a VL domain that does not comprise a Serine residue at position 12 (Kabat numbering). For example, in some examples, the anti-CCR8 antibody does not comprise a VL domain comprising the amino acid sequence of SEQ ID NO: 69.

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 4); (v) a CDR-L2 comprising the amino acid sequence GASNLAS (SEQ ID NO: 5); and (vi) a CDR-L3 comprising the amino acid sequence QQAYYGNSFVEGT (SEQ ID NO: 6). In some aspects, the anti-CCR8 antibody is CCR8 1889 S12P (P).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40). In some aspects, the anti-CCR8 antibody is CCR8 1889 WT.

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 41); (v) a CDR-L2 comprising the amino acid sequence GASNLAS (SEQ ID NO: 42); and (vi) a CDR-L3 comprising the amino acid sequence QQAYYGNSFVEGT (SEQ ID NO: 43). In some aspects, the anti-CCR8 antibody is CCR8 1889 S12P.I29V (PV).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 44); (v) a CDR-L2 comprising the amino acid sequence GASNLAS (SEQ ID NO: 45); and (vi) a CDR-L3 comprising the amino acid sequence QQAYYGNSFVEGT (SEQ ID NO: 46). In some aspects, the anti-CCR8 antibody is CCR8 1889 S12P.A32I (PI).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANALA (SEQ ID NO: 47); (v) a CDR-L2 comprising the amino acid sequence GASNLAS (SEQ ID NO: 48); and (vi) a CDR-L3 comprising the amino acid sequence QQAYYGNSFVSGT (SEQ ID NO: 49). In some aspects, the anti-CCR8 antibody is CCR8 1889 S12P.E95dS (PS).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 50); (v) a CDR-L2 comprising the amino acid sequence GASNLAS (SEQ ID NO: 51); and (vi) a CDR-L3 comprising the amino acid sequence QQAYYGNSFVEGT (SEQ ID NO: 52). In some aspects, the anti-CCR8 antibody is CCR8 1889 S12P.I29V.A32I (PVI).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANALA (SEQ ID NO: 53); (v) a CDR-L2 comprising the amino acid sequence GASNLAS (SEQ ID NO: 54); and (vi) a CDR-L3 comprising the amino acid sequence QQAYYGNSFVSGT (SEQ ID NO: 55). In some aspects, the anti-CCR8 antibody is CCR8 1889 S12P.I29V.E95dS (PVS).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENIANILA (SEQ ID NO: 56); (v) a CDR-L2 comprising the amino acid sequence GASNLAS (SEQ ID NO: 57); and (vi) a CDR-L3 comprising the amino acid sequence QQAYYGNSFVSGT (SEQ ID NO: 58). In some aspects, the anti-CCR8 antibody is CCR8 1889 S12P.A32I.E95dS (PIS).

In some aspects, the anti-CCR8 antibody comprises the following six CDRs: (i) a CDR-H1 comprising the amino acid sequence TYAMG (SEQ ID NO: 1); (ii) a CDR-H2 comprising the amino acid sequence LIHRSGRTYYATWAKG (SEQ ID NO: 2); (iii) a CDR-H3 comprising the amino acid sequence SYPDYSATASI (SEQ ID NO: 3); (iv) a CDR-L1 comprising the amino acid sequence QASENVANILA (SEQ ID NO: 59); (v) a CDR-L2 comprising the amino acid sequence GASNLAS (SEQ ID NO: 60); and (vi) a CDR-L3 comprising the amino acid sequence QQAYYGNSFVSGT (SEQ ID NO: 61). In some aspects, the anti-CCR8 antibody is CCR8 1889 S12P.I29V.A32I. E95dS (PVIS).

In some aspects, the anti-CCR8 antibody comprises the following six CDRs: (i) a CDR-H1 comprising the amino acid sequence TYAMG (SEQ ID NO: 62); (ii) a CDR-H2 comprising the amino acid sequence LIHRSGRTAYATWAKG (SEQ ID NO: 63); (iii) a CDR-H3 comprising the amino acid sequence SYPDYSATASI (SEQ ID NO: 64); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40). In some aspects, the anti-CCR8 antibody is CCR8 1889 Y58A (A).

In some aspects, the anti-CCR8 antibody comprises the following six CDRs: (i) a CDR-H1 comprising the amino acid sequence TYAMG (SEQ ID NO: 65); (ii) a CDR-H2 comprising the amino acid sequence LIHRSGRDAYATWAKG (SEQ ID NO: 66); (iii) a CDR-H3 comprising the amino acid sequence SYPDYSATASI (SEQ ID NO: 67); (iv) a CDR-L1 comprising the amino acid sequence QASENIANALA (SEQ ID NO: 38); (v) a CDR-L2 comprising the amino acid sequence GASNLAS (SEQ ID NO: 39); and (vi) a CDR-L3 comprising the amino acid sequence QQAYYGNSFVEGT (SEQ ID NO: 40). In some aspects, the anti-CCR8 antibody is CCR8 1889 T57D.Y58A (DA).

In some embodiments, the anti-CCR8 antibody comprises one or more of the following eight FRs: (i) an FR-H1 comprising the amino acid sequence of SEQ ID NO: 9; (ii) an FR-H2 comprising the amino acid sequence of SEQ ID NO: 10; (iii) an FR-H3 comprising the amino acid sequence of SEQ ID NO: 11; (iv) an FR-H4 comprising the amino acid sequence of SEQ ID NO: 12; (v) an FR-L1 comprising the amino acid sequence of SEQ ID NO: 13; (vi) an FR-L2 comprising the amino acid sequence of SEQ ID NO: 14; (vii) an FR-L3 comprising the amino acid sequence of SEQ ID NO: 15; and/or (viii) an FR-L4 comprising the amino acid sequence of SEQ ID NO: 16.

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b). In some embodiments, the anti-CCR8 antibody comprises (a) a VH domain comprising the amino acid sequence of SEQ ID NO: 7; (b) a VL domain comprising the amino acid sequence of SEQ ID NO: 8; or (c) a VH domain as in (a) and a VL domain as in (b). In some embodiments, the anti-CCR8 antibody comprises a VH domain comprising the amino acid sequence of SEQ ID NO: 7 and a VL domain comprising the amino acid sequence of SEQ ID NO: 8. In some aspects, the anti-CCR8 antibody is CCR8 1889 S12P (P).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 69; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 69. In some aspects, the anti-CCR8 antibody is CCR8 1889 WT.

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 70; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 70; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 70. In some aspects, the anti-CCR8 antibody is CCR8 1889 S12P.I29V (PV).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 71; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 71; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 71. In some aspects, the anti-CCR8 antibody is CCR8 1889 S12P.A32I (PI).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 72; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 72; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 72. In some aspects, the anti-CCR8 antibody is CCR8 1889 S12P.E95dS (PS).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 73; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 73; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 73. In some aspects, the anti-CCR8 antibody is CCR8 1889 S12P.I29V.A32I (PVI).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 74; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 74; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 74. In some aspects, the anti-CCR8 antibody is CCR8 1889 S12P.I29V.E95dS (PVS).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 75; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 75; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 75. In some aspects, the anti-CCR8 antibody is CCR8 1889 S12P.A32I.E95dS (PIS).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 68; (b) a VL domain comprising an amino acid sequence having at least 80%, 85%, 90%, 95%, 96%, 97%, 98%, 99%, or 100% sequence identity to the amino acid sequence of SEQ ID NO: 76; or (c) a VH domain as in (a) and a VL domain as in (b). In some embodiments, the anti-CCR8 antibody comprises (a) a VH domain comprising the amino acid sequence of SEQ ID NO: 68; (b) a VL domain comprising the amino acid sequence of SEQ ID NO: 76; or (c) a VH domain as in (a) and a VL domain as in (b). In some embodiments, the anti-CCR8 antibody comprises a VH domain comprising the amino acid sequence of SEQ ID NO: 68 and a VL domain comprising the amino acid sequence of SEQ ID NO: 76. In some aspects, the anti-CCR8 antibody is CCR8 1889 S12P.I29V.A32I. E95dS (PVIS).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 77; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b). In some embodiments, the anti-CCR8 antibody comprises (a) a VH domain comprising the amino acid sequence of SEQ ID NO: 77; (b) a VL domain comprising the amino acid sequence of SEQ ID NO: 69; or (c) a VH domain as in (a) and a VL domain as in (b). In some embodiments, the anti-CCR8 antibody comprises a VH domain comprising the amino acid sequence of SEQ ID NO: 77 and a VL domain comprising the amino acid sequence of SEQ ID NO: 69. In some aspects, the anti-CCR8 antibody is CCR8 1889 Y58A (A).

In some aspects, the anti-CCR8 antibody comprises (a) a VH domain comprising an amino acid sequence having at least 80%, 85%, 90%, 95%, 96%, 97%, 98%, 99%, or 100% sequence identity to the amino acid sequence of SEQ ID NO: 78; (b) a VL domain comprising an amino acid sequence having at least 80%, 85%, 90%, 95%, 96%, 97%, 98%, 99%, or 100% sequence identity to the amino acid sequence of SEQ ID NO: 69; or (c) a VH domain as in (a) and a VL domain as in (b). In some embodiments, the anti-CCR8 antibody comprises (a) a VH domain comprising the amino acid sequence of SEQ ID NO: 78; (b) a VL domain comprising the amino acid sequence of SEQ ID NO: 69; or (c) a VH domain as in (a) and a VL domain as in (b). In some embodiments, the anti-CCR8 antibody comprises a VH domain comprising the amino acid sequence of SEQ ID NO: 78 and a VL domain comprising the amino acid sequence of SEQ ID NO: 69. In some aspects, the anti-CCR8 antibody is CCR8 1889 T57D.Y58A (DA).

The antibodies described herein may include any one or combination of the properties described further in Section C below.

### C. Properties of Multispecific Antigen-Binding Molecules and/or Antibodies

### 1. Antibody Fragments

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) provided herein is an antibody fragment. In certain aspects, a multispecific (e.g., bispecific) antigen-binding molecule provided herein includes an antibody fragment.

Any suitable antibody fragment may be used. In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide, typically of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 2. Chimeric and Humanized Antigen-Binding Molecules

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) provided herein is a chimeric multispecific antigen-binding molecule or multispecific antibody. In certain aspects, a multispecific (e.g., bispecific) antigen-binding molecule or antibody (e.g., anti-CCR8 antibody) provided herein includes a chimeric antigen-binding molecule or antibody.

Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antigen-binding molecule or antibody is a humanized antigen-binding molecule or antibody. Typically, a non-human antigen-binding molecule or antibody is humanized to reduce immunogenicity to humans, while retaining the specificity and affinity of the parental non-human antibody. Generally, a humanized antigen-binding molecule or antibody comprises one or more variable domains in which the CDRs (or portions thereof) are derived from a non-human antigen-binding molecule or antibody, and FRs (or portions thereof) are derived from human antibody sequences. A humanized antigen-binding molecule or antibody optionally will also comprise at least a portion of a human constant region. In some aspects, some FR residues in a humanized antigen-binding molecule or antibody are substituted with corresponding residues from a non-human antigen-binding molecule or antibody (e.g., the antibody from which the CDR residues are derived), e.g., to restore or improve antibody specificity or affinity.

Humanized antigen-binding molecules and antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 3. Human Antigen-Binding Molecules

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) provided herein is a human multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or antibody (e.g., anti-CCR8 antibody). Human antigen-binding molecules or antibodies can be produced using various techniques known in the art. Human antigen-binding molecules or antibodies are described generally in van Dijk and van de Winkel, Curr. Opin. Pharmacol. 5:368-74 (2001) and Lonberg, Curr. Opin. Immunol. 20:450-459 (2008).

Human antigen-binding molecules or antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antigen-binding molecules or antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antigen-binding molecules or antibodies can also be made by hybridoma-based methods. Human myeloma and mouse-human heteromyeloma cell lines for the production of human monoclonal antigen-binding molecules or antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antigen-binding molecules or antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antigen-binding molecules or antibodies may also be generated by isolating variable domain sequences selected from human-derived phage display libraries. Such variable domain sequences may then be combined with a desired human constant domain. Techniques for selecting human antibodies from antibody libraries are described below.

### 4. Multispecific Antigen-Binding Molecules

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is disclosed herein. “Multispecific antigen-binding molecules” and “multispecific antibodies” are monoclonal antigen-binding molecules or antibodies, respectively, that have binding specificities for at least two different sites, i.e., different epitopes on different antigens or different epitopes on the same antigen. In certain aspects, the multispecific antigen-binding molecule or antibody has three or more binding specificities. In certain aspects, one of the binding specificities is for CCR8 and the other specificity is for any other antigen. In certain aspects, bispecific antigen-binding molecules or antibodies may bind to two (or more) different epitopes of CCR8. Multispecific (e.g., bispecific) antigen-binding molecules or antibodies may also be used to localize cytotoxic agents or cells to cells which express CCR8. Multispecific antigen-binding molecules or antibodies may be prepared as full-length antigen-binding molecules or antibodies or antibody fragments. In certain aspects, the other antigen is an activating T cell antigen. In some aspects, the activating T cell antigen is CD3. In some aspects, the CD3 is human or cyno CD3.

Techniques for making multispecific antigen-binding molecules or antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

### 5. Antibody Variants

In certain aspects, amino acid sequence variants of the multispecific antigen-binding molecules or antibodies provided herein are contemplated. For example, it may be desirable to alter the binding affinity and/or other biological properties of the multispecific antigen-binding molecule or antibody (e.g., anti-CCR8 antibody). Amino acid sequence variants of a multispecific antigen-binding molecule or antibody may be prepared by introducing appropriate modifications into the nucleotide sequence encoding the antigen-binding molecule or antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain aspects, antibody variants having one or more amino acid substitutions are provided. Sites of interest for substitutional mutagenesis include the CDRs and FRs.

In one aspect, the VL sequence of the antigen-binding molecule or antibody disclosed herein comprises a V4M mutation, a P43A mutation, a F46L mutation, a C90Q mutation, or a combination thereof. In one aspect, the VH sequence of the antibodies disclosed herein comprises a G49S mutation, a K71R mutation, a S73N mutation, or a combination thereof. In one aspect, the VL sequence of the antigen-binding molecules or antibodies disclosed herein comprises a Y2I mutation. In one aspect, the VH sequence of the antigen-binding molecules or antibodies disclosed herein comprises a S73N mutation, a V78L mutation, a T76N mutation, a F91Y mutation, and a P105Q mutation, or a combination thereof (e.g., numbered according to Kabat).

Conservative substitutions are shown in Table 3 under the heading of “conservative substitutions”. More substantial changes are provided in Table 3 under the heading of “exemplary substitutions”, and as further described below in reference to amino acid side chain classes. Amino acid substitutions may be introduced into an antigen-binding molecule or antibody of interest and the products screened for a desired activity, e.g., retained/improved antigen binding, decreased immunogenicity, or improved ADCC or CDC.

TABLE 3
Contemplated amino acid substitutions

| Original | | Conservative |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antigen-binding molecule or antibody (e.g., a humanized or human antigen-binding molecule or antibody). Generally, the resulting variant(s) selected for further study will have modifications (e.g., improvements) in certain biological properties (e.g., increased affinity, reduced immunogenicity) relative to the parent antigen-binding molecule or antibody and/or will have substantially retained certain biological properties of the parent antigen-binding molecule or antibody. An exemplary substitutional variant is an affinity matured antigen-binding molecule or antibody, which may be conveniently generated, e.g., using phage display-based affinity maturation techniques such as those described herein. Briefly, one or more CDR residues are mutated and the variant antigen-binding molecules or antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) provided herein is altered to increase or decrease the extent to which the antibody is glycosylated. Addition or deletion of glycosylation sites to a multispecific antigen-binding molecule or an antibody may be conveniently accomplished by altering the amino acid sequence such that one or more glycosylation sites is created or removed.

Where the multispecific antigen-binding molecule or antibody comprises an Fc region, the oligosaccharide attached thereto may be altered. Native antigen-binding molecules or antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody as described herein may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure, and such antibodies are further referred to herein as an “afucosylated antibodies.” In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e., no fucosylated oligosaccharides are present). In certain embodiments, the proportion of afucosylation is between about 65% to about 100%, between about 80% to about 100%, or between about 80% to about 95%. The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies, e.g., Asn 299. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

In one aspect, the present disclosure provides afucosylated antibody variants that have enhanced FcγRIIIa receptor binding. In one aspect, the present disclosure provides afucosylated antibody variants that have enhanced antibody-dependent cellular cytotoxicity (ADCC). In one aspect, the present disclosure provides afucosylated antibody variants that have antibody-dependent cellular phagocytosis (ADCP) activities.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see, e.g., US2004259150, US2005031613, US2004132140, US2004110282). See also Pereira et al., MABS (2018) 693-711.

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antigen-binding molecule or antibody variant that possesses some but not all effector functions, which make it a desirable candidate for applications in which the half-life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antigen-binding molecule or antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain aspects, an antigen-binding molecule or antibody variant comprises an Fc region with one or more amino acid substitutions which improve ADCC, e.g., substitutions at positions 298, 333, and/or 334 of the Fc region (EU numbering of residues).

In certain aspects, an antigen-binding molecule or antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antigen-binding molecule or antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In certain aspects, the Fc region comprises a modification configured to promote the association of the first Fc subunit with the second Fc subunit. “Knob-in-hole” engineering of multispecific antigen-binding molecule or antibodies may be utilized to generate a first arm containing a knob and a second arm containing the hole into which the knob of the first arm may bind. The knob of the multispecific antibodies of the invention may be a monovalent arm (e.g., anti-CCR8 arm) in one embodiment. Alternatively, the knob of the multispecific antigen-binding molecules or antibodies of the invention may be a bivalent arm. The hole of the multispecific antigen-binding molecules or antibodies of the invention may be a monovalent arm in one embodiment. Alternatively, the hole of the multispecific antigen-binding molecules or antibodies of the invention may be a bivalent arm. Multispecific antigen-binding molecules and antibodies may also be engineered using immunoglobulin crossover (also known as Fab domain exchange or CrossMab format) technology (see e.g., WO 2009/080253; Schaefer et al., Proc. Natl. Acad. Sci. USA, 108:11187-11192 (2011)). Multispecific antigen-binding molecules or antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229:81 (1985)); or by using leucine zippers to produce bispecific antibodies (see, e.g., Kostelny et al., J. Immunol., 148 (5): 1547-1553 (1992)).

An amino acid residue in the CH3 domain of the second Fc subunit may be replaced with an amino acid residue having a larger side chain volume, thereby generating a protuberance (e.g., a knob) within the CH3 domain of the second Fc subunit which is positionable in a cavity (e.g., a hole) within the CH3 domain of the first Fc subunit, and an amino acid residue in the CH3 domain of the first Fc subunit may be replaced with an amino acid residue having a smaller side chain volume, thereby generating a cavity (e.g., a hole) within the CH3 domain of the first Fc subunit within which the protuberance (e.g., a knob) within the CH3 domain of the second Fc subunit may be positionable. In some embodiments, the CH3 domain of the second Fc subunit comprises the amino acid substitution of T366, and the CH3 domain of the first Fc subunit comprises amino acid substitutions at one, two, or all three of T366, L368, and/or Y407. In some embodiments, the CH3 domain of the second Fc subunit comprises the amino acid substitution of T366W, and the CH3 domain of the first Fc subunit comprises one, two, or all three amino acid substitutions of T366S, L368A, and/or Y407V.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) comprises an Fc region with one or more amino acid substitutions which improve FcγR binding (and thereby improve effector function), e.g., substitutions at positions. In certain aspects, the antibody variant comprises an Fc region with at least one amino acid substitutions of G236A, 1332E, S298A, E333A, K334A, S239D, A330L, F243L, R292P, Y300L, V305I, P396L, L235V, L234Y, L235Q, G236W, S239M, H268D, D270E, K326D, A330M, K334E (See, e.g., Liu et al., Antibodies (Basel) (2020); 9 (4): 64).

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g., Dall'Acqua, W. F., et al. J. Immunol 169 (2002) 5171-5180). Residues I253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) comprises an Fc region with one or more amino acid substitutions, which reduce FcRn binding, e.g., substitutions at positions 253, and/or 310, and/or 435 of the Fc-region (EU numbering of residues). In certain aspects, the antigen-binding molecule or antibody comprises an Fc region with the amino acid substitutions at positions 253, 310 and 435. In one aspect, the substitutions are 1253A, H310A and H435A in an Fc region derived from a human IgG1 Fc-region. See, e.g., Grevys, A., et al., J. Immunol. 194 (2015) 5497-5508.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) comprises an Fc region with one or more amino acid substitutions, which reduce FcRn binding, e.g., substitutions at positions 310, and/or 433, and/or 436 of the Fc region (EU numbering of residues). In certain aspects, the antigen-binding molecule or antibody comprises an Fc region with the amino acid substitutions at positions 310, 433 and 436. In one aspect, the substitutions are H310A, H433A and Y436A in an Fc region derived from a human IgG₁ Fc-region. (See, e.g., WO 2014/177460 AI).

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) comprises an Fc region with one or more amino acid substitutions which increase FcRn binding, e.g., substitutions at positions 252, and/or 254, and/or 256 of the Fc region (EU numbering of residues). In certain aspects, the antigen-binding molecule or antibody comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or the antibody (e.g., anti-CCR8 antibody) as reported herein can be a complete C-terminus ending with the amino acid residues PGK. The C-terminus of the heavy chain can be a shortened C-terminus in which one or two of the C terminal amino acid residues have been removed. In one aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) comprising a heavy chain including a C-terminal CH3 domain, as specified herein, comprises a C-terminal glycine residue (G446, EU index numbering of amino acid positions). In one aspect of all aspects as reported herein, an antibody comprising a heavy chain including a C-terminal CH3 domain, as specified herein, comprises a C-terminal proline residue (P445, EU index numbering of amino acid positions).

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### e) Antibody Derivatives

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) provided herein may be further modified to contain additional nonproteinaceous moieties that are known in the art and readily available. The moieties suitable for derivatization of the antigen-binding molecule or antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### D. Recombinant Methods and Compositions

A multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) may be produced using recombinant methods and compositions, e.g., as described in U.S. Pat. No. 4,816,567 and in U.S. Publication No. 2013/0078249, each of which is incorporated herein by reference in its entirety. In one embodiment, isolated nucleic acid (e.g., a polynucleotide) encoding a bispecific antigen-binding molecule or an antibody described herein is provided. In one embodiment, isolated nucleic acid (e.g., a polynucleotide) encoding a bispecific antigen-binding molecule described herein is provided. Such nucleic acid may encode an amino acid sequence comprising the VL and/or an amino acid sequence comprising a VH of the bispecific antigen-binding molecule (e.g., the light and/or heavy chains of the either arm of the bispecific antigen-binding molecule) or antibody. In a further embodiment, one or more vectors (e.g., expression vectors) comprising such nucleic acid are provided.

In one aspect, the invention provides an isolated polynucleotide or a set of isolated polynucleotides comprising a nucleic acid sequence that is at least 85%, at least 90%, at least 95%, or at least 99% identical to the nucleic acid sequence of any one of SEQ ID NOs: 85-89. In one aspect, the invention provides an isolated polynucleotide or a set of isolated polynucleotides comprising a nucleic acid sequence that is at least 85%, at least 90%, at least 95%, or at least 99% identical to the nucleic acid sequence of SEQ ID NO: 85. In one aspect, the invention provides an isolated polynucleotide or a set of isolated polynucleotides comprising a nucleic acid sequence that is at least 85%, at least 90%, at least 95%, or at least 99% identical to the nucleic acid sequence of SEQ ID NO: 86. In one aspect, the invention provides an isolated polynucleotide or a set of isolated polynucleotides comprising a nucleic acid sequence that is at least 85%, at least 90%, at least 95%, or at least 99% identical to the nucleic acid sequence of SEQ ID NO: 87. In one aspect, the invention provides an isolated polynucleotide or a set of isolated polynucleotides comprising a nucleic acid sequence that is at least 85%, at least 90%, at least 95%, or at least 99% identical to the nucleic acid sequence of SEQ ID NO: 88. In one aspect, the invention provides an isolated polynucleotide or a set of isolated polynucleotides comprising a nucleic acid sequence that is at least 85%, at least 90%, at least 95%, or at least 99% identical to the nucleic acid sequence of SEQ ID NO: 89.

In one aspect, the invention provides isolated polynucleotide or a set of isolated polynucleotides comprising the nucleic acid sequence of any one of SEQ ID NOs: 85-89. In one aspect, the invention provides isolated polynucleotide or a set of isolated polynucleotides comprising the nucleic acid sequence of SEQ ID NO: 85. In one aspect, the invention provides isolated polynucleotide or a set of isolated polynucleotides comprising the nucleic acid sequence of SEQ ID NO: 86. In one aspect, the invention provides isolated polynucleotide or a set of isolated polynucleotides comprising the nucleic acid sequence of SEQ ID NO: 87. In one aspect, the invention provides isolated polynucleotide or a set of isolated polynucleotides comprising the nucleic acid sequence of SEQ ID NO: 88. In one aspect, the invention provides isolated polynucleotide or a set of isolated polynucleotides comprising the nucleic acid sequence of SEQ ID NO: 89.

In some instances, the amino acid sequence of SEQ ID NO: 33 is encoded by a nucleic acid sequence that is at least 85%, at least 90%, at least 95%, at least 99% identical, or is 100% identical to the nucleic acid sequence of SEQ ID NO: 86. In some instances, the amino acid sequence of SEQ ID NO: 34 is encoded by a nucleic acid sequence that is at least 85%, at least 90%, at least 95%, at least 99% identical, or is 100% identical to the nucleic acid sequence of SEQ ID NO: 85. In some instances, the amino acid sequence of SEQ ID NO: 35 is encoded by a nucleic acid sequence that is at least 85%, at least 90%, at least 95%, at least 99% identical, or is 100% identical to the nucleic acid sequence of SEQ ID NO: 88. In some instances, the amino acid sequence of SEQ ID NO: 36 is encoded by a nucleic acid sequence that is at least 85%, at least 90%, at least 95%, at least 99% identical, or is 100% identical to the nucleic acid sequence of SEQ ID NO: 87. In some instances, the amino acid sequence of SEQ ID NO: 90 is encoded by a nucleic acid sequence that is at least 85%, at least 90%, at least 95%, at least 99% identical, or is 100% identical to the nucleic acid sequence of SEQ ID NO: 89.

Polynucleotides encoding an antibody (e.g., an anti-CCR8 antibody) or a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) of the invention may be expressed as a single polynucleotide molecule or as multiple (e.g., two or more) polynucleotides that are co-expressed.

Polypeptides encoded by polynucleotides that are co-expressed may associate through, e.g., disulfide bonds or other means to form a functional bispecific antigen-binding molecule. For example, a light chain portion of an antigen binding moiety may be encoded by a separate polynucleotide from the portion of the bispecific antigen-binding molecule comprising the heavy chain portion of the antigen binding moiety, an Fc domain subunit and optionally another antigen binding moiety. When co-expressed, the heavy chain polypeptides will associate with the light chain polypeptides to form the antigen binding moiety. In another example, the portion of the bispecific antigen-binding molecule comprising one of the two Fc domain subunits and optionally one or more antigen-binding moieties could be encoded by a separate polynucleotide from the portion of the T cell activating bispecific antigen binding molecule comprising the other of the two Fc domain subunits and optionally an antigen binding moiety. When co-expressed, the Fc domain subunits will associate to form the Fc domain.

In certain embodiments, an isolated polynucleotide of the invention encodes a fragment of a bispecific antigen-binding molecule comprising a first and a second antigen-binding domain, and an Fc domain consisting of two subunits. In one embodiment, an isolated polynucleotide of the invention encodes the first antigen binding moiety and a subunit of the Fc domain. In another embodiment, an isolated polynucleotide of the invention encodes the heavy chain of the second antigen binding moiety and a subunit of the Fc domain. In a more specific embodiment, the isolated polynucleotide encodes a polypeptide, wherein a Fab heavy chain shares a C-terminal peptide bond with an Fc domain subunit. In yet another embodiment, an isolated polynucleotide of the invention encodes the heavy chain of the third antigen-binding domain, the heavy chain of the second antigen binding moiety, and a subunit of the Fc domain. In some embodiments, the light chains of the second and third antigen-binding moieties are co-expressed and associate with the heavy chain regions to form Fab domains.

In a further embodiment, a host cell comprising such nucleic acid is provided. In one such embodiment, a host cell comprises (e.g., has been transformed with): (1) a vector comprising a nucleic acid that encodes an amino acid sequence comprising at least one VL of the bispecific antigen-binding molecule and an amino acid sequence comprising at least one VH of the bispecific antigen-binding molecule, or (2) a first vector comprising a nucleic acid that encodes an amino acid sequence comprising a VL of the bispecific antigen-binding molecule and a second vector comprising a nucleic acid that encodes an amino acid sequence comprising a VH of the bispecific antigen-binding molecule. In one embodiment, the host cell is eukaryotic, e.g., a Chinese Hamster Ovary (CHO) cell or lymphoid cell. In one embodiment, a method of making a bispecific antigen-binding molecule is provided, wherein the method comprises culturing a host cell comprising a nucleic acid encoding the bispecific antigen-binding molecule, as provided above, under conditions suitable for expression of the antibody, and optionally recovering the antibody from the host cell (or host cell culture medium).

For recombinant production of a bispecific antigen-binding molecule, nucleic acids encoding the bispecific antigen-binding molecule, e.g., as described above, is isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the bispecific antigen-binding molecule) or produced by recombinant methods or obtained by chemical synthesis.

For recombinant production of an antibody (e.g., a bispecific) antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J.P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N. Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A.M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### One-Cell Approach for Multispecific Antigen-Binding Molecule Production

In one approach, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) can be produced by culturing host cells that have been co-transfected with two plasmids, each encoding one of the two arms of the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)). Transfection of host cells (e.g., bacterial, mammalian, or insect cells) may be performed in a 96-well plate format. To screen for anti-CCR8/anti-CD3 TDB production, approximately 2,000 to 3,000 clones may be picked and assessed by ELISA and intact IgG homogeneous time resolved fluorescence (HTRF) for their ability to bind the target antigen, CCR8. Clones producing anti-CCR8/anti-CD3 TDBs capable of binding to CCR8, or a fragment thereof, may be selected for expansion and further screening (e.g., for binding to CD3). Top clones can then be selected for further analysis based on percent bispecific antibodies (bsAbs) produced, titer, and performance qualification (PQ).

An exemplary one-cell approach that can be used to produce a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) of the invention is described in International Patent Application No. PCT/US16/28850 or U.S. Patent Application Publication No. 2018/0177873, each of which is incorporated herein by reference in its entirety.

For example, provided herein is a method for producing a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) disclosed herein, comprising (a) introducing one or more polynucleotide molecules encoding a multispecific antigen-binding molecule into a host cell, wherein the multispecific antigen-binding molecule comprises a) a first heavy chain/light chain pair which comprises a first heavy chain polypeptide (H1) and a first light chain polypeptide (L1) comprising a first Fab molecule (Fab_{A}), e.g., that specifically binds to CCR8, and b) a second heavy chain/light chain pair which comprises a second heavy chain polypeptide (H2), a second light chain polypeptide (L2), and a third light chain polypeptide (L3) comprising a second Fab molecule (Fab_{B1}), e.g., that specifically binds to CD3, and a third Fab molecule (Fab_{B2}), e.g., that specifically binds to CCR8, wherein H1 comprises a heavy chain variable domain (VH_{A1}) and a heavy chain constant domain (CH1), H2 comprises two heavy chain variable domains (VH_{B1}) and (VH_{B2}), and each L1, L2, and L3 comprises a light chain variable domain (VL) and a light chain constant domain (CL), wherein: (i) an amino acid at S183 (EU numbering) in the heavy chain of each of the Fab_{A} and the Fab_{B2} is replaced with a positively charged residue, an amino acid at Q39 (Kabat numbering) in the heavy chain of each of the Fab_{A} and the Fab_{B2} is replaced with a negatively charged residue, an amino acid at V133 (EU numbering) in the light chain of each of the Fab_{A} and the Fab_{B2} is replaced with a negatively charged residue, and an amino acid at Q38 (Kabat numbering) in the light chain of each of the Fab_{A} and the Fab_{B2} is replaced with a positively charged residue; and an amino acid at S183 (EU numbering) in the heavy chain of the Fab_{B1} is replaced with a negatively charged residue, an amino acid at Q39 (Kabat numbering) in the heavy chain of the Fab_{B1} is replaced with a positively charged residue, an amino acid at V133 (EU numbering) in the light chain of the Fab_{B1} is replaced with a positively charged residue, and an amino acid at Q38 (Kabat numbering) in the light chain of the Fab_{B1} of H2 is replaced with a negatively charged residue; or (ii) an amino acid at S183 (EU numbering) in the heavy chain of each of the Fab_{A} and the Fab_{B2} is replaced with a negatively charged residue, an amino acid at Q39 (Kabat numbering) in the heavy chain of each of the Fab_{A} and the Fab_{B2} is replaced with a positively charged residue, an amino acid at V133 (EU numbering) in the light chain of each of the Fab_{A} and the Fab_{B2} is replaced with a positively charged residue, and an amino acid at Q38 (Kabat numbering) in the light chain of each of the Fab_{A} and the Fab_{B2} is replaced with a negatively charged residue; and an amino acid at S183 (EU numbering) in the heavy chain of each of the Fab_{B1} is replaced with a positively charged residue, an amino acid at Q39 (Kabat numbering) in the heavy chain of the Fab_{B1} is replaced with a negatively charged residue, an amino acid at V133 (EU numbering) in the light chain of the Fab_{B1} is replaced with a negatively charged residue, and an amino acid at Q38 (Kabat numbering) in the light chain of the Fab_{B1} of H2 is replaced with a positively charged residue; and (b) culturing the host cell under conditions and for a time sufficient to produce the antigen-binding protein. In some embodiments, the positively charged residue is selected from R and K and the negatively charged residue is selected from D and E. In some embodiments, the positively charged residue is K. In some embodiments, the negatively charged residue is E. In some embodiments, the first and third Fab molecules (e.g., Fab and Fab_{B2}) each specifically binds to CCR8 and the second Fab molecule (Fab_{B1}) specifically binds to CD3.

In some embodiments, the multispecific antigen-binding molecule described herein comprises a first Fab molecule (Fab_{A}) and a third Fab molecule (Fab_{B2}) that each specifically binds to CCR8 comprising Q39E (Kabat numbering) and S183K (EU numbering) substitutions in the heavy chain and Q38K (Kabat numbering) and V133E (EU numbering) substitutions in the light chain; and a second Fab molecule (Fab_{B1}) that specifically binds to CD3 comprising Q39K (Kabat numbering) and S183E (EU numbering) substitution in the heavy chain and Q38E (Kabat numbering) and V133K substitutions (EU numbering) in the light chain.

In some embodiments, the multispecific antigen-binding molecule described herein comprises a first Fab molecule (Fab_{A}) and a third Fab molecule (Fab_{B2}) that each specifically binds to CCR8 comprising Q39K (Kabat numbering) and S183E (EU numbering) substitutions in the heavy chain and Q38E (Kabat numbering) and V133K (EU numbering) substitutions in the light chain; and a second Fab molecule (Fab_{B1}) that specifically binds to CD3 comprising Q39E (Kabat numbering) and S183K (EU numbering) substitution in the heavy chain and Q38K (Kabat numbering) and V133E (EU numbering) substitutions in the light chain.

In a particular example, a polynucleotide encoding the amino acid sequence of H1 comprises the nucleic acid sequence of SEQ ID NO: 86; a polynucleotide encoding the amino acid sequences of each of L1 and L3 comprises the nucleic acid sequence of SEQ ID NO: 85; a polynucleotide encoding the amino acid sequence of H2 comprises the nucleic acid sequence of SEQ ID NO: 88; and a polynucleotide encoding the amino acid sequence of L2 comprises the nucleic acid sequence of SEQ ID NO: 87. In another particular example, a polynucleotide encoding the amino acid sequence of H1 comprises the nucleic acid sequence of SEQ ID NO: 86; a polynucleotide encoding the amino acid sequences of each of L1 and L3 comprises the nucleic acid sequence of SEQ ID NO: 85; a polynucleotide encoding the amino acid sequence of H2 comprises the nucleic acid sequence of SEQ ID NO: 89; and a polynucleotide encoding the amino acid sequence of L2 comprises the nucleic acid sequence of SEQ ID NO: 87.

In one example, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) of the invention may be produced as described in Dillon et al. MAbs 9:213-230, 2017.

### Two-Cell Approach for Multispecific Antigen-Binding Molecule Production

Alternatively, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) can be produced by culturing the antibody hemimers (e.g., half-antibodies) separately (i.e., in two different cell lines) using high-cell density fermentation and then isolating each half-antibody independently by Protein A chromatography. The purified half-antibodies can then be combined, for example, at a 1:1 molar ratio and incubated in 50 mM Tris, pH 8.5 in the presence of 2 mM DTT for 4 hours to allow annealing and the reduction of disulfides in the hinge region. Dialysis against the same buffer without DTT for 24-48 hours resulted in the formation of the inter-chain disulfide bonds.

TDBs may be alternatively produced by transfection of two plasmids, each encoding the distinct arms of the TDB, into separate host cells. The host cells may be co-cultured or cultured separately. Transfection of host cells may be performed in a 96-well plate format. To screen for TDB production, 2,000 to 3,000 clones may be picked and assessed by ELISA and intact IgG homogeneous time resolved fluorescence (HTRF) for their ability to bind a selected antigen (e.g., CCR8). Clones producing TDBs capable of binding to CCR8, or a fragment thereof, may be selected for expansion and further screening. Top clones are selected for further analysis based on percent bispecific antibodies (bsAbs) produced, titer, and PQ.

### Exemplary Production Methods

In one example, TDBs may be produced by a co-culture strategy using E. coli cells expressing one half-antibody (hole) and E. coli cells expressing the second half-antibody (knob) were grown together in shaker flasks at a predetermined ratio such that it produced similar amounts of each half-antibody (see, Spiess et al., Nat. Biotechnol. 31 (8): 753-8 (2013); PCT Pub. No. WO 2011/069104, which is incorporated herein by reference in its entirety). The co-cultured bacterial broth is then harvested, the cells disrupted in a microfluidizer and the antibodies purified by Protein A affinity. It is observed that during microfluidizing and protein A capture the two arms anneal and form the hinge inter-chain disulfide bridges.

In another example, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is produced as previously described (Junttila et al. Cancer Res. 74:5561-5571, 2014; Sun et al. Science Trans. Med. 7: 287ra270, 2015). Briefly, the two half-antibodies, i.e., containing “knob” or “hole” mutations in their CH3 domains, are expressed by transient transfection of CHO cells and then affinity purified with Protein A. Equal amounts of the two half-antibodies are incubated with a 200 molar excess of reduced glutathione at pH 8.5 overnight at 32° C. to drive the formation of the knob-hole disulfide bonds. The assembled multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is purified from contaminants through hydrophobic interaction chromatography. The purified anti-CCR8/anti-CD3 TDBs are characterized for purity by mass spectrometry, size exclusion chromatography (SEC), and/or gel electrophoresis.

### E. Assays

In some aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) provided herein may be identified, screened for, or characterized for their physical/chemical properties and/or biological activities by various assays known in the art.

### 1. Binding Assays and Other Assays

In one aspect, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) as described herein is tested for its antigen binding activity, e.g., by known methods such as ELISA, Western blot, etc.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for antigen-binding activity. In an exemplary method to test for antigen-binding activity, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) may be labeled via amine conjugation using ALEXA FLUOR™ 488 with an incorporation of ˜4 fluorophores/molecule. One million Chinese hamster ovary (CHO) cells stably expressing either human or cynomolgus CCR8 (˜60,000 and 30,000 receptors/cell, respectively) are seeded at a concentration of 1 million cells/mL onto a Multidish 2×2 Petri Dish (Ridgeview Instruments AB, Sweden) to form a circular drop of ˜ 3 cm diameter in chambers A and C of the dish. Untransfected CHO cells are seeded in opposing chambers (B and D) in the same manner. Cells are left undisturbed to adhere for 4 hours. Excess media is aspirated, fresh growth media is added, and cells are incubated overnight at 37° C., 5% CO₂. The following day, media is aspirated and fresh media is added. The cells are incubated at room temperature for 20 minutes prior to starting the assay. The plate is loaded onto a LIGANDTRACER® Green instrument (Ridgeview Instruments AB) for real-time fluorescence measurement. After the fluorescent signal stabilized (15-30 minutes), labeled bispecific antigen-binding molecule is added in two increasing concentrations (1 nM and 10 nM) until curvature is obtained. Antibody (Ab) dissociation is recorded by aspirating the media and adding fresh media with 10 nM of unlabeled Ab to prevent rebinding of labeled Ab. The Ab dissociation is measured overnight to obtain accurate off-rates. Untransfected CHO cells are used to subtract the non-specific binding. Specific binding traces are analyzed using Trace Drawer 1.7 (Ridgeview Instruments) software and fit with a 1-to-2 with bulk binding model to determine association (kₐ) and dissociation (k_{d}) rate constants. The equilibrium dissociation constants (K_{D}) for each binding event are calculated as the ratio of the dissociation and association rates, k_{d}/kₐ.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for binding to sulfated GPCRs (e.g., CCR2, CCR3, CCR4, CCR5, CXCR4, ACKR2, ACKR4, or CCR8, among others) by flow cytometry. In an exemplary method to test for the binding of a multispecific antigen-binding molecule described herein to sulfated GPCRs, HEK293 cells are seeded at 0.6 million in 1 mL of DMEM medium with 10% fetal calf serum (FCS) per well in 12-well cell culture plate and cultured overnight at 37° C. in 5% CO₂ incubator. Cells are then transfected with various DNA constructs encoding said sulfated GPCRs, which all have C-terminal Myc-tag, using TRANSIT-X2® (Mirus Cat. #MIR6000) with reagent:DNA=3:1 for 24 hours. Transfected HEK293 cells are harvested and stained with the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)), the antibody (e.g., anti-CCR8 antibody), or isotype control antibody anti-gD in FACS buffer (PBS with 0.5% BSA and 2 mM EDTA) (at 10 μg/mL final concentration) at 4° C. for 30 min, then washed twice with FACS buffer. Cells are then stained with ALEXA FLUOR® 647-anti-Human IgG (Jackson Immunol Lab, 109-606-170, 1:500) at 4° C. for 15 min. Cells are washed twice with FACS buffer. Cells are then fixed and permeabilized with Fixation/Permeabilization Kit (BD Bioscience, Cat. #554714) and followed by ALEXA FLUOR® 488 Anti-Myc tag antibody (9E10, ab202008, 1:100) staining at 4° C. for 30 min. Cells are washed twice and analyzed on FACSCELESTA® and data is analyzed with FLOWJO® software.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for binding affinity to human or cyno CCR8. In an exemplary method to test for the binding affinity of a multispecific antigen-binding molecule described herein to human or cyno CCR8, CCR8-TDB is labeled via amine conjugation using ALEXA FLUOR® 488 with an incorporation of about 4 fluorophores/molecule. 1 million CHO cells stably expressing either human or cynomolgus CCR8 (about 60,000 and about 30,000 receptors/cell, respectively) are seeded at a concentration of 1 million cells/mL onto a Multidish 2×2 Petri Dish (Ridgeview Instruments AB, Sweden) to form a circular drop of about 3 cm diameter in chambers A and C of the dish. Untransfected CHO cells are seeded in opposing chambers (B and D) in the same manner. Cells are left undisturbed to adhere for 4 hours. Excess media is aspirated, fresh growth media is added, and cells are incubated overnight at 37° C., 5% CO₂. The following day, media is aspirated, and fresh media is added. The cells are incubated at room temperature for 20 minutes prior to starting the assay. The plate is loaded onto a LIGANDTRACER® Green (Ridgeview Instruments AB) for real-time fluorescence measurement. After the fluorescent signal stabilizes (15-30 minutes), labeled multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or antibody (e.g., anti-CCR8 antibody) is added in two increasing concentrations (1 nM and 10 nM) until curvature is obtained. Antibody (Ab) dissociation is recorded by aspirating the media and adding fresh media with 10 nM of unlabeled CCR8-TDB to prevent rebinding of labeled Ab. The Ab dissociation is measured overnight to obtain accurate off-rates. Untransfected CHO cells are used to subtract the non-specific binding. Specific binding traces are analyzed using TraceDrawer 1.7 (Ridgeview Instruments) software to determine the association-(kₐ) and dissociation-(k_{d}) rate constants. The equilibrium dissociation constants (K_{D}) for each binding event are calculated as the ratio of the dissociation and association rates, k_{d}/kₐ. Three independent experiments are performed for each cell line.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for binding affinity to human or cyno CD3 by surface plasmon resonance (SPR) on a BIACORE™ T200 instrument (Cytiva; Marlborough, MA). In an exemplary kinetics measurements for the binding affinity of a multispecific antigen-binding molecule described herein toward human CD3, the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or the antibody (e.g., anti-CCR8 antibody) is captured by mouse anti-human Fc antibody (Cytiva, cat #BR-1008-39) coated on CM5 biosensor chips to achieve approximately 250 response units (RU). Human CD3-epsilon/delta dimer (linked by murine Fc) is injected in three-fold serial dilutions from 1800 nM to 0 nM in HBS-P buffer (10 mM HEPES, 150 mM NaCl, 0.05% polysorbate 20, pH 7.4) at 37° C. with a flow rate of 100 μL/min. For cyno CD3 kinetics measurements, biotinylated cyno CD3 epsilon peptide (Pyr-DGNEEMGSITQTPYQVSISGTTVILTKK-biotin-amide, SEQ ID NO: 122) is captured on CAP chip (Cytiva, cat #28920234). CCR8-TDB is injected in three-fold serial dilutions from 1800 nM to 0 nM in HBS-P buffer at 37° C. with a flow rate of 100 μL/min. Association rates (kₐ) and dissociation rates (k_{d}) are calculated using a 1:1 Langmuir binding model (BIACORE™ T200 Evaluation Software version 3.1). The equilibrium dissociation constant (K_{D}) is calculated as the ratio k_{d}/kₐ.

### 2. Activity Assays

In one aspect, assays are provided for identifying a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) having biological activity. Biological activity may include, e.g., antibody-dependent target cell killing, Treg depletion, pharmacokinetics, stimulation of cytokine release, tumor growth inhibition, and the like.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is tested for its target cell killing activity. An anti-CCR8/anti-CD3 bispecific antigen-binding molecule may be tested for its target cell killing activity on CCR8.CHO cells. In an exemplary method to test for the target cell killing activity of the anti-CCR8/anti-CD3 bispecific antigen-binding molecule described herein on CCR8.CHO cells, human peripheral blood mononuclear cells (PBMC) are isolated from healthy donor blood using a FICOLL® density gradient centrifugation with LYMPHOPREP® solution (STEMCELL Technologies). CD8+ T cells are magnetically enriched using human CD8+ T Cell isolation kit (Miltenyi Biotec). Human CCR8-expressing Chinese Hamster Ovary (CHO-hCCR8-Ga15) cells are plated in the black, clear-bottomed 96-well plates at density of 10,000 cells per well. CD8+ T cells and CHO-hCCR8 cells were cocultured at a 3:1 ratio with anti-CCR8/CD3-TDB at indicated concentrations. After 48 hours, the plates were washed twice with phosphate-buffered saline (PBS). 100 UL of CELLTITER-GLO® Luminescent Cell Viability reagent (Promega cat #G7570) is added and plates were read on a luminometer as described in the instructions. The target cell killing activity is calculated as: {(reading without TDB-reading with TDB)/(reading without TDB)}×100%.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for its ability to deplete CCR8+ Treg cells. In an exemplary method to test for the ability of a multispecific antigen-binding molecule described herein to deplete CCR8+ Treg cells, peripheral blood mononuclear cells (PBMC) are isolated from healthy donor blood using a FICOLL® density gradient (GE Healthcare). T cells are magnetically enriched using human Pan T Cell isolation kit (Miltenyi Biotec). T cells (1×10⁵ cells/well) were incubated for 48 hours at 37° C. in culture media with indicated concentrations of individual bispecific antigen-binding molecules or antibodies in a U-bottom 96-well plate. After 2 days, cells are harvested and sequentially stained with fixable viability dye (ThermoFisher Scientific), blocked with human FcR-blocking reagent (Miltenyi Biotec), stained with surface markers, and then intracellular marker using the Foxp3 transcription factor staining buffer set (ThermoFisher Scientific) following the manufacturer's protocols. Samples are acquired by flow cytometry (Symphony, BD) and analyzed using FLOWJO® software package (BD Biosciences). Cell numbers were quantified with COUNTBRIGHT® absolute counting beads (ThermoFisher Scientific).

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for its ability to inhibit tumor growth. In an exemplary method, the efficacy of anti-CCR8/anti-CD3 TDBs to inhibit tumor growth is investigated in mouse breast cancer syngeneic model, E0771, and mouse colorectal syngeneic model, MC-38. Animal studies using these cell lines are carried out in compliance with National Institutes of Health guidelines for the care and use of laboratory animals and were approved by the Institutional Animal Care and Use Committee (IACUC) at Genentech. To establish the models, 0.1 million tumor cells (suspended in 0.1 mL of Hanks' Balanced Salt Solution (HBSS) with MATRIGEL®) are inoculated, into the mammary fat pad #5 for the E0771 model or subcutaneously into the flank for the MC-38, of female hu.CD3E.tg.B6N mice (Genentech; Dixon, CA). Hu.CD3E.tg.B6N transgenic mice have a C57BI/6 mouse background and are genetically engineered to express human CD3. When tumors reached the desired volume (˜137 mm³), animals are divided into groups of n=5-7 with similar distribution of tumor volumes, and receive a single intravenous dose of vehicle (20 mM histidine acetate, 240 mM sucrose, 0.02% polysorbate-20, pH 5.5) or TDB through the tail vein (referred to as Day 0). Tumors are measured in two dimensions (length and width) using calipers and tumor volume was calculated using the formula: Tumor size (mm³)=0.5×(length×width×width). Tumor sizes and mouse body weights are recorded twice weekly over the course of the study. Mice whose tumor volume exceeded 2000 mm³ or whose body weight loss was 20% of their starting weight are promptly euthanized per IACUC guidelines. Data are analyzed using R statistical software system (R Foundation for Statistical Computing; Vienna, Austria), and a mixed modeling is fit within R using the nlme package (Pinheiro et al. 2013). Cubic regression splines are used to fit a non-linear profile to the time courses of log 2 tumor volume of each treatment. These non-linear profiles are then related to the treatment within the mixed model. This approach addresses both repeated measurements and modest dropouts due to any non-treatment-related removal of animals before study end.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is tested for its ability to elicit cytokine release in vivo. In an exemplary method, cytokine release following anti-CCR8/anti-CD3 TDB (T cell-dependent bispecific) treatment is investigated in mouse colorectal syngeneic model, MC-38. The animal study using this cell line is carried out in compliance with National Institutes of Health guidelines for the care and use of laboratory animals and was approved by the Institutional Animal Care and Use Committee (IACUC). To establish the model, 0.1 million tumor cells (suspended in 0.1 mL of Hanks' Balanced Salt Solution (HBSS) with MATRIGEL®) are subcutaneously inoculated into the flank of female hu.CD3E.tg.B6N mice (Genentech; Dixon, CA). Hu.CD3E.tg.B6N transgenic mice have a C57BI/6 mouse background and are genetically engineered to express human CD3. When tumors reach the desired volume (˜136 mm³), animals are divided into groups of n=5 with similar distribution of tumor volumes, and receive a single intravenous dose of vehicle (20 mM histidine acetate, 240 mM sucrose, 0.02% polysorbate-20, pH 5.5) or TDB through the tail vein (referred to as Day 0). Blood samples are collected from mice 2 hours post-dose and processed for serum. Cytokines in serum are analyzed using the mouse cytokine LUMINEX® assay (Millipore) according to the manufacturer instructions.

In certain aspects, the pharmacokinetic properties of a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested. In an exemplary method to assess the pharmacokinetic properties of a multispecific antigen-binding molecule described herein, C.B-17 severe combined immunodeficiency (SCID) mice (non-binding species) are distributed into 4 groups with n=4 mice per group. Mice receive a single intravenous (IV) bolus injection of 5 mg/kg of the anti-CCR8/anti-CD3 bispecific antigen-binding molecule or antibody. All test articles were supplied as liquid stock solutions and diluted to 0.92 mg/mL for all groups. The dose volume administered was 5.43 mL/kg for all groups. For all groups, blood is collected from each animal at 0.1667, 1, 6, 24, 72, 168, 240, 336, and 504 hours post dose. 35-40 μL of whole blood is collected via tail vein bleed into SST tubes and stored at room temperature for 30-60 minutes before being processed to serum by centrifugation (11,000 rpm for 5 minutes). The concentrations of test articles in the mouse serum are determined using a total human IgG (generic immunoglobulin pharmacokinetics (GRIP)) ELISA. Serum samples for pharmacokinetic (PK) analysis are collected at 0.1667, 1, 6, 24, 72, 168, 240, 336, and 504 hours post-dose. Samples collected at these time-points are used to plot serum concentration versus time curves. The serum concentration versus time data from each animal are analyzed using the IV bolus input model, WINNONLIN®, Version 8.2 (Certara; Princeton, NJ). The following PK parameters were estimated from the PK curves:
Cₘₐₓ: Maximum observed concentration.
AUCₐₗₗ: Area under the serum concentration versus time curve from Time 0 to time of the last measurable concentration.
AUC_{inf}: Area under the serum concentration versus time curve extrapolated to infinity
CL: Clearance (Dose/AUC_{inf}).
Vₛₛ: Volume of distribution at steady state
t_{1/2}: Terminal half-life (ln[2]/z)

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) as described herein is tested for measuring depletion of Treg cells in vivo. In an exemplary method to test the ability of a multispecific antigen-binding molecule described herein to deplete Treg cells in vivo, mice with established tumors are treated with the bispecific antigen-binding molecule, and the proportion of Treg cells, conventional CD4 T cells and CD8 T cells among leukocytes in tumors, spleen and tumor-draining lymph nodes are analyzed. To this end, tumor cells are harvested in log-phase growth and resuspended in HBSS containing MATRIGEL® at a 1:1 ratio. Mice are inoculated subcutaneously in the flank with 0.1 million tumor cells in 100 microliters of HBSS+MATRIGEL®. Tumors are monitored until they become established and reached a mean tumor volume 130-230 mm³. Mice are then randomized into treatment groups. Treatment with an anti-CCR8 or an anti-gp120 isotype control Ab is administered intravenously. Three days later mice are sacrificed and tumors, spleens and tumor-draining lymph nodes obtained for analysis. To generate single cell suspensions, tumors are minced and digested. Single cell suspensions are surface stained with fluorescently labelled anti-CD45, anti-CD4 and anti-CD8 antibodies and intracellularly stained with fluorescently labelled anti-Foxp3 antibody. Flow cytometry may be performed on a FORTESSA™ X-20 or FACSYMPHONY™ and analyzed with FLOWJO® software.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) as described herein is tested for tumor growth inhibition following anti-CCR8-mediated depletion of tumor-infiltrating Treg cells in vivo. In an exemplary method to test the ability of a multispecific antigen-binding molecule described herein to inhibit tumor growth following anti-CCR8-mediated depletion of tumor-infiltrating Treg cells in vivo, mice with established tumors are treated with a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) and are monitored for tumor growth over time.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) is tested for its ability to block CCL1-mediated activation of CCR8 by monitoring Ca²⁺ influx using Fluorescent Imaging Plate Reader (FLIPR™) assay with FDSS/μCell (Hamamatsu, Japan). In an exemplary method, CHO/hCCR8.Gna 15 cells are seeded in 384-well plate (Corning, Cat. #3764) at 10,000 cells per well in F-12K medium supplemented with 10% FBS. On the second day, the cells are loaded with fluorescence Ca²⁺ dye Fluo-8 NW (Cat #36307, AAT Bioquest) and incubated for 30 minutes at 37° C. followed by 30 minutes incubation at room temperature. Serial diluted multispecific antigen-binding molecule (e.g., a multispecific antibody; e.g., a bispecific binding molecule; e.g., anti-CCR8/anti-CD3 bispecific antibody; e.g., anti-CCR8/anti-CD3 TDB)) or antibody (e.g., anti-CCR8 antibody) and control antibody (BD, Cat. #433H) are prepared in Hank's Buffer with HEPES (HHBS) in a clear 384-well plate and hCCL1 in HHBS buffer is also aliquoted in a clear 384-well plate. The FLIPR™ assay is set up on the FDSS/μCell plate reader with continuous monitoring (reading every second) for a total 500 seconds, with antibody addition at 10 second and hCCL1 addition at 300 second. Excitation and emission are set at wavelength 485 nm and 525 nm, respectively. After the run, negative control correction is applied, and the data are normalized against the signal of hCCL1 alone (100%) and plotted as a function of antibody concentrations using GRAPHPAD PRISM®.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is tested for its ability to deplete CCR8+ Treg cells in the peripheral blood of cynomolgus monkeys. In an exemplary method to test the ability of a multispecific antigen-binding molecule described herein to deplete CCR8+ Treg cells in the peripheral blood of cynomolgus monkeys, 3 male cynomolgus monkeys per group are administered either vehicle control or anti-CCR8 TDB intravenously on Day 1 and Day 11. Cynomolgus monkeys are of Cambodian origin and approximately 2-4 kg and 2-6 years old. Animals are randomly assigned to receive either 20 mM Histidine acetate, 0.15 M NaCl, pH5.5 (control) or anti-CCR8 TDB at 10 mg/kg via 1-hour intravenous infusion at 2 mL/kg/hr. Blood samples are collected at pre-study Day −4 (Day 36 of pre-observation), Day 3, Day 7, Day 11 (pre-dose), Day 13 and Day 20. 500 μL of total blood per condition is divided into 5 separate tubes with 100 μL blood and each is lysed using 2 mL of 1× Lysing Buffer (BD Biosciences Cat. #555899) for 15 minutes at room temperature. The samples are washed with 1.5 mL Wash Buffer and centrifuged at 500×g for 5 mins at room temperature. All 5 tubes are resuspended in 200 μL of Wash Buffer, then combined into one tube and centrifuged at 500×g for 5 mins at room temperature before surface staining. All the samples are blocked with 5 μL of purified anti-human CD32 antibody (BD Biosciences Cat. #303202) and 5 μL of monocyte blocker (BD Biosciences Cat. #426103) for 10 minutes. Afterwards, cells are stained with the antibody mixture B or C surface antibodies for 45 minutes in a refrigerator while protected from the light. The composition of antibody mixture B and C are shown in Tables 4 and 5 below, respectively.

TABLE 4
Composition of antibody mixture B (for cell surface staining, 90 μL/tube)

| Name | Clone | Cat. No. | Amount/Test | | Manufacturer |
|---|---|---|---|---|---|
| Stain Buffer (FBS) | — | 554656 | 28.5 | μL | A |
| Brilliant Stain Buffer Plus | — | 566385 | 10 | μL | A |
| BUV395 Mouse Anti-NHP CD45 | D058- 1283 | 564099 | 5 | μL | A |
| BUV737 Mouse Anti-Human CD4 | OKT4 | 750977 | 5 | μL | A |
| Brilliant Violet 421 anti-human/mouse/rat | C398.4 A | 313524 | 5 | μL | B |
| CD278 (ICOS) Antibody | | | | | |
| Brilliant Violet 510 anti-human CD8 Antibody | SK1 | 344732 | 5 | μL | B |
| Brilliant Violet 650 anti-human CD127 | A019D5 | 351326 | 5 | μL | B |
| (IL-7Rα) Antibody | | | | | |
| BV786 Mouse Anti-Human CD25 | M-A251 | 563701 | 5 | μL | A |
| PE-Cy7 Mouse Anti-Human CD45RA | 5H9 | 561216 | 5 | μL | A |
| FITC anti-human CD69 Antibody | FN50 | 310904 | 5 | μL | B |
| PerCP/Cyanine5.5 anti-human CD14 Antibody | M5E2 | 301824 | 5 | μL | B |
| PE Anti-CCR8 Antibody produced in Rabbit | [CCR8 (1912)] | — | 6.5 | μL | Genentech |

A: BD Biosciences, B: Biolegend, Inc.

TABLE 5
Composition of antibody mixture C (for cell surface staining, 90 μL/tube)

| | | | Amount/ | | |
|---|---|---|---|---|---|
| Name | Clone | Cat. No. | Test | | Manufacturer |
| Stain Buffer (FBS) | — | 554656 | 27.9 | μL | A |
| Brilliant Stain Buffer Plus | — | 566385 | 10 | μL | A |
| BUV395 Mouse Anti-NHP CD45 | D058- 1283 | 564099 | 5 | μL | A |
| BUV737 Mouse Anti-Human CD4 | OKT4 | 750977 | 5 | μL | A |
| Brilliant Violet 421 anti-human/mouse/rat | C398.4 A | 313524 | 5 | μL | B |
| CD278 (ICOS) Antibody | | | | | |
| Brilliant Violet 510 anti-human CD8 Antibody | SK1 | 344732 | 5 | μL | B |
| Brilliant Violet 650 anti-human CD127 | A019D5 | 351326 | 5 | μL | B |
| (IL-7Rα) Antibody | | | | | |
| BV786 Mouse Anti-Human CD25 | M-A251 | 563701 | 5 | μL | A |
| PE-Cy7 Mouse Anti-Human CD45RA | 5H9 | 561216 | 5 | μL | A |
| FITC Mouse IgG1, κ Isotype Ctrl Antibody | MOPC- 21 | 400107 | 5 | μL | B |
| PerCP/Cyanine5.5 anti-human CD14 Antibody | M5E2 | 301824 | 5 | μL | B |
| PE Anti-CCR8 Isotype Antibody produced in Rabbit | isotype | — | 7.1 | μL | — |

A: BD Biosciences, B: Biolegend, Inc.

After the surface staining, all the samples are washed 3 times with 1.5 mL Wash Buffer and centrifuged at 500×g for 5 minutes at 4° C. At the end of the last wash, supernatant is discarded and 500 μL 1× Fix/Perm buffer (eBioscince Cat. #00-5123-43 and #00-5223-56) is added for overnight (12 to 20 hours) incubation in a refrigerator (set at 4° C.) while protected from light. On the second day, the samples are washed 3 times with 1×1.5 mL Permeabilization buffer (eBioscince Cat. #00-8333-56) and centrifuged at room temperature, 500×g for 5 minutes before incubation with Antibody mixture D (100 μL) for 60 minutes (+5 minutes) at room temperature while protected from light. The composition of antibody mixture D is shown in Table 6 below.

TABLE 6
Composition of antibody mixture D (for
intracellular staining, 100 μL/tube)

| | | | Amount/ | |
|---|---|---|---|---|
| Name | Clone | Cat. No. | Test | Manufacturer |
| Permeabilization Buffer | — | — | 80 μL | — |
| Alexa Fluor 647 anti- | 206D | 320114 | 20 μL | Biolegend, Inc. |
| human FOXP3 Antibody | | | | |

The samples are then washed 2 times with Wash Buffer and resuspended in 300 μL Stain Buffer (BD Biosciences Cat. #554656) and acquired using LSRFORTESSA™ X-20 (BD Biosciences, MUT/150) and FACSDIVA™ Software (ver.8.0.1). Analysis is performed using FLOWJO® (ver.10.7.1). Regulatory T (Treg) cells are identified as CD45+, CD14−, CD4+, CD8−, FOXP3+ and CD25 cells. GRAPHPAD PRISM® 9 is used for plotting.

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is tested for its ability to reduce CCR8 mRNA expression in the blood of cynomolgus monkeys. In an exemplary method to test the ability of a multispecific antigen-binding molecule described herein to reduce CCR8 mRNA expression in the blood of cynomolgus monkeys, 6 cynomolgus monkeys are divided into two groups treated with either CCR8 TDB (10 mg/kg) or vehicle control. Isolation of total RNA is conducted using RNEASY® Protect Animal Blood Kit (Catalog No. 73224; Qiagen, Hilden, Germany) according to manufacturer's instructions. One-step reverse transcription and pre-amplification are carried out using SUPERSCRIPTIM III One-Step RT-PCR System (Catalog No. 12574018; Thermo Fisher; Waltham, MA). PCR reaction for relative gene expression is conducted using TAQMAN™ Universal PCR Master Mix II, no UNG (Catalog No. 4440048; Thermo Fisher) and the 96.96 DYNAMIC ARRAY™ IFC (Catalog No. BMK-M-96.96; Fluidigm; South San Francisco, CA) on the BIOMARK™ HD System (Fluidigm). Threshold cycle (Ct) values are generated using Fluidigm's Real-Time PCR software. and subsequent relative gene expression data are normalized against the geometric mean of endogenous control genes and analyzed by TIBCO SPOTFIRE® (Palo Alto, CA). Graph preparation and statistical analyses are performed by using GRAPHPAD PRISM® software (San Diego, CA).

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) is tested for its ability to alter cytokine levels in cynomolgus monkeys in a repeat-dose tolerability, toxicokinetics, and pharmacodynamics study. In an exemplary repeat-dose tolerability, toxicokinetics, and pharmacodynamics study to test the ability of a multispecific antigen-binding molecule described herein to alter cytokine levels in cynomolgus monkeys, 3 male cynomolgus monkeys per group are administered either vehicle control or anti-CCR8 TDB intravenously on Day 1 and Day 11. Cynomolgus monkeys are of Cambodian origin and approximately 2-4 kg and 2-6 years old. Animals are randomly assigned to receive either 20 mM Histidine acetate, 0.15 M NaCl, pH 5.5 (control) or anti-CCR8 TDB at 10 mg/kg via 1-hour intravenous infusion at 2 mL/kg/hr. Blood samples are collected at pre-study, Day 1 (6 hours post end of infusion (EOI) of the first dose), Day 11 (6 hours post EOI of the 2nd dose), and Day 20 (a single time point). Blood (approximately 1 mL) is drawn from the femoral vein and left at room temperature for 20 to 60 minutes. Serum is obtained by centrifugation (room temperature, 1700×g, minutes) and stored in a deep freezer (−70° C. or below) until analysis. Each sample is assayed for IL-1β, IL-1RA, IL-2, IL-4, IL-5, IL-6, IL-8, IL-10, IL-12/23 (p40), IL-13, IL-17A, IFN-γ, TNF-α, MCP-1, G-CSF and GM-CSF. The concentrations (pg/mL) of each parameter are analyzed using PROCARTAPLEX™ Multiplex Immunoassays [Non-Human Primate Assays] by BIO-PLEX® 200 (Bio-Rad Laboratories, Inc.).

### F. Methods and Compositions for Diagnostics and Detection

In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) provided herein is useful for detecting the presence of CCR8 in a biological sample. The term “detecting” as used herein encompasses quantitative or qualitative detection. In certain aspects, a biological sample comprises a cell or tissue, such as tumor.

In one aspect, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., anti-CCR8 antibody) for use in a method of diagnosis or detection is provided. In a further aspect, a method of detecting the presence of CCR8 in a biological sample is provided. In certain aspects, the method comprises contacting the biological sample with an anti-CCR8/anti-CD3 bispecific antigen-binding molecule or an anti-CCR8 antibody as described herein under conditions permissive for binding of the bispecific antigen-binding molecule or anti-CCR8 antibody to CCR8, and detecting whether a complex is formed between the bispecific antigen-binding molecule or anti-CCR8 antibody and CCR8. Such method may be an in vitro or in vivo method. In one aspect, an anti-CCR8/anti-CD3 bispecific antigen-binding molecule or an anti-CCR8 antibody is used to select subjects eligible for therapy with an anti-CCR8/anti-CD3 bispecific antigen-binding molecule or an anti-CCR8 antibody, e.g., where CCR8 is a biomarker for selection of subjects.

In certain aspects, a labeled multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or a labeled antibody (e.g., anti-CCR8 antibody) is provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### G. Pharmaceutical Compositions

In a further aspect, provided are pharmaceutical compositions comprising a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) provided herein, e.g., for use in any of the below therapeutic methods. In one aspect, a pharmaceutical composition comprises a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) provided herein and a pharmaceutically acceptable carrier. In another aspect, a pharmaceutical composition comprises any of the antigen-binding molecules or antibodies provided herein and at least one additional therapeutic agent, e.g., as described below.

Pharmaceutical compositions (formulations) of an antibody or a bispecific antigen-binding molecule as described herein can be prepared by combining the antibody with pharmaceutically acceptable carriers or excipients known to the skilled person. See, for example Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1^{st} Ed., Woodhead Publishing (2015), § 4 and Falconer R.J., Biotechnology Advances (2019), 37, 107412. Exemplary pharmaceutical compositions of an antibody or a bispecific antigen-binding molecule as described herein are lyophilized, aqueous, frozen, and the like.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredient as necessary for the particular indication being treated, preferably those with complementary activities that do not adversely affect each other. For example, it may be desirable to further provide an additional therapeutic agent useful for treatment of the same disease. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### H. Therapeutic Methods and Routes of Administration

In some aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) provided herein may be used in therapeutic methods.

In one aspect, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use as a medicament is provided. In further aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use in treating cancer is provided. In certain aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use in a method of treatment is provided. In certain aspects, the present disclosure provides a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use in a method of treating a subject (e.g., a human subject) in need thereof comprising administering to the subject an effective amount of the multispecific antigen-binding molecule or antibody. In one such aspect, the method further comprises administering to the subject an effective amount of at least one additional therapeutic agent (e.g., one, two, three, four, five, or six additional therapeutic agents), e.g., as described below. In further aspects, the present disclosure provides a multispecific antigen-binding molecule or anti-CCR8 antibody for use in depleting Tregs in a tumor microenvironment. In certain aspects, the present disclosure provides a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use in a method of depleting Tregs in a tumor microenvironment in a subject comprising administering to the subject an effective amount of the multispecific antigen-binding molecule or the antibody in depletion of Tregs in the tumor microenvironment.

In a further aspect, the present disclosure provides for the use of a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) in the manufacture or preparation of a medicament. In one aspect, the medicament is for treatment of cancer. In a further aspect, the medicament is for use in a method of treating cancer comprising administering to the subject (e.g., a human subject) in need thereof an effective amount of the medicament. In one such aspect, the method further comprises administering to the subject an effective amount of at least one additional therapeutic agent, e.g., as described below. In a further aspect, the medicament is for depleting Tregs in a tumor microenvironment. In a further aspect, the medicament is for use in a method of depleting Tregs in a tumor microenvironment in a subject comprising administering to the subject an effective amount of the medicament to deplete the Tregs in the tumor microenvironment.

In a further aspect, the present disclosure provides a method for treating cancer. In one aspect, the method comprises administering to a subject (e.g., a human subject) in need thereof an effective amount of a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) in order to treat the cancer. In one such aspect, the method further comprises administering to the subject an effective amount of at least one additional therapeutic agent, as described below.

In a further aspect, the present disclosure provides a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use in depleting Treg cells, e.g., outside or in a tumor microenvironment. For example, in certain embodiments, the present disclosure provides a method for depleting Treg cells in a tumor microenvironment in a subject (e.g., a human subject) in need thereof having cancer comprising administering to the subject an effective amount of a multispecific antigen-binding molecule or an antibody sufficient to deplete the Treg cells in the tumor microenvironment, thereby treating the cancer. In certain aspects, the present disclosure provides a method for depleting Treg cells outside of a tumor microenvironment (e.g., in circulation) in a subject (e.g., a human subject) in need thereof having cancer comprising administering to the subject an effective amount of a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) sufficient to deplete the Treg cells outside the tumor microenvironment, thereby treating the cancer. Without wishing to be bound by any particular theory, by reducing the number of Treg cells outside the tumor microenvironment, the cancer is treated as the number of Treg cells infiltrating into the tumor microenvironment is reduced, thereby reducing the number of Treg cells in the tumor microenvironment.

In a further aspect, the present disclosure provides a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) for use in reducing CCR8 mRNA expression, e.g., in the blood of a subject. For example, in certain embodiments, the present disclosure provides a method for reducing CCR8 mRNA expression in the blood of a subject (e.g., a human subject) in need thereof comprising administering to the subject an effective amount of a multispecific antigen-binding molecule or an antibody sufficient to reduce CCR8 mRNA expression. In some instances, the present disclosure provides a method for reducing CCR8 mRNA expression in the blood of a subject (e.g., a human subject) having a cancer.

Exemplary cancers includes, but is not limited to, bladder cancer (e.g., urothelial cancer), blastoma, blood cancer (e.g., lymphomas such as Non-Hodgkin's, leukemias), bone cancer, brain cancer, breast cancer (e.g., triple negative breast cancer), cervical cancer, colorectal cancer (e.g., colon cancer, rectal cancer), endometrial cancer, esophageal cancer, gastric cancer, head and neck cancer (e.g., squamous cell carcinoma of the head and neck), kidney cancer (e.g., renal cell carcinoma), liver cancer (e.g., hepatocellular carcinoma), lung cancer (e.g., non-small cell lung cancer, small cell lung carcinoma), ovarian cancer, pancreatic cancer, prostate cancer, sarcoma, skin cancer (e.g., melanoma, squamous cell carcinoma), testicular cancer, and uterine cancer.

In certain aspects, the cancer is bladder cancer, blood cancer, breast cancer, cervical cancer, colorectal cancer, esophageal cancer, gastric cancer, head and neck cancer, kidney cancer, liver cancer, lung cancer, and skin cancer.

In certain aspects, the cancer is bladder cancer, breast cancer, cervical cancer, colorectal cancer, esophageal cancer, head and neck cancer, liver cancer, lung cancer, or skin cancer.

In certain aspects, the cancer is a solid tumor.

In certain aspects, the cancer expresses CCR8.

In certain aspects, the cancer is a T cell-inflamed tumor or comprises a T-cell-inflamed tumor microenvironment.

In certain aspects, the cancer comprises regulatory T cells in the tumor microenvironment, and for which exposure of the cancer to the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody), as described herein, results in depletion of the regulatory T cell in the tumor microenvironment. In a further aspect, the present disclosure provides pharmaceutical compositions comprising any of the multispecific antigen-binding molecules or the antibodies described herein, e.g., for use in any of the above therapeutic methods. In one aspect, a pharmaceutical composition comprises any of the multispecific antigen-binding molecules or the antibodies provided herein and a pharmaceutically acceptable carrier. In another aspect, a pharmaceutical composition comprises any of the antigen-binding molecules or the antibodies provided herein and at least one additional therapeutic agent, e.g., as described below.

In some aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) as described herein can be administered alone or used in a combination therapy, e.g., useful in treating cancer. For instance, the combination therapy includes administering a multispecific antigen-binding molecule or an antibody as described herein and administering at least one additional therapeutic agent (e.g., one, two, three, four, five, or six additional therapeutic agents).

The at least one additional therapeutic agent encompasses any agent that can be administered for treatment. In certain aspects, the additional therapeutic agent is an additional anti-cancer agent. Exemplary anti-cancer agents include, but are not limited to, a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a toll-like receptor (TLR) agent, a bispecific antibody, a cellular therapy, and immune cell engager. In certain aspects, the additional therapeutic agent is an immunomodulatory anti-cancer agent, e.g., a checkpoint inhibitor (CPI) such as an anti-CTLA4 antibody (e.g., ipilimumab), a PD-L1 binding antagonist, or a PD-1 binding antagonist.

In some instances, the one or more additional therapeutic agents may reduce the rate or the severity of cytokine release syndrome (CRS). In some instances, the one or more additional therapeutic agents may prevent symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #50-42-8), or methylprednisolone (CAS #: 83-43-2)) or an IL-6R antagonist (e.g., tocilizumab (CAS #: 375823-41-9), sarilumab (CAS #: 1189541-98-7), vobarilizumab (ALX-0061; CAS #: 1628814-88-9), satralizumab (SA-237; CAS #: 1535963-91-7), and variants thereof). In certain aspects, the additional therapeutic agent encompasses tocilizumab and/or corticosteroids.

A multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) as described herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some aspects, a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) as described herein can be formulated, dosed, and administered in a fashion consistent with good medical practice. Factors for consideration in this context include the particular disorder being treated, the particular subject species being treated, the clinical condition of the subject, the cause of the disorder, the site of delivery of the agent, the method of administration, the scheduling of administration, and other factors known to medical practitioners. The multispecific antigen-binding molecule or the antibody need not be, but is optionally formulated with, one or more agents currently used to treat the disorder in question. The effective amount of such other agents depends on the amount of antigen-binding molecule or antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

The multispecific antigen-binding molecule (e.g., a multispecific antibody; e.g., a bispecific binding molecule; e.g., anti-CCR8/anti-CD3 bispecific antibody; e.g., anti-CCR8/anti-CD3 TDB)) or the antibody (e.g., the anti-CCR8 antibody) is suitably administered to the subject at one time or over a series of treatments. For repeated administrations over several days or longer, depending on the condition, the treatment would generally be sustained until a desired suppression of disease symptoms occurs. However, other dosage regimens may be useful. The progress of this therapy is easily monitored by conventional techniques and assays.

Any of the methods described herein may involve monitoring a subject for cytokine release syndrome (CRS), e.g., a CRS event following commencement of any of the methods described above (e.g., following administration of a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody)). Current clinical management focuses on treating the individual signs and symptoms, providing supportive care, and attempting to dampen the inflammatory response using a high dose of corticosteroids. However, this approach is not always successful, especially in the case of late intervention. The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biology of Blood and Marrow Transplantation. 25 (4): 625-638, 2019). The ASTCT criteria is intended to be objective, easy to apply, and more accurately categorize the severity of CRS. This revised CRS grading system is shown below in Table 7.

TABLE 7
CRS Grading System

| CRS | | | | |
|---|---|---|---|---|
| Parameter | Grade 1 | Grade 2 | Grade 3 | Grade 4 |
| Fever | Temperature ≥38° C. | Temperature ≥38° C. | Temperature ≥38° C. | Temperature ≥38° C. |
| | | with | | |
| Hypotension | None | Not requiring vasopressors | Requiring a vasopressor | Requiring multiple vasopressors |
| | | | with or without vasopressin | (excluding vasopressin) |
| | | and/or | | |
| Hypoxia | None | Requiring low-glow nasal | Requiring high-flow nasal | Requiring positive pressure (e.g., |
| | | cannula or blow-by | cannula, facemask, nonrebreather | CPAP, BiPAP, intubation and |
| | | | mask or Venturi mask | mechanical ventilation) |

ASTCT = American Society for Transplantation and Cellular Therapy; BiPAP = bilevel positive airway pressure; CPAP = continuous positive airway pressure; CRS = cytokine release syndrome; CTCAE = Common Terminology Criteria for Adverse Events.

Fever is defined as a temperature ≥38° C. not attributable to any other cause. In subjects who have CRS then receive antipyretic or anticytokine therapy such as tocilizumab or steroids, fever is no longer required to grade subsequent CRS severity. In this case, CRS grading is determined by hypotension and/or hypoxia.

CRS grade is determined by the more severe event, hypotension or hypoxia not attributable to any other cause. For example, a subject with temperature of 39.5° C., hypotension requiring 1 vasopressor, and hypoxia requiring low-flow nasal cannula is classified as Grade 3 CRS.

Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

CRS is associated with elevations in a wide array of cytokines, including marked elevations in IFN-γ, IL-6, and TNF-α levels. Emerging evidence implicates IL-6, in particular, as a central mediator in CRS. IL-6 is a proinflammatory, multi-functional cytokine produced by a variety of cell types, which has been shown to be involved in a diverse array of physiological processes, including T cell activation. Regardless of the inciting agent, CRS is associated with high IL-6 levels (Nagorsen et al. Cytokine. 25 (1): 31-5, 2004; Lee et al. Blood. 124 (2): 188-95, 2014); Doesegger et al. Clin. Transl. Immunology. 4 (7): e39, 2015), and IL-6 correlates with the severity of CRS, with subjects who experience a Grade 4 or 5 CRS event having much higher IL-6 levels compared to subjects who do not experience CRS or experience milder CRS (Grades 0-3) (Chen et al. J. Immunol. Methods. 434:1-8, 2016).

Therefore, blocking the inflammatory action of IL-6 using an agent that inhibits IL-6-mediated signaling to manage CRS observed in subjects during the double-step fractionated, dose-escalation dosing regimen is an alternative to steroid treatment that would not be expected to negatively impact T cell function or diminish the efficacy or clinical benefit of TDB (e.g., anti-CCR8/anti-CD3 TDB) therapy.

Tocilizumab (ACTEMRA®/ROACTEMRA®; CAS #: 375823-41-9) is a recombinant, humanized, anti-human monoclonal antibody directed against soluble and membrane-bound IL-6R, which inhibits IL-6-mediated signaling (see, e.g., WO 1992/019579, which is incorporated herein by reference in its entirety).

If the subject has a cytokine release syndrome (CRS) event following administration of the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)), the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the event. In some instances, tocilizumab is administered intravenously to the subject as a single dose of about 8 mg/kg. In some instances, each dose of tocilizumab does not exceed 800 mg/dose. Other anti-IL-6R antibodies that could be used instead of, or in combination with, tocilizumab include sarilumab (CAS #: 1189541-98-7), vobarilizumab (ALX-0061; CAS #: 1628814-88-9), satralizumab (SA-237; CAS #: 1535963-91-7), and variants thereof.

If the subject has a CRS event that does not resolve or worsens within 24 hours of administering the IL-6R antagonist to treat the symptoms of the CRS event, and the method may further comprise administering to the subject one or more additional doses of the IL-6R antagonist (e.g., an anti-IL-6R antibody, e.g., tocilizumab) to manage the CRS event. The subject may be administered a corticosteroid, such as methylprednisolone or dexamethasone if CRS event is not managed through administration of the IL-6R antagonist.

Management of the CRS events may be tailored based on the Stage of the CRS and the presence of comorbidities. For example, if the subject has a Grade 2 cytokine release syndrome (CRS) event in the absence of comorbidities or in the presence of minimal comorbidities following administration of the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)), the method may further include treating the symptoms of the Grade 2 CRS event while suspending treatment with the multispecific antigen-binding molecule or multispecific antibody (e.g., TDB, e.g., anti-CCR8/anti-CD3 TDB). If the Grade 2 CRS event then resolves to a Grade≤1 CRS event for at least three consecutive days, the method may further include resuming treatment with the multispecific antigen-binding molecule or multispecific antibody (e.g., TDB, e.g., anti-CCR8/anti-CD3 TDB) without altering the dose. On the other hand, if the Grade 2 CRS event does not resolve or worsens to a Grade ≥3 CRS event within 24 hours of treating the symptoms of the Grade 2 CRS event, the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the Grade 2 or Grade ≥3 CRS event. In some instances, tocilizumab is administered intravenously to the subject as a single dose of about 8 mg/kg. In some instances, each dose of tocilizumab does not exceed 800 mg/dose. Other anti-IL-6R antibodies that could be used instead of, or in combination with, tocilizumab include sarilumab, vobarilizumab (ALX-0061), satralizumab (SA-237), and variants thereof.

If the subject has a grade 2, 3, or 4 CRS event in the presence of extensive comorbidities following administration of the multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)), the method may further include methods understood in the art to mitigate the CRS event, such as administering to the subject a first dose of an IL-6R antagonist (e.g., an anti-IL-6R antibody, e.g., tocilizumab (ACTEMRA®/ROACTEMRA®)) to manage the CRS event while suspending treatment with the multispecific antigen-binding molecule. Other anti-IL-6R antibodies that could be used instead of, or in combination with, tocilizumab include sarilumab, vobarilizumab (ALX-0061), satralizumab (SA-237), and variants thereof. In some instances, the method further includes administering to the subject an effective amount of a corticosteroid, such as dexamethasone (CAS #: 50-02-2), prednisone (CAS #: 53-03-2), prednisolone (CAS #50-42-8), or methylprednisolone (CAS #: 83-43-2.

In an additional embodiment, use of a mouse surrogate is contemplated, e.g., for use as an in vitro or in vivo tool molecule. For example, in one aspect, provided is a method of treating a disease in a mouse comprising administering an effective amount of the mouse surrogate antibody, as described herein, to the mouse to treat the disease. In certain embodiments, the mouse comprises a xenograft. In certain embodiments, the mouse model is a cancer model, e.g., a breast cancer (e.g., E0771) or a colorectal cancer (e.g., MC-38) model.

### I. Articles of Manufacture

In another aspect, an article of manufacture containing materials useful for the treatment, prevention and/or diagnosis of the disorders described above is provided. The article of manufacture may include a multispecific antigen-binding molecule (e.g., a multispecific antibody; e.g., a bispecific antigen-binding molecule (e.g., bispecific antibody; 2+1 TDB; e.g., anti-CCR8/anti-CD3 bispecific antigen-binding molecule; e.g., anti-CCR8/anti-CD3 bispecific antibody; e.g., anti-CCR8/anti-CD3 TDB)) or an antibody (e.g., an anti-CCR8 antibody) disclosed herein. The article of manufacture comprises a container and a label or package insert on or associated with the container. Suitable containers include, for example, bottles, vials, syringes, IV solution bags, and the like. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody as disclosed herein. The label or package insert indicates that the composition is used for treating the condition of choice. Moreover, the article of manufacture may comprise (a) a first container with a composition contained therein, wherein the composition comprises an antibody as disclosed herein; and (b) a second container with a composition contained therein, wherein the composition comprises a further cytotoxic or otherwise therapeutic agent. The article of manufacture in this aspect as described herein may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLE

The following is an example of methods and compositions of the invention. It is understood that various other embodiments may be practiced, given the general description provided above.

### Example 1. Generation and Characterization of Anti-C-C Motif Chemokine Receptor 8 (CCR8) Antigen-Binding Molecules

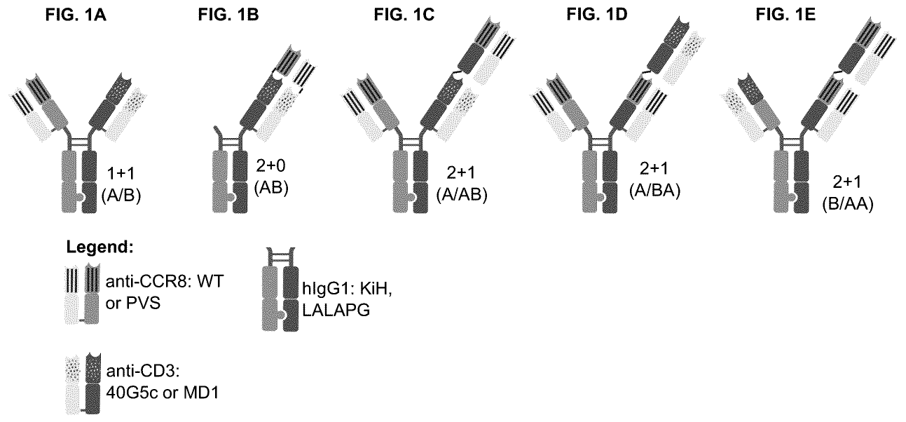

Constructs encoding for a panel of T cell-dependent bispecific antibody (TDB) formats (FIG. 1) were generated by gene synthesis. For the anti-CCR8 arms, the wild-type sequence (WT) or affinity matured sequence (PVS) were used. See, e.g., U.S. application Ser. No. 17/864,074 and PCT Application No. PCT/US2022/073671, each of which is incorporated herein by reference in its entirety. For the anti-CD3 arms, a low affinity anti-human CD3 clone 40G5c or high affinity anti-human CD3 clone MD1 were used. See, e.g., U.S. Publication Nos. US20150166661A1 and US20210179715A1 and PCT Publication Nos. WO 2015/095392 and WO 2021/119505. For the anti-mouse surrogate TDB, the sequence of an anti-mouse CD3 clone 2C11 was used (Fernandes, et al. J Biol Chem. 287 (16): 13324-13335, 2012). The Fc region for the anti-human TDBs was human IgG₁ and the Fc region for the anti-mouse surrogate TDBs was mouse IgG2a. Both Fc were effectorless versions containing the L234A, L235A, and P329G mutations (Lo, et al. J Biol Chem. 292 (9): 2900-2908, 2017). Knobs-into-hole technology with either T366W (knob) or T366S, S368A, and Y407V (hole) mutations (Ridgway, et al. Prot Eng. 9 (7): 617-621, 1996) was used to generate bispecific antibodies. Half-antibodies bearing either the knob or hole mutation were separately expressed, captured and purified to generate intermediates, which were then combined and assembled into bispecific antibody using reduction-oxidation assembly chemistry with L-reduced glutathione. Remaining leftover redox chemicals and mis-assembled species were then separated by column chromatography to yield purified bispecific antibody (Williams et al. Biotechnol. Prog. 31 (5): 1315-1322, 2015; Ovacik et al. mAbs. 11 (2): 422-433, 2018).

An effective TDB molecule requires sufficient potency to eliminate the target cells while also exhibiting low cytokine release. Since CCR8 is present at very low copy numbers on Tregs and Tregs also express CD3, it was unclear a priori whether anti-CCR8/anti-CD3 bispecific antibodies could be used for depletion of Tregs as well as which TDB format(s) and which anti-human target arms (CCR8 and CD3) would yield an effective TDB molecule. A panel of TDB formats was therefore generated to assess for activity in various assays (FIG. 1A-FIG. 1E).

Next, the binding affinity of the generated anti-CCR8 antigen-binding molecules was characterized using cell-based affinity assays. Anti-CCR8-1889/1889.aCD3.40G5c was labeled via amine conjugation using ALEXA FLUOR™ 488 with an incorporation of ˜4 fluorophores/molecule. One million Chinese hamster ovary (CHO) cells stably expressing either human or cynomolgus CCR8 (˜60,000 and 30,000 receptors/cell, respectively) were seeded at a concentration of 1 million cells/mL onto a Multidish 2×2 Petri Dish (Ridgeview Instruments AB, Sweden) to form a circular drop of ˜ 3 cm diameter in chambers A and C of the dish. Untransfected CHO cells were seeded in opposing chambers (B and D) in the same manner. Cells were left undisturbed to adhere for 4 hours. Excess media was aspirated, fresh growth media was added, and cells were incubated overnight at 37° C., 5% CO₂. The following day, media was aspirated and fresh media was added. The cells were incubated at room temperature for 20 minutes prior to starting the assay. The plate was loaded onto a LIGAND TRACER® Green instrument (Ridgeview Instruments AB) for real-time fluorescence measurement. After the fluorescent signal stabilized (15-30 minutes), labeled anti-CCR8-1889/1889.aCD3.40G5c was added in two increasing concentrations (1 nM and 10 nM) until curvature was obtained. Antibody (Ab) dissociation was recorded by aspirating the media and adding fresh media with 10 nM of unlabeled Ab to prevent rebinding of labeled Ab. The Ab dissociation was measured overnight to obtain accurate off-rates. Untransfected CHO cells were used to subtract the non-specific binding. Specific binding traces were analyzed using TraceDrawer 1.7 (Ridgeview Instruments) software and fit with a 1-to-2 with bulk binding model to determine association (kₐ) and dissociation (k_{d}) rate constants. The equilibrium dissociation constants (K_{D}) for each binding event were calculated as the ratio of the dissociation and association rates, k_{d}/kₐ. The results of the cell-based affinity assays are provided below in Table 8.

TABLE 8
Cell-based Affinities

| | | kₐ (M⁻¹s⁻¹) | k_{d} (s⁻¹) | K_{D} (pM) |
|---|---|---|---|---|
| | Valency | Mean ± S.D. | Mean ± S.D. | Mean ± S.D. |
| aCCR8-1889/1889.aCD3.40G5c | Monovalent | 3.24 ± 0.53 (×10⁵) | 1.57 ± 0.26 (×10⁻⁴) | 484 ± 11.4 |
| on human-CCR8 | Bivalent | 2.46 ± 0.32 (×10⁵) | 2.02 ± 1.56 (×10⁻⁶) | 7.78 ± 5.13 |
| aCCR8-1889/1889.aCD3.40G5c | Monovalent | 3.18 ± 2.04 (×10⁵) | 2.68 ± 1.15 (×10⁻⁴) | 1010 ± 551 |
| on cyno-CCR8 | Bivalent | 1.86 ± 0.81 (×10⁵) | 3.36 ± 2.89 (×10⁻⁶) | 22.8 ± 19.7 |

### Example 2. Characterization of Different TDB Formats

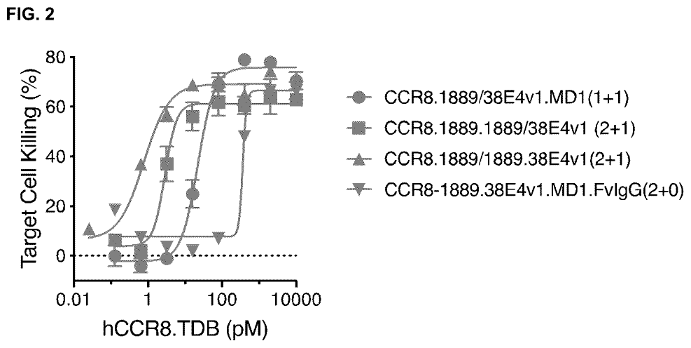

1+1 (monovalent binding to CD3 and CCR8) and 2+1 (monovalent binding to CD3 and bivalent binding to CCR8) TDB formats were compared. Different TDB formats were tested for their target cell killing activity on CCR8.CHO cells. Human peripheral blood mononuclear cells (PBMC) were isolated from healthy donor blood using a FICOLL® density gradient centrifugation with LYMPHOPREP® solution (STEMCELL Technologies). CD8+ T cells were magnetically enriched using human CD8+ T Cell isolation kit (Miltenyi Biotec). Human CCR8-expressing Chinese Hamster Ovary (CHO-hCCR8-Ga15) cells were plated in the black, clear-bottomed 96-well plates at density of 10,000 cells per well. CD8+ T cells and CHO-hCCR8 cells were cocultured at a 3:1 ratio with anti-CCR8/CD3-TDB at indicated concentrations. After 48 hours, the plates were washed twice with PBS. 100 μL of CELLTITER-GLO® Luminescent Cell Viability reagent (Promega cat #G7570) was added and plates were read on a luminometer as described in the instructions. The target cell killing activity was calculated as: {(reading without TDB-reading with TDB)/(reading without TDB)}×100%. Results are shown in FIG. 2 and are summarized below in Table 9. The 2+1 TDB (e.g., 2+1 anti-CCR8/anti-CD3 TDB; e.g., anti-CCR8 TDB) comprising a bivalent arm with different binding moieties (i.e., one targeting CD3 and one targeting CCR8) was found to perform the best (i.e., having the lowest EC50) compared to 1+1 and 2+0 formats, as well as 2+1 TDB formats having a bivalent arm that comprises two CCR8-binding moieties.

TABLE 9
EC₅₀ of Target Cell Killing Activity of Different TDB Formats

| | CCR8.1889/38E4v1.MD1 | CCR8.1889.1889/ | CCR8.1889/1889.38E4v1 | CCR8-1889.38E4v1.MD1 |
|---|---|---|---|---|
| | (1 + 1) | 38E4v1 (2 + 1) | (2 + 1) | Fv IgG (2 + 0) |
| Donor 1 (EC₅₀) | 22.24 | 2.88 | 0.78 | ~364.7 |
| Donor 2 (EC₅₀) | ~182.9 | 54.04 | 2.14 | ~195.0 |

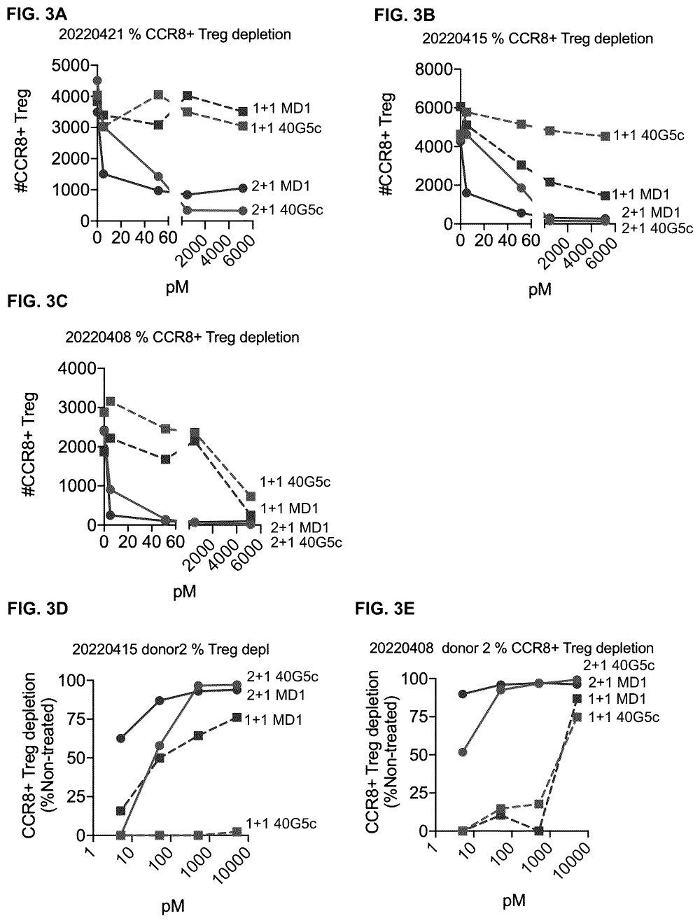

Different TDB formats were tested for their ability to deplete CCR8+ Treg cells. Peripheral blood mononuclear cells (PBMC) were isolated from healthy donor blood using a FICOLL® density gradient (GE Healthcare). T cells were magnetically enriched using human Pan T Cell isolation kit (Miltenyi Biotec). T cells (1×10⁵ cells/well) were incubated for 48 hours at 37° C. in culture media with indicated concentrations of individual anti-CCR8-CD3 T cell engagers in a U-bottom 96-well plate, 1889:40G5c (1+1 40G5), 1889: MD1 (1+1 MD1), 1889/1889:40G5c (2+1 40G5) and 1889/1889: MD1 (2+1 MD1). After 2 days, cells were harvested and sequentially stained with fixable viability dye, blocked with human FcR-blocking reagent (Miltenyi Biotec), stained with surface markers, and then intracellular marker using the Foxp3 transcription factor staining buffer set (Thermo Fisher Scientific) following the manufacturer's instruction. Samples were acquired by flow cytometry (Symphony, BD) and analyzed using FLOWJO® software package (BD Biosciences). Cell numbers were quantified with COUNTBRIGHT® absolute counting beads (ThermoFisher). Results are shown in FIG. 3A-FIG. 3E. 2+1 CCR8-TDB (1889) format showed the most potent killing of hCCR8+ CHO.

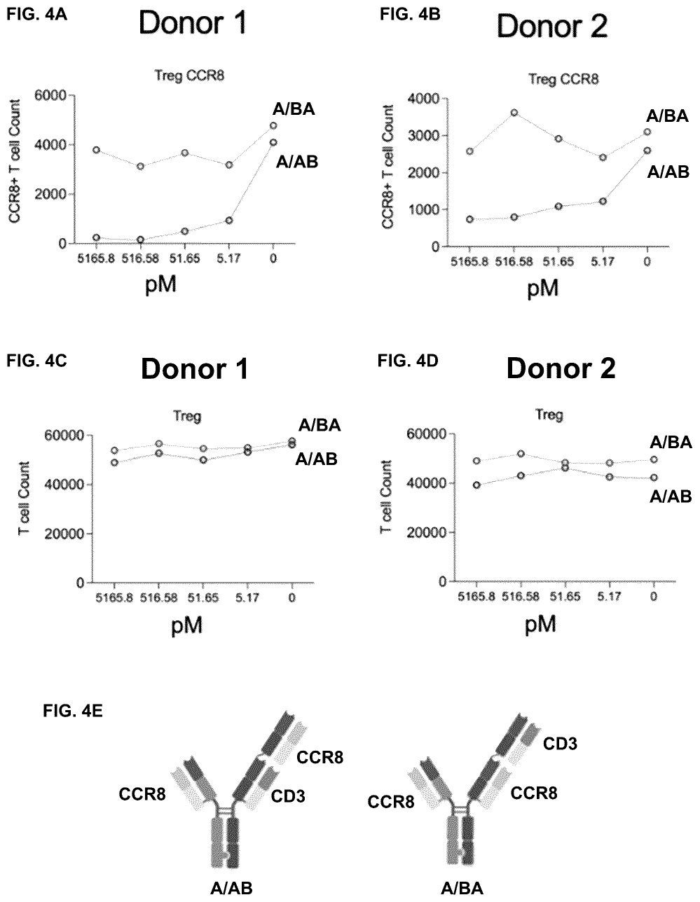

The orientation of the bivalent arm of the 2+1 TDB format bispecific antigen-binding molecule was also tested. T cells were magnetically enriched from either fresh (Donor 1: P0000745051) or frozen/thawed (Donor 2: R54911) healthy donor PBMC using human Pan T Cell isolation kit (Miltenyi Biotec). T cells (1×10⁵ cells/well) were incubated for 48 hours at 37° C. in culture media with the indicated concentrations of individual anti-CCR8-CD3 T cell engagers in a U-bottom 96-well plate, 1889/MD1:1889 (A/BA) and 1889/1889: MD1 (A/AB) anti-CCR8 TDBs. TDBs were added with three step 10-fold serial dilution with top concentration at 1 μg/mL (5.16 nM). BA and AB denote the orientation of the bivalent arm, where the antigen-binding domain represented by the preceding letter is positioned N-terminal to the antigen-binding domain represented by the succeeding letter. After 2 days, cells were harvested and sequentially stained with fixable viability dye (ThermoFisher Scientific), blocked with human FcR-blocking reagent (Miltenyi Biotec), stained with surface markers, and then intracellular marker using the Foxp3 transcription factor staining buffer set (ThermoFisher Scientific) following the manufacturer's instruction. Samples were acquired by flow cytometry (Symphony, BD) and analyzed using FLOWJO® (BD Biosciences). Cell numbers were quantified with COUNTBRIGHT® absolute counting beads (ThermoFisher Scientific). Results are shown in FIG. 4A-FIG. 4E. The A/BA 2+1 anti-CCR8 TDB was found to not deplete human PBMC CCR8+ Treg, whereas the A/AB 2+1 anti-CCR8 TDB depleted human PBMC CCR8+ Treg. Therefore, these data indicate that the A/AB 2+1 anti-CCR8 TDB orientation is unexpectedly superior for Treg depletion compared to other orientations.

### Example 3. Effect of CD3 Binding Affinity on Efficacy of Anti-CCR8 TDBs

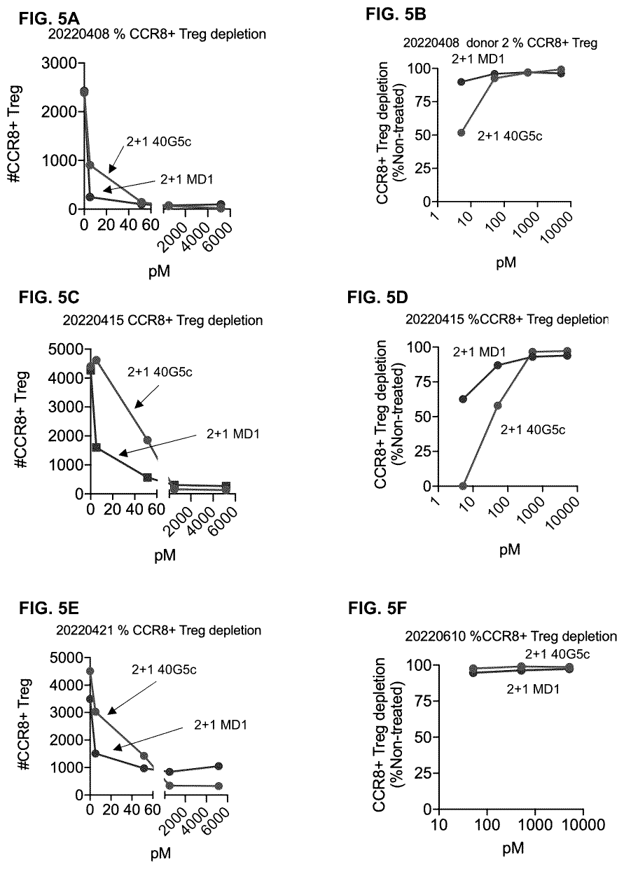

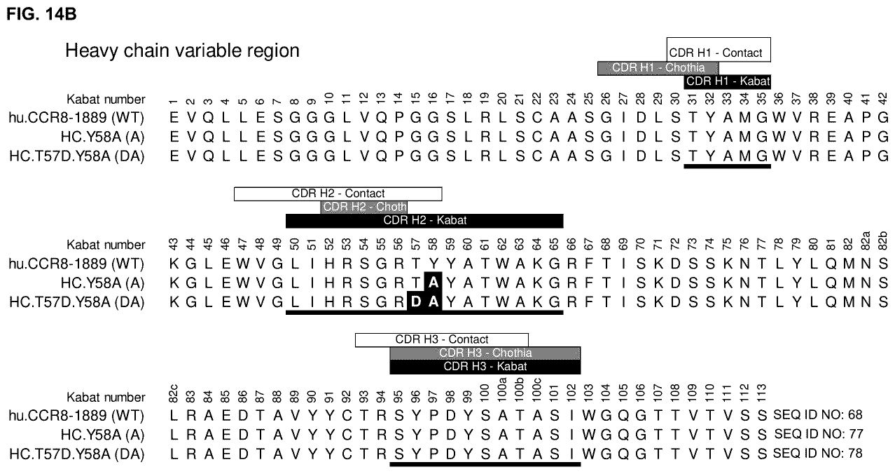

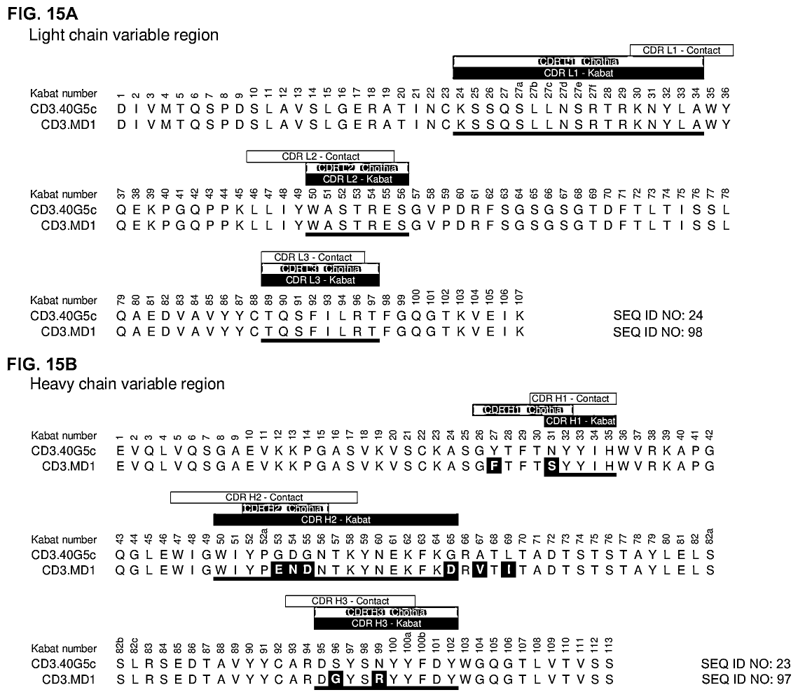

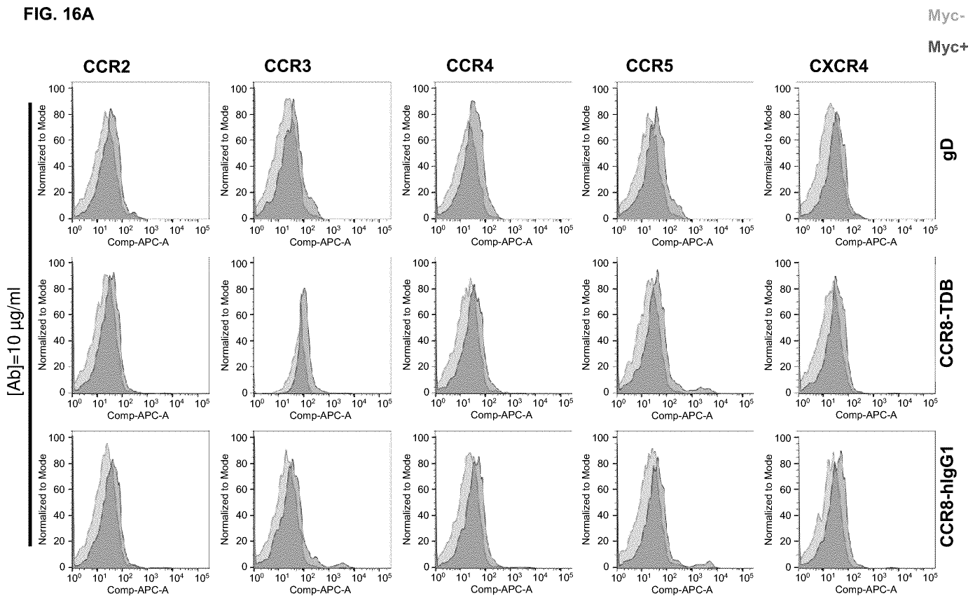

The impact of CD3 binding affinity on TDB activity was evaluated. TDB activity was first evaluated via a cell-based assay. Peripheral blood mononuclear cells (PBMC) were isolated from healthy donor blood using a FICOLL® density gradient (GE Healthcare). T cells were magnetically enriched using human Pan T Cell isolation kit (Miltenyi Biotec). T cells (1×10⁵ cells/well) were incubated for 48 hours at 37° C. in culture media with the indicated concentrations of individual anti-CCR8-CD3 T cell engagers in a U-bottom 96-well plate, 1889/1889:40G5c (2+1 40G5) and 1889/1889: MD1 (2+1 MD1). After 2 days, cells were harvested and sequentially stained with fixable viability dye (ThermoFisher Scientific), blocked with human FcR-blocking reagent (Miltenyi Biotec), stained with surface markers, and then intracellular marker using the Foxp3 transcription factor staining buffer set (ThermoFisher Scientific) following the manufacturer's protocols. Samples were acquired by flow cytometry (Symphony, BD) and analyzed using FLOWJO® software package (BD Biosciences). Cell numbers were quantified with COUNTBRIGHT® absolute counting beads (ThermoFisher Scientific). Results are shown in FIG. 5A-FIG. 5F. High affinity CD3-binding MD1 was found to only perform better at ≤50 PM doses. At higher doses, high affinity MD1 and low affinity 40G5c exhibited similar activity. Sequence alignment of the VH and VL sequences of the 40G5c CD3 antibody and the MD1 CD3 antibody is shown in FIG. 14A and FIG. 14B.

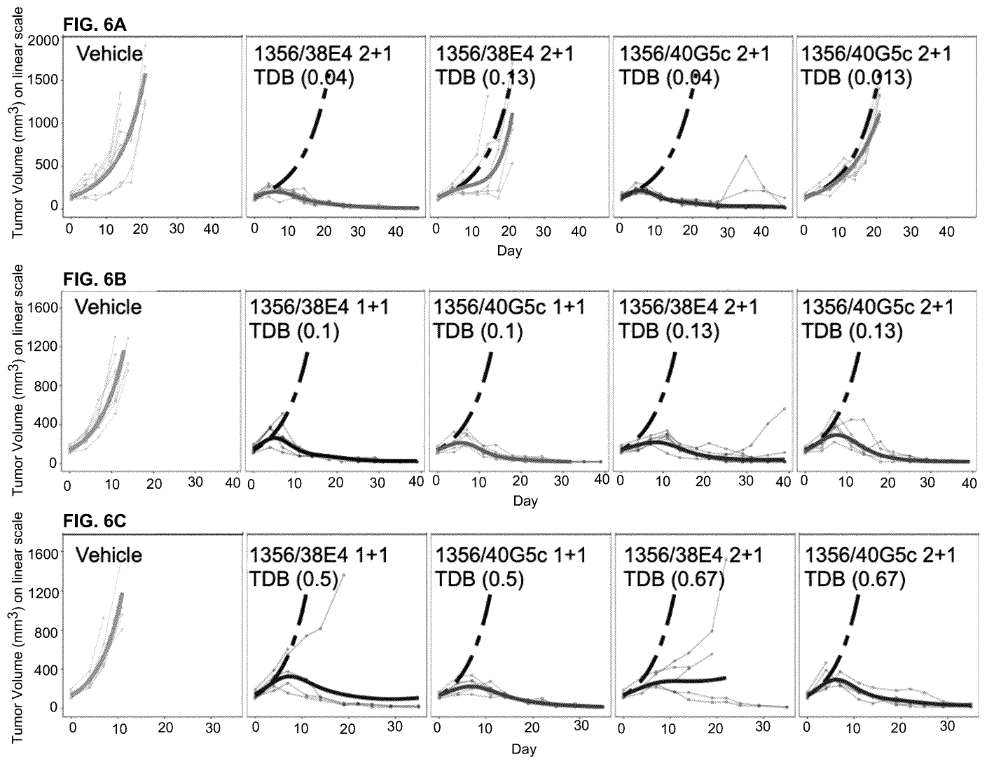

Activity of TDBs having different CD3 binding affinity was further evaluated in vivo using chimeric TDBs. The efficacy of anti-CCR8 TDBs was investigated in mouse breast cancer syngeneic model, E0771, and mouse colorectal syngeneic model, MC-38. Animal studies using these cell lines were carried out in compliance with National Institutes of Health guidelines for the care and use of laboratory animals and were approved by the Institutional Animal Care and Use Committee (IACUC) at Genentech. To establish the models, 0.1 million tumor cells (suspended in 0.1 mL of Hanks' Balanced Salt Solution (HBSS) with MATRIGEL®) were inoculated, into the mammary fat pad #5 for the E0771 model (FIG. 6A and FIG. 6B) or subcutaneously into the flank for the MC-38 model (FIG. 6C), of female hu.CD3E.tg.B6N mice (Genentech; Dixon, CA). Hu.CD3E.tg.B6N transgenic mice had a C57BI/6 mouse background and were genetically engineered to express human CD3.

When tumors reached the desired volume (˜137 mm³), animals were divided into groups of n=5-7 with similar distribution of tumor volumes, and received a single intravenous dose of vehicle (20 mM histidine acetate, 240 mM sucrose, 0.02% polysorbate-20, pH 5.5) or TDB through the tail vein (referred to as Day 0). The treatment information was not blinded during measurement. Tumors were measured in two dimensions (length and width) using calipers and tumor volume was calculated using the formula: Tumor size (mm³)=0.5×(length×width×width). Tumor sizes and mouse body weights were recorded twice weekly over the course of the study. Mice whose tumor volume exceeded 2000 mm³ or whose body weight loss was 20% of their starting weight were promptly euthanized per IACUC guidelines.

Data were analyzed using R statistical software system (R Foundation for Statistical Computing; Vienna, Austria), and a mixed modeling was fit within R using the nime package (Pinheiro et al. 2013). Cubic regression splines were used to fit a non-linear profile to the time courses of log 2 tumor volume of each treatment. These non-linear profiles were then related to the treatment within the mixed model. This approach addressed both repeated measurements and modest dropouts due to any non-treatment-related removal of animals before study end. Results were plotted in natural scale as fitted tumor volume of each group over time. In both animal models, TDBs comprising high and low CD3-binding moieties performed similarly.

To evaluate the safety of high versus low affinity CD3 binders, cytokine release following anti-CCR8 TDB was evaluated in in vivo. Cytokine release following anti-CCR8 TDB (T-cell dependent bispecific) treatment was investigated in mouse colorectal syngeneic model, MC-38. The animal study using this cell line was carried out in compliance with National Institutes of Health guidelines for the care and use of laboratory animals and was approved by the Institutional Animal Care and Use Committee (IACUC). To establish the model, 0.1 million tumor cells (suspended in 0.1 mL of Hanks' Balanced Salt Solution (HBSS) with MATRIGEL®) were subcutaneously inoculated into the flank of female hu.CD3E.tg.B6N mice (Genentech; Dixon, CA). Hu.CD3E.tg. B6N transgenic mice had a C57BI/6 mouse background and were genetically engineered to express human CD3.

When tumors reached the desired volume (˜136 mm³), animals were divided into groups of n=5 with similar distribution of tumor volumes, and received a single intravenous dose of vehicle (20 mM histidine acetate, 240 mM sucrose, 0.02% polysorbate-20, pH 5.5) or TDB through the tail vein (referred to as Day 0).

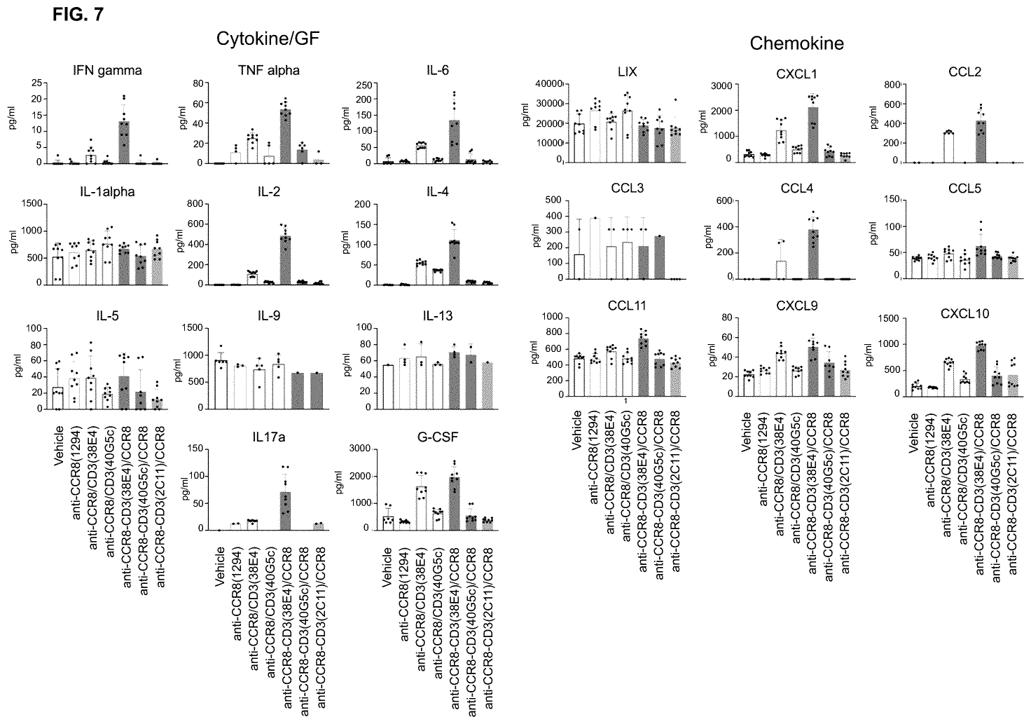

Blood samples were collected from mice 2 hours post-dose and processed for serum. Cytokines in serum were analyzed using the mouse cytokine LUMINEX® assay (Millipore) according to the manufacturer instructions. Results are shown in FIG. 7. TDBs comprising the high CD3 affinity CD3-binding moiety (MD1) was found to elicit higher systemic cytokines in mice compared to 40G5c.

### Example 4. Affinity Maturation of Anti-CCR8 Antibody Variants

To determine if a higher affinity anti-CCR8 antibody (e.g., a higher affinity binding domain that specifically binds to CCR8) could improve the potency of the TDB, affinity maturation on the parent anti-CCR8 antibody was performed. A large panel of anti-CCR8 variants containing all possible single mutations at each position of the six CDRs was generated. An initial off-rate screen by surface plasmon resonance using a surrogate sulfated peptide antigen identified four mutants (LC.129V, LC.A32I, LC.E95dS, and HC. Y58A (numbering according to Kabat)) that exhibited slower off-rates relative to the parent antibody.

A panel of 22 one-armed antibodies containing single and combined mutations was generated for subsequent analysis. Each variant contained a S12P (numbering according to Kabat) mutation to aid in future TDB purification as described below. For variants containing the E95dS mutation (numbering according to Kabat), an inert mutation in the HC (T57D) (numbering according to Kabat) was also included to conserve the net charge of the Fv region.

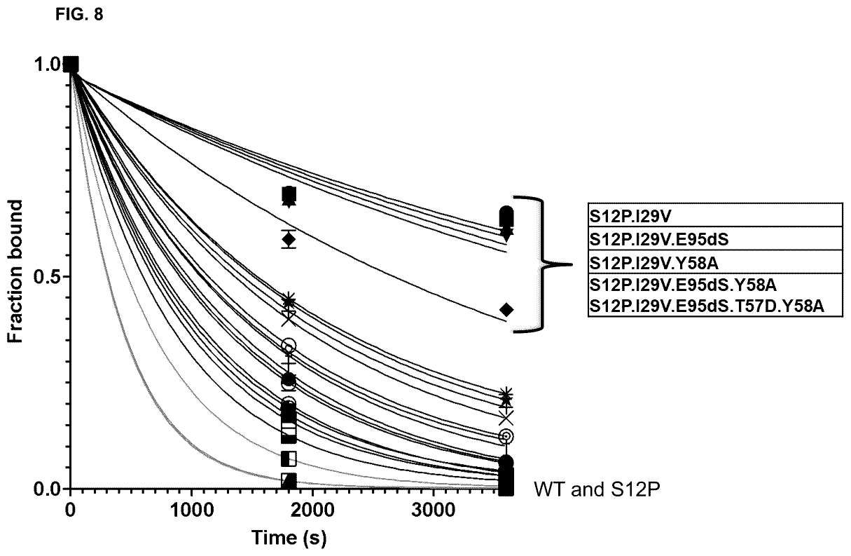

The off-rate of each anti-CCR8 antibody (parent and variants) was determined by flow cytometry. In particular, human CCR8 CHO stable cell lines were stained with anti-CCR8 antibodies (20 μg/ml) at 4° C. for 60 minutes, then washed twice with FACS buffer (PBS with 0.5% bovine serum albumin (BSA) and 0.2 mM EDTA). Competitor parental anti-CCR8 (100 μg/mL in FACS buffer) with a rabbit IgG Fc was then added. At various time points (0, 30, and 60 minutes), samples were washed twice with FACS buffer followed by staining with ALEXA FLUOR®-anti-hIgG (1:300, Jackson Immuno) at 4° C. for 15 minutes. Cells were washed twice with FACS buffer, re-suspended in FACS buffer with propidium iodide (0.5 μg/mL), and analyzed with BD FACSCELESTA® Flow Cytometer or iQue3 (Sartorius). The fraction bound of each variant was determined as the ratio of the mean fluorescence intensity at time X divided by the mean fluorescence intensity at 0 minutes (FIG. 8). This analysis identified five variants (129V, I29V.E95dS, I29V.Y58A, I29V.E95dS.Y58A, and I29V.E95dS.Y57D.Y58A (numbering according to Kabat)) with the best off-rates relative to the parent WT antibody.

The relative binding potency of the top five anti-CCR8 variants identified above was further characterized by comparison to the parental clone using flow cytometry. In particular, human CCR8 CHO and cyno (cynomolgus monkey) CCR8 CHO stable cell lines were stained with a dilution series of anti-CCR8 antibodies at room temperature for 14 hours, then washed twice with FACS buffer (PBS with 0.5% BSA and 0.2 mM EDTA). Samples were washed twice with FACS buffer followed by staining with goat Fab anti-hu IgG H+L (1:300, Jackson Immuno) at 4° C. for 15 minutes. Cells were washed twice with FACS buffer, and re-suspended in FACS buffer with propidium iodide (0.5 μg/ml) and analyzed with BD FACSCELESTA® Flow Cytometer or iQue3 (Sartorius). Titration experiments on both human CCR8 and cyno CCR8 CHO stable cell lines identified S12P.I29V.E95dS (PVS) and S12P.I29V.E95dS.T57D.Y58A (PVSDA) (numbering according to Kabat) as the most potent variants against both CCR8 targets (see Table 10 below). PVS was selected as the clone for further testing as a TDB because both clones showed similar potencies and PVS contained fewer mutations overall. Sequence alignment of the VH and VL sequences of the affinity mature variants of the 1889 antibody relative to the 1889 WT antibody is shown in FIG. 14A and FIG. 14B.

TABLE 10
Summary of Estimated Affinities for Variants

| | huCCR8 | cyCCR8 |
|---|---|---|
| Antibody | EC50 (nM) | EC50 (nM) |
| WT | 17.07 | 11.48 |
| S12P.I29V (PV) | 1.65 | 7.67 |
| S12P.I29V.E95dS (PVS) | 0.66 | 3.13 |
| S12P.I29V.Y58A (PVA) | 1.03 | 6.14 |
| S12P.I29V.E95dS.Y58A (PVSA) | 1.19 | 3.39 |
| S12P.I29V.E95dS.T57D.Y58A (PVSDA) | 0.94 | 2.24 |

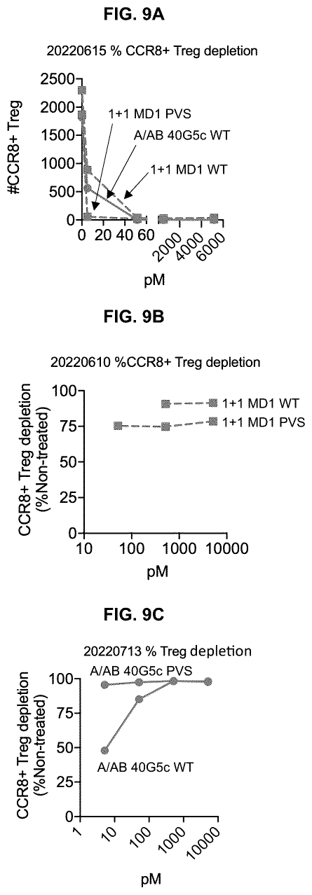

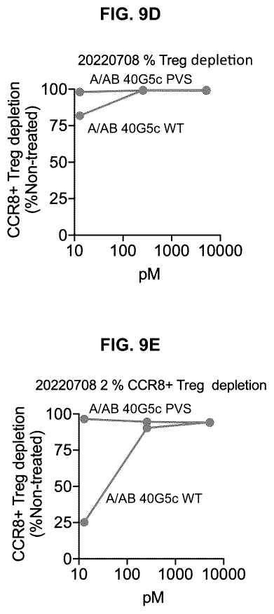

The activity of higher-affinity anti-CCR8 antibody variants was characterized in cell-based assays using both 1+1 and 2+1 TDBs. Peripheral blood mononuclear cells (PBMC) were isolated from healthy donor blood using a FICOLL® density gradient (GE Healthcare). T cells were magnetically enriched using human Pan T Cell isolation kit (Miltenyi Biotec). T cells (1×10⁵ cells/well) were incubated for 48 hours at 37° C. in culture media with indicated concentrations of individual anti-CCR8-CD3 T cell engagers in a U-bottom 96-well plate. After 2 days, cells were harvested and sequentially stained with fixable viability dye (ThermoFisher Scientific), blocked with human FcR-blocking reagent (Miltenyi Biotec), stained with surface markers, and then intracellular marker using the Foxp3 transcription factor staining buffer set (ThermoFisher Scientific) following the manufacturer's protocols. Samples were acquired by flow cytometry (Symphony, BD) and analyzed using FLOWJO® software package (BD Biosciences). Cell numbers were quantified with COUNTBRIGHT® absolute counting beads (ThermoFisher Scientific). Results are shown in FIG. 9A-FIG. 9E. Benefit of 1889 affinity maturation on TDB activity was limited and only detectable at ≤10 pM doses of TDB.

The activity of higher-affinity anti-CCR8 antibody variants was further characterized in vivo. C.B-17 severe combined immunodeficiency (SCID) mice (non-binding species) were distributed into 4 groups with n=4 mice per group. Mice received a single intravenous (IV) bolus injection of 5 mg/kg of anti-CCR8 TDB 2+1 A/AB (A: 1889 WT, B: 40G5c) in Group 1, anti-CCR8 TDB 2+1 A/AB (A: 1889 PVS, B: 40G5c) in Group 2, anti-CCR8 TDB 1+1 A/B (A: 1889 PVS, B: 40G5c) in Group 3, and anti-CCR8 1889 PVS bivalent antibody in Group 4. All test articles were supplied as liquid stock solutions and diluted to 0.92 mg/mL for all groups. The dose volume administered was 5.43 mL/kg for all groups.

For all groups, blood was collected from each animal at 0.1667, 1, 6, 24, 72, 168, 240, 336, and 504 hours post dose. 35-40 μL of whole blood was collected via tail vein bleed into SST tubes and stored at room temperature for 30-60 minutes before being processed to serum by centrifugation (11,000 rpm for 5 minutes). The concentrations of test articles in the mouse serum were determined using a total human IgG (generic immunoglobulin pharmacokinetics (GRIP)) ELISA. The minimum quantifiable concentration (MQC) was 15.6 ng/mL for anti-CCR8 TDB 2+1 A/AB (A: 1889 WT, B: 40G5c), 15.6 ng/ml for anti-CCR8 TDB 2+1 A/AB (A: 1889 PVS, B: 40G5c), 31.25 ng/ml for anti-CCR8 TDB 1+1 A/B (A: 1889 PVS, B: 40G5c), and 23.44 ng/ml for anti-CCR8 1889 PVS bivalent antibody.

Serum samples for pharmacokinetic (PK) analysis were collected at 0.1667, 1, 6, 24, 72, 168, 240, 336, and 504 hours post-dose. Samples collected at these time-points were used to plot serum concentration versus time curves. Concentrations below the medium concentration for quality control (MQC) were interpreted to be missing for graphical presentation and were excluded from the datasets used for estimation of individual PK parameters.

The serum concentration versus time data from each animal in Groups 1˜4 were analyzed using the IV bolus input model, WINNONLIN®, Version 8.2 (Certara; Princeton, NJ). The following PK parameters were estimated from the PK curves:
Cₘₐₓ: Maximum observed concentration.
AUCₐₗₗ: Area under the serum concentration versus time curve from Time 0 to time of the last measurable concentration
AUC_{inf}: Area under the serum concentration versus time curve extrapolated to infinity
CL: Clearance (Dose/AUC_{inf}).
Vₛₛ: Volume of distribution at steady state
t_{1/2}: Terminal half-life (ln[2]/z)

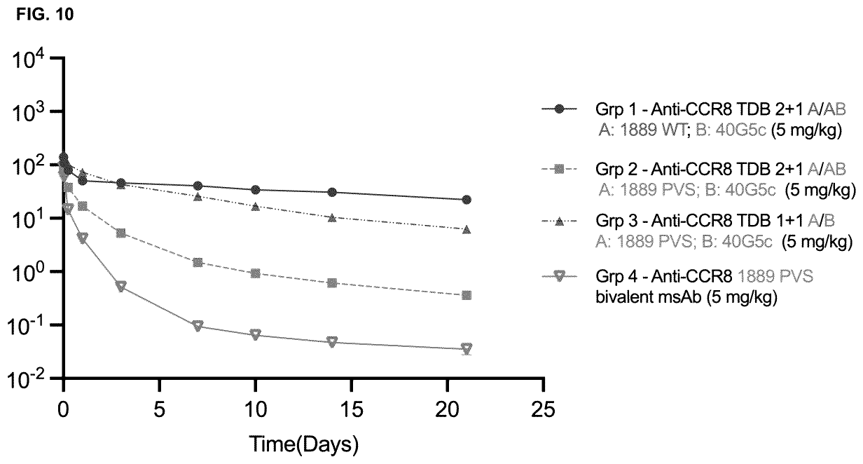

Dosing solutions were assayed for percent recovery. Dosing solutions for all groups recovered within ±20% range; therefore, nominal doses were used for PK analysis for all groups. The concentration-time profiles for all groups are shown in FIG. 10, and the corresponding non-compartmental PK parameter estimates are tabulated in Table 11.

TABLE 11
Results of NCA Analysis

| Group | 1 | 2 | 3 | 4 |
|---|---|---|---|---|
| Treatment | Anti-CCR8 TDB | Anti-CCR8 TDB | Anti-CCR8 TDB | Anti-CCR8 1889 |
| | 2 + 1 A/AB | 2 + 1 A/AB | 1 + 1 A/B | PVS bivalent |
| | A: 1889 WT; | A: 1889 PVS; | A: 1889 PVS; | msAb |
| | B: 40G5c | B: 40G5c | B: 40G5c | |
| Nominal Dose (mg/kg) | 5 | 5 | 5 | 5 |
| Cmax (μg/mL) | 138 ± 3.27 | 91.7 ± 3.78 | 164 ± 14.6 | 90 ± 7.75 |
| AUCₐₗₗ (μg/mL*day) | 770 ± 32.3 | 81.6 ± 2.99 | 527 ± 18.2 | 24.6 ± 1.57 |
| AUC_{inf} (μg/mL*day) | 1330 ± 198 | 85.7 ± 3.6 | 591 ± 14.7 | 25.3 ± 1.95 |
| CL (mL/day/kg) | 3.82 ± 0.565 | 58.4 ± 2.49 | 8.46 ± 0.21 | 198 ± 14.7 |
| Vₛₛ (mL/kg) | 90.9 ± 6.46 | 240 ± 12 | 71.3 ± 4.48 | 366 ± 161 |
| t_{1/2} (days) | 17.2 ± 3.69 | 7.93 ± 0.806 | 7.09 ± 0.739 | 11.7 ± 6.4 |

The concentration-time profiles showed fast drops in serum concentration over time for anti-CCR8 TDB 2+1 A/AB (A: 1889 PVS, B: 40G5c), anti-CCR8 TDB 1+1 A/B (A: 1889 PVS, B: 40G5c), and anti-CCR8 1889 PVS bivalent antibody. A comparison of PK parameters revealed that any test article that contains the 1889 PVS arm had abnormally fast CL values. However, in contrast, anti-CCR8 TDB 2+1 A/AB (A: 1889 WT, B: 40G5c) had CL (3.82±0.565 mL/day/kg) and Vₛₛ (90.9±6.46 mL/kg) values typical of non-binding antibody in SCID mice. These data indicated that the 1889 PVS arm may contribute to poor PK characteristics of affinity-matured anti-CCR8 TDBs.

### Example 5. S12P Mutation Enhances Purification

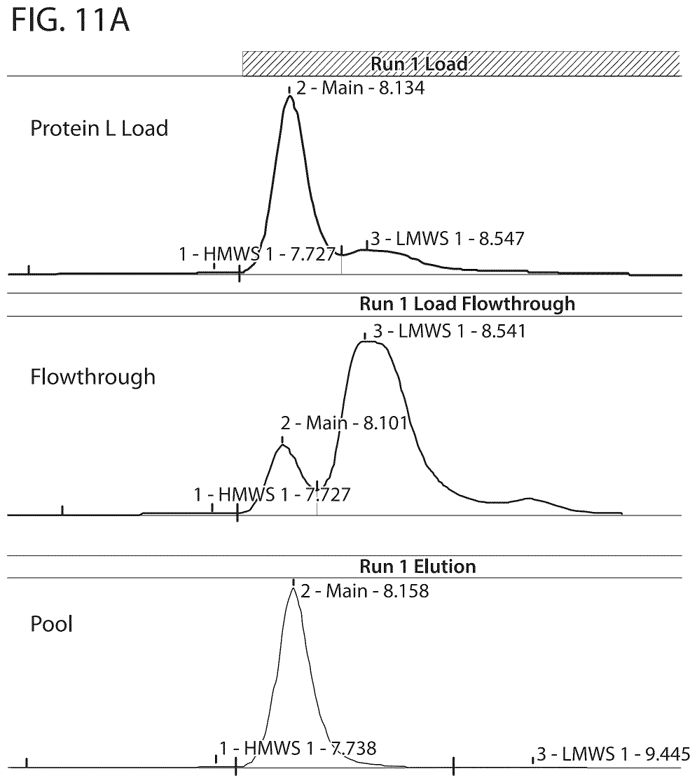

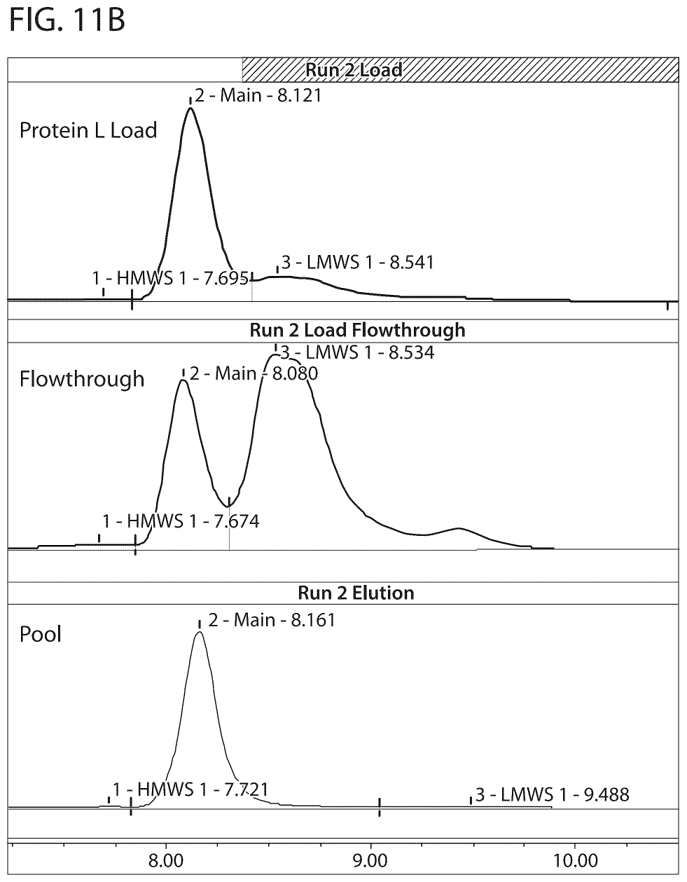

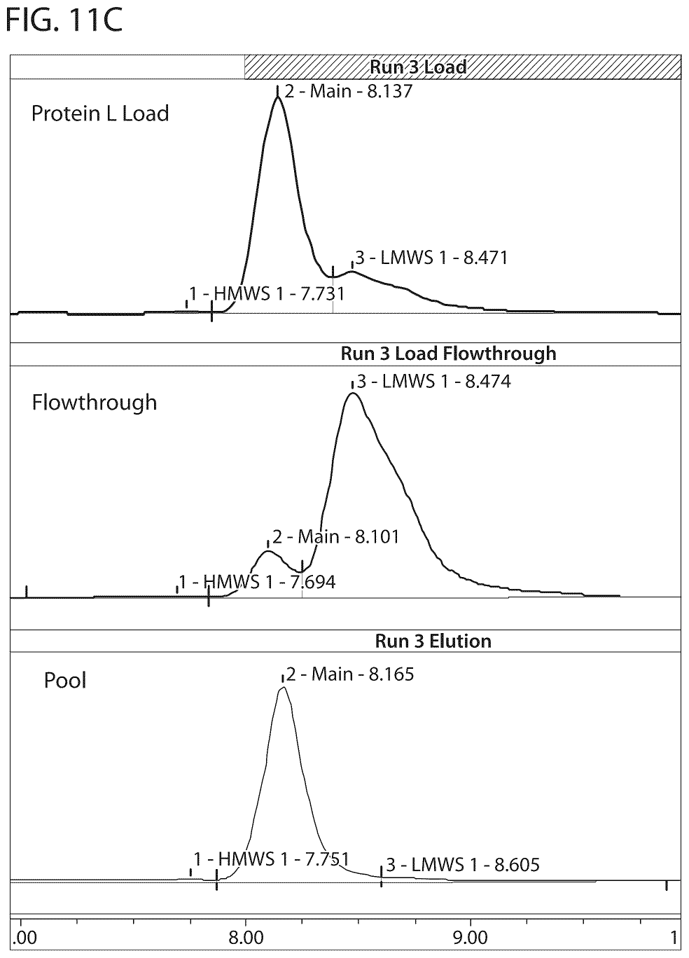

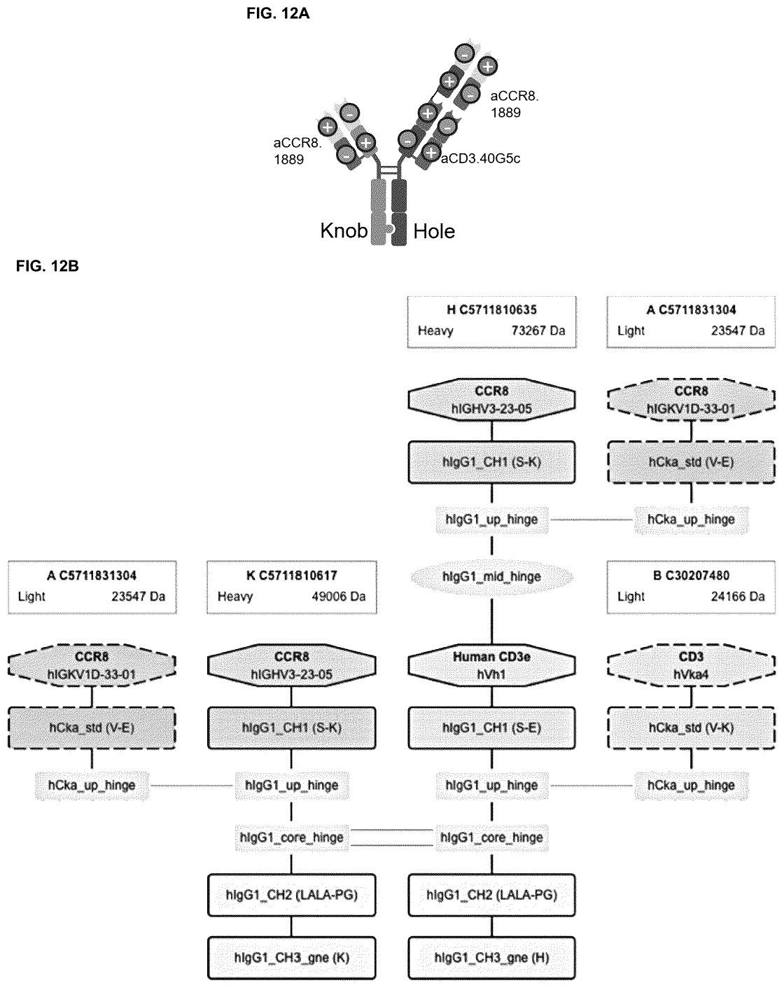

The S12P (numbering according to Kabat) mutation on the anti-CCR8 light chain was evaluated for its ability to simplify purification of generated TDBs. Size-exclusion ultra-high performance liquid chromatography (SE-UPLC) was used to evaluate this effect. Three protein feedstock pools were measured by SE-UPLC to contain 22-25% Low Molecular Weight Forms (LMWFs), and determined by concentration measurements and mass to contain 248 mg to 484 mg protein. These pools were adjusted using 1.5 M Tris base to a minimum pH of 6.5 before being further purified by Protein L affinity chromatography on CAPTO™ L column with a column volume of 36 mL. For all three experiments, this step separated the load feedstock fraction into an unbound flowthrough fraction and a pool of recovered bound fraction. The fraction of unbound protein flowing through the column was collected during the load phase and wash phase. The bound fraction of protein was washed with an equilibration buffer and then eluted from the column using a solution of acetic acid. The recovered bound fraction was collected. For all three runs, the three fractions (load, flowthrough, and pool (i.e., bound)) were analyzed by SE-UPLC. LMWF largely flowed through the column, allowing separation from the main species. Specifically: LMWF present in starting levels of 22-25% in the load, were the predominant species in the flowthrough at levels of 70%-89%, while in the pool of purified protein, LMWF was reduced to 1%-3%. Results are shown in FIG. 11A-FIG. 11C. The S12P mutation (numbering according to Kabat) enabled removal of incorrect species from a mixture.

### Example 6. Further Characterization of Anti-CCR8 TDB Using Human Dissociated Tumor Cell Culture

Based on the results above, a 1889/1889:40G5c anti-CCR8 TDB 2+1 A/AB (A: 1889 P, B: 40G5c) bispecific antigen-binding molecule containing the S12P (numbering according to Kabat) mutation was generated and evaluated. The 1889/1889:40G5c anti-CCR8 TDB 2+1 A/AB (A: 1889 P, B: 40G5c) bispecific antigen-binding molecule further contains charge modifications in the Fab heavy chains and Fab light chains to promote correct assembly of the antigen-binding moieties, as well as knob-in-hole mutations and LALA-PG mutations in the Fc domain subunits to promote association of the two farms and reduce effector function, respectively. FIG. 12A and FIG. 12B contain schematics showing the 1889/1889:40G5c anti-CCR8 TDB 2+1 A/AB (A: 1889 P, B: 40G5c) bispecific antigen-binding molecule.

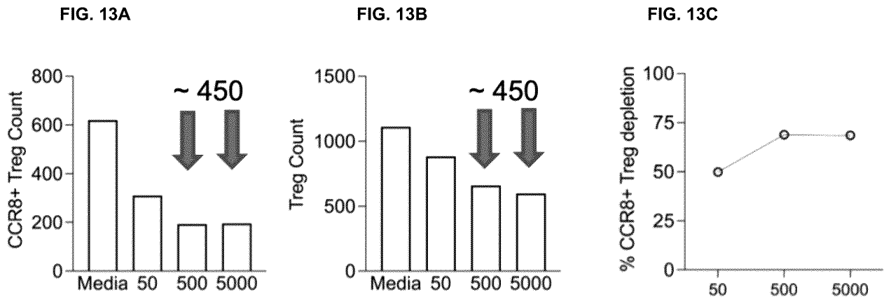

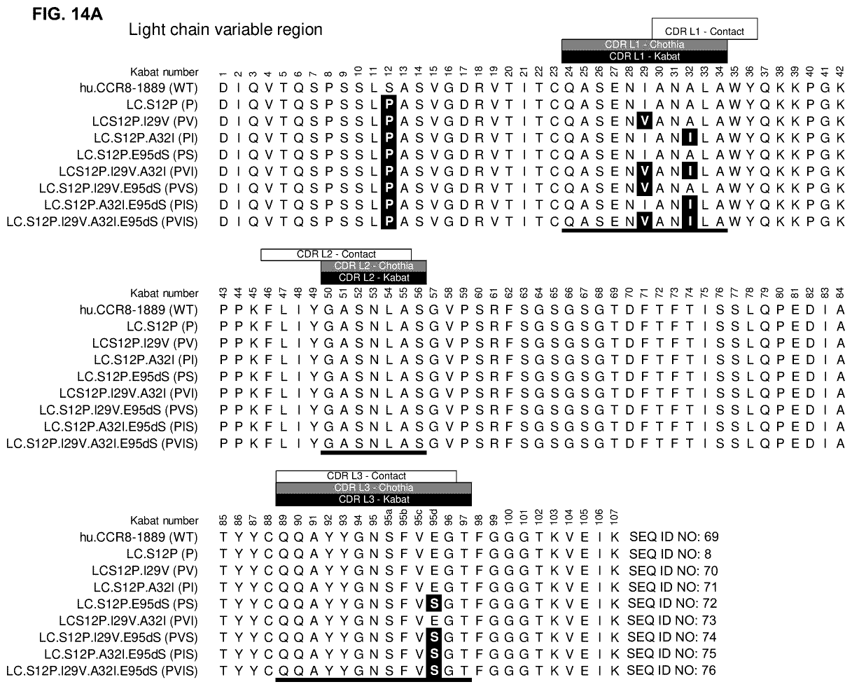

The 1889/1889:40G5c anti-CCR8 TDB 2+1 A/AB (A: 1889 P, B: 40G5c) was evaluated for its ability to deplete Treg cells from cultures comprising dissociated tumor cells. Peripheral blood mononuclear cells (PBMC) were isolated from healthy donor blood using a FICOLL® density gradient (GE Healthcare). CD8+ T cells were magnetically enriched using human CD8+ T Cell isolation kit (Miltenyi Biotec). Human bladder cancer dissociated cells (1×10⁵ cells/well, Discovery life sciences) were thawed and co-cultured with CD8+ T cells for 72 hours with aCCR8-1889/1889.aCD3.40G5c at indicated concentrations. After 72 hours, cells were harvested and sequentially stained with fixable viability dye (ThermoFisher Scientific), blocked with human FcR-blocking reagent (Miltenyi Biotec), stained surface markers, and then intracellular marker using the Foxp3 transcription factor staining buffer set (ThermoFisher Scientific) following the manufacturer's protocols. Samples were acquired by flow cytometry (Symphony, BD) and analyzed using FLOWJO® (BD Biosciences). Cell numbers were quantified with COUNTBRIGHT® absolute counting beads (ThermoFisher Scientific). The ratio of CD8+ T cells and CCR8+ Treg was 2.3. Results are shown in FIG. 13A-FIG. 13C. 2+1 A/AB TDBs were able to deplete Treg cells in cultures comprising dissociated tumor cells.

### Example 7. TDB is Selective to CCR8+ Cells

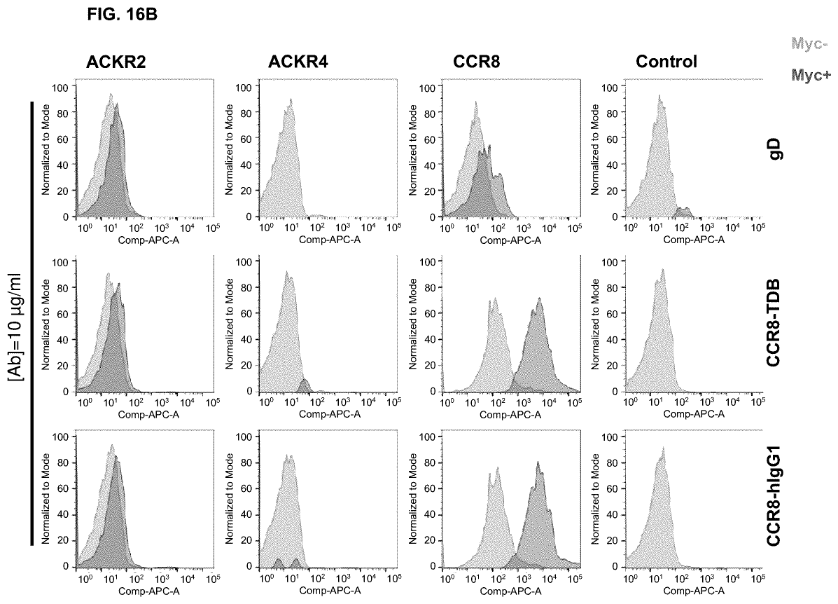

To test whether CCR8 TDB selectively binds to CCR8, HEK293 cells were seeded at 0.6 million in 1 mL of DMEM medium with 10% FCS per well in 12-well cell culture plate and cultured overnight at 37° C. in 5% CO₂ incubator. Cells were then transfected with various DNA constructs (all have C-terminal Myc-tag) using TRANSIT-X2® (Mirus Cat. #MIR6000) with reagent:DNA=3:1 for 24 hours. Transfected HEK293 cells were harvested and stained with CCR8-TDB, CCR8.hIgG1, or isotype control antibody anti-gD in FACS buffer (PBS with 0.5% BSA and 2 mM EDTA) (at 10 μg/mL final concentration) at 4° C. for 30 min, then washed twice with FACS buffer. Cells were then stained with ALEXA FLUOR® 647-anti-Human IgG (Jackson Immunol Lab, 109-606-170, 1:500) at 4° C. for 15 min. Cells were washed twice with FACS buffer. Cells were then fixed and permeabilized with Fixation/Permeabilization Kit (BD Bioscience, Cat. #554714) and followed by ALEXA FLUOR® 488 Anti-Myc tag antibody (9E10, ab202008, 1:100) staining at 4° C. for 30 min. Cells were washed twice and analyzed on FACSCELESTA® and data was analyzed with FLOWJO® software. CCR8 TDB and CCR8.hIgG1 staining on Myc-positive cells are shown in FIG. 16A and FIG. 16B. These results show that CCR8 TDB only binds to CCR8 but not any other sulfated GPCRs tested, including CCR2, CCR3, CCR4, CCR5, CXCR4, ACKR2, and ACKR4.

### Example 8. TDB does not Block CCL1-Mediated Activation of CCR8

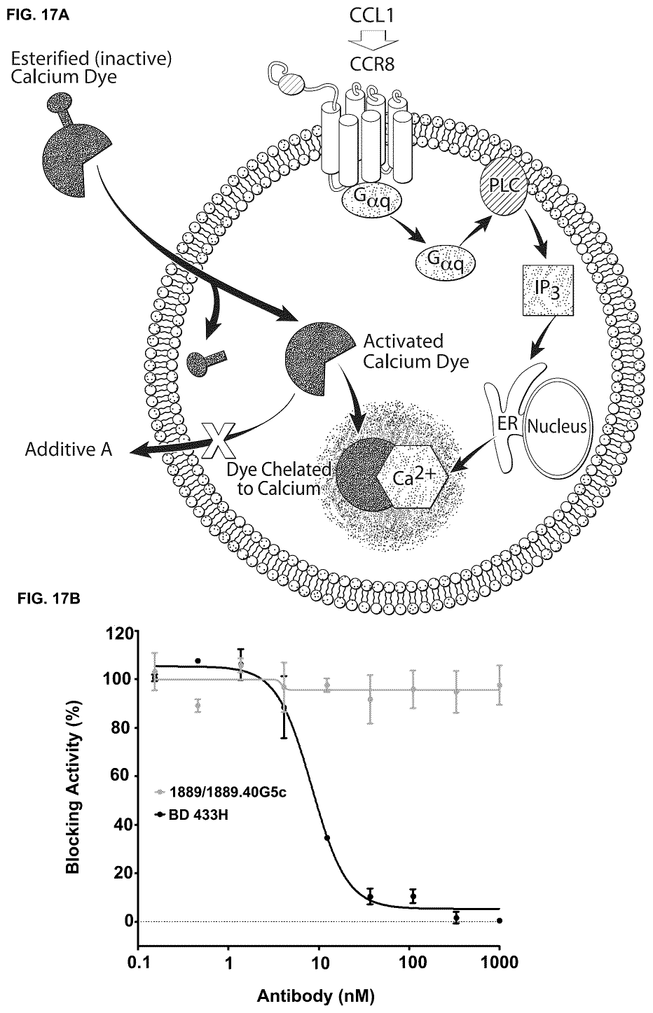

To test whether CCR8 TDB blocks CCL1-mediated activation of CCR8, CCR8 activation was monitored by Ca²⁺ influx using Fluorescent Imaging Plate Reader (FLIPR™) assay with FDSS/μCell (Hamamatsu, Japan), as depicted in FIG. 17A. CHO/hCCR8.Gna 15 cells were seeded in 384-well plate (Corning, Cat. #3764) at 10,000 cells per well in F-12K medium supplemented with 10% FBS. On the second day, the cells were loaded with fluorescence Ca²⁺ dye Fluo-8 NW (Cat #36307, AAT Bioquest) and incubated for 30 minutes at 37° C. followed by 30 minutes incubation at room temperature. Serial diluted CCR8-TDB and control antibody (BD, Cat. #433H) were prepared in Hank's Buffer with HEPES (HHBS) in a clear 384-well plate and hCCL1 in HHBS buffer was also aliquoted in a clear 384-well plate. The FLIPR™ assay was set up on the FDSS/μCell plate reader with continuous monitoring (reading every second) for a total 500 seconds, with antibody addition at 10 second and hCCL1 addition at 300 second. Excitation and emission were set at wavelength 485 nm and 525 nm, respectively. After the run, negative control correction was applied, and the data were normalized against the signal of hCCL1 alone (100%) and plotted as a function of antibody concentrations using GRAPHPAD PRISM®. The result of this experiment is shown in FIG. 17B. The result shows that CCR8 TDB does not block CCL1-mediated activation of CCR8, whereas the control antibody 433H shows dose-dependent inhibition with an IC₅₀ value of 8.4 nM.

### Example 9. Affinities of CCR8 TDB for CCR8 and CD3

To measure the affinity of CCR8 TDB for human or cyno CCR8, CCR8-TDB was labeled via amine conjugation using ALEXA FLUOR® 488 with an incorporation of about 4 fluorophores/molecule. 1 million CHO cells stably expressing either human or cynomolgus CCR8 (about 60,000 and about 30,000 receptors/cell, respectively) were seeded at a concentration of 1 million cells/mL onto a Multidish 2×2 Petri Dish (Ridgeview Instruments AB, Sweden) to form a circular drop of about 3 cm diameter in chambers A and C of the dish. Untransfected CHO cells were seeded in opposing chambers (B and D) in the same manner. Cells were left undisturbed to adhere for 4 hours. Excess media was aspirated, fresh growth media was added, and cells were incubated overnight at 37° C., 5% CO₂. The following day, media was aspirated, and fresh media was added. The cells were incubated at room temperature for 20 minutes prior to starting the assay. The plate was loaded onto a LIGANDTRACER® Green (Ridgeview Instruments AB) for real-time fluorescence measurement. After the fluorescent signal stabilized (15-30 minutes), labeled CCR8-TDB was added in two increasing concentrations (1 nM and 10 nM) until curvature was obtained. Antibody (Ab) dissociation was recorded by aspirating the media and adding fresh media with 10 nM of unlabeled CCR8-TDB to prevent rebinding of labeled Ab. The Ab dissociation was measured overnight to obtain accurate off-rates. Untransfected CHO cells were used to subtract the non-specific binding. Specific binding traces were analyzed using TraceDrawer 1.7 (Ridgeview Instruments) software to determine the association-(kₐ) and dissociation-(k_{d}) rate constants. The equilibrium dissociation constants (K_{D}) for each binding event were calculated as the ratio of the dissociation and association rates, k_{d}/kₐ. Three independent experiments were performed for each cell line. The results are shown below in Table 12. For human CCR8, the kₐ=2.46±0.32 (×10⁵ M⁻¹s⁻¹), k_{d}=2.02±1.56 (×10⁶ s⁻¹), and K_{D}=8 pM. CCR8-TDB interacted with cyno-CCR8 with kₐ=1.86±0.81 (×10⁵ M⁻¹s⁻¹), k_{d} 3.36±2.89 (×10⁻⁶ s⁻¹), and K_{D}=23 pM.

The binding interactions between CCR8-TDB and CD3 were evaluated by surface plasmon resonance (SPR) technology on a BIACORE™ T200 instrument (Cytiva; Marlborough, MA). For kinetics measurements toward human CD3 affinity, CCR8-TDBs were captured by mouse anti-human Fc antibody (Cytiva, cat #BR-1008-39) coated on CM5 biosensor chips to achieve approximately 250 response units (RU). Human CD3-epsilon/delta dimer (linked by murine Fc) was injected in three-fold serial dilutions from 1800 nM to 0 nM in HBS-P buffer (10 mM HEPES, 150 mM NaCl, 0.05% polysorbate 20, pH 7.4) at 37° C. with a flow rate of 100 μL/min. For cyno CD3 kinetics measurements, biotinylated cyno CD3 epsilon peptide (Pyr-DGNEEMGSITQTPYQVSISGTTVILTKK-biotin-amide, SEQ ID NO: 122) was captured on CAP chip (Cytiva, cat #28920234). CCR8-TDB was injected in three-fold serial dilutions from 1800 nM to 0 nM in HBS-P buffer at 37° C. with a flow rate of 100 μL/min. Association rates (kₐ) and dissociation rates (k_{d}) were calculated using a 1:1 Langmuir binding model (BIACORE™ T200 Evaluation Software version 3.1). The equilibrium dissociation constant (K_{D}) was calculated as the ratio k_{d}/kₐ. The results are shown below in Table 12. The recombinant human CD3epsilon/delta bound to CCR8-TDB with an average K_{D} value of 1.16 μM (n=3). The CCR8-TDB bound to cyno CD3 peptide with an average K_{D} value of 0.5 μM (n=3).

TABLE 12
Affinities of CCR8 TDB for CCR8 and CD3

| Target | k · on (1/M*s) | k · off (1/s) | K_{D} | |
|---|---|---|---|---|
| Human CCR8^{#} | 2.46 ± 0.32 (×10⁵) | 2.02 ± 1.56 (×10⁻⁶) | 8 | pM |
| Cyno CCR8^{#} | 1.86 ± 0.81 (×10⁵) | 3.36 ± 2.89 (×10⁻⁶) | 23 | pM |
| Human CD3* | 2.25 × 10⁵ | 0.26 | 1.16 | uM |
| Cyno CD3* | 3.94 × 10⁵ | 0.175 | 0.5 | uM |

*SPR; +37° C., huCD3ed-mIgG2a.Fc or N-terminal peptide
^{#}Cellular binding using LIGAND TRACER ®

### Example 10. CCR8 TDB Reduces CCR8+ Tregs in Cyno Peripheral Blood

To test whether CCR8 TDB reduces CCR8+ Tregs in cyno peripheral blood, a repeat-dose tolerability, toxicokinetics, and pharmacodynamics study of anti-CCR8 TDB in cynomolgus monkeys was conducted. 3 male cynomolgus monkeys per group were administered either vehicle control or anti-CCR8 TDB intravenously on Day 1 and Day 11. Cynomolgus monkeys were of Cambodian origin and approximately 2-4 kg and 2-6 years old. Animals were randomly assigned to receive either 20 mM Histidine acetate, 0.15 M NaCl, pH5.5 (control) or anti-CCR8 TDB at 10 mg/kg via 1-hour intravenous infusion at 2 mL/kg/hr. Blood samples were collected at pre-study Day −4 (Day 36 of pre-observation), Day 3, Day 7, Day 11 (pre-dose), Day 13 and Day 20. 500 μL of total blood per condition was divided into 5 separate tubes with 100 μL blood and each was lysed using 2 mL of 1× Lysing Buffer (BD biosciences Cat. #555899) for 15 minutes at room temperature. The samples were washed with 1.5 ml Wash Buffer and centrifuged at 500×g for 5 mins at room temperature. All 5 tubes were resuspended in 200 μL of Wash Buffer, then combined into one tube and centrifuged at 500×g for 5 mins at room temperature before surface staining. All the samples were blocked with 5 μL of purified anti-human CD32 antibody (BD Biosciences Cat. #303202) and 5 μL of monocyte blocker (BD Biosciences Cat. #426103) for 10 minutes. Afterwards, cells were stained with the antibody mixture B or C surface antibodies for 45 minutes in a refrigerator while protected from the light. The composition of antibody mixture B and C are shown in Tables 4 and 5 above, respectively.

After the surface staining, all the samples were washed 3 times with 1.5 mL Wash Buffer and centrifuged at 500×g for 5 minutes at 4° C. At the end of the last wash, supernatant was discarded and 500 μL 1× Fix/Perm buffer (eBioscince Cat. #00-5123-43 and #00-5223-56) was added for overnight (12 to 20 hours) incubation in a refrigerator (set at 4° C.) while protected from light. On the second day, the samples were washed 3 times with 1×1.5 mL Permeabilization buffer (eBioscince Cat. #00-8333-56) and centrifuged at room temperature, 500×g for 5 minutes before incubation with Antibody mixture D (100 μL) for 60 minutes (±5 minutes) at room temperature while protected from light. The composition of antibody mixture D is shown in Table 6 above.

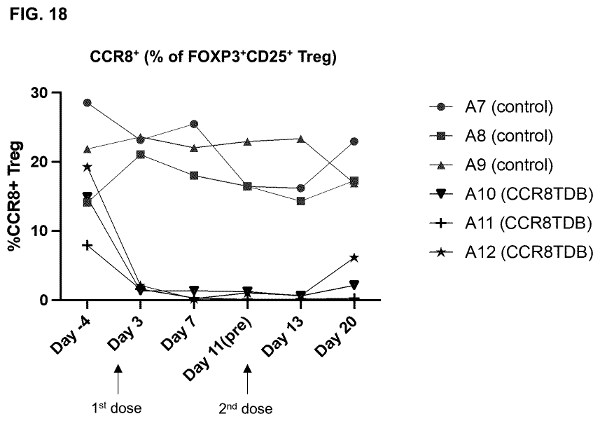

The samples were then washed 2 times with Wash Buffer and resuspended in 300 μL Stain Buffer (BD Biosciences Cat. #554656) and acquired using LSRFORTESSA™ X-20 (BD Biosciences, MUT/150) and FACSDIVA™ Software (ver.8.0.1). Analysis was performed using FLOWJO® (ver.10.7.1). Regulatory T (Treg) cells were identified as CD45+, CD14−, CD4+, CD8−, FOXP3+ and CD25 cells. GRAPHPAD PRISM® 9 was used for plotting. The results are shown in FIG. 18 and Table 13 below. These results show that CCR8 TDB induced depletion of CCR8+ regulatory T (Treg) cells in the peripheral blood of cynomolgus monkeys.

TABLE 13
Percentage of CCR*+ Treg cells in the peripheral blood
of cynomolgus monkeys treated with control or CCR8 TDB over time

| Time | A7 | A8 | A9 | A10 | A11 | A12 |
|---|---|---|---|---|---|---|
| points | (control) | (control) | (control) | (CCR8TDB) | (CCR8TDB) | (CCR8TDB) |
| Day −4 | 28.54 | 14.11 | 21.85 | 14.87 | 7.93 | 19.25 |
| Day 3 | 23.16 | 21.03 | 23.58 | 1.36 | 1.59 | 2.15 |
| Day 7 | 25.46 | 18.02 | 22.03 | 1.34 | 0.31 | 0.2 |
| Day 11(pre) | 16.44 | 16.47 | 22.95 | 1.24 | 0.11 | 1.03 |
| Day 13 | 16.19 | 14.32 | 23.34 | 0.62 | 0.15 | 0.69 |
| Day 20 | 22.95 | 17.28 | 16.89 | 2.15 | 0.26 | 6.15 |

### Example 11. CCR8 TDB Reduces CCR8 mRNA Expression in Cynomolgus Monkey Blood

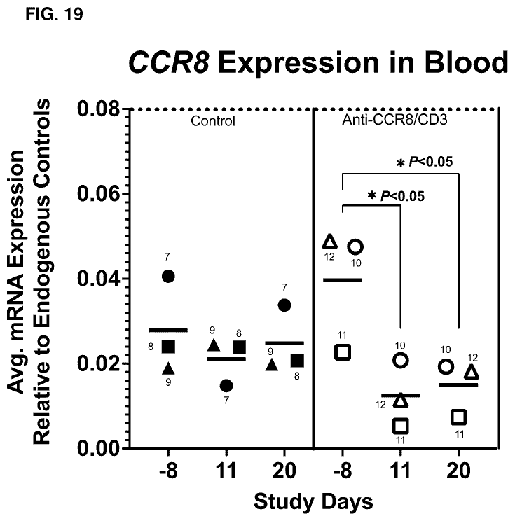

To test whether CCR8 TDB reduces CCR8 mRNA expression in cynomolgus monkey blood, 6 cynomolgus monkeys were divided into two groups treated with either CCR8 TDB (10 mg/kg) or vehicle control. Isolation of total RNA was conducted using RNEASY® Protect Animal Blood Kit (Catalog No. 73224; Qiagen, Hilden, Germany) according to manufacturer's instructions. One-step reverse transcription and pre-amplification were carried out using SUPERSCRIPT™ III One-Step RT-PCR System (Catalog No. 12574018; Thermo Fisher; Waltham, MA). PCR reaction for relative gene expression was conducted using TAQMAN™ Universal PCR Master Mix II, no UNG (Catalog No. 4440048; Thermo Fisher) and the 96.96 DYNAMIC ARRAY™ IFC (Catalog No. BMK-M-96.96; Fluidigm; South San Francisco, CA) on the BIOMARK™ HD System (Fluidigm). Threshold cycle (Ct) values were generated using Fluidigm's Real-Time PCR software, and subsequent relative gene expression data were normalized against the geometric mean of endogenous control genes and analyzed by TIBCO SPOTFIRE® (Palo Alto, CA). Graph preparation and statistical analyses were performed by using GRAPHPAD PRISM® software (San Diego, CA). The average mRNA expression of CCR8 in cynomolgus monkey blood collected at baseline (Study Day −8) and at Study Days 11 (pre-second dose) and 20 (endpoint) for each group of animals and for each individual animal is shown in Tables 14 and 15 below, respectively. As shown in FIG. 19, the CCR8 gene expressions on Day 11 (pre-second dose) and Day 20 were significantly reduced compared to baseline level on Day −8 in whole blood from cynomolgus monkeys dosed with 10 mg/kg anti-CCR8/TDB.

TABLE 14
Average mRNA Expression of CCR8 in Cynomolgus
Monkey Blood for Each Group of Animals

| | Control | | | Anti-CCR8/CD3 TDB | | |
|---|---|---|---|---|---|---|
| Study Day | Day −8 | Day 11 | Day 20 | Day −8 | Day 11 | Day 20 |
| Mean +/− | 0.0279 +/− | 0.0211 +/− | 0.0248 +/− | 0.0397 +/− | 0.0125 +/− | 0.015 +/− |
| S.D. | 0.0113 | 0.00544 | 0.0078 | 0.0147 | 0.0078 | 0.00658 |

TABLE 15
Average mRNA Expression of CCR8 in Cynomolgus
Monkey Blood for Individual Animals
Control

| | Animal 7 | | | Animal 8 | | | Animal 9 | | |
|---|---|---|---|---|---|---|---|---|---|
| Gene | Day −8 | Day 11 | Day 20 | Day −8 | Day 11 | Day 20 | Day −8 | Day 11 | Day 20 |
| CCR8 | 0.0406 | 0.0148 | 0.0338 | 0.024 | 0.0239 | 0.0207 | 0.019 | 0.0245 | 0.0199 |

Anti-CCR8 TDB

| | Animal 10 | | | Animal 11 | | | Animal 12 | | |
|---|---|---|---|---|---|---|---|---|---|
| Gene | Day −8 | Day 11 | Day 20 | Day −8 | Day 11 | Day 20 | Day −8 | Day 11 | Day 20 |
| CCR8 | 0.0475 | 0.0208 | 0.0193 | 0.0227 | 0.0053 | 0.0074 | 0.0489 | 0.0115 | 0.0182 |

### Example 12. CCR8 TDB Induces Minimal Cytokine Release in Cynomolgus Monkey

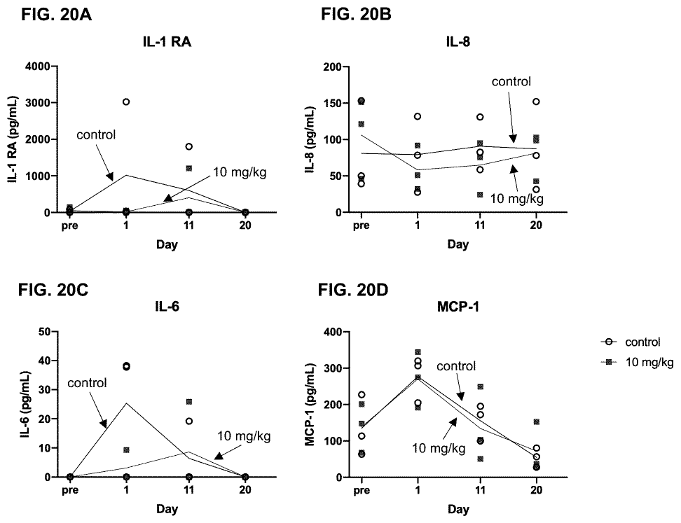

To test whether CCR8 TDB induces cytokine release in cynomolgus monkey, a repeat-dose tolerability, toxicokinetics, and pharmacodynamics study was conducted. 3 male cynomolgus monkeys per group were administered either vehicle control or anti-CCR8 TDB intravenously on Day 1 and Day 11. Cynomolgus monkeys were of Cambodian origin and approximately 2-4 kg and 2-6 years old. Animals were randomly assigned to receive either 20 mM Histidine acetate, 0.15 M NaCl, pH 5.5 (control) or anti-CCR8 TDB at 10 mg/kg via 1-hour intravenous infusion at 2 mL/kg/hr. Blood samples were collected at pre-study, Day 1 (6 hours post end of infusion (EOI) of the first dose), Day 11 (6 hours post EOI of the 2nd dose), and Day 20 (a single time point). Blood (approximately 1 mL) was drawn from the femoral vein and left at room temperature for 20 to 60 minutes. Serum was obtained by centrifugation (room temperature, 1700×g, 10 minutes) and stored in a deep freezer (−70° C. or below) until analysis. Each sample was assayed for IL-1B, IL-1RA, IL-2, IL-4, IL-5, IL-6, IL-8, IL-10, IL-12/23 (p40), IL-13, IL-17A, IFN-γ, TNF-α, MCP-1, G-CSF and GM-CSF. The concentrations (pg/mL) of each parameter were analyzed using PROCARTAPLEX™ Multiplex Immunoassays [Non-Human Primate Assays] by BIO-PLEX® 200 (Bio-Rad Laboratories, Inc.). The results are summarized in Table 16 below, and line graphs generated using GRAPHPAD PRISM® for selected cytokines (IL-1 RA, IL-8, IL-6, and MCP-1) are shown in FIG. 20A-FIG. 20D. The results show that the CCR8 TDB did not affect any cytokines examined when administered at 10 mg/kg intravenously to male cynomolgus monkeys on Day 1 and Day 11.

TABLE 16
Cytokine levels in cynomolgus monkeys treated with CCR8 TDB or control at various time points.

| | | IL-1β (pg/mL) | | | | IL-1RA (pg/mL) | | | |
|---|---|---|---|---|---|---|---|---|---|
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | BLOQ | BLOQ | BLOQ | BLOQ | BLOQ | BLOQ | BLOQ | BLOQ |
| 0 (mg/kg) | 8 | BLOQ | BLOQ | BLOQ | BLOQ | BLOQ | 31.04 | BLOQ | BLOQ |
| | 9 | BLOQ | BLOQ | BLOQ | BLOQ | 80.29 | 3024.24 | 1802.82 | BLOQ |
| | Mean | 0.00 | 0.00 | 0.00 | 0.00 | 26.76 | 1018.43 | 600.94 | 0.00 |
| | ±S.D. | 0.00 | 0.00 | 0.00 | 0.00 | 46.36 | 1737.15 | 1040.86 | 0.00 |
| Anti-CCR8 | 10 | BLOQ | BLOQ | BLOQ | BLOQ | 141.42 | 47.41 | BLOQ | BLOQ |
| TDB | 11 | 2.62 | BLOQ | BLOQ | BLOQ | BLOQ | BLOQ | 1206.44 | BLOQ |
| 10 (mg/kg) | 12 | BLOQ | BLOQ | BLOQ | BLOQ | BLOQ | BLOQ | BLOQ | BLOQ |
| | Mean | 0.87 | 0.00 | 0.00 | 0.00 | 47.14 | 15.80 | 402.15 | 0.00 |
| | ±S.D. | 1.51 | 0.00 | 0.00 | 0.00 | 81.65 | 27.37 | 696.54 | 0.00 |
| | | IL-2 (pg/mL) | | | | IL-4 (pg/mL) | | | |
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | BLOQ | BLOQ | BLOQ | BLOQ | BLOQ | BLOQ | BLOQ | BLOQ |
| 0 (mg/kg) | 8 | BLOQ | BLOQ | BLOQ | BLOQ | BLOQ | BLOQ | BLOQ | BLOQ |
| | 9 | BLOQ | BLOQ | BLOQ | BLOQ | BLOQ | BLOQ | BLOQ | BLOQ |
| | Mean | 0.00 | 0.00 | 0.00 | 0.00 | 0.00 | 0.00 | 0.00 | 0.00 |
| | ±S.D. | 0.00 | 0.00 | 0.00 | 0.00 | 0.00 | 0.00 | 0.00 | 0.00 |
| Anti-CCR8 | 10 | BLOQ | BLOQ | BLOQ | BLOQ | BLOQ | BLOQ | BLOQ | BLOQ |
| TDB | 11 | 3.69 | BLOQ | BLOQ | BLOQ | BLOQ | BLOQ | BLOQ | BLOQ |
| 10 (mg/kg) | 12 | BLOQ | BLOQ | BLOQ | BLOQ | BLOQ | BLOQ | BLOQ | BLOQ |
| | Mean | 1.23 | 0.00 | 0.00 | 0.00 | 0.00 | 0.00 | 0.00 | 0.00 |
| | ±S.D. | 2.13 | 0.00 | 0.00 | 0.00 | 0.00 | 0.00 | 0.00 | 0.00 |
| | | IL-5 (pg/mL) | | | | IL-6 (pg/mL) | | | |
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | BLOQ | BLOQ | BLOQ | BLOQ | BLOQ | BLOQ | BLOQ | BLOQ |
| 0 (mg/kg) | 8 | BLOQ | BLOQ | BLOQ | BLOQ | BLOQ | 37.79 | BLOQ | BLOQ |
| | 9 | BLOQ | BLOQ | BLOQ | BLOQ | BLOQ | 38.28 | 19.18 | BLOQ |
| | Mean | 0.00 | 0.00 | 0.00 | 0.00 | 0.00 | 25.36 | 6.39 | 0.00 |
| | ±S.D. | 0.00 | 0.00 | 0.00 | 0.00 | 0.00 | 21.96 | 11.07 | 0.00 |
| Anti-CCR8 | 10 | BLOQ | BLOQ | BLOQ | BLOQ | BLOQ | BLOQ | BLOQ | BLOQ |
| TDB | 11 | BLOQ | BLOQ | BLOQ | BLOQ | BLOQ | 9.29 | 25.87 | BLOQ |
| 10 (mg/kg) | 12 | BLOQ | BLOQ | BLOQ | BLOQ | BLOQ | BLOQ | BLOQ | BLOQ |
| | Mean | 0.00 | 0.00 | 0.00 | 0.00 | 0.00 | 3.10 | 8.62 | 0.00 |
| | ±S.D. | 0.00 | 0.00 | 0.00 | 0.00 | 0.00 | 5.36 | 14.94 | 0.00 |
| | | IL-8 (pg/mL) | | | | IL-10 (pg/mL) | | | |
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | 153.21 | 131.80 | 130.90 | 78.11 | BLOQ | BLOQ | BLOQ | BLOQ |
| 0 (mg/kg) | 8 | 50.13 | 27.52 | 58.62 | 31.38 | BLOQ | BLOQ | BLOQ | BLOQ |
| | 9 | 39.30 | 78.45 | 82.54 | 152.13 | BLOQ | BLOQ | BLOQ | BLOQ |
| | Mean | 80.88 | 79.26 | 90.69 | 87.21 | 0.00 | 0.00 | 0.00 | 0.00 |
| | ±S.D. | 62.87 | 52.14 | 36.82 | 60.89 | 0.00 | 0.00 | 0.00 | 0.00 |
| Anti-CCR8 | 10 | 45.91 | 32.04 | 24.02 | 42.52 | BLOQ | BLOQ | BLOQ | BLOQ |
| TDB | 11 | 151.24 | 91.74 | 75.47 | 102.61 | BLOQ | BLOQ | BLOQ | BLOQ |
| 10 (mg/kg) | 12 | 121.01 | 50.98 | 94.85 | 98.56 | BLOQ | BLOQ | BLOQ | BLOQ |
| | Mean | 106.05 | 58.25 | 64.78 | 81.23 | 0.00 | 0.00 | 0.00 | 0.00 |
| | ±S.D. | 54.23 | 30.51 | 36.61 | 33.58 | 0.00 | 0.00 | 0.00 | 0.00 |
| | | IL-12/23 (p40) (pg/mL) | | | | IL-13 (pg/mL) | | | |
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | 2.32 | BLOQ | 3.38 | 4.62 | BLOQ | BLOQ | BLOQ | BLOQ |
| 0 (mg/kg) | 8 | BLOQ | BLOQ | BLOQ | BLOQ | BLOQ | BLOQ | BLOQ | BLOQ |
| | 9 | 3.28 | BLOQ | BLOQ | BLOQ | BLOQ | BLOQ | BLOQ | BLOQ |
| | Mean | 1.87 | 0.00 | 1.13 | 1.54 | 0.00 | 0.00 | 0.00 | 0.00 |
| | ±S.D. | 1.69 | 0.00 | 1.95 | 2.67 | 0.00 | 0.00 | 0.00 | 0.00 |
| Anti-CCR8 | 10 | BLOQ | BLOQ | BLOQ | BLOQ | BLOQ | BLOQ | BLOQ | BLOQ |
| TDB | 11 | BLOQ | BLOQ | BLOQ | BLOQ | BLOQ | BLOQ | BLOQ | BLOQ |
| 10 (mg/kg) | 12 | BLOQ | BLOQ | BLOQ | BLOQ | BLOQ | BLOQ | BLOQ | BLOQ |
| | Mean | 0.00 | 0.00 | 0.00 | 0.00 | 0.00 | 0.00 | 0.00 | 0.00 |
| | ±S.D. | 0.00 | 0.00 | 0.00 | 0.00 | 0.00 | 0.00 | 0.00 | 0.00 |
| | | IL-17A (pg/mL) | | | | IFN-γ (pg/mL) | | | |
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | BLOQ | BLOQ | BLOQ | BLOQ | BLOQ | BLOQ | BLOQ | BLOQ |
| 0 (mg/kg) | 8 | BLOQ | BLOQ | BLOQ | BLOQ | BLOQ | BLOQ | BLOQ | BLOQ |
| | 9 | BLOQ | BLOQ | BLOQ | BLOQ | BLOQ | BLOQ | BLOQ | BLOQ |
| | Mean | 0.00 | 0.00 | 0.00 | 0.00 | 0.00 | 0.00 | 0.00 | 0.00 |
| | ±S.D. | 0.00 | 0.00 | 0.00 | 0.00 | 0.00 | 0.00 | 0.00 | 0.00 |
| Anti-CCR8 | 10 | BLOQ | BLOQ | BLOQ | BLOQ | BLOQ | BLOQ | BLOQ | BLOQ |
| TDB | 11 | BLOQ | BLOQ | BLOQ | BLOQ | 2.14 | BLOQ | BLOQ | 7.70 |
| 10 (mg/kg) | 12 | BLOQ | BLOQ | BLOQ | BLOQ | BLOQ | BLOQ | BLOQ | BLOQ |
| | Mean | 0.00 | 0.00 | 0.00 | 0.00 | 0.71 | 0.00 | 0.00 | 2.57 |
| | ±S.D. | 0.00 | 0.00 | 0.00 | 0.00 | 1.24 | 0.00 | 0.00 | 4.45 |
| | | TNF-α (pg/mL) | | | | MCP-1 (pg/mL) | | | |
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | BLOQ | BLOQ | BLOQ | BLOQ | 227.24 | 306.39 | 172.23 | 56.61 |
| 0 (mg/kg) | 8 | BLOQ | BLOQ | BLOQ | BLOQ | 63.58 | 204.70 | 99.67 | 27.73 |
| | 9 | BLOQ | BLOQ | BLOQ | BLOQ | 113.60 | 319.86 | 195.08 | 80.36 |
| | Mean | 0.00 | 0.00 | 0.00 | 0.00 | 134.81 | 276.98 | 155.66 | 54.90 |
| | ±S.D. | 0.00 | 0.00 | 0.00 | 0.00 | 83.87 | 62.96 | 49.82 | 26.36 |
| Anti-CCR8 | 10 | BLOQ | BLOQ | BLOQ | BLOQ | 147.54 | 274.52 | 102.99 | 36.36 |
| TDB | 11 | BLOQ | BLOQ | BLOQ | BLOQ | 200.94 | 343.97 | 248.83 | 152.26 |
| 10 (mg/kg) | 12 | BLOQ | BLOQ | BLOQ | BLOQ | 67.49 | 191.64 | 50.42 | 28.10 |
| | Mean | 0.00 | 0.00 | 0.00 | 0.00 | 138.66 | 270.04 | 134.08 | 72.24 |
| | ±S.D. | 0.00 | 0.00 | 0.00 | 0.00 | 67.17 | 76.26 | 102.79 | 69.42 |

| | | G-CSF (pg/mL) | | | | GM-CSF (pg/mL) | | | |
|---|---|---|---|---|---|---|---|---|---|
| | Animal | Day | | | | | | | |
| Group | No. | −8^{a)} | 1^{b)} | 11^{c)} | 20^{d)} | −8^{a)} | 1^{b)} | 11^{c)} | 20^{d)} |
| Control | 7 | BLOQ | BLOQ | BLOQ * | BLOQ # | BLOQ | BLOQ | BLOQ | BLOQ |
| 0 (mg/kg) | 8 | 10.34 | BLOQ | BLOQ * | BLOQ | BLOQ | BLOQ | BLOQ | BLOQ |
| | 9 | BLOQ | BLOQ * | BLOQ | BLOQ | BLOQ | BLOQ | BLOQ | BLOQ |
| | Mean | 3.45 | 0.00 | 0.00 | 0.00 | 0.00 | 0.00 | 0.00 | 0.00 |
| | ±S.D. | 5.97 | 0.00 | 0.00 | 0.00 | 0.00 | 0.00 | 0.00 | 0.00 |
| Anti-CCR8 | 10 | BLOQ # | BLOQ | BLOQ # | BLOQ # | BLOQ | BLOQ | BLOQ | BLOQ |
| TDB | 11 | BLOQ | BLOQ | BLOQ | BLOQ | BLOQ | BLOQ | BLOQ | BLOQ |
| 10 (mg/kg) | 12 | BLOQ | BLOQ | BLOQ * | BLOQ | BLOQ | BLOQ | BLOQ | BLOQ |
| | Mean | 0.00 | 0.00 | 0.00 | 0.00 | 0.00 | 0.00 | 0.00 | 0.00 |
| | ±S.D. | 0.00 | 0.00 | 0.00 | 0.00 | 0.00 | 0.00 | 0.00 | 0.00 |

^{a)}6 hr after other acclimation collections
^{b)}6 hr after 1st dosing (EOI)
^{c)}6 hr after 2nd dosing (EOI)
^{d)}Once
* Reanalysis data
#: Reference data
EOI: End of Infusion
BLOQ: Below the lower limit of quantification (LLOQ) were regarded as 0 pg/mL.
LLOQ (IL-1β): 2.56 pg/mL, LLOQ (IL-1RA): 17.82 pg/mL, LLOQ (IL-2): 1.73 pg/mL, LLOQ (IL-4): 5.32 pg/mL, LLOQ (IL-5): 4.13 pg/mL, LLOQ (IL-6): 6.32 pg/mL, LLOQ (IL-10): 1.72 pg/mL, LLOQ (IL-12/23 (p40)): 1.64 pg/mL, LLOQ (IL-13): 2.42 pg/mL, LLOQ (IL-17A): 5.20 pg/mL, LLOQ (IFN-γ): 1.78 pg/mL, LLOQ (TNF-α): 3.61 pg/mL, LLOQ (G-CSF): 7.06 pg/mL, LLOQ (GM-CSF): 12.13 pg/mL.

### Embodiments

Some embodiments of the technology described herein can be defined according to any of the following numbered embodiments:

1. A bispecific antigen-binding molecule comprising:
(a) a first antigen-binding domain that binds C-C motif chemokine receptor 8 (CCR8), wherein the first antigen-binding domain comprises the following six complementarity-determining regions (CDRs):
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds cluster of differentiation 3 (CD3), wherein the second antigen-binding domain comprises the following six CDRs:
   (i) a CDR-H1 comprising the amino acid sequence NYYIH (SEQ ID NO: 17);
   (ii) a CDR-H2 comprising the amino acid sequence WIYPGDGNTKYNEKFKG (SEQ ID NO: 18);
   (iii) a CDR-H3 comprising the amino acid sequence DSYSNYYFDY (SEQ ID NO: 19);
   (iv) a CDR-L1 comprising the amino acid sequence KSSQSLLNSRTRKNYLA (SEQ ID NO: 20);
   (v) a CDR-L2 comprising the amino acid sequence WASTRES (SEQ ID NO: 21); and
   (vi) a CDR-L3 comprising the amino acid sequence TQSFILRT (SEQ ID NO: 22).

2. The bispecific antigen-binding molecule of embodiment 1, wherein the first antigen-binding domain comprises a light chain variable region (VL) domain and a heavy chain variable region (VH) domain, and wherein:
(a) the VL domain comprises a proline residue at position 12 (numbering according to Kabat); and/or
(b) the VL domain comprises a lysine residue at position 38 and the VH domain comprises a glutamic acid residue at position 39 (numbering according to Kabat).

3. The bispecific antigen-binding molecule of embodiment 1 or 2, wherein the second antigen-binding domain comprises a VL domain and a VH domain, and wherein the VL domain comprises a glutamic acid residue at position 38 and the VH domain comprises a lysine residue at position 39 (numbering according to Kabat).

4. The bispecific antigen-binding molecule of any one of embodiments 1-3, wherein:
(a) the first antigen-binding domain comprises one or more of the following eight framework regions (FRs):
   (i) an FR-H1 comprising the amino acid sequence of SEQ ID NO: 9;
   (ii) an FR-H2 comprising the amino acid sequence of SEQ ID NO: 10;
   (iii) an FR-H3 comprising the amino acid sequence of SEQ ID NO: 11;
   (iv) an FR-H4 comprising the amino acid sequence of SEQ ID NO: 12;
   (v) an FR-L1 comprising the amino acid sequence of SEQ ID NO: 13;
   (vi) an FR-L2 comprising the amino acid sequence of SEQ ID NO: 14;
   (vii) an FR-L3 comprising the amino acid sequence of SEQ ID NO: 15; and/or
   (viii) an FR-L4 comprising the amino acid sequence of SEQ ID NO: 16; and/or
(b) the second antigen-binding domain comprises one or more of the following eight FRs:
   (i) an FR-H1 comprising the amino acid sequence of SEQ ID NO: 25;
   (ii) an FR-H2 comprising the amino acid sequence of SEQ ID NO: 26;
   (iii) an FR-H3 comprising the amino acid sequence of SEQ ID NO: 27;
   (iv) an FR-H4 comprising the amino acid sequence of SEQ ID NO: 28;
   (v) an FR-L1 comprising the amino acid sequence of SEQ ID NO: 29;
   (vi) an FR-L2 comprising the amino acid sequence of SEQ ID NO: 30;
   (vii) an FR-L3 comprising the amino acid sequence of SEQ ID NO: 31; and/or
   (viii) an FR-L4 comprising the amino acid sequence of SEQ ID NO: 32.

5. The bispecific antigen-binding molecule of any one of embodiments 1-4, wherein:
the first antigen-binding domain comprises:
   (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b); and/or
the second antigen-binding domain comprises:
   (a) a VH domain comprising an amino acid sequence having at least 80%, 85%, 90%, 95%, 96%, 97%, 98%, 99%, or 100% sequence identity to the amino acid sequence of SEQ ID NO: 23; (b) a VL domain comprising an amino acid sequence having at least 80%, 85%, 90%, 95%, 96%, 97%, 98%, 99%, or 100% sequence identity to the amino acid sequence of SEQ ID NO: 24; or (c) a VH domain as in (a) and a VL domain as in (b).

6. The bispecific antigen-binding molecule of any one of embodiments 1-5, wherein:
the first antigen-binding domain comprises:
   (a) a VH domain comprising the amino acid sequence of SEQ ID NO: 7; (b) a VL domain comprising the amino acid sequence of SEQ ID NO: 8; or (c) a VH domain as in (a) and a VL domain as in (b); and/or
the second antigen-binding domain comprises:
   (a) a VH domain comprising the amino acid sequence of SEQ ID NO: 23; (b) a VL domain comprising the amino acid sequence of SEQ ID NO: 24; or (c) a VH domain as in (a) and
a VL domain as in (b).

7. The bispecific antigen-binding molecule of embodiment 6, wherein:
the first antigen-binding domain comprises:
   (a) a VH domain comprising the amino acid sequence of SEQ ID NO: 7 and a VL domain comprising the amino acid sequence of SEQ ID NO: 8; and the second antigen-binding domain comprises:
   (b) a VH domain comprising the amino acid sequence of SEQ ID NO: 23 and a VL domain comprising the amino acid sequence of SEQ ID NO: 24.

8. The bispecific antigen-binding molecule of any one of embodiments 1-7, wherein the first antigen-binding domain is a Fab molecule comprising a Fab light chain and a Fab heavy chain and/or the second antigen-binding domain is a Fab molecule comprising a Fab light chain and a Fab heavy chain.

9. The bispecific antigen-binding molecule of any one of embodiments 1-8, wherein the first antigen-binding domain is a Fab molecule comprising a Fab light chain and a Fab heavy chain and the second antigen-binding domain is a Fab molecule comprising a Fab light chain and a Fab heavy chain, and wherein:
(a) the Fab light chain of the first antigen-binding domain comprises a glutamic acid residue at position 133, and the Fab heavy chain of the first antigen-binding domain comprises a lysine residue at position 183 (numbering according to Kabat); and/or
(b) the Fab light chain of the second antigen-binding domain comprises a lysine residue at position 133, and the Fab heavy chain of the second antigen-binding domain comprises a glutamic acid residue at position 183 (numbering according to Kabat).

10. The bispecific antigen-binding molecule of any one of embodiments 1-9, further comprising an Fc domain comprising a first subunit and a second subunit.

11. The bispecific antigen-binding molecule of embodiment 10, wherein the Fc domain is an IgG Fc domain.

12. The bispecific antigen-binding molecule of embodiment 11, wherein the Fc domain is an IgG₁ Fc domain.

13. The bispecific antigen-binding molecule of any one of embodiments 10-12, wherein the Fc domain is a human IgG Fc domain.

14. The bispecific antigen-binding molecule of any one of embodiments 10-13, wherein the Fc domain comprises a modification promoting the association of the first subunit and the second subunit of the Fc domain.

15. The bispecific antigen-binding molecule of any one of embodiments 1-14, wherein the bispecific antigen-binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain.

16. The bispecific antigen-binding molecule of any one of embodiments 10-14, wherein the first subunit comprises one or more heavy chain constant domains selected from a first CH2 (CH2₁) domain and/or a first CH3 (CH3₁) domain; and the second subunit comprises one or more heavy chain constant domains selected from a second CH2 (CH2₂) domain and/or a second CH3 (CH3₂) domain.

17. The bispecific antigen-binding molecule of embodiment 16, wherein at least one of the one or more heavy chain constant domains is paired with another heavy chain constant domain.

18. The bispecific antigen-binding molecule of embodiment 17, wherein the CH3₁ and CH3₂ domains each comprise a protuberance or cavity, and wherein the protuberance or cavity in the CH3₁ domain is positionable in the cavity or protuberance, respectively, in the CH3₂ domain.

19. The bispecific antigen-binding molecule of embodiment 18, wherein the CH3₁ and CH3₂ domains meet at an interface between said protuberance and cavity.

20. The bispecific antigen-binding molecule of any one of embodiments 16-19, wherein the CH2₁ and CH2₂ domains each comprise a protuberance or cavity, and wherein the protuberance or cavity in the CH2₁ domain is positionable in the cavity or protuberance, respectively, in the CH2₂ domain.

21. The bispecific antigen-binding molecule of embodiment 20, wherein the CH2₁ and CH2₂ domains meet at an interface between said protuberance and cavity.

22. The bispecific antigen-binding molecule of any one of embodiments 8-21, wherein the first antigen-binding domain and the second antigen-binding domain are each a Fab molecule and the bispecific antigen-binding molecule comprises an Fc domain comprising a first subunit and a second subunit; and wherein the first antigen-binding domain is fused at the C-terminus of the Fab heavy chain to the N-terminus of the first subunit and the second antigen-binding domain is fused at the C-terminus of the Fab heavy chain to the N-terminus of the second subunit.

23. The bispecific antigen-binding molecule of embodiment 22, wherein the first subunit comprises a tryptophan residue at position 366; and the second subunit comprises a serine residue at position 366, an alanine residue at position 368, and a valine residue at position 407 (numbered according to Kabat EU index).

24. The bispecific antigen-binding molecule of any one of embodiments 10⁻²³, wherein each of the first subunit and the second subunit comprises an alanine residue at position 234, an alanine residue at position 235, and a glycine residue at position 329 (numbering according to Kabat EU index).

25. The bispecific antigen-binding molecule of any one of embodiments 1-24, wherein the bispecific antigen-binding molecule further comprises a third antigen-binding domain that binds to CCR8.

26. The bispecific antigen-binding molecule of embodiment 25, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 4);
(v) a CDR-L2 comprising the amino acid sequence GASNLAS (SEQ ID NO: 5); and
(vi) a CDR-L3 comprising the amino acid sequence QQAYYGNSFVEGT (SEQ ID NO: 6).

27. The bispecific antigen-binding molecule of embodiment 25 or 26, wherein the third antigen-binding domain comprises a VL domain and a VH domain, and wherein:
(a) the VL domain comprises a proline residue at position 12 (numbering according to Kabat); and/or
(b) the VL domain comprises a lysine residue at position 38 and the VH domain comprises a glutamic acid residue at position 39 (numbering according to Kabat).

28. The bispecific antigen-binding molecule of any one of embodiments 25-27, wherein the third antigen-binding domain comprises one or more of the following eight FRs:
(i) an FR-H1 comprising the amino acid sequence of SEQ ID NO: 9;
(ii) an FR-H2 comprising the amino acid sequence of SEQ ID NO: 10;
(iii) an FR-H3 comprising the amino acid sequence of SEQ ID NO: 11;
(iv) an FR-H4 comprising the amino acid sequence of SEQ ID NO: 12;
(v) an FR-L1 comprising the amino acid sequence of SEQ ID NO: 13;
(vi) an FR-L2 comprising the amino acid sequence of SEQ ID NO: 14;
(vii) an FR-L3 comprising the amino acid sequence of SEQ ID NO: 15; and/or
(viii) an FR-L4 comprising the amino acid sequence of SEQ ID NO: 16.

29. The bispecific antigen-binding molecule of any one of embodiments 25-28, wherein the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 7; (b) a VL domain comprising an amino acid sequence having at least 80%, 85%, 90%, 95%, 96%, 97%, 98%, 99%, or 100% sequence identity to the amino acid sequence of SEQ ID NO: 8; or (c) a VH domain as in (a) and a VL domain as in (b).

30. The bispecific antigen-binding molecule of any one of embodiments 25-29, wherein the third antigen-binding domain comprises (a) a VH domain comprising the amino acid sequence of SEQ ID NO: 7; (b) a VL domain comprising the amino acid sequence of SEQ ID NO: 8; or (c) a VH domain as in (a) and a VL domain as in (b).

31. The bispecific antigen-binding molecule of embodiment 30, wherein the third antigen-binding domain comprises a VH domain comprising the amino acid sequence of SEQ ID NO: 7 and a VL domain comprising the amino acid sequence of SEQ ID NO: 8.

32. The bispecific antigen-binding molecule of any one of embodiments 25-31, wherein the third antigen-binding domain is a Fab molecule.

33. The bispecific antigen-binding molecule of any one of embodiments 25-32, wherein the third antigen-binding domain is a Fab molecule comprising a Fab light chain and a Fab heavy chain, and wherein the Fab light chain of the third antigen-binding domain comprises a glutamic acid residue at position 133, and the Fab heavy chain of the third antigen-binding domain comprises a lysine residue at position 183 (numbering according to Kabat).

34. The bispecific antigen-binding molecule of any one of embodiments 25-33, wherein the second antigen-binding domain and the third antigen-binding domain are fused to each other.

35. The bispecific antigen-binding molecule of embodiment 34, wherein the second antigen-binding domain and the third antigen-binding domain are fused to each other via a peptide linker.

36. The bispecific antigen-binding molecule of embodiment 35, wherein the peptide linker comprises the amino acid sequence of SEQ ID NO: 37.

37. The bispecific antigen-binding molecule of any one of embodiments 34-36, wherein the second antigen-binding domain and the third antigen-binding domain are each a Fab molecule, and wherein the third antigen-binding domain is fused at the C-terminus of the Fab heavy chain to the N-terminus of the Fab heavy chain of the second antigen-binding domain.

38. The bispecific antigen-binding molecule of any one of embodiments 25-37, wherein the bispecific antigen-binding molecule comprises an Fc domain comprising of a first subunit and a second subunit; wherein the first antigen-binding domain, the second antigen-binding domain, and the third antigen-binding domain are each a Fab molecule; wherein the first antigen-binding domain is fused at the C-terminus of the Fab heavy chain to the N-terminus of the first subunit; wherein the second antigen-binding domain is fused at the C-terminus of the Fab heavy chain to the N-terminus of the second subunit; and wherein the third antigen-binding domain is fused at the C-terminus of the Fab heavy chain to the N-terminus of the Fab heavy chain of the second antigen-binding domain.

39. The bispecific antigen-binding molecule of any one of embodiments 1-38, wherein the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 33, a polypeptide comprising an amino acid sequence having at least 80%, 85%, 90%, 95%, 96%, 97%, 98%, 99%, or 100% sequence identity to the amino acid sequence of SEQ ID NO: 34, a polypeptide comprising an amino acid sequence having at least 80%, 85%, 90%, 95%, 96%, 97%, 98%, 99%, or 100% sequence identity to the amino acid sequence of SEQ ID NO: 35, and a polypeptide comprising an amino acid sequence having at least 80%, 85%, 90%, 95%, 96%, 97%, 98%, 99%, or 100% sequence identity to the amino acid sequence of SEQ ID NO: 36.

40. The bispecific antigen-binding molecule of embodiment 39, wherein the bispecific antigen-binding molecule comprises a polypeptide comprising the amino acid sequence of SEQ ID NO: 33, a first polypeptide and a second polypeptide each comprising the amino acid sequence of SEQ ID NO: 34, a polypeptide comprising the amino acid sequence of SEQ ID NO: 35, and a polypeptide comprising the amino acid sequence of SEQ ID NO: 36.

41. The bispecific antigen-binding molecule of embodiment 40, wherein:
(i) the polypeptide comprising the amino acid sequence of SEQ ID NO: 33 is connected to the first polypeptide comprising the amino acid sequence of SEQ ID NO: 34 via a Fab heavy chain and Fab light chain interaction;
(ii) the polypeptide comprising the amino acid sequence of SEQ ID NO: 35 is connected to the second polypeptide comprising the amino acid sequence of SEQ ID NO: 34 via a Fab heavy chain and Fab light chain interaction;
(iii) the polypeptide comprising the amino acid sequence of SEQ ID NO: 35 is connected to the polypeptide comprising the amino acid sequence of SEQ ID NO: 36 via a Fab heavy chain and Fab light chain interaction; and
(iv) the polypeptide comprising the amino acid sequence of SEQ ID NO: 33 is connected to the polypeptide comprising the amino acid sequence of SEQ ID NO: 35 via a first subunit and a second subunit of an Fc domain.

42. An isolated polynucleotide or a set of isolated polynucleotides encoding the bispecific antigen-binding molecule of any one of embodiments 1-41.

43. A vector or a set of vectors comprising the isolated polynucleotide or the set of isolated polynucleotides of embodiment 42.

44. A host cell or a set of host cells comprising (i) the isolated polynucleotide or the set of isolated polynucleotides of embodiment 42 or (ii) the vector or the set of vectors of embodiment 43.

45. A method of producing a bispecific antigen-binding molecule that binds to CCR8 and CD3, comprising the steps of (a) culturing the host cell or the set of host cells of embodiment 44 under conditions suitable for the expression of the bispecific antigen-binding molecule.

46. The method of embodiment 45, further comprising recovering the bispecific antigen-binding molecule.

47. A bispecific antigen-binding molecule that binds to CCR8 and CD3 produced by the method of embodiment 45 or 46.

48. A pharmaceutical composition comprising the bispecific antigen-binding molecule of any one of embodiments 1-41 and 47 and a pharmaceutically acceptable carrier.

49. The bispecific antigen-binding molecule of any one of embodiments 1-41 and 47 or the pharmaceutical composition of embodiment 48 for use as a medicament.

50. Use of the bispecific antigen-binding molecule of any one of embodiments 1-41 and 47 or the pharmaceutical composition of embodiment 48 in the manufacture of a medicament.

51. The bispecific antigen-binding molecule of any one of embodiments 1-41 and 47 or the pharmaceutical composition of embodiment 48 for use in the treatment of a cancer.

52. Use of the bispecific antigen-binding molecule of any one of embodiments 1-41 and 47 or the pharmaceutical composition of embodiment 48 for the treatment of a cancer in a subject in need thereof.

53. Use of the bispecific antigen-binding molecule of any one of embodiments 1-41 and 47 or the pharmaceutical composition of embodiment 48 for treating a cancer in a subject in need thereof.

54. A method of treating a cancer in a subject, comprising administering to the subject an effective amount of the bispecific antigen-binding molecule of any one of embodiments 1-41 and 47 or the pharmaceutical composition of embodiment 48.

55. The bispecific antigen-binding molecule for use, pharmaceutical composition for use, use, or method of any one of embodiments 51-54, wherein the cancer is selected from the group consisting of bladder cancer, blastoma, blood cancer, bone cancer, brain cancer, breast cancer, cervical cancer, colorectal cancer, endometrial cancer, esophageal cancer, gastric cancer, head and neck cancer, kidney cancer, liver cancer, lung cancer, ovarian cancer, pancreatic cancer, prostate cancer, sarcoma, skin cancer, testicular cancer, and uterine cancer.

56. Use of the bispecific antigen-binding molecule of any one of embodiments 1-41 and 47 or the pharmaceutical composition of embodiment 48 for depleting regulatory T cells.

57. A method of depleting regulatory T cells in a tumor microenvironment in a subject having cancer comprising administering to the subject an effective amount of the bispecific antigen-binding molecule of any one of embodiments 1-41 and 47 or the pharmaceutical composition of embodiment 48 sufficient to deplete the regulatory T cells in the tumor microenvironment.

58. A method of depleting regulatory T cells outside of a tumor microenvironment in a subject having cancer comprising administering to the subject an effective amount of the bispecific antigen-binding molecule of any one of embodiments 1-41 and 47 or the pharmaceutical composition of embodiment 48 sufficient to deplete the regulatory T cells outside of the tumor microenvironment.

59. The use or method of embodiment 56 or 57, wherein the regulatory T cells present in the tumor microenvironment of the cancer are depleted.

60. The use or method of embodiment 56 or 58, wherein the regulatory T cells outside of the tumor microenvironment of the cancer are depleted.

61. An in vitro method of depleting regulatory T cells from a cancer cell population, comprising contacting the cell population with the bispecific antigen-binding molecule of any one of embodiments 1-41 and 47 or the pharmaceutical composition of embodiment 48 in an amount sufficient to deplete the regulatory T cells from the cell population.

62. Use of the bispecific antigen-binding molecule of any one of embodiments 1-41 and 47 or the pharmaceutical composition of embodiment 48 for reducing CCR8 mRNA expression.

63. A method of reducing CCR8 mRNA expression in the blood of a subject comprising administering to the subject an effective amount of the bispecific antigen-binding molecule of any one of embodiments 1-41 and 47 or the pharmaceutical composition of embodiment 48 sufficient to reduce CCR8 mRNA expression in the blood.

64. The bispecific antigen-binding molecule for use, pharmaceutical composition for use, use, or method of any one of embodiments 51-63, further comprising administering an additional therapeutic agent to the subject.

65. The bispecific antigen-binding molecule for use, pharmaceutical composition for use, use, or method of embodiment 64, wherein the additional therapeutic agent is an anti-cancer agent.

66. The bispecific antigen-binding molecule for use, pharmaceutical composition for use, use, or method of embodiment 65, wherein the anti-cancer agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a toll-like receptor (TLR) agent, a bispecific antibody, a cellular therapy, and an immune cell engager.

67. The bispecific antigen-binding molecule for use, pharmaceutical composition for use, use, or method of embodiment 65 or 66, wherein the anti-cancer agent is a PD-L1 binding antagonist.

68. The bispecific antigen-binding molecule for use, pharmaceutical composition for use, use, or method of embodiment 67, wherein the PD-L1 binding antagonist is atezolizumab.

69. The bispecific antigen-binding molecule for use, pharmaceutical composition for use, use, or method of embodiment 64, wherein the additional therapeutic agent is tocilizumab or a corticosteroid.

70. Use of a bispecific antigen-binding molecule or a pharmaceutical composition comprising the bispecific antigen-binding molecule thereof for depleting regulatory T cells, wherein the bispecific antigen-binding molecule comprises:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds an activating T cell antigen.

71. A method of depleting regulatory T cells in a tumor microenvironment in a subject having cancer comprising administering to the subject an effective amount of a bispecific antigen-binding molecule or a pharmaceutical composition comprising the bispecific antigen-binding molecule thereof sufficient to deplete the regulatory T cells in the tumor microenvironment, wherein the bispecific antigen-binding molecule comprises:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds an activating T cell antigen.

72. A method of depleting regulatory T cells outside of a tumor microenvironment in a subject having cancer comprising administering to the subject an effective amount of a bispecific antigen-binding molecule or a pharmaceutical composition comprising the bispecific antigen-binding molecule thereof sufficient to deplete the regulatory T cells outside of the tumor microenvironment, wherein the bispecific antigen-binding molecule comprises:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds an activating T cell antigen.

73. The use or method of embodiment 70 or 71, wherein the regulatory T cells present in the tumor microenvironment of the cancer are depleted.

74. The use or method of embodiment 70 or 72, wherein the regulatory T cells outside of the tumor microenvironment of the cancer are depleted.

75. An in vitro method of depleting regulatory T cells from a cancer cell population, comprising contacting the cell population with a bispecific antigen-binding molecule or a pharmaceutical composition comprising the bispecific antigen-binding molecule thereof in an amount sufficient to deplete the regulatory T cells from the cell population, wherein the bispecific antigen-binding molecule comprises:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds an activating T cell antigen.

76. Use of a bispecific antigen-binding molecule or a pharmaceutical composition comprising the bispecific antigen-binding molecule thereof for reducing CCR8 mRNA expression, wherein the bispecific antigen-binding molecule comprises:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds an activating T cell antigen.

77. A method of reducing CCR8 mRNA expression in the blood of a subject comprising administering to the subject an effective amount of a bispecific antigen-binding molecule or a pharmaceutical composition comprising the bispecific antigen-binding molecule thereof sufficient to reduce CCR8 mRNA expression in the blood, wherein the bispecific antigen-binding molecule comprises:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 4);
   (v) a CDR-L2 comprising the amino acid sequence GASNLAS (SEQ ID NO: 5); and
   (vi) a CDR-L3 comprising the amino acid sequence QQAYYGNSFVEGT (SEQ ID NO: 6); and
(b) a second antigen-binding domain that binds an activating T cell antigen.

78. The bispecific antigen-binding molecule for use, pharmaceutical composition for use, use, or method of any one of embodiments 70-77, wherein the activating T cell antigen is CD3.

79. The bispecific antigen-binding molecule for use, pharmaceutical composition for use, use, or method of any one of embodiments 70-78, further comprising administering an additional therapeutic agent to the subject.

80. The bispecific antigen-binding molecule for use, pharmaceutical composition for use, use, or method of embodiment 79, wherein the additional therapeutic agent is an anti-cancer agent.

81. The bispecific antigen-binding molecule for use, pharmaceutical composition for use, use, or method of embodiment 80, wherein the anti-cancer agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a TLR agent, a bispecific antibody, a cellular therapy, and an immune cell engager.

82. The bispecific antigen-binding molecule for use, pharmaceutical composition for use, use, or method of embodiment 80 or 81, wherein the anti-cancer agent is a PD-L1 binding antagonist.

83. The bispecific antigen-binding molecule for use, pharmaceutical composition for use, use, or method of embodiment 82, wherein the PD-L1 binding antagonist is atezolizumab.

84. The bispecific antigen-binding molecule for use, pharmaceutical composition for use, use, or method of embodiment 79, wherein the additional therapeutic agent is tocilizumab or a corticosteroid.

85. A bispecific antigen-binding molecule comprising:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRX₁X₂YATWAKG (SEQ ID NO: 82), wherein X1 is T or D and X2 is Y or A;
   (iii) a CDR-H3 comprising the amino acid sequence SYPDYSATASI (SEQ ID NO: 3);
   (iv) a CDR-L1 comprising the amino acid sequence QASENX₃ANX₄LA (SEQ ID NO: 83), wherein X3 is I or V and X4 is A or I;
   (v) a CDR-L2 comprising the amino acid sequence GASNLAS (SEQ ID NO: 5); and
   (vi) a CDR-L3 comprising the amino acid sequence QQAYYGNSFVX₅GT (SEQ ID NO: 84), wherein X5 is E or S; and
(b) a second antigen-binding domain that binds CD3, wherein the second antigen-binding domain comprises the following six CDRs:
   (i) a CDR-H1 comprising the amino acid sequence NYYIH (SEQ ID NO: 17) or SYYIH (SEQ ID NO: 91);
   (ii) a CDR-H2 comprising the amino acid sequence WIYPGDGNTKYNEKFKG (SEQ ID NO: 18) or WIYPENDNTKYNEKFKD (SEQ ID NO: 92);
   (iii) a CDR-H3 comprising the amino acid sequence DSYSNYYFDY (SEQ ID NO: 19) or DGYSRYYFDY (SEQ ID NO: 93);
   (iv) a CDR-L1 comprising the amino acid sequence KSSQSLLNSRTRKNYLA (SEQ ID NO: 20);
   (v) a CDR-L2 comprising the amino acid sequence WASTRES (SEQ ID NO: 21); and
   (vi) a CDR-L3 comprising the amino acid sequence TQSFILRT (SEQ ID NO: 22).

86. A bispecific antigen-binding molecule comprising:
(a) a first antigen-binding domain that binds CCR8, wherein the first antigen-binding domain comprises the following six CDRs:
   (i) a CDR-H1 comprising the amino acid sequence TYAMG (SEQ ID NO: 1);
   (ii) a CDR-H2 comprising the amino acid sequence LIHRSGRTYYATWAKG (SEQ ID NO: 2), LIHRSGRTAYATWAKG (SEQ ID NO: 63), or LIHRSGRDAYATWAKG (SEQ ID NO: 66);
   (iii) a CDR-H3 comprising the amino acid sequence SYPDYSATASI (SEQ ID NO: 3);
   (iv) a CDR-L1 comprising the amino acid sequence QASENIANALA (SEQ ID NO: 38), QASENVANALA (SEQ ID NO: 41), QASENIANILA (SEQ ID NO: 44), or QASENVANILA (SEQ ID NO: 50);
   (v) a CDR-L2 comprising the amino acid sequence GASNLAS (SEQ ID NO: 39); and
   (vi) a CDR-L3 comprising the amino acid sequence QQAYYGNSFVEGT (SEQ ID NO: 40) or QQAYYGNSFVSGT (SEQ ID NO: 49); and
(b) a second antigen-binding domain that binds CD3, wherein the second antigen-binding domain comprises the following six CDRs:
   (i) a CDR-H1 comprising the amino acid sequence NYYIH (SEQ ID NO: 17) or SYYIH (SEQ ID NO: 91);
   (ii) a CDR-H2 comprising the amino acid sequence WIYPGDGNTKYNEKFKG (SEQ ID NO: 18) or WIYPENDNTKYNEKFKD (SEQ ID NO: 92);
   (iii) a CDR-H3 comprising the amino acid sequence DSYSNYYFDY (SEQ ID NO: 19) or DGYSRYYFDY (SEQ ID NO: 93);
   (iv) a CDR-L1 comprising the amino acid sequence KSSQSLLNSRTRKNYLA (SEQ ID NO: 20);
   (v) a CDR-L2 comprising the amino acid sequence WASTRES (SEQ ID NO: 21); and
   (vi) a CDR-L3 comprising the amino acid sequence TQSFILRT (SEQ ID NO: 22).

87. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 38);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40).

88. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANALA (SEQ ID NO: 41);
(v) a CDR-L2 comprising the amino acid sequence GASNLAS (SEQ ID NO: 42); and
(vi) a CDR-L3 comprising the amino acid sequence QQAYYGNSFVEGT (SEQ ID NO: 43).

89. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANILA (SEQ ID NO: 44);
(v) a CDR-L2 comprising the amino acid sequence GASNLAS (SEQ ID NO: 45); and
(vi) a CDR-L3 comprising the amino acid sequence QQAYYGNSFVEGT (SEQ ID NO: 46).

90. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 47);
(v) a CDR-L2 comprising the amino acid sequence GASNLAS (SEQ ID NO: 48); and
(vi) a CDR-L3 comprising the amino acid sequence QQAYYGNSFVSGT (SEQ ID NO: 49).

91. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANILA (SEQ ID NO: 50);
(v) a CDR-L2 comprising the amino acid sequence GASNLAS (SEQ ID NO: 51); and
(vi) a CDR-L3 comprising the amino acid sequence QQAYYGNSFVEGT (SEQ ID NO: 52).

92. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANALA (SEQ ID NO: 53);
(v) a CDR-L2 comprising the amino acid sequence GASNLAS (SEQ ID NO: 54); and
(vi) a CDR-L3 comprising the amino acid sequence QQAYYGNSFVSGT (SEQ ID NO: 55).

93. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANILA (SEQ ID NO: 56);
(v) a CDR-L2 comprising the amino acid sequence GASNLAS (SEQ ID NO: 57); and
(vi) a CDR-L3 comprising the amino acid sequence QQAYYGNSFVSGT (SEQ ID NO: 58).

94. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANILA (SEQ ID NO: 59);
(v) a CDR-L2 comprising the amino acid sequence GASNLAS (SEQ ID NO: 60); and
(vi) a CDR-L3 comprising the amino acid sequence QQAYYGNSFVSGT (SEQ ID NO: 61).

95. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 62);
(ii) a CDR-H2 comprising the amino acid sequence LIHRSGRTAYATWAKG (SEQ ID NO: 63);
(iii) a CDR-H3 comprising the amino acid sequence SYPDYSATASI (SEQ ID NO: 64);
(iv) a CDR-L1 comprising the amino acid sequence QASENIANALA (SEQ ID NO: 38);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40).

96. The bispecific antigen-binding molecule of embodiment 85 or 86, wherein the first antigen-binding domain comprises the following six CDRs:
(i) a CDR-H1 comprising the amino acid sequence TYAMG (SEQ ID NO: 65);
(ii) a CDR-H2 comprising the amino acid sequence LIHRSGRDAYATWAKG (SEQ ID NO: 66);
(iii) a CDR-H3 comprising the amino acid sequence SYPDYSATASI (SEQ ID NO: 67);
(iv) a CDR-L1 comprising the amino acid sequence QASENIANALA (SEQ ID NO: 38);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40).

97. The bispecific antigen-binding molecule of any one of embodiments 85-96, wherein the first antigen-binding domain comprises a light chain variable region (VL) domain and a heavy chain variable region (VH) domain, and wherein:
(a) the VL domain comprises a proline residue at position 12 (numbering according to Kabat); and/or
(b) the VL domain comprises a lysine residue at position 38 and the VH domain comprises a glutamic acid residue at position 39; or the VL domain comprises a glutamic acid residue at position 38 and the VH domain comprises a lysine residue at position 39 (numbering according to Kabat).

98. The bispecific antigen-binding molecule of any one of embodiments 85-97, wherein the second antigen-binding domain comprises a VL domain and a VH domain, and wherein the VL domain comprises a glutamic acid residue at position 38 and the VH domain comprises a lysine residue at position 39; or the VL domain comprises a lysine residue at position 38 and the VH domain comprises a glutamic acid residue at position 39 (numbering according to Kabat).

99. The bispecific antigen-binding molecule of any one of embodiments 85-98, wherein:
the first antigen-binding domain comprises: (a) a VH domain comprising an amino acid sequence having at least 80%, 85%, 90%, 95%, 96%, 97%, 98%, 99%, or 100% sequence identity to the amino acid sequence of any one of SEQ ID NOs: 68, 77, 78, or 107; (b) a VL domain comprising an amino acid sequence having at least 80%, 85%, 90%, 95%, 96%, 97%, 98%, 99%, or 100% sequence identity to the amino acid sequence of any one of SEQ ID NOs: 69-76 or 108; or (c) a VH domain as in (a) and a VL domain as in (b); and/or
the second antigen-binding domain comprises:
(a) a VH domain comprising an amino acid sequence having at least 80%, 85%, 90%, 95%, 96%, 97%, 98%, 99%, or 100% sequence identity to the amino acid sequence of SEQ ID NO: 23, 97, 109, or 111; (b) a VL domain comprising an amino acid sequence having at least 80%, 85%, 90%, 95%, 96%, 97%, 98%, 99%, or 100% sequence identity to the amino acid sequence of SEQ ID NO: 24, 98, 110, or 112; or (c) a VH domain as in (a) and a VL domain as in (b)

100. The bispecific antigen-binding molecule of any one of embodiments 85-99, wherein:
the first antigen-binding domain comprises:
   (a) a VH domain comprising the amino acid sequence of any one of SEQ ID NOs: 68, 77, 78, or 107; (b) a VL domain comprising the amino acid sequence of any one of SEQ ID NOs: 69-76 or 108; or (c) a VH domain as in (a) and a VL domain as in (b); and/or
the second antigen-binding domain comprises:
   (a) a VH domain comprising the amino acid sequence of SEQ ID NO: 23, 97, 109, or 111; (b) a VL domain comprising the amino acid sequence of SEQ ID NO: 24, 98, 110, or 112; or (c) a VH domain as in (a) and a VL domain as in (b).

101. The bispecific antigen-binding molecule of embodiment 100, wherein:
the first antigen-binding domain comprises:
   (a) a VH domain comprising the amino acid sequence of any one of SEQ ID NOs: 68, 77, 78, or 107 and a VL domain comprising the amino acid sequence of any one of SEQ ID NOs: 69-76 or 108; and
the second antigen-binding domain comprises:
   (b) a VH domain comprising the amino acid sequence of SEQ ID NO: 23, 97, 109, or 111 and a VL domain comprising the amino acid sequence of SEQ ID NO: 24, 98, 110, or 112.

102. The bispecific antigen-binding molecule of any one of embodiments 85-101, wherein the first antigen-binding domain is a Fab molecule comprising a Fab light chain and a Fab heavy chain and/or the second antigen-binding domain is a Fab molecule comprising a Fab light chain and a Fab heavy chain.

103. The bispecific antigen-binding molecule of any one of embodiments 85-102, wherein the first antigen-binding domain is a Fab molecule comprising a Fab light chain and a Fab heavy chain and the second antigen-binding domain is a Fab molecule comprising a Fab light chain and a Fab heavy chain, and wherein:
(a) the Fab light chain of the first antigen-binding domain comprises a glutamic acid residue at position 133, and the Fab heavy chain of the first antigen-binding domain comprises a lysine residue at position 183; or the Fab light chain of the first antigen-binding domain comprises a lysine residue at position 133, and the Fab heavy chain of the first antigen-binding domain comprises a glutamic acid residue at position 183 (numbering according to Kabat); and/or
(b) the Fab light chain of the second antigen-binding domain comprises a lysine residue at position 133, and the Fab heavy chain of the second antigen-binding domain comprises a glutamic acid residue at position 183; or the Fab light chain of the first antigen-binding domain comprises a glutamic acid residue at position 133, and the Fab heavy chain of the first antigen-binding domain comprises a lysine residue at position 183 (numbering according to Kabat).

104. The bispecific antigen-binding molecule of any one of embodiments 85-103, further comprising an Fc domain comprising a first subunit and a second subunit.

105. The bispecific antigen-binding molecule of embodiment 104, wherein the Fc domain is an IgG Fc domain.

106. The bispecific antigen-binding molecule of embodiment 105, wherein the Fc domain is an IgG1 Fc domain.

107. The bispecific antigen-binding molecule of any one of embodiments 104-106, wherein the Fc domain is a human IgG Fc domain.

108. The bispecific antigen-binding molecule of any one of embodiments 104-107, wherein the Fc domain comprises a modification promoting the association of the first subunit and the second subunit of the Fc domain.

109. The bispecific antigen-binding molecule of any one of embodiments 85-108, wherein the bispecific antigen-binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain.

110. The bispecific antigen-binding molecule of any one of embodiments 104-108, wherein the first subunit comprises one or more heavy chain constant domains selected from a first CH2 (CH2₁) domain and/or a first CH3 (CH3₁) domain; and the second subunit comprises one or more heavy chain constant domains selected from a second CH2 (CH2₂) domain and/or a second CH3 (CH3₂) domain.

111. The bispecific antigen-binding molecule of embodiment 110, wherein at least one of the one or more heavy chain constant domains is paired with another heavy chain constant domain.

112. The bispecific antigen-binding molecule of embodiment 111, wherein the CH3₁ and CH3₂ domains each comprise a protuberance or cavity, and wherein the protuberance or cavity in the CH3₁ domain is positionable in the cavity or protuberance, respectively, in the CH3₂ domain.

113. The bispecific antigen-binding molecule of embodiment 112, wherein the CH3₁ and CH3₂ domains meet at an interface between said protuberance and cavity.

114. The bispecific antigen-binding molecule of any one of embodiments 110-113, wherein the CH2₁ and CH2₂ domains each comprise a protuberance or cavity, and wherein the protuberance or cavity in the CH2₁ domain is positionable in the cavity or protuberance, respectively, in the CH2₂ domain.

115. The bispecific antigen-binding molecule of embodiment 114, wherein the CH2₁ and CH2₂ domains meet at an interface between said protuberance and cavity.

116. The bispecific antigen-binding molecule of any one of embodiments 102-115, wherein the first antigen-binding domain and the second antigen-binding domain are each a Fab molecule and the bispecific antigen-binding molecule comprises an Fc domain comprising a first subunit and a second subunit; and wherein the first antigen-binding domain is fused at the C-terminus of the Fab heavy chain to the N-terminus of the first subunit and the second antigen-binding domain is fused at the C-terminus of the Fab heavy chain to the N-terminus of the second subunit.

117. The bispecific antigen-binding molecule of embodiment 116, wherein the first subunit comprises a tryptophan residue at position 366; and the second subunit comprises a serine residue at position 366, an alanine residue at position 368, and a valine residue at position 407 (numbered according to Kabat EU index).

118. The bispecific antigen-binding molecule of any one of embodiments 104-117, wherein each of the first subunit and the second subunit comprises an alanine residue at position 234, an alanine residue at position 235, and a glycine residue at position 329 (numbering according to Kabat EU index).

119. The bispecific antigen-binding molecule of any one of embodiments 85-118, wherein the bispecific antigen-binding molecule further comprises a third antigen-binding domain that binds to CCR8.

120. The bispecific antigen-binding molecule of embodiment 119, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRX₁X₂YATWAKG (SEQ ID NO: 82), wherein X1 is T or D and X2 is Y or A;
(iii) a CDR-H3 comprising the amino acid sequence SYPDYSATASI (SEQ ID NO: 3);
(iv) a CDR-L1 comprising the amino acid sequence QASENX₃ANX₄LA (SEQ ID NO: 83), wherein X3 is I or V and X4 is A or I;
(v) a CDR-L2 comprising the amino acid sequence GASNLAS (SEQ ID NO: 5); and
(vi) a CDR-L3 comprising the amino acid sequence QQAYYGNSFVX₅GT (SEQ ID NO: 84), wherein X5 is E or S.

121. The bispecific antigen-binding molecule of embodiment 119, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2), LIHRSGRTAYATWAKG (SEQ ID NO: 63), or LIHRSGRDAYATWAKG (SEQ ID NO: 66);
(iii) a CDR-H3 comprising the amino acid sequence SYPDYSATASI (SEQ ID NO: 3);
(iv) a CDR-L1 comprising the amino acid sequence QASENIANALA (SEQ ID NO: 38), QASENVANALA (SEQ ID NO: 41), QASENIANILA (SEQ ID NO: 44), or QASENVANILA (SEQ ID NO: 50);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40) or QQAYYGNSFVSGT (SEQ ID NO: 49).

122. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 38);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40).

123. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANALA (SEQ ID NO: 41);
(v) a CDR-L2 comprising the amino acid sequence GASNLAS (SEQ ID NO: 42); and
(vi) a CDR-L3 comprising the amino acid sequence QQAYYGNSFVEGT (SEQ ID NO: 43).

124. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANILA (SEQ ID NO: 44);
(v) a CDR-L2 comprising the amino acid sequence GASNLAS (SEQ ID NO: 45); and
(vi) a CDR-L3 comprising the amino acid sequence QQAYYGNSFVEGT (SEQ ID NO: 46).

125. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 47);
(v) a CDR-L2 comprising the amino acid sequence GASNLAS (SEQ ID NO: 48); and
(vi) a CDR-L3 comprising the amino acid sequence QQAYYGNSFVSGT (SEQ ID NO: 49).

126. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANILA (SEQ ID NO: 50);
(v) a CDR-L2 comprising the amino acid sequence GASNLAS (SEQ ID NO: 51); and
(vi) a CDR-L3 comprising the amino acid sequence QQAYYGNSFVEGT (SEQ ID NO: 52).

127. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANALA (SEQ ID NO: 53);
(v) a CDR-L2 comprising the amino acid sequence GASNLAS (SEQ ID NO: 54); and
(vi) a CDR-L3 comprising the amino acid sequence QQAYYGNSFVSGT (SEQ ID NO: 55).

128. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANILA (SEQ ID NO: 56);
(v) a CDR-L2 comprising the amino acid sequence GASNLAS (SEQ ID NO: 57); and
(vi) a CDR-L3 comprising the amino acid sequence QQAYYGNSFVSGT (SEQ ID NO: 58).

129. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENVANILA (SEQ ID NO: 59);
(v) a CDR-L2 comprising the amino acid sequence GASNLAS (SEQ ID NO: 60); and
(vi) a CDR-L3 comprising the amino acid sequence QQAYYGNSFVSGT (SEQ ID NO: 61).

130. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 62);
(ii) a CDR-H2 comprising the amino acid sequence LIHRSGRTAYATWAKG (SEQ ID NO: 63);
(iii) a CDR-H3 comprising the amino acid sequence SYPDYSATASI (SEQ ID NO: 64);
(iv) a CDR-L1 comprising the amino acid sequence QASENIANALA (SEQ ID NO: 38);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40).

131. The bispecific antigen-binding molecule of embodiment 120 or 121, wherein the third antigen-binding domain comprises the following six CDRs:
(i) a CDR-H1 comprising the amino acid sequence TYAMG (SEQ ID NO: 65);
(ii) a CDR-H2 comprising the amino acid sequence LIHRSGRDAYATWAKG (SEQ ID NO: 66);
(iii) a CDR-H3 comprising the amino acid sequence SYPDYSATASI (SEQ ID NO: 67);
(iv) a CDR-L1 comprising the amino acid sequence QASENIANALA (SEQ ID NO: 38);
(v) a CDR-L2 comprising the amino acid sequence GASNLAS (SEQ ID NO: 39); and
(vi) a CDR-L3 comprising the amino acid sequence QQAYYGNSFVEGT (SEQ ID NO: 40).

132. The bispecific antigen-binding molecule of any one of embodiments 119-131, wherein the third antigen-binding domain comprises a VL domain and a VH domain, and wherein:
(a) the VL domain comprises a proline residue at position 12 (numbering according to Kabat); and/or
(b) the VL domain comprises a lysine residue at position 38 and the VH domain comprises a glutamic acid residue at position 39; or the VL domain comprises a glutamic acid residue at position 38 and the VH domain comprises a lysine residue at position 39 (numbering according to Kabat).

133. The bispecific antigen-binding molecule of any one of embodiments 119-132, wherein the third antigen-binding domain comprises (a) a VH domain comprising an amino acid sequence having at least 80%, 85%, 90%, 95%, 96%, 97%, 98%, 99%, or 100% sequence identity to the amino acid sequence of SEQ ID NO: 68, 77, or 78; (b) a VL domain comprising an amino acid sequence having at least 80%, 85%, 90%, 95%, 96%, 97%, 98%, 99%, or 100% sequence identity to the amino acid sequence of SEQ ID NO: 69-76; or (c) a VH domain as in (a) and a VL domain as in (b).

134. The bispecific antigen-binding molecule of any one of embodiments 119-133, wherein the third antigen-binding domain comprises (a) a VH domain comprising the amino acid sequence of SEQ ID NO: 68, 77, or 78; (b) a VL domain comprising the amino acid sequence of SEQ ID NO: 69-76; or (c) a VH domain as in (a) and a VL domain as in (b).

135. The bispecific antigen-binding molecule of embodiment 134, wherein the third antigen-binding domain comprises a VH domain comprising the amino acid sequence of SEQ ID NO: 68, 77, or 78 and a VL domain comprising the amino acid sequence of SEQ ID NO: 69-76.

136. The bispecific antigen-binding molecule of any one of embodiments 119-135, wherein the third antigen-binding domain is a Fab molecule.

137. The bispecific antigen-binding molecule of any one of embodiments 119-136, wherein the third antigen-binding domain is a Fab molecule comprising a Fab light chain and a Fab heavy chain, and wherein the Fab light chain of the third antigen-binding domain comprises a glutamic acid residue at position 133, and the Fab heavy chain of the third antigen-binding domain comprises a lysine residue at position 183; or the Fab light chain of the third antigen-binding domain comprises a lysine residue at position 133, and the Fab heavy chain of the third antigen-binding domain comprises a glutamic acid residue at position 183 (numbering according to Kabat).

138. The bispecific antigen-binding molecule of any one of embodiments 119-137, wherein the second antigen-binding domain and the third antigen-binding domain are fused to each other.

139. The bispecific antigen-binding molecule of embodiment 138, wherein the second antigen-binding domain and the third antigen-binding domain are fused to each other via a peptide linker.

140. The bispecific antigen-binding molecule of embodiment 139, wherein the peptide linker comprises the amino acid sequence of SEQ ID NO: 37.

141. The bispecific antigen-binding molecule of any one of embodiments 138-140, wherein the second antigen-binding domain and the third antigen-binding domain are each a Fab molecule, and wherein the third antigen-binding domain is fused at the C-terminus of the Fab heavy chain to the N-terminus of the Fab heavy chain of the second antigen-binding domain.

142. The bispecific antigen-binding molecule of any one of embodiments 119-141, wherein the bispecific antigen-binding molecule comprises an Fc domain comprising of a first subunit and a second subunit; wherein the first antigen-binding domain, the second antigen-binding domain, and the third antigen-binding domain are each a Fab molecule; wherein the first antigen-binding domain is fused at the C-terminus of the Fab heavy chain to the N-terminus of the first subunit; wherein the second antigen-binding domain is fused at the C-terminus of the Fab heavy chain to the N-terminus of the second subunit; and wherein the third antigen-binding domain is fused at the C-terminus of the Fab heavy chain to the N-terminus of the Fab heavy chain of the second antigen-binding domain.

143. A bispecific antigen-binding molecule comprising a first antigen-binding domain that binds CCR8, a second antigen-binding domain that binds CD3, and a third antigen-binding domain that binds CCR8.

144. The bispecific antigen-binding molecule of embodiment 143 wherein the first antigen-binding domain, the second antigen-binding domain, and the third antigen-binding molecule are each a Fab molecule, each comprising a Fab heavy chain and a Fab light chain, and the bispecific antigen-binding molecule comprises an Fc domain comprising a first subunit and a second subunit.

145. The bispecific antigen-binding molecule of embodiment 144, wherein the first antigen-binding domain is fused at the C-terminus of the Fab heavy chain to the N-terminus of the first subunit, the second antigen-binding domain is fused at the C-terminus of the Fab heavy chain to the N-terminus of the second subunit, and the second antigen-binding domain and the third antigen-binding domain are fused to each other.

146. The bispecific antigen-binding molecule of embodiment 145, wherein the second antigen-binding domain and the third antigen-binding domain are fused to each other via a peptide linker.

147. The bispecific antigen-binding molecule of any one of embodiments 85-146, wherein the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 117, a polypeptide comprising an amino acid sequence having at least 80%, 85%, 90%, 95%, 96%, 97%, 98%, 99%, or 100% sequence identity to the amino acid sequence of SEQ ID NO: 118, a polypeptide comprising an amino acid sequence having at least 80%, 85%, 90%, 95%, 96%, 97%, 98%, 99%, or 100% sequence identity to the amino acid sequence of SEQ ID NO: 119, and a polypeptide comprising an amino acid sequence having at least 80%, 85%, 90%, 95%, 96%, 97%, 98%, 99%, or 100% sequence identity to the amino acid sequence of SEQ ID NO: 120.

148. The bispecific antigen-binding molecule of embodiment 147, wherein the bispecific antigen-binding molecule comprises a polypeptide comprising the amino acid sequence of SEQ ID NO: 117, a first polypeptide and a second polypeptide each comprising the amino acid sequence of SEQ ID NO: 118, a polypeptide comprising the amino acid sequence of SEQ ID NO: 119, and a polypeptide comprising the amino acid sequence of SEQ ID NO: 120.

149. The bispecific antigen-binding molecule of embodiment 148, wherein:
(i) the polypeptide comprising the amino acid sequence of SEQ ID NO: 117 is connected to the first polypeptide comprising the amino acid sequence of SEQ ID NO: 118 via a Fab heavy chain and Fab light chain interaction;
(ii) the polypeptide comprising the amino acid sequence of SEQ ID NO: 119 is connected to the second polypeptide comprising the amino acid sequence of SEQ ID NO: 118 via a Fab heavy chain and Fab light chain interaction;
(iii) the polypeptide comprising the amino acid sequence of SEQ ID NO: 119 is connected to the polypeptide comprising the amino acid sequence of SEQ ID NO: 120 via a Fab heavy chain and Fab light chain interaction; and
(iv) the polypeptide comprising the amino acid sequence of SEQ ID NO: 117 is connected to the polypeptide comprising the amino acid sequence of SEQ ID NO: 119 via a first subunit and a second subunit of an Fc domain.

150. The bispecific antigen-binding molecule of any one of embodiments 85-146, wherein the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 33, a polypeptide comprising an amino acid sequence having at least 80%, 85%, 90%, 95%, 96%, 97%, 98%, 99%, or 100% sequence identity to the amino acid sequence of SEQ ID NO: 34, a polypeptide comprising an amino acid sequence having at least 80%, 85%, 90%, 95%, 96%, 97%, 98%, 99%, or 100% sequence identity to the amino acid sequence of SEQ ID NO: 90, and a polypeptide comprising an amino acid sequence having at least 80%, 85%, 90%, 95%, 96%, 97%, 98%, 99%, or 100% sequence identity to the amino acid sequence of SEQ ID NO: 36.

151. The bispecific antigen-binding molecule of embodiment 150, wherein the bispecific antigen-binding molecule comprises a polypeptide comprising the amino acid sequence of SEQ ID NO: 33, a first polypeptide and a second polypeptide each comprising the amino acid sequence of SEQ ID NO: 34, a polypeptide comprising the amino acid sequence of SEQ ID NO: 90, and a polypeptide comprising the amino acid sequence of SEQ ID NO: 36.

152. The bispecific antigen-binding molecule of embodiment 151, wherein:
(i) the polypeptide comprising the amino acid sequence of SEQ ID NO: 33 is connected to the first polypeptide comprising the amino acid sequence of SEQ ID NO: 34 via a Fab heavy chain and Fab light chain interaction;
(ii) the polypeptide comprising the amino acid sequence of SEQ ID NO: 90 is connected to the second polypeptide comprising the amino acid sequence of SEQ ID NO: 34 via a Fab heavy chain and Fab light chain interaction;
(iii) the polypeptide comprising the amino acid sequence of SEQ ID NO: 90 is connected to the polypeptide comprising the amino acid sequence of SEQ ID NO: 36 via a Fab heavy chain and Fab light chain interaction; and
(iv) the polypeptide comprising the amino acid sequence of SEQ ID NO: 33 is connected to the polypeptide comprising the amino acid sequence of SEQ ID NO: 90 via a first subunit and a second subunit of an Fc domain.

153. The bispecific antigen-binding molecule of any one of embodiments 85-146, wherein the bispecific antigen-binding molecule comprises a polypeptide comprising an amino acid sequence having at least 80%, 85%, 90%, 95%, 96%, 97%, 98%, 99%, or 100% sequence identity to the amino acid sequence of SEQ ID NO: 117, a polypeptide comprising an amino acid sequence having at least 80%, 85%, 90%, 95%, 96%, 97%, 98%, 99%, or 100% sequence identity to the amino acid sequence of SEQ ID NO: 118, a polypeptide comprising an amino acid sequence having at least 80%, 85%, 90%, 95%, 96%, 97%, 98%, 99%, or 100% sequence identity to the amino acid sequence of SEQ ID NO: 121, and a polypeptide comprising an amino acid sequence having at least 80%, 85%, 90%, 95%, 96%, 97%, 98%, 99%, or 100% sequence identity to the amino acid sequence of SEQ ID NO: 120.

154. The bispecific antigen-binding molecule of embodiment 153, wherein the bispecific antigen-binding molecule comprises a polypeptide comprising the amino acid sequence of SEQ ID NO: 117, a first polypeptide and a second polypeptide each comprising the amino acid sequence of SEQ ID NO: 118, a polypeptide comprising the amino acid sequence of SEQ ID NO: 121, and a polypeptide comprising the amino acid sequence of SEQ ID NO: 120.

155. The bispecific antigen-binding molecule of embodiment 154, wherein:
(i) the polypeptide comprising the amino acid sequence of SEQ ID NO: 117 is connected to the first polypeptide comprising the amino acid sequence of SEQ ID NO: 118 via a Fab heavy chain and Fab light chain interaction;
(ii) the polypeptide comprising the amino acid sequence of SEQ ID NO: 121 is connected to the second polypeptide comprising the amino acid sequence of SEQ ID NO: 118 via a Fab heavy chain and Fab light chain interaction;
(iii) the polypeptide comprising the amino acid sequence of SEQ ID NO: 121 is connected to the polypeptide comprising the amino acid sequence of SEQ ID NO: 120 via a Fab heavy chain and Fab light chain interaction; and
(iv) the polypeptide comprising the amino acid sequence of SEQ ID NO: 117 is connected to the polypeptide comprising the amino acid sequence of SEQ ID NO: 121 via a first subunit and a second subunit of an Fc domain.

156. An isolated polynucleotide or a set of isolated polynucleotides encoding the bispecific antigen-binding molecule of any one of embodiments 85-155.

157. An isolated polynucleotide or a set of isolated polynucleotides comprising a nucleic acid sequence that is at least 85%, at least 90%, at least 95%, or at least 99% identical to the nucleic acid sequence of any one of SEQ ID NOs: 85-89.

158. An isolated polynucleotide or a set of isolated polynucleotides comprising the nucleic acid sequence of any one of SEQ ID NOs: 85-89.

159. A vector or a set of vectors comprising the isolated polynucleotide or the set of isolated polynucleotides of any one of embodiments 156-158.

160. A host cell or a set of host cells comprising (i) the isolated polynucleotide or the set of isolated polynucleotides of any one of embodiments 156-158 or (ii) the vector or the set of vectors of embodiment 159.

161. A method of producing a bispecific antigen-binding molecule that binds to CCR8 and CD3, comprising the steps of (a) culturing the host cell or the set of host cells of embodiment 156 under conditions suitable for the expression of the bispecific antigen-binding molecule.

162. The method of embodiment 161, further comprising recovering the bispecific antigen-binding molecule.

163. A bispecific antigen-binding molecule that binds to CCR8 and CD3 produced by the method of embodiment 161 or 162.

164. A pharmaceutical composition comprising the bispecific antigen-binding molecule of any one of embodiments 85-155 and 163 and a pharmaceutically acceptable carrier.

165. The bispecific antigen-binding molecule of any one of embodiments 85-155 and 163 or the pharmaceutical composition of embodiment 164 for use as a medicament.

166. Use of the bispecific antigen-binding molecule of any one of embodiments 85-155 and 163 or the pharmaceutical composition of embodiment 164 in the manufacture of a medicament.

167. The bispecific antigen-binding molecule of any one of embodiments 85-155 and 163 or the pharmaceutical composition of embodiment 164 for use in the treatment of a cancer.

168. Use of the bispecific antigen-binding molecule of any one of embodiments 85-155 and 163 or the pharmaceutical composition of embodiment 164 for the treatment of a cancer in a subject in need thereof.

169. Use of the bispecific antigen-binding molecule of any one of embodiments 85-155 and 163 or the pharmaceutical composition of embodiment 164 for treating a cancer in a subject in need thereof.

170. A method of treating a cancer in a subject, comprising administering to the subject an effective amount of the bispecific antigen-binding molecule of any one of embodiments 85-155 and 163 or the pharmaceutical composition of embodiment 164.

171. The bispecific antigen-binding molecule for use, pharmaceutical composition for use, use, or method of any one of embodiments 167-170, wherein the cancer is selected from the group consisting of bladder cancer, blastoma, blood cancer, bone cancer, brain cancer, breast cancer, cervical cancer, colorectal cancer, endometrial cancer, esophageal cancer, gastric cancer, head and neck cancer, kidney cancer, liver cancer, lung cancer, ovarian cancer, pancreatic cancer, prostate cancer, sarcoma, skin cancer, testicular cancer, and uterine cancer.

172. Use of the bispecific antigen-binding molecule of any one of embodiments 85-155 and 163 or the pharmaceutical composition of embodiment 164 for depleting regulatory T cells.

173. A method of depleting regulatory T cells in a tumor microenvironment in a subject having cancer comprising administering to the subject an effective amount of the bispecific antigen-binding molecule of any one of embodiments 85-155 and 163 or the pharmaceutical composition of embodiment 164 sufficient to deplete the regulatory T cells in the tumor microenvironment.

174. A method of depleting regulatory T cells outside of a tumor microenvironment in a subject having cancer comprising administering to the subject an effective amount of the bispecific antigen-binding molecule of any one of embodiments 85-155 and 163 or the pharmaceutical composition of embodiment 164 sufficient to deplete the regulatory T cells outside of the tumor microenvironment.

175. The use or method of embodiment 172 or 173, wherein the regulatory T cells present in the tumor microenvironment of the cancer are depleted.

176. The use or method of embodiment 172 or 174, wherein the regulatory T cells outside of the tumor microenvironment of the cancer are depleted.

177. An in vitro method of depleting regulatory T cells from a cancer cell population, comprising contacting the cell population with the bispecific antigen-binding molecule of any one of embodiments 85-155 and 163 or the pharmaceutical composition of embodiment 164 in an amount sufficient to deplete the regulatory T cells from the cell population.

178. Use of the bispecific antigen-binding molecule of any one of embodiments 85-155 and 163 or the pharmaceutical composition of embodiment 164 for reducing CCR8 mRNA expression.

179. A method of reducing CCR8 mRNA expression in the blood of a subject comprising administering to the subject an effective amount of the bispecific antigen-binding molecule of any one of embodiments 85-155 and 163 or the pharmaceutical composition of embodiment 164 sufficient to reduce CCR8 mRNA expression.

180. The bispecific antigen-binding molecule for use, pharmaceutical composition for use, use, or method of any one of embodiments 167-179, further comprising administering an additional therapeutic agent to the subject.

181. The bispecific antigen-binding molecule for use, pharmaceutical composition for use, use, or method of embodiment 180, wherein the additional therapeutic agent is an anti-cancer agent.

182. The bispecific antigen-binding molecule for use, pharmaceutical composition for use, use, or method of embodiment 181, wherein the anti-cancer agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a TLR agent, a bispecific antibody, a cellular therapy, and an immune cell engager.

183. The bispecific antigen-binding molecule for use, pharmaceutical composition for use, use, or method of embodiment 181 or 182, wherein the anti-cancer agent is a PD-L1 binding antagonist.

184. The bispecific antigen-binding molecule for use, pharmaceutical composition for use, use, or method of embodiment 183, wherein the PD-L1 binding antagonist is atezolizumab.

185. The bispecific antigen-binding molecule for use, pharmaceutical composition for use, use, or method of embodiment 180, wherein the additional therapeutic agent is tocilizumab or a corticosteroid.

186. Use of a bispecific antigen-binding molecule or a pharmaceutical composition comprising the bispecific antigen-binding molecule thereof for depleting regulatory T cells, wherein the bispecific antigen-binding molecule comprises: (a) a first antigen-binding domain that binds CCR8; and (b) a second antigen-binding domain that binds an activating T cell antigen.

187. A method of depleting regulatory T cells in a tumor microenvironment in a subject having cancer comprising administering to the subject an effective amount of a bispecific antigen-binding molecule or a pharmaceutical composition comprising the bispecific antigen-binding molecule thereof sufficient to deplete the regulatory T cells in the tumor microenvironment, wherein the bispecific antigen-binding molecule comprises: (a) a first antigen-binding domain that binds CCR8; and (b) a second antigen-binding domain that binds an activating T cell antigen.

188. A method of depleting regulatory T cells outside of a tumor microenvironment in a subject having cancer comprising administering to the subject an effective amount of a bispecific antigen-binding molecule or a pharmaceutical composition comprising the bispecific antigen-binding molecule thereof sufficient to deplete the regulatory T cells outside of the tumor microenvironment, wherein the bispecific antigen-binding molecule comprises: (a) a first antigen-binding domain that binds CCR8; and (b) a second antigen-binding domain that binds an activating T cell antigen.

189. The use or method of embodiment 186 or 187, wherein the regulatory T cells present in the tumor microenvironment of the cancer are depleted.

190. The use or method of embodiment 186 or 188, wherein the regulatory T cells outside of the tumor microenvironment of the cancer are depleted.

191. An in vitro method of depleting regulatory T cells from a cancer cell population, comprising contacting the cell population with a bispecific antigen-binding molecule or a pharmaceutical composition comprising the bispecific antigen-binding molecule thereof in an amount sufficient to deplete the regulatory T cells from the cell population, wherein the bispecific antigen-binding molecule comprises: (a) a first antigen-binding domain that binds CCR8; and (b) a second antigen-binding domain that binds an activating T cell antigen.

192. Use of a bispecific antigen-binding molecule or a pharmaceutical composition comprising the bispecific antigen-binding molecule thereof for reducing CCR8 mRNA expression, wherein the bispecific antigen-binding molecule comprises: (a) a first antigen-binding domain that binds CCR8; and (b) a second antigen-binding domain that binds an activating T cell antigen.

193. A method of reducing CCR8 mRNA expression in the blood of a subject comprising administering to the subject an effective amount of a bispecific antigen-binding molecule or a pharmaceutical composition comprising the bispecific antigen-binding molecule thereof sufficient to reduce CCR8 mRNA expression, wherein the bispecific antigen-binding molecule comprises: (a) a first antigen-binding domain that binds CCR8; and (b) a second antigen-binding domain that binds an activating T cell antigen.

194. The bispecific antigen-binding molecule for use, pharmaceutical composition for use, use, or method of any one of embodiments 186-193, wherein the activating T cell antigen is CD3.

195. The bispecific antigen-binding molecule for use, pharmaceutical composition for use, use, or method of any one of embodiments 186-194, further comprising administering an additional therapeutic agent to the subject.

196. The bispecific antigen-binding molecule for use, pharmaceutical composition for use, use, or method of embodiment 195, wherein the additional therapeutic agent is an anti-cancer agent.

197. The bispecific antigen-binding molecule for use, pharmaceutical composition for use, use, or method of embodiment 196, wherein the anti-cancer agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a TLR agent, a bispecific antibody, a cellular therapy, and an immune cell engager.

198. The bispecific antigen-binding molecule for use, pharmaceutical composition for use, use, or method of embodiment 196 or 197, wherein the anti-cancer agent is a PD-L1 binding antagonist.

199. The bispecific antigen-binding molecule for use, pharmaceutical composition for use, use, or method of embodiment 198, wherein the PD-L1 binding antagonist is atezolizumab.

200. The bispecific antigen-binding molecule for use, pharmaceutical composition for use, use, or method of embodiment 195, wherein the additional therapeutic agent is tocilizumab or a corticosteroid.

201. The use or method of any one of embodiments 52-54, 57, 58, 71, 72, 168-170, 173, 174, 187, and 188, wherein the subject has reduced CCR8 mRNA expression.

### Listing of Sequences

Sequences disclosed herein are provided below in Table 17. Sequences corresponding to SEQ ID NOs: 1-78 and 82-121 are synthetic constructs. SEQ ID NO: 79 is a human sequence (e.g., Homo sapiens). SEQ ID NO: 80 is a cynomolgus monkey (e.g., Macaca fascicularis) sequence. SEQ ID NO: 81 is a mouse (e.g., Mus musculus) sequence.

TABLE 17
Listing of Sequences Disclosed Herein

| | | |
|---|---|---|
| 1 | CCR8 1889 | TYAMG |
| | S12P CDR-H1 | |
| 2 | CCR8 1889 | LIHRSGRTYYATWAKG |
| | S12P CDR-H2 | |
| 3 | CCR8 1889 | SYPDYSATASI |
| | S12P CDR-H3 | |
| 4 | CCR8 1889 | QASENIANALA |
| | S12P CDR-L1 | |
| 5 | CCR8 1889 | GASNLAS |
| | S12P CDR-L2 | |
| 6 | CCR8 1889 | QQAYYGNSFVEGT |
| | S12P CDR-L3 | |
| 7 | CCR8 1889 | EVQLLESGGGLVQPGGSLRLSCAASGIDLSTYAMGWVREAPGKGL |
| | S12P VH | EWVGLIHRSGRTYYATWAKGRFTISKDSSKNTLYLQMNSLRAEDTA |
| | | VYYCTRSYPDYSATASIWGQGTTVTVSS |
| 8 | CCR8 1889 | DIQVTQSPSSLPASVGDRVTITCQASENIANALAWYQKKPGKPPKF |
| | S12P VL | LIYGASNLASGVPSRFSGSGSGTDFTFTISSLQPEDIATYYCQQAYY |
| | | GNSFVEGTFGGGTKVEIK |
| 9 | CCR8 1889 | EVQLLESGGGLVQPGGSLRLSCAASGIDLS |
| | S12P FR-H1 | |
| 10 | CCR8 1889 | WVREAPGKGLEWVG |
| | S12P FR-H2 | |
| 11 | CCR8 1889 | RFTISKDSSKNTLYLQMNSLRAEDTAVYYCTR |
| | S12P FR-H3 | |
| 12 | CCR8 1889 | WGQGTTVTVSS |
| | S12P FR-H4 | |
| 13 | CCR8 1889 | DIQVTQSPSSLPASVGDRVTITC |
| | S12P FR-L1 | |
| 14 | CCR8 1889 | WYQKKPGKPPKFLIY |
| | S12P FR-L2 | |
| 15 | CCR8 1889 | GVPSRFSGSGSGTDFTFTISSLQPEDIATYYC |
| | S12P FR-L3 | |
| 16 | CCR8 1889 | FGGGTKVEIK |
| | S12P FR-L4 | |
| 17 | CD3 40G5C | NYYIH |
| | CDR-H1 | |
| 18 | CD3 40G5C | WIYPGDGNTKYNEKFKG |
| | CDR-H2 | |
| 19 | CD3 40G5C | DSYSNYYFDY |
| | CDR-H3 | |
| 20 | CD3 40G5C | KSSQSLLNSRTRKNYLA |
| | CDR-L1 | |
| 21 | CD3 40G5C | WASTRES |
| | CDR-L2 | |
| 22 | CD3 40G5C | TQSFILRT |
| | CDR-L3 | |
| 23 | CD3 40G5C VH | EVQLVQSGAEVKKPGASVKVSCKASGYTFTNYYIHWVRKAPGQGL |
| | | EWIGWIYPGDGNTKYNEKFKGRATLTADTSTSTAYLELSSLRSEDT |
| | | AVYYCARDSYSNYYFDYWGQGTLVTVSS |
| 24 | CD3 40G5C VL | DIVMTQSPDSLAVSLGERATINCKSSQSLLNSRTRKNYLAWYQEKP |
| | | GQPPKLLIYWASTRESGVPDRFSGSGSGTDFTLTISSLQAEDVAVY |
| | | YCTQSFILRTFGQGTKVEIK |
| 25 | CD3 40G5C FR- | EVQLVQSGAEVKKPGASVKVSCKASGYTFT |
| | H1 | |
| 26 | CD3 40G5C FR- | WVRKAPGQGLEWIG |
| | H2 | |
| 27 | CD3 40G5C FR- | RATLTADTSTSTAYLELSSLRSEDTAVYYCAR |
| | H3 | |
| 28 | CD3 40G5C FR- | WGQGTLVTVSS |
| | H4 | |
| 29 | CD3 40G5C FR- | DIVMTQSPDSLAVSLGERATINC |
| | L1 | |
| 30 | CD3 40G5C FR- | WYQEKPGQPPKLLIY |
| | L2 | |
| 31 | CD3 40G5C FR- | GVPDRFSGSGSGTDFTLTISSLQAEDVAVYYC |
| | L3 | |
| 32 | CD3 40G5C FR- | FGQGTKVEIK |
| | L4 | |
| 33 | CCR8 knob: | EVQLLESGGGLVQPGGSLRLSCAASGIDLSTYAMGWVREAPGKGL |
| | | EWVGLIHRSGRTYYATWAKGRFTISKDSSKNTLYLQMNSLRAEDTA |
| | | VYYCTRSYPDYSATASIWGQGTTVTVSSASTKGPSVFPLAPSSKST |
| | | SGGTAALGCLVKDYFPEPVTVSWNSGALTSGVHTFPAVLQSSGLY |
| | | SLKSVVTVPSSSLGTQTYICNVNHKPSNTKVDKKVEPKSCDKTHTC |
| | | PPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALGAPIEKTISKAKGQPREPQVYTLPPSREEMTKNQ |
| | | VSLWCLVKGFYPSDIAVEWESNGQPENNYKTTPPVLDSDGSFFLY |
| | | SKLTVDKSRWQQGNVFSCSVMHEALHNHYTQKSLSLSPGK |
| 34 | CCR8 LC (P): | DIQVTQSPSSLPASVGDRVTITCQASENIANALAWYQKKPGKPPKF |
| | | LIYGASNLASGVPSRFSGSGSGTDFTFTISSLQPEDIATYYCQQAYY |
| | | GNSFVEGTFGGGTKVEIKRTVAAPSVFIFPPSDEQLKSGTASVECLL |
| | | NNFYPREAKVQWKVDNALQSGNSQESVTEQDSKDSTYSLSSTLTL |
| | | SKADYEKHKVYACEVTHQGLSSPVTKSFNRGEC |
| 35 | CCR8-CD3 | EVQLLESGGGLVQPGGSLRLSCAASGIDLSTYAMGWVREAPGKGL |
| | (40G5c) stacked | EWVGLIHRSGRTYYATWAKGRFTISKDSSKNTLYLQMNSLRAEDTA |
| | Fab HC hole: | VYYCTRSYPDYSATASIWGQGTTVTVSSASTKGPSVFPLAPSSKST |
| | | SGGTAALGCLVKDYFPEPVTVSWNSGALTSGVHTFPAVLQSSGLY |
| | | SLKSVVTVPSSSLGTQTYICNVNHKPSNTKVDKKVEPKSCDKTHTE |
| | | VQLVQSGAEVKKPGASVKVSCKASGYTFTNYYIHWVRKAPGQGLE |
| | | WIGWIYPGDGNTKYNEKFKGRATLTADTSTSTAYLELSSLRSEDTA |
| | | VYYCARDSYSNYYFDYWGQGTLVTVSSASTKGPSVFPLAPSSKST |
| | | SGGTAALGCLVKDYFPEPVTVSWNSGALTSGVHTFPAVLQSSGLY |
| | | SLESVVTVPSSSLGTQTYICNVNHKPSNTKVDKKVEPKSCDKTHTC |
| | | PPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALGAPIEKTISKAKGQPREPQVYTLPPSREEMTKNQ |
| | | VSLSCAVKGFYPSDIAVEWESNGQPENNYKTTPPVLDSDGSFFLVS |
| | | KLTVDKSRWQQGNVFSCSVMHEALHNHYTQKSLSLSPGK |
| 36 | CD3 LC: | DIVMTQSPDSLAVSLGERATINCKSSQSLLNSRTRKNYLAWYQEKP |
| | | GQPPKLLIYWASTRESGVPDRFSGSGSGTDFTLTISSLQAEDVAVY |
| | | YCTQSFILRTFGQGTKVEIKRTVAAPSVFIFPPSDEQLKSGTASVKC |
| | | LLNNFYPREAKVQWKVDNALQSGNSQESVTEQDSKDSTYSLSSTL |
| | | TLSKADYEKHKVYACEVTHQGLSSPVTKSFNRGEC |
| 37 | Peptide Linker | DKTHT |
| 38 | hu.CCR8-1889 | QASENIANALA |
| | (WT) CDR-L1 | |
| 39 | hu.CCR8-1889 | GASNLAS |
| | (WT) CDR-L2 | |
| 40 | hu.CCR8-1889 | QQAYYGNSFVEGT |
| | (WT) CDR-L3 | |
| 41 | LCS12P.I29V | QASENVANALA |
| | (PV) CDR-L1 | |
| 42 | LCS12P.I29V | GASNLAS |
| | (PV) CDR-L2 | |
| 43 | LCS12P.I29V | QQAYYGNSFVEGT |
| | (PV) CDR-L3 | |
| 44 | LC.S12P.A32I | QASENIANILA |
| | (PI) CDR-L1 | |
| 45 | LC.S12P.A32I | GASNLAS |
| | (PI) CDR-L2 | |
| 46 | LC.S12P.A32I | QQAYYGNSFVEGT |
| | (PI) CDR-L3 | |
| 47 | LC.S12P.E95dS | QASENIANALA |
| | (PS) CDR-L1 | |
| 48 | LC.S12P.E95dS | GASNLAS |
| | (PS) CDR-L2 | |
| 49 | LC.S12P.E95dS | QQAYYGNSFVSGT |
| | (PS) CDR-L3 | |
| 50 | LCS12P.I29V.A32I | QASENVANILA |
| | (PVI) CDR-L1 | |
| 51 | LCS12P.I29V.A32I | GASNLAS |
| | (PVI) CDR-L2 | |
| 52 | LCS12P.I29V.A32I | QQAYYGNSFVEGT |
| | (PVI) CDR-L3 | |
| 53 | LC.S12P.I29V.E95dS | QASENVANALA |
| | (PVS) CDR-L1 | |
| 54 | LC.S12P.I29V.E95dS | GASNLAS |
| | (PVS) CDR-L2 | |
| 55 | LC.S12P.I29V.E95dS | QQAYYGNSFVSGT |
| | (PVS) CDR-L3 | |
| 56 | LC.S12P.A32I.E95dS | QASENIANILA |
| | (PIS) CDR-L1 | |
| 57 | LC.S12P.A32I.E95dS | GASNLAS |
| | (PIS) CDR-L2 | |
| 58 | LC.S12P.A32I.E95dS | QQAYYGNSFVSGT |
| | (PIS) CDR-L3 | |
| 59 | LC.S12P.I29V.A32I. | QASENVANILA |
| | E95dS (PVIS) | |
| | CDR-L1 | |
| 60 | LC.S12P.I29V.A32I. | GASNLAS |
| | E95dS (PVIS) | |
| | CDR-L2 | |
| 61 | LC.S12P.I29V.A32I. | QQAYYGNSFVSGT |
| | E95dS (PVIS) | |
| | CDR-L3 | |
| 62 | HC.Y58A (A) | TYAMG |
| | CDR-H1 | |
| 63 | HC.Y58A (A) | LIHRSGRTAYATWAKG |
| | CDR-H2 | |
| 64 | HC.Y58A (A) | SYPDYSATASI |
| | CDR-H3 | |
| 65 | HC.T57D.Y58A | TYAMG |
| | (DA) CDR-H1 | |
| 66 | HC.T57D.Y58A | LIHRSGRDAYATWAKG |
| | (DA) CDR-H2 | |
| 67 | HC.T57D.Y58A | SYPDYSATASI |
| | (DA) CDR-H3 | |
| 68 | hu.CCR8-1889 | EVQLLESGGGLVQPGGSLRLSCAASGIDLSTYAMGWVREAPGKGL |
| | (WT) VH | EWVGLIHRSGRTYYATWAKGRFTISKDSSKNTLYLQMNSLRAEDTA |
| | | VYYCTRSYPDYSATASIWGQGTTVTVSS |
| 69 | hu.CCR8-1889 | DIQVTQSPSSLSASVGDRVTITCQASENIANALAWYQKKPGKPPKF |
| | (WT) VL | LIYGASNLASGVPSRFSGSGSGTDFTFTISSLQPEDIATYYCQQAYY |
| | | GNSFVEGTFGGGTKVEIK |
| 70 | LCS12P.I29V | DIQVTQSPSSLPASVGDRVTITCQASENVANALAWYQKKPGKPPKF |
| | (PV) VL | LIYGASNLASGVPSRFSGSGSGTDFTFTISSLQPEDIATYYCQQAYY |
| | | GNSFVEGTFGGGTKVEIK |
| 71 | LC.S12P.A32I | DIQVTQSPSSLPASVGDRVTITCQASENIANILAWYQKKPGKPPKFLI |
| | (PI) VL | YGASNLASGVPSRFSGSGSGTDFTFTISSLQPEDIATYYCQQAYYG |
| | | NSFVEGTFGGGTKVEIK |
| 72 | LC.S12P.E95dS | DIQVTQSPSSLPASVGDRVTITCQASENIANALAWYQKKPGKPPKF |
| | (PS) VL | LIYGASNLASGVPSRFSGSGSGTDFTFTISSLQPEDIATYYCQQAYY |
| | | GNSFVSGTFGGGTKVEIK |
| 73 | LCS12P.I29V.A32I | DIQVTQSPSSLPASVGDRVTITCQASENVANILAWYQKKPGKPPKF |
| | (PVI) VL | LIYGASNLASGVPSRFSGSGSGTDFTFTISSLQPEDIATYYCQQAYY |
| | | GNSFVEGTFGGGTKVEIK |
| 74 | LC.S12P.I29V.E95dS | DIQVTQSPSSLPASVGDRVTITCQASENVANALAWYQKKPGKPPKF |
| | (PVS) VL | LIYGASNLASGVPSRFSGSGSGTDFTFTISSLQPEDIATYYCQQAYY |
| | | GNSFVSGTFGGGTKVEIK |
| 75 | LC.S12P.A32I.E95dS | DIQVTQSPSSLPASVGDRVTITCQASENIANILAWYQKKPGKPPKFLI |
| | (PIS) VL | YGASNLASGVPSRFSGSGSGTDFTFTISSLQPEDIATYYCQQAYYG |
| | | NSFVSGTFGGGTKVEIK |
| 76 | LC.S12P.I29V.A32I. | DIQVTQSPSSLPASVGDRVTITCQASENVANILAWYQKKPGKPPKF |
| | E95dS (PVIS) | LIYGASNLASGVPSRFSGSGSGTDFTFTISSLQPEDIATYYCQQAYY |
| | VL | GNSFVSGTFGGGTKVEIK |
| 77 | HC.Y58A (A) VH | EVQLLESGGGLVQPGGSLRLSCAASGIDLSTYAMGWVREAPGKGL |
| | | EWVGLIHRSGRTAYATWAKGRFTISKDSSKNTLYLQMNSLRAEDTA |
| | | VYYCTRSYPDYSATASIWGQGTTVTVSS |
| 78 | HC.T57D.Y58A | EVQLLESGGGLVQPGGSLRLSCAASGIDLSTYAMGWVREAPGKGL |
| | (DA) VH | EWVGLIHRSGRDAYATWAKGRFTISKDSSKNTLYLQMNSLRAEDT |
| | | AVYYCTRSYPDYSATASIWGQGTTVTVSS |
| 79 | human CCR8 | MDYTLDLSVTTVTDYYYPDIFSSPCDAELIQTNGKLLLAVFYCLLFVF |
| | SEQ ID NO: 79 | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFYQVASEDGVLQCYSFYNQQ |
| | | TLKWKIFTNFKMNILGLLIPFTIFMFCYIKILHQLKRCQNHNKTKAIRLV |
| | | LIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCE |
| | | KSSSCQQHSSRSSSVDYIL |
| 80 | cyno CCR8 | MDYTLDPSMTTMTDYYYPDSLSSPCDGELIQRNDKLLLAVFYCLLF |
| | SEQ ID NO: 80 | VFSLLGNSLVILVLVVCKKLRNITDIYLLNLALSDLLFVFSFPFQTYYQ |
| | | LDQWVFGTVMCKVVSGFYYIGFYSSMFFITLMSVDRYLAVVHAVYA |
| | | IKVRTIRMGTTLSLVVWLTAIMATIPLLVFYQVASEDGVLQCYSFYNQ |
| | | QTLKWKIFTNFEMNILGLLIPFTIFMFCYIKILHQLKRCQNHNKTKAIR |
| | | LVLIVVIASLLFWVPFNVVLFLTSLHSMHILDGCSISQQLNYATHVTEII |
| | | SFTHCCVNPVIYAFVGEKFKKHLSEIFQKSCSHIFIYLGRQMPRESC |
| | | EKSSSCQQHSFRSSSIDYIL |
| 81 | mouse CCR8 | MDYTMEPNVTMTDYYPDFFTAPCDAEFLLRGSMLYLAILYCVLFVL |
| | SEQ ID NO: 81 | GLLGNSLVILVLVGCKKLRSITDIYLLNLAASDLLFVLSIPFQTHNLLD |
| | | QWVFGTAMCKVVSGLYYIGFFSSMFFITLMSVDRYLAIVHAVYAIKV |
| | | RTASVGTALSLTVWLAAVTATIPLMVFYQVASEDGMLQCFQFYEEQ |
| | | SLRWKLFTHFEINALGLLLPFAILLFCYVRILQQLRGCLNHNRTRAIKL |
| | | VLTVVIVSLLFWVPFNVALFLTSLHDLHILDGCATRQRLALAIHVTEVI |
| | | SFTHCCVNPVIYAFIGEKFKKHLMDVFQKSCSHIFLYLGRQMPVGAL |
| | | ERQLSSNQRSSHSSTLDDIL |
| 82 | CCR8 1889 | LIHRSGRX₁X₂YATWAKG |
| | CDR-H2 variable | wherein X₁ is T or D and X₂ is Y or A |
| | sequence | |
| 83 | CCR8 1889 | QASENX₃ANX₄LA |
| | CDR-L1 variable | Wherein X₃ is I or V and X₄ is A or I |
| | sequence | |
| 84 | CCR8 1889 | QQAYYGNSFVX₅GT |
| | CDR-L3 variable | wherein X₅ is E or S |
| | sequence | |
| 85 | Nucleic acid | ATGGGATGGTCATGTATCATCCTTTTTCTAGTAGCAACTGCAACC |
| | encoding CCR8 | GGTGTACATTCAGACATCCAGGTGACCCAGTCTCCATCCTCCCT |
| | LC | GCCTGCATCTGTAGGAGACAGAGTCACCATCACTTGCCAGGCG |
| | | AGTGAGAACATTGCCAACGCTTTAGCTTGGTATCAGAAGAAACC |
| | | AGGGAAACCCCCTAAGTTCCTGATCTACGGTGCATCCAATTTGG |
| | | CATCAGGGGTCCCATCAAGGTTCAGTGGAAGTGGATCTGGGAC |
| | | AGATTTTACTTTCACCATCAGCAGCCTGCAGCCTGAAGATATTGC |
| | | AACATATTACTGTCAACAGGCTTATTATGGCAATAGTTTTGTGGA |
| | | GGGCACTTTCGGCGGAGGTACCAAGGTGGAGATCAAACGAACT |
| | | GTGGCTGCACCATCTGTCTTCATCTTCCCGCCATCTGATGAGCA |
| | | GTTGAAATCTGGAACTGCTTCTGTTGAATGCCTGCTGAATAACTT |
| | | CTATCCCAGAGAGGCCAAAGTACAGTGGAAGGTGGATAACGCC |
| | | CTCCAATCGGGTAACTCCCAGGAGAGTGTCACAGAGCAGGACA |
| | | GCAAGGACAGCACCTACAGCCTCAGCAGCACCCTGACGCTGAG |
| | | CAAAGCAGACTACGAGAAACACAAAGTCTACGCCTGCGAAGTCA |
| | | CCCATCAGGGCCTGAGCTCGCCCGTCACAAAGAGCTTCAACAG |
| | | GGGAGAGTGT |
| 86 | Nucleic acid | ATGGGATGGTCATGTATCATCCTTTTTCTAGTAGCAACTGCAACC |
| | encoding CCR8 | GGTGTACATTCAGAGGTGCAACTGTTGGAGTCTGGGGGAGGCT |
| | Knob | TGGTACAGCCTGGGGGGTCCCTGAGACTCTCCTGTGCAGCCTC |
| | | TGGAATCGACTTGAGCACCTATGCCATGGGCTGGGTCCGCGAG |
| | | GCTCCAGGGAAGGGGCTGGAGTGGGTCGGACTTATTCATAGGA |
| | | GTGGTAGGACATACTACGCAACCTGGGCGAAGGGCCGGTTCAC |
| | | CATCTCCAAAGACTCCTCCAAGAACACGCTGTATCTGCAAATGA |
| | | ACAGCCTGAGAGCCGAGGACACGGCCGTATATTACTGTACGAG |
| | | AAGTTATCCCGACTACTCCGCCACTGCGAGCATCTGGGGGCAA |
| | | GGGACCACGGTCACCGTCTCGAGTGCCTCCACCAAGGGCCCAT |
| | | CGGTCTTCCCCCTGGCACCCTCCTCCAAGAGCACCTCTGGGGG |
| | | CACAGCGGCCCTGGGCTGCCTGGTCAAGGACTACTTCCCCGAG |
| | | CCGGTGACGGTGTCGTGGAACTCAGGCGCCCTGACCAGCGGC |
| | | GTGCACACCTTCCCGGCTGTCCTACAGTCCTCAGGACTCTACTC |
| | | CCTCAAAAGCGTGGTGACTGTGCCCTCTAGCAGCTTGGGCACC |
| | | CAGACCTACATCTGCAACGTGAATCACAAGCCCAGCAACACCAA |
| | | GGTGGACAAGAAAGTTGAGCCCAAATCTTGTGACAAAACTCACA |
| | | CATGCCCACCGTGCCCAGCACCTGAAGCCGCGGGGGGACCGT |
| | | CAGTCTTCCTCTTCCCCCCAAAACCCAAGGACACCCTCATGATC |
| | | TCCCGGACCCCTGAGGTCACATGCGTGGTGGTGGACGTGAGCC |
| | | ACGAAGACCCTGAGGTCAAGTTCAACTGGTACGTGGACGGCGT |
| | | GGAGGTGCATAATGCCAAGACAAAGCCGCGGGAGGAGCAGTAC |
| | | AACAGCACGTACCGGGTGGTCAGCGTCCTCACCGTCCTGCACC |
| | | AGGACTGGCTGAATGGCAAGGAGTACAAGTGCAAGGTCTCCAA |
| | | CAAAGCCCTCGGAGCCCCCATCGAGAAAACCATCTCCAAAGCC |
| | | AAAGGGCAGCCCCGAGAACCACAGGTGTACACCCTGCCCCCAT |
| | | CCCGGGAAGAGATGACCAAGAACCAGGTCAGCCTGTGGTGCCT |
| | | GGTCAAAGGCTTCTATCCCAGCGACATCGCCGTGGAGTGGGAG |
| | | AGCAATGGGCAGCCGGAGAACAACTACAAGACCACGCCTCCCG |
| | | TGCTGGACTCCGACGGCTCCTTCTTCCTCTACAGCAAGCTCACC |
| | | GTGGACAAGAGCAGGTGGCAGCAGGGGAACGTCTTCTCATGCT |
| | | CCGTGATGCATGAGGCTCTGCACAACCACTACACGCAGAAGAG |
| | | CCTCTCCCTGTCTCCGGGTAAA |
| 87 | Nucleic acid | ATGGGATGGTCATGTATCATCCTTTTTCTAGTAGCAACTGCAACC |
| | encoding CD3 LC | GGTGTACATTCAGATATCGTGATGACCCAGAGCCCAGATAGCCT |
| | (40G5c/MD1): | GGCCGTGAGCCTGGGCGAACGCGCCACCATCAACTGCAAAAGC |
| | | AGCCAGAGCCTGCTGAACAGCCGCACCCGCAAAAACTATCTGG |
| | | CCTGGTATCAGGAAAAACCAGGCCAGCCACCAAAACTGCTGATC |
| | | TATTGGGCCAGCACCCGCGAAAGCGGCGTGCCAGATCGCTTCA |
| | | GCGGCAGCGGCAGCGGCACCGATTTCACCCTGACCATCAGCAG |
| | | CCTGCAGGCCGAAGATGTGGCCGTGTATTATTGCACCCAGAGC |
| | | TTCATCCTGCGCACCTTCGGCCAGGGTACCAAGGTGGAGATCA |
| | | AACGAACTGTGGCTGCACCATCTGTCTTCATCTTCCCGCCATCT |
| | | GATGAGCAGTTGAAATCTGGAACTGCTTCTGTTAAATGCCTGCT |
| | | GAATAACTTCTATCCCAGAGAGGCCAAAGTACAGTGGAAGGTGG |
| | | ATAACGCCCTCCAATCGGGTAACTCCCAGGAGAGTGTCACAGA |
| | | GCAGGACAGCAAGGACAGCACCTACAGCCTCAGCAGCACCCTG |
| | | ACGCTGAGCAAAGCAGACTACGAGAAACACAAAGTCTACGCCTG |
| | | CGAAGTCACCCATCAGGGCCTGAGCTCGCCCGTCACAAAGAGC |
| | | TTCAACAGGGGAGAGTGT |
| 88 | Nucleic acid | ATGGGATGGTCATGTATCATCCTTTTTCTAGTAGCAACTGCAACC |
| | encoding CCR8- | GGTGTACATTCAGAGGTGCAACTGTTGGAGTCTGGGGGAGGCT |
| | 40G5c hole: | TGGTACAGCCTGGGGGGTCCCTGAGACTCTCCTGTGCAGCCTC |
| | | TGGAATCGACTTGAGCACCTATGCCATGGGCTGGGTCCGCGAG |
| | | GCTCCAGGGAAGGGGCTGGAGTGGGTCGGACTTATTCATAGGA |
| | | GTGGTAGGACATACTACGCAACCTGGGCGAAGGGCCGGTTCAC |
| | | CATCTCCAAAGACTCCTCCAAGAACACGCTGTATCTGCAAATGA |
| | | ACAGCCTGAGAGCCGAGGACACGGCCGTATATTACTGTACGAG |
| | | AAGTTATCCCGACTACTCCGCCACTGCGAGCATCTGGGGGCAA |
| | | GGGACCACGGTCACCGTGAGCTCCGCCAGCACCAAAGGCCCCA |
| | | GCGTGTTCCCCCTGGCCCCCAGCTCCAAGAGCACCAGCGGCG |
| | | GAACCGCCGCTCTGGGCTGCCTGGTGAAGGACTACTTCCCCGA |
| | | GCCCGTGACCGTGAGCTGGAACAGCGGCGCCCTGACCAGCGG |
| | | CGTGCACACCTTCCCCGCCGTGCTGCAGAGCTCCGGCCTGTAC |
| | | AGCCTGAAGTCCGTGGTCACCGTGCCCAGCTCCTCTCTGGGCA |
| | | CCCAGACCTACATCTGCAACGTGAACCACAAGCCCAGCAACACC |
| | | AAGGTGGACAAGAAGGTGGAGCCCAAGAGCTGCGACAAGACCC |
| | | ACACCGAGGTGCAGCTGGTGCAGAGCGGCGCCGAGGTGAAGA |
| | | AGCCCGGCGCCAGCGTGAAGGTGAGCTGCAAGGCCAGCGGCT |
| | | ACACCTTCACCAACTACTATATCCACTGGGTGAGAAAGGCCCCC |
| | | GGCCAGGGCCTGGAGTGGATCGGCTGGATCTACCCCGGCGAC |
| | | GGCAACACCAAGTACAACGAGAAGTTCAAGGGCAGAGCCACCC |
| | | TGACCGCCGACACCAGCACCAGCACCGCCTACCTGGAGCTGAG |
| | | CTCCCTGAGAAGCGAGGACACCGCCGTGTACTATTGCGCCAGA |
| | | GACAGCTACAGCAACTACTATTTCGACTACTGGGGCCAGGGCAC |
| | | CCTGGTGACCGTCTCGAGTGCCTCCACCAAGGGCCCATCGGTC |
| | | TTCCCCCTGGCACCCTCCTCCAAGAGCACCTCTGGGGGCACAG |
| | | CGGCCCTGGGCTGCCTGGTCAAGGACTACTTCCCCGAGCCGGT |
| | | GACGGTGTCGTGGAACTCAGGCGCCCTGACCAGCGGCGTGCA |
| | | CACCTTCCCGGCTGTCCTACAGTCCTCAGGACTCTACTCCCTCG |
| | | AAAGCGTGGTGACTGTGCCCTCTAGCAGCTTGGGCACCCAGAC |
| | | CTACATCTGCAACGTGAATCACAAGCCCAGCAACACCAAGGTGG |
| | | ACAAGAAAGTTGAGCCCAAATCTTGTGACAAAACTCACACATGC |
| | | CCACCGTGCCCAGCACCTGAAGCCGCGGGGGGACCGTCAGTC |
| | | TTCCTCTTCCCCCCAAAACCCAAGGACACCCTCATGATCTCCCG |
| | | GACCCCTGAGGTCACATGCGTGGTGGTGGACGTGAGCCACGAA |
| | | GACCCTGAGGTCAAGTTCAACTGGTACGTGGACGGCGTGGAGG |
| | | TGCATAATGCCAAGACAAAGCCGCGGGAGGAGCAGTACAACAG |
| | | CACGTACCGGGTGGTCAGCGTCCTCACCGTCCTGCACCAGGAC |
| | | TGGCTGAATGGCAAGGAGTACAAGTGCAAGGTCTCCAACAAAG |
| | | CCCTCGGAGCCCCCATCGAGAAAACCATCTCCAAAGCCAAAGG |
| | | GCAGCCCCGAGAACCACAGGTGTACACCCTGCCCCCATCCCGG |
| | | GAAGAGATGACCAAGAACCAGGTCAGCCTGAGCTGCGCGGTCA |
| | | AAGGCTTCTATCCCAGCGACATCGCCGTGGAGTGGGAGAGCAA |
| | | TGGGCAGCCGGAGAACAACTACAAGACCACGCCTCCCGTGCTG |
| | | GACTCCGACGGCTCCTTCTTCCTCGTCAGCAAGCTCACCGTGGA |
| | | CAAGAGCAGGTGGCAGCAGGGGAACGTCTTCTCATGCTCCGTG |
| | | ATGCATGAGGCTCTGCACAACCACTACACGCAGAAGAGCCTCTC |
| | | CCTGTCTCCGGGTAAA |
| 89 | Nucleic acid | ATGGGATGGTCATGTATCATCCTTTTTCTAGTAGCAACTGCAACC |
| | encoding CCR8- | GGTGTACATTCAGAGGTGCAACTGTTGGAGTCTGGGGGAGGCT |
| | MD1 hole | TGGTACAGCCTGGGGGGTCCCTGAGACTCTCCTGTGCAGCCTC |
| | | TGGAATCGACTTGAGCACCTATGCCATGGGCTGGGTCCGCGAG |
| | | GCTCCAGGGAAGGGGCTGGAGTGGGTCGGACTTATTCATAGGA |
| | | GTGGTAGGACATACTACGCAACCTGGGCGAAGGGCCGGTTCAC |
| | | CATCTCCAAAGACTCCTCCAAGAACACGCTGTATCTGCAAATGA |
| | | ACAGCCTGAGAGCCGAGGACACGGCCGTATATTACTGTACGAG |
| | | AAGTTATCCCGACTACTCCGCCACTGCGAGCATCTGGGGGCAA |
| | | GGGACCACGGTCACCGTGAGCTCCGCCAGCACCAAAGGCCCCA |
| | | GCGTGTTCCCCCTGGCCCCCAGCTCCAAGAGCACCAGCGGCG |
| | | GAACCGCCGCTCTGGGCTGCCTGGTGAAGGACTACTTCCCCGA |
| | | GCCCGTGACCGTGAGCTGGAACAGCGGCGCCCTGACCAGCGG |
| | | CGTGCACACCTTCCCCGCCGTGCTGCAGAGCTCCGGCCTGTAC |
| | | AGCCTGAAGTCCGTGGTCACCGTGCCCAGCTCCTCTCTGGGCA |
| | | CCCAGACCTACATCTGCAACGTGAACCACAAGCCCAGCAACACC |
| | | AAGGTGGACAAGAAGGTGGAGCCCAAGAGCTGCGACAAGACCC |
| | | ACACCGAAGTGCAGCTGGTGCAGAGCGGCGCCGAAGTGAAAAA |
| | | ACCAGGCGCCAGCGTGAAAGTGAGCTGCAAAGCCAGCGGCTTC |
| | | ACCTTCACCAGCTATTATATCCACTGGGTGCGCAAGGCCCCAGG |
| | | CCAGGGCCTGGAATGGATCGGCTGGATCTATCCAGAAAACGAT |
| | | AACACCAAATATAACGAAAAATTCAAAGATCGCGTGACCATCACC |
| | | GCCGATACCAGCACCAGCACCGCCTATCTGGAACTGAGCAGCC |
| | | TGCGCAGCGAAGATACCGCCGTGTATTATTGCGCCCGCGATGG |
| | | CTATAGCCGCTATTATTTCGATTATTGGGGCCAGGGCACCCTGG |
| | | TGACCGTCTCGAGTGCCTCCACCAAGGGCCCATCGGTCTTCCC |
| | | CCTGGCACCCTCCTCCAAGAGCACCTCTGGGGGCACAGCGGCC |
| | | CTGGGCTGCCTGGTCAAGGACTACTTCCCCGAGCCGGTGACGG |
| | | TGTCGTGGAACTCAGGCGCCCTGACCAGCGGCGTGCACACCTT |
| | | CCCGGCTGTCCTACAGTCCTCAGGACTCTACTCCCTCGAAAGCG |
| | | TGGTGACTGTGCCCTCTAGCAGCTTGGGCACCCAGACCTACATC |
| | | TGCAACGTGAATCACAAGCCCAGCAACACCAAGGTGGACAAGA |
| | | AAGTTGAGCCCAAATCTTGTGACAAAACTCACACATGCCCACCG |
| | | TGCCCAGCACCTGAAGCCGCGGGGGGACCGTCAGTCTTCCTCT |
| | | TCCCCCCAAAACCCAAGGACACCCTCATGATCTCCCGGACCCCT |
| | | GAGGTCACATGCGTGGTGGTGGACGTGAGCCACGAAGACCCTG |
| | | AGGTCAAGTTCAACTGGTACGTGGACGGCGTGGAGGTGCATAA |
| | | TGCCAAGACAAAGCCGCGGGAGGAGCAGTACAACAGCACGTAC |
| | | CGGGTGGTCAGCGTCCTCACCGTCCTGCACCAGGACTGGCTGA |
| | | ATGGCAAGGAGTACAAGTGCAAGGTCTCCAACAAAGCCCTCGG |
| | | AGCCCCCATCGAGAAAACCATCTCCAAAGCCAAAGGGCAGCCC |
| | | CGAGAACCACAGGTGTACACCCTGCCCCCATCCCGGGAAGAGA |
| | | TGACCAAGAACCAGGTCAGCCTGAGCTGCGCGGTCAAAGGCTT |
| | | CTATCCCAGCGACATCGCCGTGGAGTGGGAGAGCAATGGGCAG |
| | | CCGGAGAACAACTACAAGACCACGCCTCCCGTGCTGGACTCCG |
| | | ACGGCTCCTTCTTCCTCGTCAGCAAGCTCACCGTGGACAAGAGC |
| | | AGGTGGCAGCAGGGGAACGTCTTCTCATGCTCCGTGATGCATG |
| | | AGGCTCTGCACAACCACTACACGCAGAAGAGCCTCTCCCTGTCT |
| | | CCGGGTAAA |
| 90 | CCR8-CD3 | EVQLLESGGGLVQPGGSLRLSCAASGIDLSTYAMGWVREAPGKGL |
| | (MD1) stacked | EWVGLIHRSGRTYYATWAKGRFTISKDSSKNTLYLQMNSLRAEDTA |
| | fab HC hole | VYYCTRSYPDYSATASIWGQGTTVTVSSASTKGPSVFPLAPSSKST |
| | | SGGTAALGCLVKDYFPEPVTVSWNSGALTSGVHTFPAVLQSSGLY |
| | | SLKSVVTVPSSSLGTQTYICNVNHKPSNTKVDKKVEPKSCDKTHTE |
| | | VQLVQSGAEVKKPGASVKVSCKASGFTFTSYYIHWVRKAPGQGLE |
| | | WIGWIYPENDNTKYNEKFKDRVTITADTSTSTAYLELSSLRSEDTAV |
| | | YYCARDGYSRYYFDYWGQGTLVTVSSASTKGPSVFPLAPSSKSTS |
| | | GGTAALGCLVKDYFPEPVTVSWNSGALTSGVHTFPAVLQSSGLYS |
| | | LESVVTVPSSSLGTQTYICNVNHKPSNTKVDKKVEPKSCDKTHTCP |
| | | PCPAPEAAGGPSVFLFPPKPKDTLMISRTPEVTCVVVDVSHEDPEV |
| | | KFNWYVDGVEVHNAKTKPREEQYNSTYRVVSVLTVLHQDWLNGK |
| | | EYKCKVSNKALGAPIEKTISKAKGQPREPQVYTLPPSREEMTKNQV |
| | | SLSCAVKGFYPSDIAVEWESNGQPENNYKTTPPVLDSDGSFFLVSK |
| | | LTVDKSRWQQGNVFSCSVMHEALHNHYTQKSLSLSPGK |
| 91 | CD3 MD1 CDR- | SYYIH |
| | H1 | |
| 92 | CD3 MD1 CDR- | WIYPENDNTKYNEKFKD |
| | H2 | |
| 93 | CD3 MD1 CDR- | DGYSRYYFDY |
| | H3 | |
| 94 | CD3 MD1 CDR- | KSSQSLLNSRTRKNYLA |
| | L1 | |
| 95 | CD3 MD1 CDR- | WASTRES |
| | L2 | |
| 96 | CD3 MD1 CDR- | TQSFILRT |
| | L3 | |
| 97 | CD3 MD1 VH | EVQLVQSGAEVKKPGASVKVSCKASGFTFTSYYIHWVRKAPGQGL |
| | | EWIGWIYPENDNTKYNEKFKDRVTITADTSTSTAYLELSSLRSEDTA |
| | | VYYCARDGYSRYYFDYWGQGTLVTVSS |
| 98 | CD3 MD1 VL | DIVMTQSPDSLAVSLGERATINCKSSQSLLNSRTRKNYLAWYQEKP |
| | | GQPPKLLIYWASTRESGVPDRFSGSGSGTDFTLTISSLQAEDVAVY |
| | | YCTQSFILRTFGQGTKVEIK |
| 99 | CD3 MD1 FR-H1 | EVQLVQSGAEVKKPGASVKVSCKASGFTFT |
| 100 | CD3 MD1 FR-H2 | WVRKAPGQGLEWIG |
| 101 | CD3 MD1 FR-H3 | RVTITADTSTSTAYLELSSLRSEDTAVYYCAR |
| 102 | CD3 MD1 FR-H4 | WGQGTLVTVSS |
| 103 | CD3 MD1 FR-L1 | DIVMTQSPDSLAVSLGERATINC |
| 104 | CD3 MD1 FR-L2 | WYQEKPGQPPKLLIY |
| 105 | CD3 MD1 FR-L3 | GVPDRFSGSGSGTDFTLTISSLQAEDVAVYYC |
| 106 | CD3 MD1 FR-L4 | FGQGTKVEIK |
| 107 | Charge Swap | EVQLLESGGGLVQPGGSLRLSCAASGIDLSTYAMGWVRKAPGKGL |
| | CCR8 1889 | EWVGLIHRSGRTYYATWAKGRFTISKDSSKNTLYLQMNSLRAEDTA |
| | S12P VH | VYYCTRSYPDYSATASIWGQGTTVTVSS |
| 108 | Charge Swap | DIQVTQSPSSLPASVGDRVTITCQASENIANALAWYQEKPGKPPKF |
| | CCR8 1889 | LIYGASNLASGVPSRFSGSGSGTDFTFTISSLQPEDIATYYCQQAYY |
| | S12P VL | GNSFVEGTFGGGTKVEIK |
| 109 | Charge Swap | EVQLVQSGAEVKKPGASVKVSCKASGYTFTNYYIHWVREAPGQGL |
| | CD3 40G5c VH | EWIGWIYPGDGNTKYNEKFKGRATLTADTSTSTAYLELSSLRSEDT |
| | | AVYYCARDSYSNYYFDYWGQGTLVTVSS |
| 110 | Charge Swap | DIVMTQSPDSLAVSLGERATINCKSSQSLLNSRTRKNYLAWYQKKP |
| | CD3 40G5c VL | GQPPKLLIYWASTRESGVPDRFSGSGSGTDFTLTISSLQAEDVAVY |
| | | YCTQSFILRTFGQGTKVEIK |
| 111 | Charge Swap | EVQLVQSGAEVKKPGASVKVSCKASGFTFTSYYIHWVREAPGQGL |
| | CD3 MD1 VH | EWIGWIYPENDNTKYNEKFKDRVTITADTSTSTAYLELSSLRSEDTA |
| | | VYYCARDGYSRYYFDYWGQGTLVTVSS |
| 112 | Charge Swap | DIVMTQSPDSLAVSLGERATINCKSSQSLLNSRTRKNYLAWYQKKP |
| | CD3 MD1 VL | GQPPKLLIYWASTRESGVPDRFSGSGSGTDFTLTISSLQAEDVAVY |
| | | YCTQSFILRTFGQGTKVEIK |
| 113 | Charge Swap | WVRKAPGKGLEWVG |
| | CCR8 1889 | |
| | S12P FR-H2 | |
| 114 | Charge Swap | WYQEKPGKPPKFLIY |
| | CCR8 1889 | |
| | S12P FR-L2 | |
| 115 | Charge Swap | WVREAPGQGLEWIG |
| | CD3 40G5c and | |
| | MD1 FR-H2 | |
| 116 | Charge Swap | WYQKKPGQPPKLLIY |
| | CD3 40G5c and | |
| | MD1 FR-L2 | |
| 117 | Charge Swap | EVQLLESGGGLVQPGGSLRLSCAASGIDLSTYAMGWVRKAPGKGL |
| | CCR8 knob: | EWVGLIHRSGRTYYATWAKGRFTISKDSSKNTLYLQMNSLRAEDTA |
| | | VYYCTRSYPDYSATASIWGQGTTVTVSSASTKGPSVFPLAPSSKST |
| | | SGGTAALGCLVKDYFPEPVTVSWNSGALTSGVHTFPAVLQSSGLY |
| | | SLESVVTVPSSSLGTQTYICNVNHKPSNTKVDKKVEPKSCDKTHTC |
| | | PPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALGAPIEKTISKAKGQPREPQVYTLPPSREEMTKNQ |
| | | VSLWCLVKGFYPSDIAVEWESNGQPENNYKTTPPVLDSDGSFFLY |
| | | SKLTVDKSRWQQGNVFSCSVMHEALHNHYTQKSLSLSPGK |
| 118 | Charge Swap | DIQVTQSPSSLPASVGDRVTITCQASENIANALAWYQEKPGKPPKF |
| | CCR8 LC (P): | LIYGASNLASGVPSRFSGSGSGTDFTFTISSLQPEDIATYYCQQAYY |
| | | GNSFVEGTFGGGTKVEIKRTVAAPSVFIFPPSDEQLKSGTASVKCLL |
| | | NNFYPREAKVQWKVDNALQSGNSQESVTEQDSKDSTYSLSSTLTL |
| | | SKADYEKHKVYACEVTHQGLSSPVTKSFNRGEC |
| 119 | Charge Swap | EVQLLESGGGLVQPGGSLRLSCAASGIDLSTYAMGWVRKAPGKGL |
| | CCR8-CD3 | EWVGLIHRSGRTYYATWAKGRFTISKDSSKNTLYLQMNSLRAEDTA |
| | (40G5c) stacked | VYYCTRSYPDYSATASIWGQGTTVTVSSASTKGPSVFPLAPSSKST |
| | Fab HC hole: | SGGTAALGCLVKDYFPEPVTVSWNSGALTSGVHTFPAVLQSSGLY |
| | | SLESVVTVPSSSLGTQTYICNVNHKPSNTKVDKKVEPKSCDKTHTE |
| | | VQLVQSGAEVKKPGASVKVSCKASGYTFTNYYIHWVREAPGQGLE |
| | | WIGWIYPGDGNTKYNEKFKGRATLTADTSTSTAYLELSSLRSEDTA |
| | | VYYCARDSYSNYYFDYWGQGTLVTVSSASTKGPSVFPLAPSSKST |
| | | SGGTAALGCLVKDYFPEPVTVSWNSGALTSGVHTFPAVLQSSGLY |
| | | SLKSVVTVPSSSLGTQTYICNVNHKPSNTKVDKKVEPKSCDKTHTC |
| | | PPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALGAPIEKTISKAKGQPREPQVYTLPPSREEMTKNQ |
| | | VSLSCAVKGFYPSDIAVEWESNGQPENNYKTTPPVLDSDGSFFLVS |
| | | KLTVDKSRWQQGNVFSCSVMHEALHNHYTQKSLSLSPGK |
| 120 | Charge Swap | DIVMTQSPDSLAVSLGERATINCKSSQSLLNSRTRKNYLAWYQKKP |
| | CD3 LC: | GQPPKLLIYWASTRESGVPDRFSGSGSGTDFTLTISSLQAEDVAVY |
| | | YCTQSFILRTFGQGTKVEIKRTVAAPSVFIFPPSDEQLKSGTASVEC |
| | | LLNNFYPREAKVQWKVDNALQSGNSQESVTEQDSKDSTYSLSSTL |
| | | TLSKADYEKHKVYACEVTHQGLSSPVTKSFNRGEC |
| 121 | Charge Swap | EVQLLESGGGLVQPGGSLRLSCAASGIDLSTYAMGWVRKAPGKGL |
| | CCR8-CD3 | EWVGLIHRSGRTYYATWAKGRFTISKDSSKNTLYLQMNSLRAEDTA |
| | (MD1) stacked | VYYCTRSYPDYSATASIWGQGTTVTVSSASTKGPSVFPLAPSSKST |
| | Fab HC hole | SGGTAALGCLVKDYFPEPVTVSWNSGALTSGVHTFPAVLQSSGLY |
| | | SLESVVTVPSSSLGTQTYICNVNHKPSNTKVDKKVEPKSCDKTHTE |
| | | VQLVQSGAEVKKPGASVKVSCKASGFTFTSYYIHWVREAPGQGLE |
| | | WIGWIYPENDNTKYNEKFKDRVTITADTSTSTAYLELSSLRSEDTAV |
| | | YYCARDGYSRYYFDYWGQGTLVTVSSASTKGPSVFPLAPSSKSTS |
| | | GGTAALGCLVKDYFPEPVTVSWNSGALTSGVHTFPAVLQSSGLYS |
| | | LKSVVTVPSSSLGTQTYICNVNHKPSNTKVDKKVEPKSCDKTHTCP |
| | | PCPAPEAAGGPSVFLFPPKPKDTLMISRTPEVTCVVVDVSHEDPEV |
| | | KFNWYVDGVEVHNAKTKPREEQYNSTYRVVSVLTVLHQDWLNGK |
| | | EYKCKVSNKALGAPIEKTISKAKGQPREPQVYTLPPSREEMTKNQV |
| | | SLSCAVKGFYPSDIAVEWESNGQPENNYKTTPPVLDSDGSFFLVSK |
| | | LTVDKSRWQQGNVFSCSVMHEALHNHYTQKSLSLSPGK |
| 122 | Biotinylated cyno | DGNEEMGSITQTPYQVSISGTTVILTKK |
| | CD3 epsilon | wherein the first D is modified with pyroglutamate, |
| | peptide | and the last K is modified with biotin-amide |

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A bispecific antigen-binding molecule comprising:
(a) a first antigen-binding domain that binds C-C motif chemokine receptor 8 (CCR8), wherein the first antigen-binding domain comprises the following six complementarity-determining regions (CDRs):
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 4);
(v) a CDR-L2 comprising the amino acid sequence GASNLAS (SEQ ID NO: 5); and
(vi) a CDR-L3 comprising the amino acid sequence QQAYYGNSFVEGT (SEQ ID NO: 6); and
(b) a second antigen-binding domain that binds cluster of differentiation 3 (CD3), wherein the second antigen-binding domain comprises the following six CDRs:
(i) a CDR-H1 comprising the amino acid sequence NYYIH (SEQ ID NO: 17);
(ii) a CDR-H2 comprising the amino acid sequence WIYPGDGNTKYNEKFKG (SEQ ID NO: 18);
(iii) a CDR-H3 comprising the amino acid sequence DSYSNYYFDY (SEQ ID NO: 19);
(iv) a CDR-L1 comprising the amino acid sequence KSSQSLLNSRTRKNYLA (SEQ ID NO: 20);
(v) a CDR-L2 comprising the amino acid sequence WASTRES (SEQ ID NO: 21); and
(vi) a CDR-L3 comprising the amino acid sequence TQSFILRT (SEQ ID NO: 22).

2. The bispecific antigen-binding molecule of claim 1, wherein:
(A) the first antigen-binding domain comprises a light chain variable region (VL) domain and a heavy chain variable region (VH) domain, and wherein:
(a) the VL domain comprises a proline residue at position 12 (numbering according to Kabat); and/or
(b) the VL domain comprises a lysine residue at position 38 and the VH domain comprises a glutamic acid residue at position 39 (numbering according to Kabat); and/or
the second antigen-binding domain comprises a VL domain and a VH domain, and wherein the VL domain comprises a glutamic acid residue at position 38 and the VH domain comprises a lysine residue at position 39 (numbering according to Kabat);
(B)
(a) the first antigen-binding domain comprises one or more of the following eight framework regions (FRs):
(i) an FR-H1 comprising the amino acid sequence of SEQ ID NO: 9;
(ii) an FR-H2 comprising the amino acid sequence of SEQ ID NO: 10;
(iii) an FR-H3 comprising the amino acid sequence of SEQ ID NO: 11;
(iv) an FR-H4 comprising the amino acid sequence of SEQ ID NO: 12;
(v) an FR-L1 comprising the amino acid sequence of SEQ ID NO: 13:
(vi) an FR-L2 comprising the amino acid sequence of SEQ ID NO: 14;
(vii) an FR-L3 comprising the amino acid sequence of SEQ ID NO: 15; and/or
(viii) an FR-L4 comprising the amino acid sequence of SEQ ID NO: 16; and/or
(b) the second antigen-binding domain comprises one or more of the following eight FRs;
(i) an FR-H1 comprising the amino acid sequence of SEQ ID NO: 25;
(ii) an FR-H2 comprising the amino acid sequence of SEQ ID NO: 26;
(iii) an FR-H3 comprising the amino acid sequence of SEQ ID NO: 27;
(iv) an FR-H4 comprising the amino acid sequence of SEQ ID NO: 28;
(v) an FR-L1 comprising the amino acid sequence of SEQ ID NO: 29;
(vi) an FR-L2 comprising the amino acid sequence of SEQ ID NO: 30;
(vii) an FR-L3 comprising the amino acid sequence of SEQ ID NO: 31; and/or
(viii) an FR-L4 comprising the amino acid sequence of SEQ ID NO: 32;
(C)
(a) the first antigen-binding domain comprises:
(i) a VH domain comprising an amino acid sequence having at least 80% sequence identity to the amino acid sequence of SEQ ID NO: 7:
(ii) a VL domain comprising an amino acid sequence having at least 80% sequence identity to the amino acid sequence of SEQ ID NO: 8; or
(iii) a VH domain as in (i) and a VL domain as in (ii); and/or
(b) the second antigen-binding domain comprises:
(i) a VH domain comprising an amino acid sequence having at least 80% sequence identity to the amino acid sequence of SEQ ID NO: 23;
(ii) a VL domain comprising an amino acid sequence having at least 80% sequence identity to the amino acid sequence of SEQ ID NO: 24; or
(iii) a VH domain as in (i) and a VL domain as in (ii);
(D) the first antigen-binding domain is a Fab molecule comprising a Fab light chain and a Fab heavy chain and the second antigen-binding domain is a Fab molecule comprising a Fab light chain and a Fab heavy chain, and wherein:
(a) the Fab light chain of the first antigen-binding domain comprises a glutamic acid residue at position 133, and the Fab heavy chain of the first antigen-binding domain comprises a lysine residue at position 183 (numbering according to Kabat); and/or
(b) the Fab light chain of the second antigen-binding domain comprises a lysine residue at position 133, and the Fab heavy chain of the second antigen-binding domain comprises a glutamic acid residue at position 183 (numbering according to Kabat);
(E) the bispecific antigen-binding molecule comprises an Fc domain comprising a first subunit and a second subunit; and/or
(F) the bispecific antigen-binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain.

3-5. (canceled)

6. The bispecific antigen-binding molecule of claim 2, wherein:
the first antigen-binding domain comprises:
(a) a VH domain comprising the amino acid sequence of SEQ ID NO: 7; (b) a VL domain comprising the amino acid sequence of SEQ ID NO: 8; or (c) a VH domain as in (a) and a VL domain as in (b); and/or
the second antigen-binding domain comprises:
(a) a VH domain comprising the amino acid sequence of SEQ ID NO: 23; (b) a VL domain comprising the amino acid sequence of SEQ ID NO: 24; or (c) a VH domain as in (a) and a VL domain as in (b).

7-10. (canceled)

11. The bispecific antigen-binding molecule of claim 1, further comprising an Fc domain comprising a first subunit and a second subunit, wherein:
(a) the Fc domain is an IgG Fc domain;
(b) the Fc domain is a human IgG Fc domain;
(c) the Fc domain comprises a modification promoting the association of the first subunit and the second subunit of the Fc domain;
(d) the first subunit comprises one or more heavy chain constant domains selected from a first CH2 (CH2₁) domain and/or a first CH3 (CH3₁) domain; and the second subunit comprises one or more heavy chain constant domains selected from a second CH2 (CH2₂) domain and/or a second CH3 (CH3₂) domain;
(e) the first antigen-binding domain and the second antigen-binding domain are each a Fab molecule and the bispecific antigen-binding molecule comprises an Fc domain comprising a first subunit and a second subunit; and wherein the first antigen-binding domain is fused at the C-terminus of the Fab heavy chain to the N-terminus of the first subunit and the second antigen-binding domain is fused at the C-terminus of the Fab heavy chain to the N-terminus of the second subunit; and/or
(f) each of the first subunit and the second subunit comprises an alanine residue at position 234, an alanine residue at position 235, and a glycine residue at position 329 (numbering according to Kabat EU index).

12-16. (canceled)

17. The bispecific antigen-binding molecule of claim 11, wherein the first subunit comprises one or more heavy chain constant domains selected from a CH2₁ domain and/or a CH3₁ domain, wherein the second subunit comprises one or more heavy chain constant domains selected from a CH2₂ domain and/or a CH3₂ domain, wherein at least one of the one or more heavy chain constant domains is paired with another heavy chain constant domain, and wherein:
(a) the CH3₁ and CH3₂ domains each comprise a protuberance or cavity, wherein the protuberance or cavity in the CH3₁ domain is positionable in the cavity or protuberance, respectively, in the CH3₂ domain, and wherein the CH3₁ and CH3₂ domains meet at an interface between said protuberance and cavity;
(b) the CH2₁ and CH2₂ domains each comprise a protuberance or cavity, wherein the protuberance or cavity in the CH2₁ domain is positionable in the cavity or protuberance, respectively, in the CH2₂ domain, and wherein the CH2₁ and CH2₂ domains meet at an interface between said protuberance and cavity; and/or
(c) the first subunit comprises a tryptophan residue at position 366; and the second subunit comprises a serine residue at position 366, an alanine residue at position 368, and a valine residue at position 407 (numbered according to Kabat EU index).

18-24. (canceled)

25. The bispecific antigen-binding molecule of claim 1, wherein the bispecific antigen-binding molecule further comprises a third antigen-binding domain that binds to CCR8.

26. The bispecific antigen-binding molecule of claim 25, wherein:
(A) the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 4);
(v) a CDR-L2 comprising the amino acid sequence GASNLAS (SEQ ID NO: 5); and
(vi) a CDR-L3 comprising the amino acid sequence QQAYYGNSFVEGT (SEQ ID NO: 6);
(B) the third antigen-binding domain comprises a VL domain and a VH domain, and wherein:
(a) the VL domain comprises a proline residue at position 12 (numbering according to Kabat); and/or
(b) the VL domain comprises a lysine residue at position 38 and the VH domain comprises a glutamic acid residue at position 39 (numbering according to Kabat);
(C) the third antigen-binding domain comprises one or more of the following eight FRs:
(i) an FR-H1 comprising the amino acid sequence of SEQ ID NO: 9;
(ii) an FR-H2 comprising the amino acid sequence of SEQ ID NO: 10;
(iii) an FR-H3 comprising the amino acid sequence of SEQ ID NO: 11;
(iv) an FR-H4 comprising the amino acid sequence of SEQ ID NO: 12;
(v) an FR-L1 comprising the amino acid sequence of SEQ ID NO: 13;
(vi) an FR-L2 comprising the amino acid sequence of SEQ ID NO: 14;
(vii) an FR-L3 comprising the amino acid sequence of SEQ ID NO: 15; and/or
(viii) an FR-L4 comprising the amino acid sequence of SEQ ID NO: 16:
(D) the third antigen-binding domain comprises (a) a VH domain comprising an amino acid sequence having at least 80% sequence identity to the amino acid sequence of SEQ ID NO: 7: (b) a VL domain comprising an amino acid sequence having at least 80% sequence identity to the amino acid sequence of SEQ ID NO: 8; or (c) a VH domain as in (a) and a VL domain as in (b);
(E) the third antigen-binding domain is a Fab molecule comprising a Fab light chain and a Fab heavy chain, and wherein the Fab light chain of the third antigen-binding domain comprises a glutamic acid residue at position 133, and the Fab heavy chain of the third antigen-binding domain comprises a lysine residue at position 183 (numbering according to Kabat);
(F) the second antigen-binding domain and the third antigen-binding domain are fused to each other; and/or
(G) the bispecific antigen-binding molecule comprises an Fc domain comprising of a first subunit and a second subunit; wherein the first antigen-binding domain, the second antigen-binding domain, and the third antigen-binding domain are each a Fab molecule; wherein the first antigen-binding domain is fused at the C-terminus of the Fab heavy chain to the N-terminus of the first subunit; wherein the second antigen-binding domain is fused at the C-terminus of the Fab heavy chain to the N-terminus of the second subunit; and wherein the third antigen-binding domain is fused at the C-terminus of the Fab heavy chain to the N-terminus of the Fab heavy chain of the second antigen-binding domain.

27-29. (canceled)

30. The bispecific antigen-binding molecule of claim 26, wherein the third antigen-binding domain comprises (a) a VH domain comprising the amino acid sequence of SEQ ID NO: 7; (b) a VL domain comprising the amino acid sequence of SEQ ID NO: 8; or (c) a VH domain as in (a) and a VL domain as in (b).

31-34. (canceled)

35. The bispecific antigen-binding molecule of claim 26, wherein:
(a) the second antigen-binding domain and the third antigen-binding domain are fused to each other via a peptide linker; and/or
(b) the second antigen-binding domain and the third antigen-binding domain are each a Fab molecule, and wherein the third antigen-binding domain is fused at the C-terminus of the Fab heavy chain to the N-terminus of the Fab heavy chain of the second antigen-binding domain.

36. The bispecific antigen-binding molecule of claim 35, wherein the peptide linker comprises the amino acid sequence of SEQ ID NO: 37.

37-38. (canceled)

39. The bispecific antigen-binding molecule of claim 1, wherein the bispecific antigen-binding molecule comprises a polypeptide comprising an amino acid sequence having at least 80% sequence identity to the amino acid sequence of SEQ ID NO: 33, a polypeptide comprising an amino acid sequence having at least 80% sequence identity to the amino acid sequence of SEQ ID NO: 34, a polypeptide comprising an amino acid sequence having at least 80% sequence identity to the amino acid sequence of SEQ ID NO: 35, and a polypeptide comprising an amino acid sequence having at least 80% sequence identity to the amino acid sequence of SEQ ID NO: 36.

40. The bispecific antigen-binding molecule of claim 39, wherein the bispecific antigen-binding molecule comprises a polypeptide comprising the amino acid sequence of SEQ ID NO: 33, a first polypeptide and a second polypeptide each comprising the amino acid sequence of SEQ ID NO: 34, a polypeptide comprising the amino acid sequence of SEQ ID NO: 35, and a polypeptide comprising the amino acid sequence of SEQ ID NO: 36.

41. The bispecific antigen-binding molecule of claim 40, wherein:
(i) the polypeptide comprising the amino acid sequence of SEQ ID NO: 33 is connected to the first polypeptide comprising the amino acid sequence of SEQ ID NO: 34 via a Fab heavy chain and Fab light chain interaction;
(ii) the polypeptide comprising the amino acid sequence of SEQ ID NO: 35 is connected to the second polypeptide comprising the amino acid sequence of SEQ ID NO: 34 via a Fab heavy chain and Fab light chain interaction;
(iii) the polypeptide comprising the amino acid sequence of SEQ ID NO: 35 is connected to the polypeptide comprising the amino acid sequence of SEQ ID NO: 36 via a Fab heavy chain and Fab light chain interaction; and
(iv) the polypeptide comprising the amino acid sequence of SEQ ID NO: 33 is connected to the polypeptide comprising the amino acid sequence of SEQ ID NO: 35 via a first subunit and a second subunit of an Fc domain.

42. An isolated polynucleotide or a set of isolated polynucleotides encoding the bispecific antigen-binding molecule of claim 1.

43. A vector or a set of vectors comprising the isolated polynucleotide or the set of isolated polynucleotides of claim 42.

44. A host cell or a set of host cells comprising the isolated polynucleotide or the set of isolated polynucleotides of claim 42.

45. A method of producing a bispecific antigen-binding molecule that binds to CCR8 and CD3, comprising the steps of (a) culturing the host cell or the set of host cells of claim 44 under conditions suitable for the expression of the bispecific antigen-binding molecule; and (b) recovering the bispecific antigen binding molecule.

46. (canceled)

47. A bispecific antigen-binding molecule that binds to CCR8 and CD3 produced by the method of claim 45.

48. A pharmaceutical composition comprising the bispecific antigen-binding molecule of claim 1 and a pharmaceutically acceptable carrier.

49-53. (canceled)

54. A method of treating a cancer in a subject, comprising administering to the subject an effective amount of the bispecific antigen-binding molecule of claim 1.

55. The method of claim 54, wherein the cancer is selected from the group consisting of bladder cancer, blastoma, blood cancer, bone cancer, brain cancer, breast cancer, cervical cancer, colorectal cancer, endometrial cancer, esophageal cancer, gastric cancer, head and neck cancer, kidney cancer, liver cancer, lung cancer, ovarian cancer, pancreatic cancer, prostate cancer, sarcoma, skin cancer, testicular cancer, and uterine cancer.

56. (canceled)

57. A method of depleting regulatory T cells in a tumor microenvironment in a subject having cancer comprising administering to the subject an effective amount of the bispecific antigen-binding molecule of claim 1 sufficient to deplete the regulatory T cells in the tumor microenvironment.

58. A method of depleting regulatory T cells outside of a tumor microenvironment in a subject having cancer comprising administering to the subject an effective amount of the bispecific antigen-binding molecule of claim 1 sufficient to deplete the regulatory T cells outside of the tumor microenvironment.

59. The method of claim 57, wherein the regulatory T cells present in the tumor microenvironment of the cancer are depleted.

60. The use of method of claim 58, wherein the regulatory T cells outside of the tumor microenvironment of the cancer are depleted.

61. An in vitro method of depleting regulatory T cells from a cancer cell population, comprising contacting the cell population with the bispecific antigen-binding molecule of claim 1 in an amount sufficient to deplete the regulatory T cells from the cell population.

62. (canceled)

63. A method of reducing CCR8 mRNA expression in the blood of a subject comprising administering to the subject an effective amount of the bispecific antigen-binding molecule of claim 1 sufficient to reduce CCR8 mRNA expression in the blood.

64. The method of claim 54, further comprising administering an additional therapeutic agent to the subject.

65. The method of claim 64, wherein the additional therapeutic agent is:
(a) an anti-cancer agent comprising a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a toll-like receptor (TLR) agent, a bispecific antibody, a cellular therapy, or an immune cell engager; and/or
(b) tocilizumab or a corticosteroid.

66. (canceled)

67. The method of claim 65, wherein the additional therapeutic agent is an anti-cancer agent, and wherein the anti-cancer agent is a PD-L1 binding antagonist.

68. The method of claim 67, wherein the PD-L1 binding antagonist is atezolizumab.

69-93. (canceled)

94. A bispecific antigen-binding molecule comprising:
(a) a first antigen-binding domain that binds C-C motif chemokine receptor 8 (CCR8), wherein the first antigen-binding domain comprises the following six complementarity-determining regions (CDRs):
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 4);
(v) a CDR-L2 comprising the amino acid sequence GASNLAS (SEQ ID NO: 5); and
(vi) a CDR-L3 comprising the amino acid sequence QQAYYGNSFVEGT (SEQ ID NO: 6);
(b) a second antigen-binding domain that binds cluster of differentiation 3 (CD3), wherein the second antigen-binding domain comprises the following six CDRs:
(i) a CDR-H1 comprising the amino acid sequence NYYIH (SEQ ID NO: 17);
(ii) a CDR-H2 comprising the amino acid sequence WIYPGDGNTKYNEKFKG (SEQ ID NO: 18);
(iii) a CDR-H3 comprising the amino acid sequence DSYSNYYFDY (SEQ ID NO: 19);
(iv) a CDR-L1 comprising the amino acid sequence KSSQSLLNSRTRKNYLA (SEQ ID NO: 20);
(v) a CDR-L2 comprising the amino acid sequence WASTRES (SEQ ID NO: 21); and
(vi) a CDR-L3 comprising the amino acid sequence TQSFILRT (SEQ ID NO: 22); and
(c) a third antigen-binding domain that binds CCR8, wherein the third antigen-binding domain comprises the following six CDRs:
(i) a CDR-H1 comprising the amino acid sequence TYAMG (SEQ ID NO: 1);
(ii) a CDR-H2 comprising the amino acid sequence LIHRSGRTYYATWAKG (SEQ ID NO: 2);
(iii) a CDR-H3 comprising the amino acid sequence SYPDYSATASI (SEQ ID NO: 3);
(iv) a CDR-L1 comprising the amino acid sequence QASENIANALA (SEQ ID NO: 4);
(v) a CDR-L2 comprising the amino acid sequence GASNLAS (SEQ ID NO: 5); and
(vi) a CDR-L3 comprising the amino acid sequence QQAYYGNSFVEGT (SEQ ID NO: 6),
wherein:
(A)
(1) the first antigen-binding domain comprises a VL domain and a VH domain, wherein the VL domain comprises a proline residue at position 12 and a lysine residue at position 38, and the VH domain comprises a glutamic acid residue at position 39 (numbering according to Kabat);
(2) the second antigen-binding domain comprises a VL domain and a VH domain, wherein the VL domain comprises a glutamic acid residue at position 38, and the VH domain comprises a lysine residue at position 39 (numbering according to Kabat); and
(3) the third antigen-binding domain comprises a VL domain and a VH domain, wherein the VL domain comprises a proline residue at position 12 and a lysine residue at position 38, and the VH domain comprises a glutamic acid residue at position 39 (numbering according to Kabat);
(B) the first antigen-binding domain is a Fab molecule comprising a Fab light chain and a Fab heavy chain, the second antigen-binding domain is a Fab molecule comprising a Fab light chain and a Fab heavy chain, and the third antigen-binding domain is a Fab molecule comprising a Fab light chain and a Fab heavy chain, and wherein:
(1) the Fab light chain of the first antigen-binding domain comprises a glutamic acid residue at position 133, and the Fab heavy chain of the first antigen-binding domain comprises a lysine residue at position 183 (numbering according to Kabat);
(2) the Fab light chain of the second antigen-binding domain comprises a lysine residue at position 133, and the Fab heavy chain of the second antigen-binding domain comprises a glutamic acid residue at position 183 (numbering according to Kabat); and
(3) the Fab light chain of the third antigen-binding domain comprises a glutamic acid residue at position 133, and the Fab heavy chain of the third antigen-binding domain comprises a lysine residue at position 183 (numbering according to Kabat); and
(C) the bispecific antigen-binding molecule further comprises an IgG Fc domain comprising a first subunit and a second subunit, wherein the first antigen-binding domain is fused at the C-terminus of the Fab heavy chain to the N-terminus of the first subunit, wherein the second antigen-binding domain is fused at the C-terminus of the Fab heavy chain to the N-terminus of the second subunit, wherein the third antigen-binding domain is fused at the C-terminus of the Fab heavy chain to the N-terminus of the Fab heavy chain of the second antigen-binding domain, and wherein:
(1) each of the first subunit and the second subunit comprises an alanine residue at position 234, an alanine residue at position 235, and a glycine residue at position 329 (numbering according to Kabat EU index); and
(2) the first subunit comprises a tryptophan residue at position 366; and the second subunit comprises a serine residue at position 366, an alanine residue at position 368, and a valine residue at position 407 (numbered according to Kabat EU index).

95. The bispecific antigen-binding molecule of claim 94, wherein:
(a) the VH domain of first antigen-binding domain comprises the amino acid sequence of SEQ ID NO: 7 and the VL domain of first antigen-binding domain comprises the amino acid sequence of SEQ ID NO: 8;
(b) the VH domain of the second antigen-binding domain comprises the amino acid sequence of SEQ ID NO: 23 and the VL domain of the second antigen-binding domain comprises the amino acid sequence of SEQ ID NO: 24; and
(c) the VH domain of the third antigen-binding domain comprises the amino acid sequence of SEQ ID NO: 7 and the VL domain of the third antigen-binding domain comprises the amino acid sequence of SEQ ID NO: 8.


SEQUENCE LISTING (122 sequences)

SEQ ID NO: 1 - protein, 5 aa, synthetic construct
TYAMG

SEQ ID NO: 2 - protein, 16 aa, synthetic construct
LIHRSGRTYYATWAKG

SEQ ID NO: 3 - protein, 11 aa, synthetic construct
SYPDYSATASI

SEQ ID NO: 4 - protein, 11 aa, synthetic construct
QASENIANALA

SEQ ID NO: 5 - protein, 7 aa, synthetic construct
GASNLAS

SEQ ID NO: 6 - protein, 13 aa, synthetic construct
QQAYYGNSFVEGT

SEQ ID NO: 7 - protein, 119 aa, synthetic construct
EVQLLESGGGLVQPGGSLRLSCAASGIDLSTYAMGWVREAPGKGLEWVGLIHRSGRTYYATWAKGRFTISKDSSKNTLYLQMNSLRAEDTAVYYCTRSYPDYSATASIWGQGTTVTVSS

SEQ ID NO: 8 - protein, 111 aa, synthetic construct
DIQVTQSPSSLPASVGDRVTITCQASENIANALAWYQKKPGKPPKFLIYGASNLASGVPSRFSGSGSGTDFTFTISSLQPEDIATYYCQQAYYGNSFVEGTFGGGTKVEIK

SEQ ID NO: 9 - protein, 30 aa, synthetic construct
EVQLLESGGGLVQPGGSLRLSCAASGIDLS

SEQ ID NO: 10 - protein, 14 aa, synthetic construct
WVREAPGKGLEWVG

SEQ ID NO: 11 - protein, 32 aa, synthetic construct
RFTISKDSSKNTLYLQMNSLRAEDTAVYYCTR

SEQ ID NO: 12 - protein, 11 aa, synthetic construct
WGQGTTVTVSS

SEQ ID NO: 13 - protein, 23 aa, synthetic construct
DIQVTQSPSSLPASVGDRVTITC

SEQ ID NO: 14 - protein, 15 aa, synthetic construct
WYQKKPGKPPKFLIY

SEQ ID NO: 15 - protein, 32 aa, synthetic construct
GVPSRFSGSGSGTDFTFTISSLQPEDIATYYC

SEQ ID NO: 16 - protein, 10 aa, synthetic construct
FGGGTKVEIK

SEQ ID NO: 17 - protein, 5 aa, synthetic construct
NYYIH

SEQ ID NO: 18 - protein, 17 aa, synthetic construct
WIYPGDGNTKYNEKFKG

SEQ ID NO: 19 - protein, 10 aa, synthetic construct
DSYSNYYFDY

SEQ ID NO: 20 - protein, 17 aa, synthetic construct
KSSQSLLNSRTRKNYLA

SEQ ID NO: 21 - protein, 7 aa, synthetic construct
WASTRES

SEQ ID NO: 22 - protein, 8 aa, synthetic construct
TQSFILRT

SEQ ID NO: 23 - protein, 119 aa, synthetic construct
EVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSS

SEQ ID NO: 24 - protein, 112 aa, synthetic construct
DIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRKAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQEKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 449 aa, synthetic construct
EVQLLESGGGLVQPGGSLRLSCAASGIDLSTYAMGWVREAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 218 aa, synthetic construct
DIQVTQSPSSLPASVGDRVTITCQASENIANALAWYQKKPGKPPKFLIYGASNLASGVPSRFSGSGSGTDFTFTISSLQPEDIATYYCQQAYYGNSFVEGTFGGGTKVEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 676 aa, synthetic construct
EVQLLESGGGLVQPGGSLRLSCAASGIDLSTYAMGWVREAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTEVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 5 aa, synthetic construct
DKTHT

SEQ ID NO: 38 - protein, 11 aa, synthetic construct
QASENIANALA

SEQ ID NO: 39 - protein, 7 aa, synthetic construct
GASNLAS

SEQ ID NO: 40 - protein, 13 aa, synthetic construct
QQAYYGNSFVEGT

SEQ ID NO: 41 - protein, 11 aa, synthetic construct
QASENVANALA

SEQ ID NO: 42 - protein, 7 aa, synthetic construct
GASNLAS

SEQ ID NO: 43 - protein, 13 aa, synthetic construct
QQAYYGNSFVEGT

SEQ ID NO: 44 - protein, 11 aa, synthetic construct
QASENIANILA

SEQ ID NO: 45 - protein, 7 aa, synthetic construct
GASNLAS

SEQ ID NO: 46 - protein, 13 aa, synthetic construct
QQAYYGNSFVEGT

SEQ ID NO: 47 - protein, 11 aa, synthetic construct
QASENIANALA

SEQ ID NO: 48 - protein, 7 aa, synthetic construct
GASNLAS

SEQ ID NO: 49 - protein, 13 aa, synthetic construct
QQAYYGNSFVSGT

SEQ ID NO: 50 - protein, 11 aa, synthetic construct
QASENVANILA

SEQ ID NO: 51 - protein, 7 aa, synthetic construct
GASNLAS

SEQ ID NO: 52 - protein, 13 aa, synthetic construct
QQAYYGNSFVEGT

SEQ ID NO: 53 - protein, 11 aa, synthetic construct
QASENVANALA

SEQ ID NO: 54 - protein, 7 aa, synthetic construct
GASNLAS

SEQ ID NO: 55 - protein, 13 aa, synthetic construct
QQAYYGNSFVSGT

SEQ ID NO: 56 - protein, 11 aa, synthetic construct
QASENIANILA

SEQ ID NO: 57 - protein, 7 aa, synthetic construct
GASNLAS

SEQ ID NO: 58 - protein, 13 aa, synthetic construct
QQAYYGNSFVSGT

SEQ ID NO: 59 - protein, 11 aa, synthetic construct
QASENVANILA

SEQ ID NO: 60 - protein, 7 aa, synthetic construct
GASNLAS

SEQ ID NO: 61 - protein, 13 aa, synthetic construct
QQAYYGNSFVSGT

SEQ ID NO: 62 - protein, 5 aa, synthetic construct
TYAMG

SEQ ID NO: 63 - protein, 16 aa, synthetic construct
LIHRSGRTAYATWAKG

SEQ ID NO: 64 - protein, 11 aa, synthetic construct
SYPDYSATASI

SEQ ID NO: 65 - protein, 5 aa, synthetic construct
TYAMG

SEQ ID NO: 66 - protein, 16 aa, synthetic construct
LIHRSGRDAYATWAKG

SEQ ID NO: 67 - protein, 11 aa, synthetic construct
SYPDYSATASI

SEQ ID NO: 68 - protein, 119 aa, synthetic construct; identical to SEQ ID NO: 7

SEQ ID NO: 69 - protein, 111 aa, synthetic construct
DIQVTQSPSSLSASVGDRVTITCQASENIANALAWYQKKPGKPPKFLIYGASNLASGVPSRFSGSGSGTDFTFTISSLQPEDIATYYCQQAYYGNSFVEGTFGGGTKVEIK

SEQ ID NO: 70 - protein, 111 aa, synthetic construct
DIQVTQSPSSLPASVGDRVTITCQASENVANALAWYQKKPGKPPKFLIYGASNLASGVPSRFSGSGSGTDFTFTISSLQPEDIATYYCQQAYYGNSFVEGTFGGGTKVEIK

SEQ ID NO: 71 - protein, 111 aa, synthetic construct
DIQVTQSPSSLPASVGDRVTITCQASENIANILAWYQKKPGKPPKFLIYGASNLASGVPSRFSGSGSGTDFTFTISSLQPEDIATYYCQQAYYGNSFVEGTFGGGTKVEIK

SEQ ID NO: 72 - protein, 111 aa, synthetic construct
DIQVTQSPSSLPASVGDRVTITCQASENIANALAWYQKKPGKPPKFLIYGASNLASGVPSRFSGSGSGTDFTFTISSLQPEDIATYYCQQAYYGNSFVSGTFGGGTKVEIK

SEQ ID NO: 73 - protein, 111 aa, synthetic construct
DIQVTQSPSSLPASVGDRVTITCQASENVANILAWYQKKPGKPPKFLIYGASNLASGVPSRFSGSGSGTDFTFTISSLQPEDIATYYCQQAYYGNSFVEGTFGGGTKVEIK

SEQ ID NO: 74 - protein, 111 aa, synthetic construct
DIQVTQSPSSLPASVGDRVTITCQASENVANALAWYQKKPGKPPKFLIYGASNLASGVPSRFSGSGSGTDFTFTISSLQPEDIATYYCQQAYYGNSFVSGTFGGGTKVEIK

SEQ ID NO: 75 - protein, 111 aa, synthetic construct
DIQVTQSPSSLPASVGDRVTITCQASENIANILAWYQKKPGKPPKFLIYGASNLASGVPSRFSGSGSGTDFTFTISSLQPEDIATYYCQQAYYGNSFVSGTFGGGTKVEIK

SEQ ID NO: 76 - protein, 111 aa, synthetic construct
DIQVTQSPSSLPASVGDRVTITCQASENVANILAWYQKKPGKPPKFLIYGASNLASGVPSRFSGSGSGTDFTFTISSLQPEDIATYYCQQAYYGNSFVSGTFGGGTKVEIK

SEQ ID NO: 77 - protein, 119 aa, synthetic construct
EVQLLESGGGLVQPGGSLRLSCAASGIDLSTYAMGWVREAPGKGLEWVGLIHRSGRTAYATWAKGRFTISKDSSKNTLYLQMNSLRAEDTAVYYCTRSYPDYSATASIWGQGTTVTVSS

SEQ ID NO: 78 - protein, 119 aa, synthetic construct
EVQLLESGGGLVQPGGSLRLSCAASGIDLSTYAMGWVREAPGKGLEWVGLIHRSGRDAYATWAKGRFTISKDSSKNTLYLQMNSLRAEDTAVYYCTRSYPDYSATASIWGQGTTVTVSS

SEQ ID NO: 79 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 80 - protein, 355 aa, Macaca fascicularis
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 81 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 82 - protein, 16 aa, synthetic construct
  VARIANT 8: note = T or D
  VARIANT 9: note = Y or A
LIHRSGRXXYATWAKG

SEQ ID NO: 83 - protein, 11 aa, synthetic construct
  VARIANT 6: note = I or V
  VARIANT 9: note = A or I
QASENXANXLA

SEQ ID NO: 84 - protein, 13 aa, synthetic construct
  VARIANT 11: note = E or S
QQAYYGNSFVXGT

SEQ ID NO: 85 - DNA, 711 bp, synthetic construct
  source 1..711: mol_type = other DNA
atgggatggtcatgtatcatcctttttctagtagcaactgcaaccggtgtacattcagacatccaggtgacccagtctccatcctccctgcctgcatctgtaggagacagagtcaccatcacttgccaggcgagtgagaacattgccaacgctttagcttggtatcagaagaaaccagggaaaccccctaagttcctgatctacggtgcatccaatttggcatcaggggtcccatcaaggttcagtggaagtggatctgggacagattttactttcaccatcagcagcctgcagcctgaagatattgcaacatattactgtcaacaggcttattatggcaatagttttgtggagggcactttcggcggaggtaccaaggtggagatcaaacgaactgtggctgcaccatctgtcttcatcttcccgccatctgatgagcagttgaaatctggaactgcttctgttgaatgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 86 - DNA, 1404 bp, synthetic construct
  source 1..1404: mol_type = other DNA
atgggatggtcatgtatcatcctttttctagtagcaactgcaaccggtgtacattcagaggtgcaactgttggagtctgggggaggcttggtacagcctggggggtccctgagactctcctgtgcagcctctggaatcgacttgagcacctatgccatgggctgggtccgcgaggctccagggaaggggctggagtgggtcggacttattcataggagtggtaggacatactacgcaacctgggcgaagggccggttcaccatctccaaagactcctccaagaacacgctgtatctgcaaatgaacagcctgagagccgaggacacggccgtatattactgtacgagaagttatcccgactactccgccactgcgagcatctgggggcaagggaccacggtcaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcaaaagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggaagagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - DNA, 714 bp, synthetic construct
  source 1..714: mol_type = other DNA
atgggatggtcatgtatcatcctttttctagtagcaactgcaaccggtgtacattcagatatcgtgatgacccagagcccagatagcctggccgtgagcctgggcgaacgcgccaccatcaactgcaaaagcagccagagcctgctgaacagccgcacccgcaaaaactatctggcctggtatcaggaaaaaccaggccagccaccaaaactgctgatctattgggccagcacccgcgaaagcggcgtgccagatcgcttcagcggcagcggcagcggcaccgatttcaccctgaccatcagcagcctgcaggccgaagatgtggccgtgtattattgcacccagagcttcatcctgcgcaccttcggccagggtaccaaggtggagatcaaacgaactgtggctgcaccatctgtcttcatcttcccgccatctgatgagcagttgaaatctggaactgcttctgttaaatgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 88 - DNA, 2085 bp, synthetic construct
  source 1..2085: mol_type = other DNA
atgggatggtcatgtatcatcctttttctagtagcaactgcaaccggtgtacattcagaggtgcaactgttggagtctgggggaggcttggtacagcctggggggtccctgagactctcctgtgcagcctctggaatcgacttgagcacctatgccatgggctgggtccgcgaggctccagggaaggggctggagtgggtcggacttattcataggagtggtaggacatactacgcaacctgggcgaagggccggttcaccatctccaaagactcctccaagaacacgctgtatctgcaaatgaacagcctgagagccgaggacacggccgtatattactgtacgagaagttatcccgactactccgccactgcgagcatctgggggcaagggaccacggtcaccgtgagctccgccagcaccaaaggccccagcgtgttccccctggcccccagctccaagagcaccagcggcggaaccgccgctctgggctgcctggtgaaggactacttccccgagcccgtgaccgtgagctggaacagcggcgccctgaccagcggcgtgcacaccttccccgccgtgctgcagagctccggcctgtacagcctgaagtccgtggtcaccgtgcccagctcctctctgggcacccagacctacatctgcaacgtgaaccacaagcccagcaacaccaaggtggacaagaaggtggagcccaagagctgcgacaagacccacaccgaggtgcagctggtgcagagcggcgccgaggtgaagaagcccggcgccagcgtgaaggtgagctgcaaggccagcggctacaccttcaccaactactatatccactgggtgagaaaggcccccggccagggcctggagtggatcggctggatctaccccggcgacggcaacaccaagtacaacgagaagttcaagggcagagccaccctgaccgccgacaccagcaccagcaccgcctacctggagctgagctccctgagaagcgaggacaccgccgtgtactattgcgccagagacagctacagcaactactatttcgactactggggccagggcaccctggtgaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcgaaagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggaagagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 89 - DNA, 2085 bp, synthetic construct
  source 1..2085: mol_type = other DNA
atgggatggtcatgtatcatcctttttctagtagcaactgcaaccggtgtacattcagaggtgcaactgttggagtctgggggaggcttggtacagcctggggggtccctgagactctcctgtgcagcctctggaatcgacttgagcacctatgccatgggctgggtccgcgaggctccagggaaggggctggagtgggtcggacttattcataggagtggtaggacatactacgcaacctgggcgaagggccggttcaccatctccaaagactcctccaagaacacgctgtatctgcaaatgaacagcctgagagccgaggacacggccgtatattactgtacgagaagttatcccgactactccgccactgcgagcatctgggggcaagggaccacggtcaccgtgagctccgccagcaccaaaggccccagcgtgttccccctggcccccagctccaagagcaccagcggcggaaccgccgctctgggctgcctggtgaaggactacttccccgagcccgtgaccgtgagctggaacagcggcgccctgaccagcggcgtgcacaccttccccgccgtgctgcagagctccggcctgtacagcctgaagtccgtggtcaccgtgcccagctcctctctgggcacccagacctacatctgcaacgtgaaccacaagcccagcaacaccaaggtggacaagaaggtggagcccaagagctgcgacaagacccacaccgaagtgcagctggtgcagagcggcgccgaagtgaaaaaaccaggcgccagcgtgaaagtgagctgcaaagccagcggcttcaccttcaccagctattatatccactgggtgcgcaaggccccaggccagggcctggaatggatcggctggatctatccagaaaacgataacaccaaatataacgaaaaattcaaagatcgcgtgaccatcaccgccgataccagcaccagcaccgcctatctggaactgagcagcctgcgcagcgaagataccgccgtgtattattgcgcccgcgatggctatagccgctattatttcgattattggggccagggcaccctggtgaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcgaaagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggaagagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 90 - protein, 676 aa, synthetic construct
EVQLLESGGGLVQPGGSLRLSCAASGIDLSTYAMGWVREAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTEVQLVQSGAEVKKPGASVKVSCKASGFTFTSYYIHWVRKAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 5 aa, synthetic construct
SYYIH

SEQ ID NO: 92 - protein, 17 aa, synthetic construct
WIYPENDNTKYNEKFKD

SEQ ID NO: 93 - protein, 10 aa, synthetic construct
DGYSRYYFDY

SEQ ID NO: 94 - protein, 17 aa, synthetic construct
KSSQSLLNSRTRKNYLA

SEQ ID NO: 95 - protein, 7 aa, synthetic construct
WASTRES

SEQ ID NO: 96 - protein, 8 aa, synthetic construct
TQSFILRT

SEQ ID NO: 97 - protein, 119 aa, synthetic construct
EVQLVQSGAEVKKPGASVKVSCKASGFTFTSYYIHWVRKAPGQGLEWIGWIYPENDNTKYNEKFKDRVTITADTSTSTAYLELSSLRSEDTAVYYCARDGYSRYYFDYWGQGTLVTVSS

SEQ ID NO: 98 - protein, 112 aa, synthetic construct; identical to SEQ ID NO: 24

SEQ ID NO: 99 - protein, 30 aa, synthetic construct
EVQLVQSGAEVKKPGASVKVSCKASGFTFT

SEQ ID NO: 100 - protein, 14 aa, synthetic construct
WVRKAPGQGLEWIG

SEQ ID NO: 101 - protein, 32 aa, synthetic construct
RVTITADTSTSTAYLELSSLRSEDTAVYYCAR

SEQ ID NO: 102 - protein, 11 aa, synthetic construct
WGQGTLVTVSS

SEQ ID NO: 103 - protein, 23 aa, synthetic construct
DIVMTQSPDSLAVSLGERATINC

SEQ ID NO: 104 - protein, 15 aa, synthetic construct
WYQEKPGQPPKLLIY

SEQ ID NO: 105 - protein, 32 aa, synthetic construct; identical to SEQ ID NO: 31

SEQ ID NO: 106 - protein, 10 aa, synthetic construct
FGQGTKVEIK

SEQ ID NO: 107 - protein, 119 aa, synthetic construct
EVQLLESGGGLVQPGGSLRLSCAASGIDLSTYAMGWVRKAPGKGLEWVGLIHRSGRTYYATWAKGRFTISKDSSKNTLYLQMNSLRAEDTAVYYCTRSYPDYSATASIWGQGTTVTVSS

SEQ ID NO: 108 - protein, 111 aa, synthetic construct
DIQVTQSPSSLPASVGDRVTITCQASENIANALAWYQEKPGKPPKFLIYGASNLASGVPSRFSGSGSGTDFTFTISSLQPEDIATYYCQQAYYGNSFVEGTFGGGTKVEIK

SEQ ID NO: 109 - protein, 119 aa, synthetic construct
EVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSS

SEQ ID NO: 110 - protein, 112 aa, synthetic construct
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIK

SEQ ID NO: 111 - protein, 119 aa, synthetic construct
EVQLVQSGAEVKKPGASVKVSCKASGFTFTSYYIHWVREAPGQGLEWIGWIYPENDNTKYNEKFKDRVTITADTSTSTAYLELSSLRSEDTAVYYCARDGYSRYYFDYWGQGTLVTVSS

SEQ ID NO: 112 - protein, 112 aa, synthetic construct; identical to SEQ ID NO: 110

SEQ ID NO: 113 - protein, 14 aa, synthetic construct
WVRKAPGKGLEWVG

SEQ ID NO: 114 - protein, 15 aa, synthetic construct
WYQEKPGKPPKFLIY

SEQ ID NO: 115 - protein, 14 aa, synthetic construct
WVREAPGQGLEWIG

SEQ ID NO: 116 - protein, 15 aa, synthetic construct
WYQKKPGQPPKLLIY

SEQ ID NO: 117 - protein, 449 aa, synthetic construct
EVQLLESGGGLVQPGGSLRLSCAASGIDLSTYAMGWVRKAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 218 aa, synthetic construct
DIQVTQSPSSLPASVGDRVTITCQASENIANALAWYQEKPGKPPKFLIYGASNLASGVPSRFSGSGSGTDFTFTISSLQPEDIATYYCQQAYYGNSFVEGTFGGGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 676 aa, synthetic construct
EVQLLESGGGLVQPGGSLRLSCAASGIDLSTYAMGWVRKAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTEVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 120 - protein, 219 aa, synthetic construct
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 676 aa, synthetic construct
EVQLLESGGGLVQPGGSLRLSCAASGIDLSTYAMGWVRKAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTEVQLVQSGAEVKKPGASVKVSCKASGFTFTSYYIHWVREAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 122 - protein, 28 aa, synthetic construct
  MOD_RES 1: note = Pyroglutamyl-aspartate
  MOD_RES 28: note = Lysine-biotin-amide
DGNEEMGSITQTPYQVSISGTTVILTKK